(19) United States
(12) Patent Application Publication
(10) Pub. No.: US 2023/0041563 A1
(43) Pub. Date: Feb. 9, 2023

(54) N-(1,3,4-OXADIAZOL-2-YL)ARYLCARBOXAMIDES OR SALTS THEREOF, PREPARATION METHODS, HERBICIDAL COMPOSITIONS AND USES THEREOF

(71) Applicant: QINGDAO KINGAGROOT CHEMICAL COMPOUND CO., LTD., QINGDAO, SHANDONG (CN)

(72) Inventors: Lei LIAN, QINGDAO, SHANDONG (CN); Rongbao HUA, QINGDAO, SHANDONG (CN); Xuegang PENG, QINGDAO, SHANDONG (CN); De ZHAO, QINGDAO, SHANDONG (CN); Qi CUI, QINGDAO, SHANDONG (CN)

(21) Appl. No.: 17/298,303

(22) PCT Filed: Nov. 27, 2019
(86) PCT No.: PCT/CN2019/121224

§ 371 Date: May 28, 2021

(30) Foreign Application Priority Data
Nov. 30, 2018 (CN) 201811455280.X
Oct. 23, 2019 (CN) 201911014193.5

(51) Int. Cl. C07D 271/113; C07D 413/12; A01P 13/00; A01N 43/84; A01N 43/82; A01N 25/32; C07D 417/12; C07F 9/653; C07F 7/08; A01N 47/36; A01N 43/90; A01N 43/54; A01N 47/02; A01N 37/38; A01N 43/76; A01N 43/42; A01N 43/40; A01N 37/40; A01N 33/18; A01N 47/12; A01N 47/16; A01N 35/06; A01N 43/36; A01N 37/26; A01N 43/653; A01N 43/78; A01N 57/12; A01N 43/713; A01N 43/70; A01N 43/88; A01N 43/707; A01N 37/34; A01N 43/80; A01N 43/86; A01N 43/20; A01N 57/28; A01N 55/00

(52) U.S. Cl. CPC C07D 271/113; A01N 55/00; A01P 13/00; A01N 43/84; A01N 43/82; A01N 25/32; C07D 417/12; C07F 9/65318; C07F 7/0812; A01N 47/36; A01N 43/90; A01N 43/54; A01N 47/02; A01N 37/38; A01N 43/76; A01N 43/42; A01N 43/40; A01N 37/40; A01N 33/18; A01N 47/12; A01N 47/16; A01N 35/06; A01N 43/36; A01N 37/26; A01N 43/653; A01N 43/78; A01N 57/12; A01N 43/713; A01N 43/70; A01N 43/88; A01N 43/707; A01N 37/34; A01N 43/80; A01N 43/86; A01N 43/20; A01N 57/28; C07D 413/12

(57) ABSTRACT

The invention belongs to the technical field of agricultural chemicals, and particularly relates to an N-(1,3,4-oxadiazol-2-yl)arylcarboxamide compound or a salt thereof, a preparation method, a herbicidal composition and a use thereof. The compound is as shown in the following Formula I:

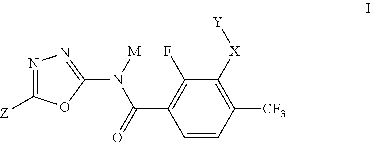

wherein X represents O, S, SO, SO₂ or NR₁; Y represents halogen, cyano, cyanoalkyl, carboxyl, nitro, etc.; or —X—Y represents unsubstituted or substituted five- or six-membered heterocyclyl or heteroaryl; Z represents hydrogen, halogen, cyano, OR₄, -alkyl-OR₄, —O-alkyl-N(R₅)₂, etc., M represents hydrogen, OR₆, SR₆, COR₆, COOR₆, CON(R₇)₂, etc. The compound has advantages of a low dosage for use, excellent herbicidal activity, and higher crop safety, especially good selectivity for key crops such as rice.

### TECHNICAL FIELD

The invention belongs to the technical field of agricultural chemicals, and particularly relates to an N-(1,3,4-oxadiazol-2-yl)arylcarboxamide or a salt thereof, a preparation method, a herbicidal composition and a use thereof.

### BACKGROUND ART

The prevention and control of weeds is a crucial step in the realization of high-efficiency agricultural processes. On the market, there are a wide variety of herbicides, for example, WO2014086746A1, WO2016146561A1, WO2014086734A1, WO2013017559A1, WO2017144402A1, WO2012126932A1, WO2012028579A1, etc., disclose certain arylcarboxamides and their use as herbicides. However, due to the continuous expansion of the market, the resistance of weeds, the service life of chemicals and the economical efficiency of chemicals, as well as the increasing emphasis on the environment, especially the serious occurrence of resistance to mainstream weed herbicides (e.g., herbicides with the inhibition mechanism of ALS such as Penoxsulam, Bispyribac-sodium, Pyribenzoxim, and Nicosulfuron, herbicides with ACCe-based inhibition mechanism such as Cyhalofop-butyl, Clethodim, Quizalofop-p-ethyl, and Metamifop, as well as Glyphosate and the like) in the market, crops such as wheat, corn, rice, cotton and soybean encounter a serious challenge and lack effective chemicals to control weeds with resistance. This requires scientists to continuously research and develop new high-efficiency, safe, economic herbicides with different action modes.

In addition, many chiral herbicides have been developed in the market, such as aryloxyphenoxypropionic acid herbicides including Quizalofop-p-ethyl, Fluazifop-p-butyl, Cyhalofop-butyl, Metamifop, and Fenoxaprop-p-ethyl, aryloxypropionic acid herbicides including MCPA propionic acid and 2,4-D propionic acid, chloroamide herbicides including S-metolachlor), and the development of these chiral herbicides has greatly reduced the use of ineffective isomers, and provided better protection to environmental safety. However, there is not a sulfur-containing chiral herbicide that has been commercialized. The present application surprisingly finds an arylcarboxamide compound, a sulfur-containing chiral herbicide, which will has great commercial values.

### CONTENTS OF THE INVENTION

The present invention provides an N-(1,3,4-oxadiazol-2-yl)arylcarboxamide compound or a salt thereof, a preparation method, a herbicidal composition and a use thereof, wherein the compound has advantages of a low dosage for use, excellent herbicidal activity, higher crop safety, and especially good selectivity for key crops such as rice.

The technical solution adopted by the present invention is as follows:

The present invention provides an N-(1,3,4-oxadiazol-2-yl)arylcarboxamide compound or a salt thereof, which has the following structural formula:

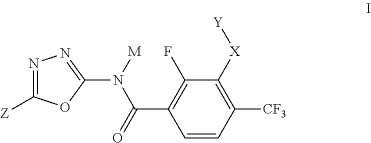

wherein, X represents O, S, SO, SO₂ or NR₁;

Y represents halogen, cyano, cyanoalkyl, carboxy, nitro, N(R₂)₂, -alkyl-N(R₂)₂, CON(R₂)₂, -alkyl-CON(R₂)₂, -alkyl-N(R₂)₃⁺I⁻, hydroxyalkyl, alkyl substituted by amino and carboxy, OR₃, SR₃, -alkyl-SOR₃, -alkyl-OR₃, -alkyl-SR₃, COR₃, COOR₃, -alkyl-COR₄, -alkyl-COOR₄, -alkyl-OCOR₃, Si(R₃)₃, -alkyl-O—Si(R₃)₃, -alkyl-O—N═C(R₃)₂, alkyl, haloalkyl, unsubstituted or substituted alkenyl, unsubstituted or substituted alkynyl, unsubstituted or substituted cycloalkyl, unsubstituted or substituted cycloalkylalkyl, unsubstituted or substituted heterocyclyl, unsubstituted or substituted aryl, unsubstituted or substituted heteroaryl, unsubstituted or substituted heterocyclylalkyl, unsubstituted or substituted arylalkyl, or unsubstituted or substituted heteroarylalkyl, wherein the substituted alkenyl or the substituted alkynyl is alkenyl or alkynyl substituted by at least one group selected from the group consisting of halogen, cyano, cycloalkyl, alkylcarbonyl, alkoxycarbonyl, alkoxy, alkylthio, alkylsulfinyl, alkylsulfonyl, and trialkylsilyl;

or, —X—Y represents an unsubstituted or substituted five- or six-membered heterocyclyl or heteroaryl, wherein the heterocyclyl or heteroaryl contains, besides C atom and N atom at 1-position, 0 to 3 of the following atoms or groups as a component of the ring: O, N, NR₁, SO₂, C═O;

Z represents hydrogen, halogen, cyano, OR₄, -alkyl-OR₄, —O-alkyl-N(R₅)₂, -alkyl-O-alkyl-N(R₅)₂, N(R₅)₂, -alkyl-(C═O)ₘ—N(R₅)₂, —NH-alkyl-N(R₅)₂, halogen-free or halogen-containing alkyl, halogen-free or halogen-containing alkenyl, halogen-free or halogen-containing alkynyl, halogen-free or halogen-containing alkoxycarbonyl, halogen-free or halogen-containing alkoxycarbonylalkyl, halogen-free or halogen-containing alkylcarbonyl, halogen-free or halogen-containing alkylcarbonylalkyl, halogen-free or halogen-containing alkylcarbonyloxyalkyl, halogen-free or halogen-containing alkylthio, halogen-free or halogen-containing alkylsulfinyl, halogen-free or halogen-containing alkyl sulfonyl, unsubstituted or substituted cycloalkyl, unsubstituted or substituted heterocyclyl, unsubstituted or substituted aryl, unsubstituted or substituted heteroaryl, unsubstituted or substituted heterocyclylalkyl, unsubstituted or substituted arylalkyl, unsubstituted or substituted heteroarylalkyl, unsubstituted or substituted heterocyclylcarbonylalkyl, unsubstituted or substituted arylcarbonylalkyl, unsubstituted or substituted heteroarylcarbonylalkyl, unsubstituted or substituted heterocyclylcarbonyloxyalkyl, unsubstituted or substituted arylcarbonyloxyalkyl, or unsubstituted or substituted heteroarylcarbonyloxyalkyl;

M represents hydrogen, OR₆, SR₆, COR₆, COOR₆, OCOR₆, CON(R₇)₂, N(R₇)₂, NR₈COOR₆, NR₈CON(R₇)₂, -alkyl-R, halogen-free or halogen-containing alkyl, halogen-free or halogen-containing alkenyl, halogen-free or halogen-containing alkynyl, halogen-free or halogen-containing cycloalkyl, unsubstituted or substituted arylalkyl, or unsubstituted or substituted heteroarylalkyl;

R represents halogen-free or halogen-containing alkenyl, halogen-free or halogen-containing alkynyl, halogen-free or halogen-containing cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)—N(R₁₃)₂;

R₄, R₆, and R₁₁ each independently represent hydrogen, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing cycloalkenyl, halogen-free or halogen-containing alkoxyalkyl, unsubstituted or substituted heterocyclyl, unsubstituted or substituted heterocyclylalkyl, unsubstituted or substituted heterocyclyloxyalkyl, unsubstituted or substituted aryl, unsubstituted or substituted arylalkyl, unsubstituted or substituted aryloxyalkyl, unsubstituted or substituted heteroaryl, unsubstituted or substituted heteroarylalkyl, or unsubstituted or substituted heteroaryloxyalkyl;

R₃ and R₁₂ each independently represent halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing cycloalkenyl, halogen-free or halogen-containing alkoxyalkyl, unsubstituted or substituted heterocyclyl, unsubstituted or substituted heterocyclylalkyl, unsubstituted or substituted aryl, unsubstituted or substituted arylalkyl, unsubstituted or substituted heteroaryl, or unsubstituted or substituted heteroarylalkyl;

R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(P)ᵣR₂₂, alkyl, haloalkyl, alkenyl, haloalkenyl, alkynyl, haloalkynyl, cycloalkyl, cycloalkenyl, halocycloalkyl, alkoxyalkyl, cycloalkylalkyl, aryl, arylalkyl, aryloxy, aryloxyalkyl, arylalkyloxy, arylcarbonyl, aryl sulfonyl, heteroaryl, heteroarylalkyl, heteroaryloxy, heteroaryloxyalkyl, heteroarylalkyloxy, heteroarylcarbonyl, heteroarylsulfonyl, heterocyclyl, heterocyclylalkyl, heterocyclyloxy, heterocyclyloxyalkyl, heterocyclylalkyloxy, heterocyclylcarbonyl, heterocyclylsulfonyl, aryl-NR₂₁-alkyl, heteroaryl-NR₂₁-alkyl, heterocyclyl-NR₂₁-alkyl, wherein the last 35 groups as mentioned are each substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and alkoxyalkoxycarbonyl;

R₂₁ independently represents hydrogen, alkyl, alkenyl, alkynyl, cycloalkyl, or cycloalkylalkyl;

R₂₂ independently represents alkyl, alkenyl, alkynyl, cycloalkyl, or cycloalkylalkyl;

m represents 0 or 1;

n and r each independently represent 0, 1 or 2; and

when X represents S or SO, Y is not methyl.

Preferably, X represents O, S, SO, SO₂ or NR₁;

Y represents halogen, cyano, cyano-(C₁-C₈)alkyl, carboxy, nitro, N(R₂)₂, —(C₁-C₈)alkyl-N(R₂)₂, CON(R₂)₂, —(C₁-C₈)alkyl-CON(R₂)₂, —(C₁-C₈)alkyl-N(R₂)₃⁺I⁻, hydroxy(C₁-C₈)alkyl, (C₁-C₈)alkyl substituted by amino and carboxy, OR₃, SR₃, —(C₁-C₈)alkyl-SOR₃, —(C₁-C₈)alkyl-OR₃, —(C₁-C₈)alkyl-SR₃, COR₃, COOR₃, —(C₁-C₈)alkyl-COR₄, —(C₁-C₈)alkyl-COOR₄, —(C₁-C₈)alkyl-OCOR₃, Si(R₃)₃, —(C₁-C₈)alkyl-O—Si(R₃)₃, —(C₁-C₈)alkyl-O—N═C(R₃)₂, C₁-C₈alkyl, haloC₁-C₈alkyl; unsubstituted or substituted C₂-C₈ alkenyl, unsubstituted or substituted C₂-C₈ alkynyl, unsubstituted or substituted C₃-C₈ cycloalkyl, unsubstituted or substituted C₃-C₈cycloalkyl C₁-C₈alkyl, unsubstituted or substituted heterocyclyl, unsubstituted or substituted aryl, unsubstituted or substituted heteroaryl, unsubstituted or substituted heterocyclyl C₁-C₈alkyl, unsubstituted or substituted aryl C₁-C₈alkyl, unsubstituted or substituted heteroaryl C₁-C₈alkyl, wherein the substituted C₂-C₈ alkenyl or the substituted C₂-C₈ alkynyl is C₂-C₈ alkenyl or C₂-C₈ alkynyl substituted by at least one group selected from the group consisting of halogen, cyano, C₃-C₈ cycloalkyl, C₁-C₈ alkylcarbonyl, C₁-C₈ alkoxycarbonyl, C₁-C₈ alkoxy, C₁-C₈ alkylthio, C₁-C₈ alkylsulfinyl, C₁-C₈ alkylsulfonyl and tri(C₁-C₈)alkylsilyl, the substituted C₃-C₈ cycloalkyl or the substituted (C₃-C₈)cycloalkyl(C₁-C₈)alkyl is C₃-C₈ cycloalkyl or (C₃-C₈)cycloalkyl(C₁-C₈)alkyl substituted by at least one group selected from the group consisting of C₁-C₈ alkyl, halogen and phenyl;

or, —X—Y represents

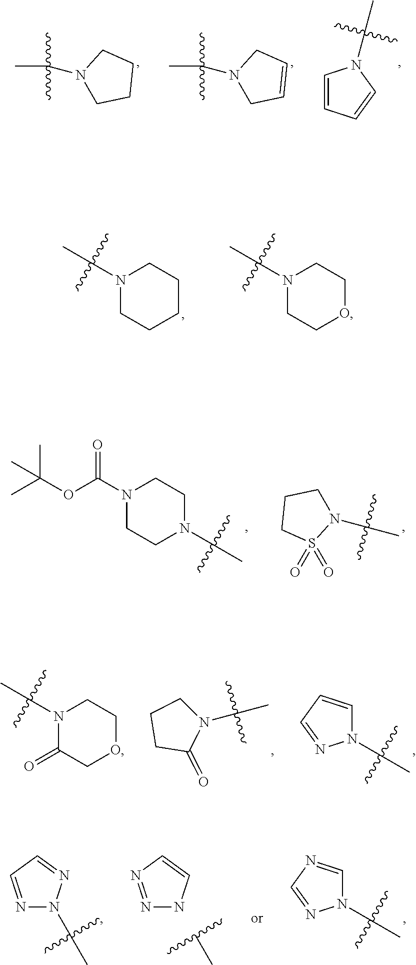

which is unsubstituted or substituted by at least one group selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing alkoxy, halogen-free or halogen-containing alkylthio, halogen-free or halogen-containing alkoxyalkyl, halogen-free or halogen-containing alkylthioalkyl, halogen-free or halogen-containing alkylcarbonyl, halogen-free or halogen-containing alkoxycarbonyl, halogen-free or halogen-containing alkylsulfinyl, halogen-free or halogen-containing alkylsulfonyl, halogen-free or halogen-containing alkylamino, halogen-free or halogen-containing dialkylamino, halogen-free or halogen-containing alkylacyloxy, wherein the substituted phenyl, the substituted benzyl or the substituted phenoxy is phenyl, benzyl or phenoxy substituted by at least one group selected from the group consisting of halogen, alkyl and alkoxy;

Z represents hydrogen, halogen, cyano, OR₄, —(C₁-C₈)alkyl-OR₄, —O—(C₁-C₈)alkyl-N(R₅)₂, —(C₁-C₈)alkyl-O—(C₁-C₈)alkyl-N(R₅)₂, N(R₅)₂, —(C₁-C₈)alkyl-(C═O)ₘN(R₅)₂, —NH—(C₁-C₈)alkyl-N(R₅)₂, halogen-free or halogen-containing C₁-C₈ alkyl, halogen-free or halogen-containing C₂-C₈ alkenyl, halogen-free or halogen-containing C₂-C₈ alkynyl, halogen-free or halogen-containing C₁-C₈ alkoxy carbonyl, halogen-free or halogen-containing C₁-C₈alkoxycarbonyl C₁-C₈alkyl, halogen-free or halogen-containing C₁-C₈alkyl carbonyl, halogen-free or halogen-containing C₁-C₈alkyl carbonyl C₁-C₈alkyl, halogen-free or halogen-containing C₁-C₈alkyl carbonyloxy C₁-C₈alkyl, halogen-free or halogen-containing C₁-C₈ alkylthio, halogen-free or halogen-containing C₁-C₈alkylsulfinyl, halogen-free or halogen-containing C₁-C₈alkylsulfonyl, unsubstituted or substituted C₃-C₈ cycloalkyl, unsubstituted or substituted heterocyclyl, unsubstituted or substituted aryl, unsubstituted or substituted heteroaryl, unsubstituted or substituted heterocyclyl C₁-C₈alkyl, unsubstituted or substituted aryl C₁-C₈alkyl, unsubstituted or substituted heteroaryl C₁-C₈alkyl, unsubstituted or substituted heterocyclylcarbonyl C₁-C₈ alkyl, unsubstituted or substituted arylcarbonyl C₁-C₈ alkyl, unsubstituted or substituted heteroarylcarbonyl C₁-C₈alkyl, unsubstituted or substituted heterocyclylcarbonyloxy C₁-C₈ alkyl, unsubstituted or substituted arylcarbonyloxy C₁-C₈ alkyl, or unsubstituted or substituted heteroarylcarbonyloxy C₁-C₈alkyl, wherein the substituted C₃-C₈ cycloalkyl is C₃-C₈ cycloalkyl substituted by at least one group selected from the group consisting of C₁-C₈ alkyl, halogen and phenyl;

M represents hydrogen, OR₆, SR₆, COR₆, COOR₆, OCOR₆, CON(R₇)₂, N(R₇)₂, NR₈COOR₆, NR₈CON(R₇)₂, —(C₁-C₈)alkyl-R, halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, unsubstituted or substituted aryl C₁-C₈alkyl, or unsubstituted or substituted heteroaryl C₁-C₈ alkyl;

R represents halogen-free or halogen-containing C₂-C₈alkenyl, halogen-free or halogen-containing C₂-C₈alkynyl, halogen-free or halogen-containing C₃-C₈cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)-MR₁₃)₂;

R₄, R₆ and Ru each independently represent hydrogen, halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, halogen-free or halogen-containing C₃-C₈cycloalkyl C₁-C₈alkyl, halogen-free or halogen-containing C₃-C₈cycloalkenyl, halogen-free or halogen-containing C₁-C₈alkoxy C₁-C₈alkyl, unsubstituted or substituted heterocyclyl, unsubstituted or substituted heterocyclyl C₁-C₈alkyl, unsubstituted or substituted heterocyclyloxy C₁-C₈alkyl, unsubstituted or substituted aryl, unsubstituted or substituted aryl C₁-C₈alkyl, unsubstituted or substituted aryloxy C₁-C₈alkyl, unsubstituted or substituted heteroaryl, unsubstituted or substituted heteroaryl C₁-C₈alkyl, unsubstituted or substituted heteroaryloxy C₁-C₈alkyl;

R₃ and R₁₂ each independently represent halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, halogen-free or halogen-containing C₃-C₈cycloalkyl C₁-C₈alkyl, halogen-free or halogen-containing C₃-C₈cycloalkenyl, halogen-free or halogen-containing C₁-C₈alkoxy C₁-C₈alkyl, unsubstituted or substituted heterocyclyl, unsubstituted or substituted heterocyclyl C₁-C₈alkyl, unsubstituted or substituted aryl, unsubstituted or substituted aryl C₁-C₈alkyl, unsubstituted or substituted heteroaryl, unsubstituted or substituted heteroaryl C₁-C₈alkyl;

R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, C₁-C₈alkyl, haloC₁-C₈alkyl, C₂-C₈alkenyl, haloC₂-Csalkenyl, C₂-C₈alkynyl, haloC₂-C₈alkynyl, C₃-C₈cycloalkyl, C₃-C₈cycloalkenyl, haloC₃-C₈cycloalkyl, C₁-C₈alkoxy C₁-C₈alkyl, C₃-C₈cycloalkyl C₁-C₈alkyl, aryl, aryl C₁-C₈alkyl, aryloxy, aryloxy C₁-C₈alkyl, aryl C₁-C₈alkyloxy, arylcarbonyl, arylsulfonyl, heteroaryl, heteroaryl C₁-C₈alkyl, heteroaryloxy, heteroaryloxy C₁-C₈alkyl, heteroaryl C₁-C₈alkyloxy, heteroarylcarbonyl, heteroarylsulfonyl, heterocyclyl, heterocyclyl C₁-C₈alkyl, heterocyclyloxy, heterocyclyloxy C₁-C₈alkyl, heterocyclyl C₁-C₈alkyloxy, heterocyclylcarbonyl, heterocyclylsulfonyl, aryl-NR₂₁—(C₁-C₈)alkyl, heteroaryl-NR₂₁—(C₁-C₈)alkyl, or heterocyclyl-NR₂₁—(C₁-C₈)alkyl, wherein the last 35 groups as mentioned each are independently substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₈alkoxy C₁-C₈alkoxycarbonyl;

R₂₁ independently represents hydrogen, C₁-C₈alkyl, C₂-C₈alkenyl, C₂-C₈alkynyl, C₃-C₈cycloalkyl, or C₃-C₈cycloalkyl C₁-C₈alkyl;

R₂₂ independently represents C₁-C₈alkyl, C₂-C₈alkenyl, C₂-C₈alkynyl, C₃-C₈cycloalkyl, or C₃-C₈cycloalkyl C₁-C₈alkyl;

m represents 0 or 1;

n and r each independently represent 0, 1 or 2;

wherein, the “heterocyclyl” refers to

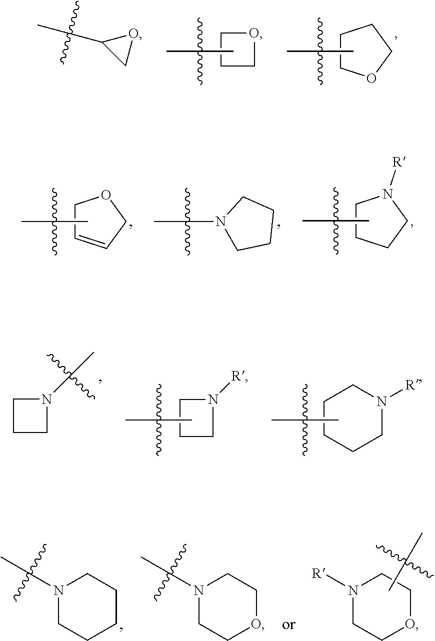

which has 0, 1 or 2 oxo groups; the “aryl” refers to phenyl or naphthyl, the “heteroaryl” refers to

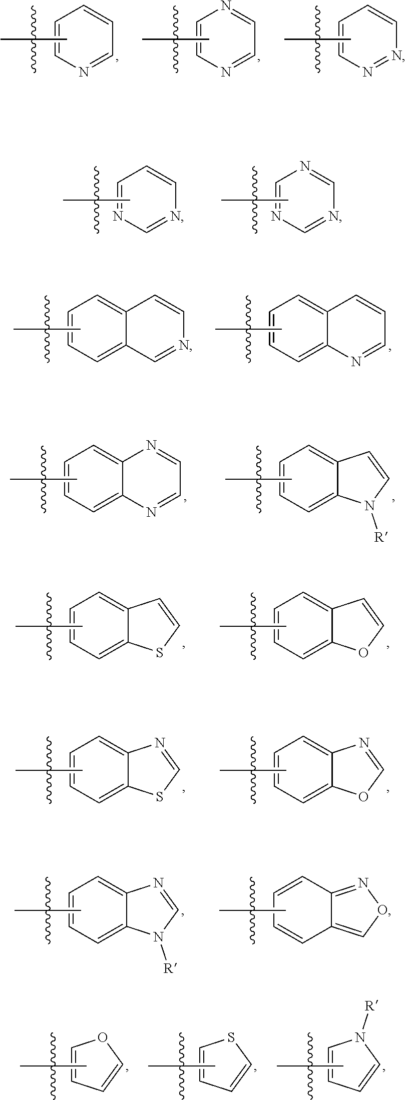

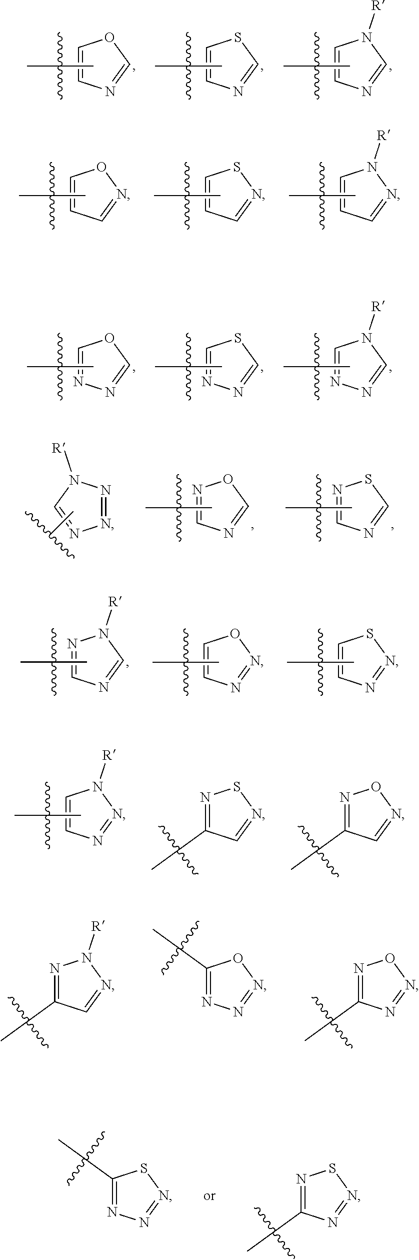

wherein the heterocyclyl, the aryl or the heteroaryl is unsubstituted or substituted by at least one group selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing OR″, halogen-free or halogen-containing SR″, halogen-free or halogen-containing -alkyl-OR″, halogen-free or halogen-containing -alkyl-SR″, halogen-free or halogen-containing COR″, halogen-free or halogen-containing COOR″, halogen-free or halogen-containing COSR″, halogen-free or halogen-containing SOR″, halogen-free or halogen-containing SO₂R″, halogen-free or halogen-containing OCOR″, halogen-free or halogen-containing SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted with at least one group selected from the group consisting of halogen, alkyl and alkoxy, the substituted amino or the substituted aminocarbonyl is amino or aminocarbonyl substituted by one or two groups selected from the group consisting of hydrogen, alkyl, alkenyl, alkynyl, cycloalkyl, cycloalkylalkyl, COR″, SO₂R″, and OR″;

R′ independently represents hydrogen, nitro, hydroxy, amino, halogen-containing or halogen-free alkyl, halogen-containing or halogen-free alkenyl, halogen-containing or halogen-free alkynyl, halogen-containing or halogen-free cycloalkyl, halogen-containing or halogen-free cycloalkenyl, halogen-containing or halogen-free cycloalkylalkyl, halogen-containing or halogen-free alkoxy, halogen-containing or halogen-free alkenyloxy, halogen-containing or halogen-free alkynyloxy, halogen-containing or halogen-free cycloalkyloxy, halogen-containing or halogen-free alkoxyalkyl, halogen-containing or halogen-free alkoxycarbonyl, halogen-containing or halogen-free alkylthiocarbonyl, halogen-containing or halogen-free alkylsulfonyl, halogen-containing or halogen-free alkylsulfonylalkyl, halogen-containing or halogen-free alkylcarbonyl, halogen-containing or halogen-free alkylcarbonylalkyl, halogen-containing or halogen-free alkylacyloxy, halogen-containing or halogen-free alkylamino, halogen-containing or halogen-free alkylaminocarbonyl, halogen-containing or halogen-free alkoxyaminocarbonyl, halogen-containing or halogen-free alkoxycarbonylalkyl, halogen-containing or halogen-free alkylaminocarbonylalkyl, halogen-containing or halogen-free trialkylsilyl, or halogen-containing or halogen-free dialkylphosphonyl;

R″ independently represents hydrogen, halogen-containing or halogen-free alkyl, halogen-containing or halogen-free alkenyl, halogen-containing or halogen-free alkynyl, halogen-containing or halogen-free cycloalkyl, or halogen-containing or halogen-free cycloalkylalkyl.

More preferably, X represents O, S, SO, SO₂ or NR₁;

Y represents halogen, cyano, cyano C₁-C₆alkyl, carboxy, nitro, N(R₂)₂, —(C₁-C₆)alkyl-N(R₂)₂, CON(R₂)₂, —(C₁-C₆)alkyl-CON(R₂)₂, —(C₁-C₆)alkyl-N(R₂)₃⁺I⁻, hydroxy C₁-C₆alkyl, C₁-C₆alkyl substituted by amino and carboxy, OR₃, SR₃, —(C₁-C₆)alkyl-SOR₃, —(C₁-C₆)alkyl-OR₃, —(C₁-C₆)alkyl-SR₃, COR₃, COOR₃, —(C₁-C₆)alkyl-COR₄, —(C₁-C₆)alkyl-COOR₄, —(C₁-C₆)alkyl-OCOR₃, Si(R₃)₃, —(C₁-C₆)alkyl-O—Si(R₃)₃, —(C₁-C₆)alkyl-O—N═C(R₃)₂, C₁-C₆alkyl, haloC₁-C₆alkyl, unsubstituted or substituted C₂-C₆ alkenyl, unsubstituted or substituted C₂-C₆ alkynyl, unsubstituted or substituted C₃-C₆ cycloalkyl, unsubstituted or substituted C₃-C₆cycloalkyl C₁-C₆alkyl, unsubstituted or substituted heterocyclyl, unsubstituted or substituted aryl, unsubstituted or substituted heteroaryl, unsubstituted or substituted heterocyclyl C₁-C₆alkyl, unsubstituted or substituted aryl C₁-C₆alkyl, unsubstituted or substituted heteroaryl C₁-C₆alkyl, wherein the substituted C₂-C₆ alkenyl or the substituted C₂-C₆ alkynyl is C₂-C₆ alkenyl or C₂-C₆ alkynyl substituted by at least one group selected from the group consisting of halogen, cyano, C₃-C₆ cycloalkyl, C₁-C₆ alkyl carbonyl, C₁-C₆ alkoxy carbonyl, C₁-C₆ alkoxy, C₁-C₆ alkylthio, C₁-C₆ alkyl sulfinyl, C₁-C₆ alkyl sulfonyl and tri(C₁-C₆)alkylsilyl, the substituted C₃-C₆ cycloalkyl or the substituted C₃-C₆cycloalkyl C₁-C₆alkyl is C₃-C₆ cycloalkyl or C₃-C₆cycloalkyl C₁-C₆alkyl substituted by at least one group selected from the group consisting of C₁-C₆ alkyl, halogen and phenyl;

or, —X—Y represents

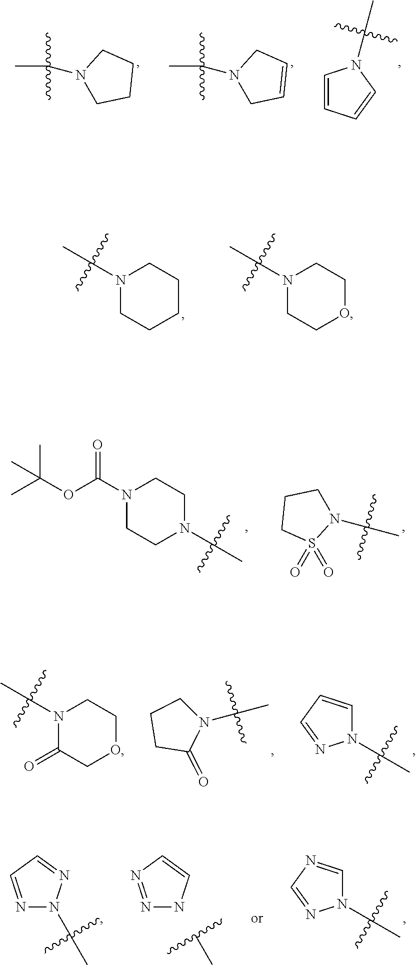

which is unsubstituted or substituted by 1, 2 or 3 groups selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-containing or halogen-free C₁-C₈ alkyl, halogen-containing or halogen-free C₂-C₈ alkenyl, halogen-containing or halogen-free C₂-C₈ alkynyl, halogen-containing or halogen-free C₃-C₈ cycloalkyl, halogen-containing or halogen-free C₃-C₈cycloalkyl C₁-C₈alkyl, halogen-containing or halogen-free C₁-C₈ alkoxy, halogen-containing or halogen-free C₁-C₈ alkylthio, halogen-containing or halogen-free C₁-C₈alkoxy C₁-C₈alkyl, halogen-containing or halogen-free C₁-C₈alkylthio C₁-C₈alkyl, halogen-containing or halogen-free C₁-C₈alkyl carbonyl, halogen-containing or halogen-free C₁-C₈alkoxy carbonyl, halogen-containing or halogen-free C₁-C₈alkyl sulfinyl, halogen-containing or halogen-free C₁-C₈alkyl sulfonyl, halogen-containing or halogen-free C₁-C₈alkyl amino, halogen-containing or halogen-free di(C₁-C₈)alkyl amino, and halogen-containing or halogen-free C₁-C₈alkyl acyloxy, wherein the substituted phenyl, the substituted benzyl or the substituted phenoxy is phenyl, benzyl or phenoxy substituted by 1, 2 or 3 groups selected from the group consisting of halogen, C₁-C₈ alkyl and C₁-C₈ alkoxy;

Z represents hydrogen, halogen, cyano, OR₄, —(C₁-C₆)alkyl-OR₄, —O—(C₁-C₆)alkyl-N(R₅)₂, —(C₁-C₆)alkyl-O—(C₁-C₆)alkyl-N(R₅)₂, N(R₅)₂, —(C₁-C₆)alkyl-(C═O)ₘ—N(R₅)₂, —NH—(C₁-C₆)alkyl-N(R₅)₂, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₁-C₆alkoxy carbonyl, halogen-free or halogen-containing C₁-C₆alkoxy carbonyl C₁-C₆alkyl, halogen-free or halogen-containing C₁-C₆alkyl carbonyl, halogen-free or halogen-containing C₁-C₆alkyl carbonyl C₁-C₆alkyl, halogen-free or halogen-containing C₁-C₆alkyl carbonyloxy C₁-C₆alkyl, halogen-free or halogen-containing C₁-C₆ alkylthio, halogen-free or halogen-containing C₁-C₆alkyl sulfinyl, halogen-free or halogen-containing C₁-C₆alkyl sulfonyl, unsubstituted or substituted C₃-C₆ cycloalkyl, unsubstituted or substituted heterocyclyl, unsubstituted or substituted aryl, unsubstituted or substituted heteroaryl, unsubstituted or substituted heterocyclyl C₁-C₆alkyl, unsubstituted or substituted aryl C₁-C₆alkyl, unsubstituted or substituted heteroaryl C₁-C₆alkyl, unsubstituted or substituted heterocyclyl carbonyl C₁-C₆alkyl, unsubstituted or substituted aryl carbonyl C₁-C₆alkyl, unsubstituted or substituted heteroaryl carbonyl C₁-C₆alkyl, unsubstituted or substituted heterocyclyl carbonyloxy C₁-C₆alkyl, unsubstituted or substituted aryl carbonyloxy C₁-C₆alkyl, or unsubstituted or substituted heteroaryl carbonyloxy C₁-C₆alkyl, wherein the substituted C₃-C₆ cycloalkyl is C₃-C₆ cycloalkyl substituted by at least one group selected from the group consisting of C₁-C₆ alkyl, halogen and phenyl;

M represents hydrogen, OR₆, SR₆, COR₆, COOR₆, OCOR₆, CON(R₇)₂, N(R₇)₂, NR₈COOR₆, NR₈CON(R₇)₂, —(C₁-C₆)alkyl-R, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, unsubstituted or substituted aryl C₁-C₆alkyl, or unsubstituted or substituted heteroaryl C₁-C₆alkyl;

R represents halogen-free or halogen-containing C₂-C₆ alkenyl, halogen-free or halogen-containing C₂-C₆ alkynyl, halogen-free or halogen-containing C₃-C₆ cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)-MR₁₃)₂;

R₄, R₆ and R₁₁ each independently represent hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆cycloalkyl C₁-C₆alkyl, halogen-free or halogen-containing C₃-C₆ cycloalkenyl, C₁-C₆alkoxy C₁-C₆alkyl, unsubstituted or substituted heterocyclyl, unsubstituted or substituted heterocyclyl C₁-C₆alkyl, unsubstituted or substituted heterocyclyloxy C₁-C₆alkyl, unsubstituted or substituted aryl, unsubstituted or substituted aryl C₁-C₆alkyl, unsubstituted or substituted aryloxy C₁-C₆alkyl, unsubstituted or substituted heteroaryl, unsubstituted or substituted heteroaryl C₁-C₆alkyl, or unsubstituted or substituted heteroaryloxy C₁-C₆alkyl;

R₃ and R₁₂ each independently represent halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆cycloalkyl C₁-C₆alkyl, halogen-free or halogen-containing C₃-C₆ cycloalkenyl, halogen-free or halogen-containing C₁-C₆alkoxy C₁-C₆alkyl, unsubstituted or substituted heterocyclyl, unsubstituted or substituted heterocyclyl C₁-C₆alkyl, unsubstituted or substituted aryl, unsubstituted or substituted aryl C₁-C₆alkyl, unsubstituted or substituted heteroaryl, unsubstituted or substituted heteroaryl C₁-C₆alkyl;

R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, C₁-C₆ alkyl, halo C₁-C₆alkyl, C₂-C₆ alkenyl, halo C₂-C₆alkenyl, C₂-C₆ alkynyl, halo C₂-C₆alkynyl, C₃-C₆ cycloalkyl, C₃-C₆ cycloalkenyl, halo C₃-C₆cycloalkyl, C₁-C₆alkoxy C₁-C₆alkyl, C₃-C₆cycloalkyl C₁-C₆alkyl, aryl, aryl C₁-C₆alkyl, aryloxy, aryloxy C₁-C₆alkyl, aryl C₁-C₆alkyloxy, arylcarbonyl, aryl sulfonyl, heteroaryl, heteroaryl C₁-C₆alkyl, heteroaryloxy, heteroaryloxy C₁-C₆alkyl, heteroaryl C₁-C₆alkyloxy, heteroarylcarbonyl, heteroarylsulfonyl, heterocyclyl, heterocyclyl C₁-C₆alkyl, heterocyclyloxy, heterocyclyloxy C₁-C₆alkyl, heterocyclyl C₁-C₆alkyloxy, heterocyclylcarbonyl, heterocyclylsulfonyl, aryl-NR₂₁—(C₁-C₆)alkyl, heteroaryl-NR₂₁—(C₁-C₆)alkyl, heterocyclyl-NR₂₁—(C₁-C₆)alkyl, wherein the last 35 groups as mentioned are each substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₆alkoxy C₁-C₆alkoxy carbonyl;

R₂₁ independently represents hydrogen, C₁-C₆ alkyl, C₂-C₆ alkenyl, C₂-C₆ alkynyl, C₃-C₆ cycloalkyl, or C₃-C₆cycloalkyl C₁-C₆alkyl;

R₂₂ independently represents C₁-C₆ alkyl, C₂-C₆ alkenyl, C₂-C₆ alkynyl, C₃-C₆ cycloalkyl, or C₃-C₆cycloalkyl C₁-C₆alkyl;

m represents 0 or 1;

n and r each independently represent 0, 1 or 2;

wherein, the “heterocyclyl” refers to

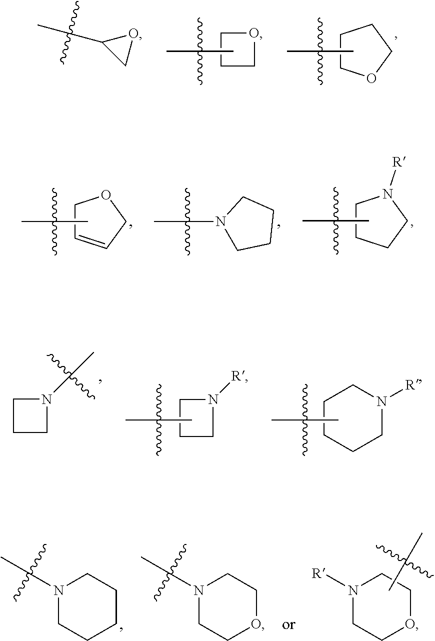

which has 0, 1 or 2 oxo groups, the “aryl” refers to phenyl or naphthyl, and “heteroaryl” refers to

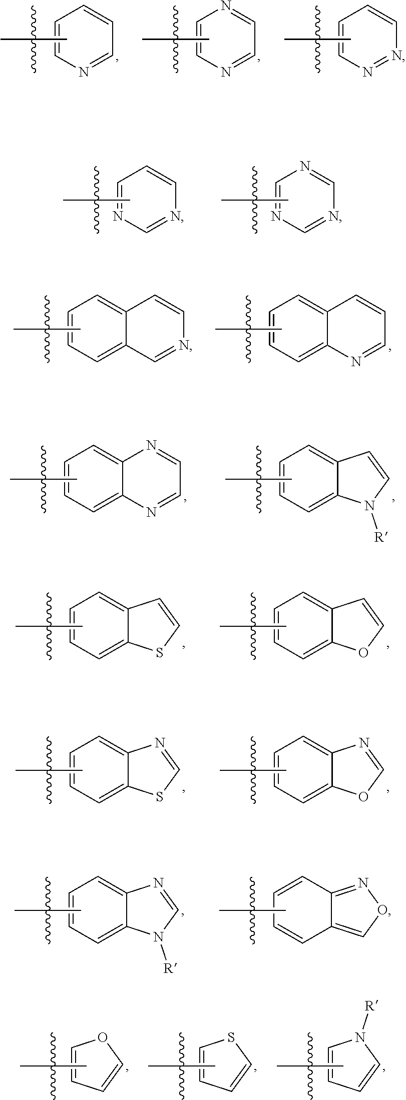

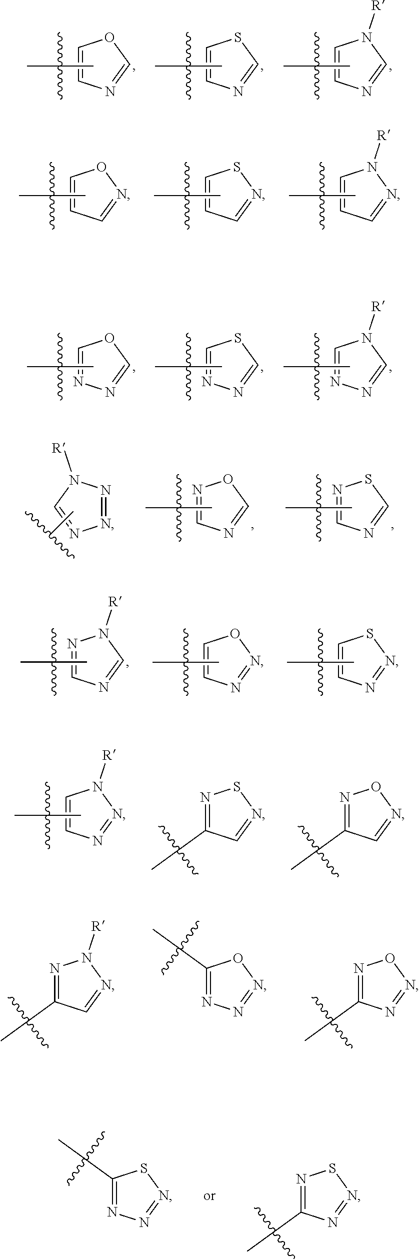

which is unsubstituted or substituted by 0, 1, 2 or 3 groups selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-containing or halogen-free C₁-C₈ alkyl, halogen-containing or halogen-free C₂-C₈ alkenyl, halogen-containing or halogen-free C₂-C₈ alkynyl, halogen-containing or halogen-free C₃-C₈ cycloalkyl, halogen-containing or halogen-free C₃-C₈ cycloalkyl C₁-C₈ alkyl, halogen-containing or halogen-free OR″, halogen-containing or halogen-free SR″, halogen-containing or halogen-free —(C₁-C₈)alkyl-OR″, halogen-containing or halogen-free —(C₁-C₈)alkyl-SR″, halogen-containing or halogen-free COR″, halogen-containing or halogen-free COOR″, halogen-containing or halogen-free COSR″, halogen-containing or halogen-free SOR″, halogen-containing or halogen-free SO₂R″, halogen-containing or halogen-free OCOR″, halogen-containing or halogen-free SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted by 1, 2 or 3 groups selected from halogen, C₁-C₈ alkyl and C₁-C₈ alkoxy, the substituted amino or the substituted aminocarbonyl is amino or aminocarbonyl substituted by one or two groups selected from hydrogen, C₁-C₈ alkyl, C₂-C₈ alkenyl, C₂-C₈ alkynyl, C₃-C₈ cycloalkyl, C₃-C₈cycloalkyl C₁-C₈alkyl, COR″, SO₂R″ and OR″;

R′ independently represents hydrogen, nitro, hydroxy, amino, fluorine-, chlorine-, bromine-free or fluorine-, chlorine-, bromine-containing C₁-C₈ alkyl, fluorine-, chlorine-, bromine-free or fluorine-, chlorine-, bromine-containing C₂-C₈ alkenyl, fluorine-, chlorine-, bromine-free or fluorine-, chlorine-, bromine-containing C₂-C₈ alkynyl, fluorine-, chlorine-, bromine-free or fluorine-, chlorine-, bromine-containing C₃-C₈ cycloalkyl, fluorine-, chlorine-, bromine-free or fluorine-, chlorine-, bromine-containing C₃-C₈ cycloalkenyl, fluorine-, chlorine-, bromine-free or fluorine-, chlorine-, bromine-containing C₃-C₈cycloalkyl C₁-C₈alkyl, fluorine-, chlorine-, bromine-free or fluorine-, chlorine-, bromine-containing C₁-C₈alkoxy, fluorine-, chlorine-, bromine-free or fluorine-, chlorine-, bromine-containing C₂-C₈ alkenyloxy, fluorine-, chlorine-, bromine-free or fluorine-, chlorine-, bromine-containing C₂-C₈ alkynyloxy, fluorine-, chlorine-, bromine-free or fluorine-, chlorine-, bromine-containing C₃-C₈ cycloalkyloxy, fluorine-, chlorine-, bromine-free or fluorine-, chlorine-, bromine-containing C₁-C₈alkoxy C₁-C₈alkyl, fluorine-, chlorine-, bromine-free or fluorine-, chlorine-, bromine-containing C₁-C₈alkoxy carbonyl, fluorine-, chlorine-, bromine-free or fluorine-, chlorine-, bromine-containing C₁-C₈alkylthio carbonyl, fluorine-, chlorine-, bromine-free or fluorine-, chlorine-, bromine-containing C₁-C₈alkylsulfonyl, fluorine-, chlorine-, bromine-free or fluorine-, chlorine-, bromine-containing C₁-C₈alkylsulfonyl C₁-C₈alkyl, fluorine-, chlorine-, bromine-free or fluorine-, chlorine-, bromine-containing C₁-C₈alkyl carbonyl, fluorine-, chlorine-, bromine-free or fluorine-, chlorine-, bromine-containing C₁-C₈alkyl carbonyl C₁-C₈alkyl, fluorine-, chlorine-, bromine-free or fluorine-, chlorine-, bromine-containing C₁-C₈alkyl acyloxy, fluorine-, chlorine-, bromine-free or fluorine-, chlorine-, bromine-containing C₁-C₈alkylamino, fluorine-, chlorine-, bromine-free or fluorine-, chlorine-, bromine-containing C₁-C₈alkylamino carbonyl, fluorine-, chlorine-, bromine-free or fluorine-, chlorine-, bromine-containing C₁-C₈alkoxyamino carbonyl, fluorine-, chlorine-, bromine-free or fluorine-, chlorine-, bromine-containing C₁-C₈alkoxy carbonyl C₁-C₈alkyl, fluorine-, chlorine-, bromine-free or fluorine-, chlorine-, bromine-containing C₁-C₈alkylamino carbonyl C₁-C₈alkyl, fluorine-, chlorine-, bromine-free or fluorine-, chlorine-, bromine-containing tri(C₁-C₈)alkylsilyl, or fluorine-, chlorine-, bromine-free or fluorine-, chlorine-, bromine-containing di(C₁-C₈)alkylphosphonyl;

R″ independently represents hydrogen, halogen-free or halogen-containing C₁-C₈ alkyl, halogen-free or halogen-containing C₂-C₈ alkenyl, halogen-free or halogen-containing C₂-C₈ alkynyl, halogen-free or halogen-containing C₃-C₈ cycloalkyl or halogen-free or halogen-containing C₃-C₈cycloalkyl C₁-C₈alkyl.

Further preferably, X represents O, S, SO, SO₂ or NR₁;

Y represents halogen, cyano, cyano C₁-C₂ alkyl, carboxy, nitro, N(R₂)₂, —(C₁-C₂)alkyl-N(R₂)₂, CON(R₂)₂, —(C₁-C₂)alkyl-CON(R₂)₂, —(C₁-C₂)alkyl-N(R₂)₃⁺I⁻, hydroxy C₁-C₂ alkyl, C₁-C₂ alkyl substituted by amino and carboxy, OR₃, SR₃, —(C₁-C₂)alkyl-SOR₃, —(C₁-C₂)alkyl-OR₃, —(C₁-C₂)alkyl-SR₃, COR₃, COOR₃, —(C₁-C₂)alkyl-COR₄, —(C₁-C₂)alkyl-COOR₄, —(C₁-C₂)alkyl-OCOR₃, Si(R₃)₃, —(C₁-C₂)alkyl-O—Si(R₃)₃, —(C₁-C₂)alkyl-O—N═C(R₃)₂, C₁-C₆ alkyl, halo C₁-C₆ alkyl; unsubstituted or substituted C₂-C₆ alkenyl, unsubstituted or substituted C₂-C₆ alkynyl, unsubstituted or substituted C₃-C₆ cycloalkyl, unsubstituted or substituted C₃-C₆ cycloalkyl C₁-C₂ alkyl, unsubstituted or substituted heterocyclyl, unsubstituted or substituted aryl, unsubstituted or substituted heteroaryl, unsubstituted or substituted heterocyclyl C₁-C₂ alkyl, unsubstituted or substituted aryl C₁-C₂ alkyl, or unsubstituted or substituted heteroaryl C₁-C₂ alkyl, wherein the substituted C₂-C₆ alkenyl or substituted C₂-C₆ alkynyl is C₂-C₆ alkenyl or C₂-C₆ alkynyl substituted by at least one group selected from halogen, cyano, C₃-C₆ cycloalkyl, C₁-C₆ alkyl carbonyl, C₁-C₆ alkoxy carbonyl, C₁-C₆ alkoxy, C₁-C₆ alkylthio, C₁-C₆ alkylsulfinyl, C₁-C₆ alkylsulfonyl, and tri(C₁-C₆)alkylsilyl, the substituted C₃-C₆ cycloalkyl or the substituted C₃-C₆ cycloalkyl C₁-C₂ alkyl is C₃-C₆ cycloalkyl or C₃-C₆ cycloalkyl C₁-C₂ alkyl substituted by at least one group selected from C₁-C₆ alkyl, halogen and phenyl;

or, —X—Y represents

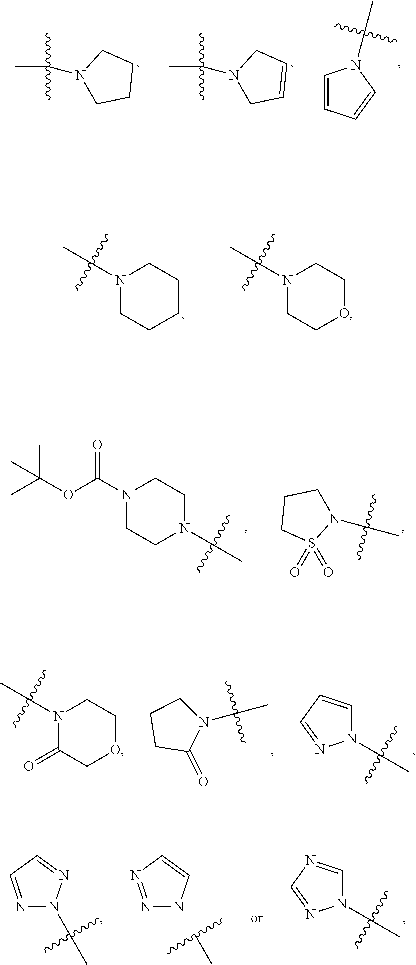

which is unsubstituted or substituted by 1, 2 or 3 groups selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₁-C₆ alkoxy, halogen-free or halogen-containing C₁-C₆ alkylthio, halogen-free or halogen-containing C₁-C₆ alkoxy C₁-C₂ alkyl, halogen-free or halogen-containing C₁-C₆ alkylthio C₁-C₂ alkyl, halogen-free or halogen-containing C₁-C₆ alkyl carbonyl, halogen-free or halogen-containing C₁-C₆ alkoxy carbonyl, halogen-free or halogen-containing C₁-C₆ alkyl sulfinyl, halogen-free or halogen-containing C₁-C₆ alkyl sulfonyl, halogen-free or halogen-containing C₁-C₆ alkyl amino, halogen-free or halogen-containing di(C₁-C₆)alkyl amino, or halogen-free or halogen-containing C₁-C₆ alkyl acyloxy, wherein the substituted phenyl, the substituted benzyl or the substituted phenoxy is phenyl, benzyl or phenoxy substituted by 1, 2 or 3 groups selected from halogen, C₁-C₆ alkyl, and C₁-C₆ alkoxy;

Z represents hydrogen, halogen, cyano, OR₄, —(C₁-C₂)alkyl-OR₄, —O—(C₁-C₂)alkyl-N(R₅)₂, —(C₁-C₂)alkyl-O—(C₁-C₂)alkyl-N(R₅)₂, N(R₅)₂, —(C₁-C₂)alkyl-(C═O)ₘ—N(R₅)₂, —NH—(C₁-C₂)alkyl-N(R₅)₂, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₁-C₆ alkoxy carbonyl, halogen-free or halogen-containing C₁-C₆ alkoxy carbonyl C₁-C₂ alkyl, halogen-free or halogen-containing C₁-C₆ alkyl carbonyl, halogen-free or halogen-containing C₁-C₆ alkyl carbonyl C₁-C₂ alkyl, halogen-free or halogen-containing C₁-C₆ alkyl carbonyloxy C₁-C₂ alkyl, halogen-free or halogen-containing C₁-C₆ alkylthio, halogen-free or halogen-containing C₁-C₆ alkyl sulfinyl, halogen-free or halogen-containing C₁-C₆ alkyl sulfonyl, unsubstituted or substituted C₃-C₆ cycloalkyl, unsubstituted or substituted heterocyclyl, unsubstituted or substituted aryl, unsubstituted or substituted heteroaryl, unsubstituted or substituted heterocyclyl C₁-C₂ alkyl, unsubstituted or substituted aryl C₁-C₂ alkyl, unsubstituted or substituted heteroaryl C₁-C₂ alkyl, unsubstituted or substituted heterocyclylcarbonyl C₁-C₂ alkyl, unsubstituted or substituted aryl carbonyl C₁-C₂ alkyl, unsubstituted or substituted heteroaryl carbonyl C₁-C₂ alkyl, unsubstituted or substituted heterocyclyl carbonyloxy C₁-C₂ alkyl, unsubstituted or substituted aryl carbonyloxy C₁-C₂ alkyl, or unsubstituted or substituted heteroaryl carbonyloxy C₁-C₂ alkyl, wherein the substituted C₃-C₆ cycloalkyl is C₃-C₆ cycloalkyl substituted by at least one group selected from C₁-C₆ alkyl, halogen and phenyl;

M represents hydrogen, OR₆, SR₆, COR₆, COOR₆, OCOR₆, CON(R₇)₂, N(R₇)₂, NR₈COOR₆, NR₈CON(R₇)₂, —(C₁-C₂)alkyl-R, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, unsubstituted or substituted aryl C₁-C₂ alkyl, or unsubstituted or substituted heteroaryl C₁-C₂ alkyl;

R represents halogen-free or halogen-containing C₂-C₆ alkenyl, halogen-free or halogen-containing C₂-C₆ alkynyl, halogen-free or halogen-containing C₃-C₆ cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, or —O—(C═O)—N(R₁₃)₂;

R₄, R₆ and R₁₁ each independently represent hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₃-C₆ cycloalkenyl, halogen-free or halogen-containing C₁-C₆ alkoxy C₁-C₂ alkyl, unsubstituted or substituted heterocyclyl, unsubstituted or substituted heterocyclyl C₁-C₂ alkyl, unsubstituted or substituted heterocyclyloxy C₁-C₂ alkyl, unsubstituted or substituted aryl, unsubstituted or substituted aryl C₁-C₂ alkyl, unsubstituted or substituted aryloxy C₁-C₂ alkyl, unsubstituted or substituted heteroaryl, unsubstituted or substituted heteroaryl C₁-C₂ alkyl, or unsubstituted or substituted heteroaryloxy C₁-C₂ alkyl;

R₃ and R₁₂ each independently represent halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₃-C₆ cycloalkenyl, halogen-free or halogen-containing C₁-C₆ alkoxy C₁-C₂ alkyl, unsubstituted or substituted heterocyclyl, unsubstituted or substituted heterocyclyl C₁-C₂ alkyl, unsubstituted or substituted aryl, unsubstituted or substituted aryl C₁-C₂ alkyl, unsubstituted or substituted heteroaryl, or unsubstituted or substituted heteroaryl C₁-C₂ alkyl;

R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, C₁-C₆ alkyl, halo C₁-C₆ alkyl, C₂-C₆ alkenyl, halo C₂-C₆ alkenyl, C₂-C₆ alkynyl, halo C₂-C₆ alkynyl, C₃-C₆ cycloalkyl, C₃-C₆ cycloalkenyl, halo C₃-C₆ cycloalkyl, C₁-C₆ alkoxy C₁-C₂ alkyl, C₃-C₆ cycloalkyl C₁-C₂ alkyl, aryl, aryl C₁-C₂ alkyl, aryloxy, aryloxy C₁-C₂ alkyl, aryl C₁-C₂ alkyloxy, arylcarbonyl, arylsulfonyl, heteroaryl, heteroaryl C₁-C₂ alkyl, heteroaryloxy, heteroaryloxy C₁-C₂ alkyl, heteroaryl C₁-C₂ alkyloxy, heteroaryl carbonyl, heteroaryl sulfonyl, heterocyclyl, heterocyclyl C₁-C₂ alkyl, heterocyclyloxy, heterocyclyloxy C₁-C₂ alkyl, heterocyclyl C₁-C₂ alkyloxy, heterocyclyl carbonyl, heterocyclyl sulfonyl, aryl-NR₂₁—(C₁-C₂)alkyl, heteroaryl-NR₂₁—(C₁-C₂)alkyl, heterocyclyl-NR₂₁—(C₁-C₂)alkyl, wherein the last 35 groups as mentioned are each substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₆ alkoxy C₁-C₂ alkoxy carbonyl;

R₂₁ independently represents hydrogen, C₁-C₆ alkyl, C₂-C₆ alkenyl, C₂-C₆ alkynyl, C₃-C₆ cycloalkyl, or C₃-C₆ cycloalkyl C₁-C₂ alkyl;

R₂₂ independently represents C₁-C₆ alkyl, C₂-C₆ alkenyl, C₂-C₆ alkynyl, C₃-C₆ cycloalkyl, or C₃-C₆cycloalkyl C₁-C₂ alkyl;

m represents 0 or 1;

n and r each independently represent 0, 1 or 2;

wherein, the “heterocyclyl” refers to

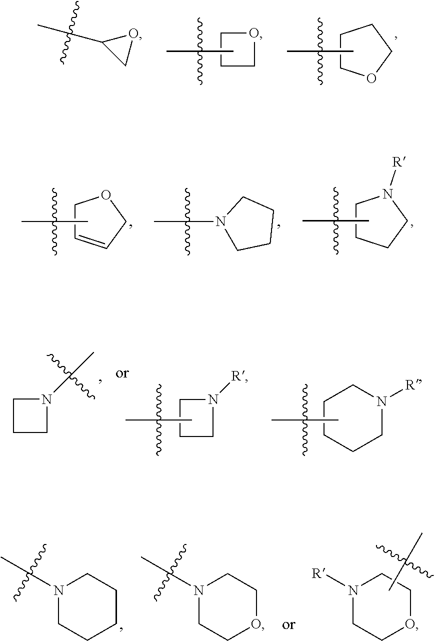

which has 0, 1 or 2 oxo groups, the “aryl” refers to phenyl or naphthyl, and the “heteroaryl” refers to

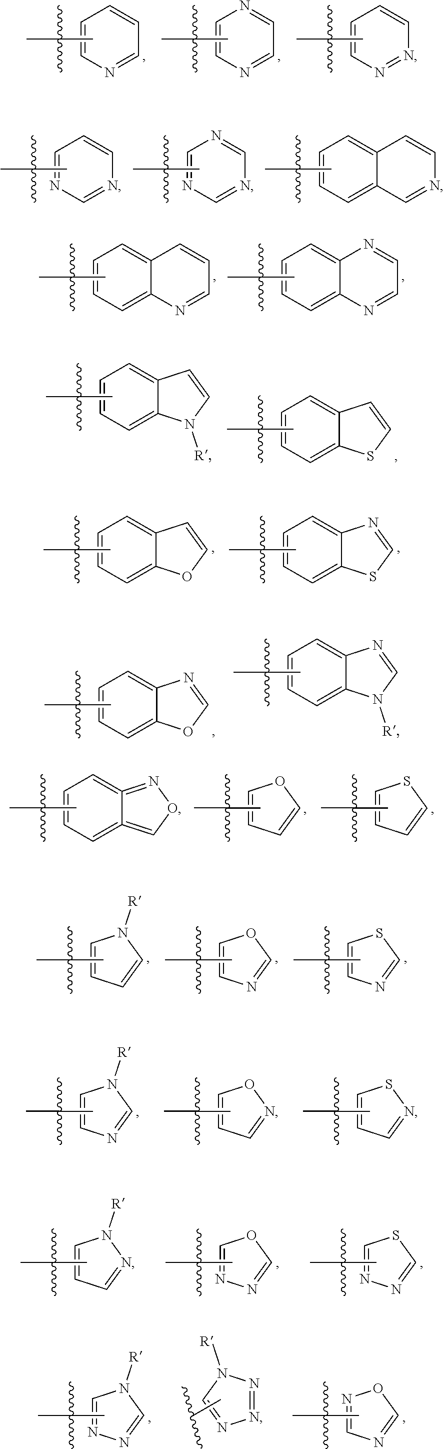

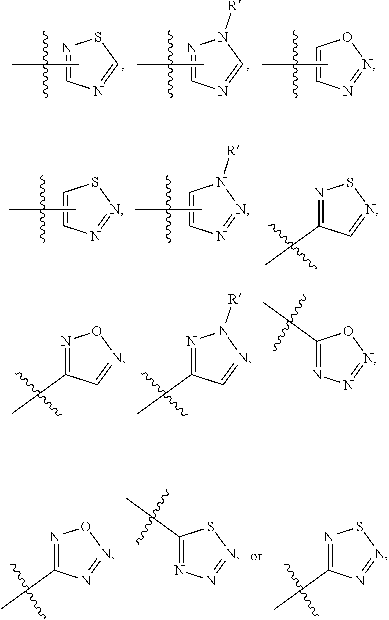

which is unsubstituted or substituted by 0, 1, 2 or 3 groups selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-containing or halogen-free C₁-C₆ alkyl, halogen-containing or halogen-free C₂-C₆ alkenyl, halogen-containing or halogen-free C₂-C₆ alkynyl, halogen-containing or halogen-free C₃-C₆ cycloalkyl, halogen-containing or halogen-free C₃-C₆ cycloalkyl C₁-C₂ alkyl, halogen-containing or halogen-free OR″, halogen-containing or halogen-free SR″, halogen-containing or halogen-free —(C₁-C₂)alkyl-OR″, halogen-containing or halogen-free —(C₁-C₂ alkyl-SR″, halogen-containing or halogen-free COR″, halogen-containing or halogen-free COOR″, halogen-containing or halogen-free COSR″, halogen-containing or halogen-free SOR″, halogen-containing or halogen-free SO₂R″, halogen-containing or halogen-free OCOR″, halogen-containing or halogen-free SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted by 1, 2 or 3 groups selected from halogen, C₁-C₆ alkyl and C₁-C₆ alkoxy, the substituted amino or the substituted aminocarbonyl is amino or aminocarbonyl substituted by one or two groups selected from hydrogen, C₁-C₆ alkyl, C₂-C₆ alkenyl, C₂-C₆ alkynyl, C₃-C₆ cycloalkyl, C₃-C₆ cycloalkyl C₁-C₂ alkyl, COR″, SO₂R″ and OR″;

R′ independently represents hydrogen, nitro, hydroxy, amino, fluorine-, chlorine-, bromine-free or fluorine-, chlorine-, bromine-containing C₁-C₆ alkyl, fluorine-, chlorine-, bromine-free or fluorine-, chlorine-, bromine-containing C₂-C₆ alkenyl, fluorine-, chlorine-, bromine-free or fluorine-, chlorine-, bromine-containing C₂-C₆ alkynyl, fluorine-, chlorine-, bromine-free or fluorine-, chlorine-, bromine-containing C₃-C₆ cycloalkyl, fluorine-, chlorine-, bromine-free or fluorine-, chlorine-, bromine-containing C₃-C₆ cycloalkenyl, fluorine-, chlorine-, bromine-free or fluorine-, chlorine-, bromine-containing C₃-C₈ cycloalkyl C₁-C₂ alkyl, fluorine-, chlorine-, bromine-free or fluorine-, chlorine-, bromine-containing C₁-C₆ alkoxy, fluorine-, chlorine-, bromine-free or fluorine-, chlorine-, bromine-containing C₂-C₆ alkenyloxy, fluorine-, chlorine-, bromine-free or fluorine-, chlorine-, bromine-containing C₂-C₆ alkynyloxy, fluorine-, chlorine-, bromine-free or fluorine-, chlorine-, bromine-containing C₃-C₆ cycloalkyloxy, fluorine-, chlorine-, bromine-free or fluorine-, chlorine-, bromine-containing C₁-C₆ alkoxy C₁-C₂ alkyl, fluorine-, chlorine-, bromine-free or fluorine-, chlorine-, bromine-containing C₁-C₆ alkoxy carbonyl, fluorine-, chlorine-, bromine-free or fluorine-, chlorine-, bromine-containing C₁-C₆ alkylthio carbonyl, fluorine-, chlorine-, bromine-free or fluorine-, chlorine-, bromine-containing C₁-C₆ alkylsulfonyl, fluorine-, chlorine-, bromine-free or fluorine-, chlorine-, bromine-containing C₁-C₆ alkylsulfonyl C₁-C₂ alkyl, fluorine-, chlorine-, bromine-free or fluorine-, chlorine-, bromine-containing C₁-C₆ alkyl carbonyl, fluorine-, chlorine-, bromine-free or fluorine-, chlorine-, bromine-containing C₁-C₆ alkyl carbonyl C₁-C₂ alkyl, fluorine-, chlorine-, bromine-free or fluorine-, chlorine-, bromine-containing C₁-C₆ alkyl acyloxy, fluorine-, chlorine-, bromine-free or fluorine-, chlorine-, bromine-containing C₁-C₆ alkylamino, fluorine-, chlorine-, bromine-free or fluorine-, chlorine-, bromine-containing C₁-C₆ alkylamino carbonyl, fluorine-, chlorine-, bromine-free or fluorine-, chlorine-, bromine-containing C₁-C₆ alkoxyamino carbonyl, fluorine-, chlorine-, bromine-free or fluorine-, chlorine-, bromine-containing C₁-C₆ alkoxy carbonyl C₁-C₂ alkyl, fluorine-, chlorine-, bromine-free or fluorine-, chlorine-, bromine-containing C₁-C₆ alkylamino carbonyl C₁-C₂ alkyl, fluorine-, chlorine-, bromine-free or fluorine-, chlorine-, bromine-containing tri(C₁-C₆)alkylsilyl, or fluorine-, chlorine-, bromine-free or fluorine-, chlorine-, bromine-containing di(C₁-C₆)alkylphosphonyl;

R″ independently represents hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl or halogen-free or halogen-containing C₃-C₆ cycloalkyl C₁-C₂ alkyl.

Further preferably, X represents S, SO or SO₂;

Y represents C₂-C₈ alkyl, halo C₁-C₈ alkyl, C₂-C₈ alkenyl, C₂-C₈ alkynyl, C₁-C₈ alkoxy C₁-C₈ alkyl, hydroxy C₁-C₈ alkyl, cyano, cyano C₁-C₈ alkyl, amino C₁-C₈ alkyl, C₁-C₈ alkyl amino C₁-C₈ alkyl, C₁-C₈ alkyl carbonyl, C₃-C₈ cycloalkyl, C₃-C₈ cycloalkyl C₁-C₈ alkyl, di(C₁-C₈)alkylamino carbonyl C₁-C₈ alkyl, C₁-C₈ alkyl carbonyl C₁-C₈ alkyl, C₁-C₈ alkoxy carbonyl C₁-C₈ alkyl, C₁-C₈ alkyl carbonyloxy C₁-C₈ alkyl, C₁-C₈ alkyl sulfinyl C₁-C₈ alkyl, tri(C₁-C₈)alkylsiloxy C₁-C₈ alkyl, heterocyclyl, heterocyclyl C₁-C₈ alkyl, phenyl, heteroaryl, heteroaryl C₁-C₈ alkyl, —(C₁-C₈)alkyl-O—N═C(R′″)₂;

wherein the heterocyclyl is

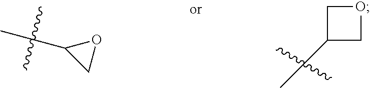

the heteroaryl is

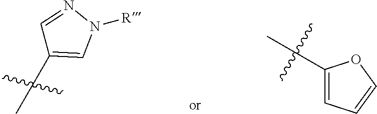

which is unsubstituted or substituted by C₁-C₈ alkyl; and R′″ independently represents C₁-C₈ alkyl;

Z represents hydrogen, or C₁-C₈ alkyl;

M represents hydrogen, or C₁-C₈ alkoxy carbonyloxy C₁-C₈ alkyl;

preferably, X represents S, SO or SO₂;

Y represents C₂-C₆ alkyl, halo C₁-C₆ alkyl, C₂-C₆ alkenyl, C₂-C₆ alkynyl, C₁-C₆ alkoxy C₁-C₆ alkyl, hydroxy C₁-C₆ alkyl, cyano, cyano C₁-C₆ alkyl, amino C₁-C₆ alkyl, C₁-C₆ alkylamino C₁-C₆ alkyl, C₁-C₆ alkyl carbonyl, C₃-C₆ cycloalkyl, C₃-C₆ cycloalkyl C₁-C₆ alkyl, di(C₁-C₆)alkylamino carbonyl C₁-C₆ alkyl, C₁-C₆ alkyl carbonyl C₁-C₆ alkyl, C₁-C₆ alkoxy carbonyl C₁-C₆ alkyl, C₁-C₆ alkyl carbonyloxy C₁-C₆ alkyl, C₁-C₆ alkyl sulfinyl C₁-C₆ alkyl, tri(C₁-C₆)alkylsiloxy C₁-C₆ alkyl, heterocyclyl, heterocyclyl C₁-C₆ alkyl, phenyl, heteroaryl, heteroaryl C₁-C₆ alkyl, —(C₁-C₆)alkyl-O—N═C(R′″)₂;

wherein, the heterocyclyl is

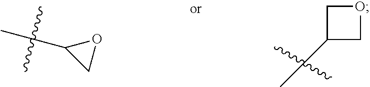

the heteroaryl is

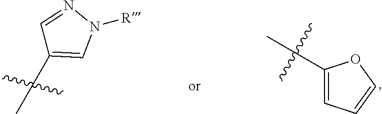

which is unsubstituted or substituted by C₁-C₆ alkyl; and R′″ independently represents C₁-C₆ alkyl;

Z represents hydrogen, or C₁-C₆alkyl;

M represents hydrogen, or C₁-C₆ alkoxy carbonyloxy C₁-C₆ alkyl.

Further preferably, the compound is preferably

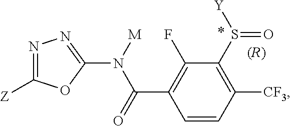

wherein Y, Z and M are as defined above.

In the definition of the compound represented by the above Formula I and all of the following structural formulas, the technical terms used, whether used alone or used in compound word, represent the following substituents: an alkyl having more than two carbon atoms may be linear or branched. For example, the alkyl in the compound word “-alkyl-N(R₂)₂” may be —CH₂—, —CH₂CH₂—, —CH(CH₃)—, and the like. The alkyl is, for example, C₁ alkyl: methyl; C₂ alkyl: ethyl; C₃ alkyl: propyl such as n-propyl or isopropyl; C₄ alkyl: butyl such as n-butyl, isobutyl, tert-butyl or 2-butyl; C₅ alkyl: pentyl such as n-pentyl; C₆ alkyl: hexyl such as n-hexyl, isohexyl and 1,3-dimethylbutyl. Similarly, the alkenyl is, for example, allyl, 1-methylprop-2-en-1-yl, 2-methylprop-2-en-1-yl, but-2-en-1-yl, butyl-3-en-1-yl, 1-methylbut-3-en-1-yl and 1-methylbut-2-en-1-yl. The alkynyl is, for example, propargyl, but-2-yn-1-yl, but-3-yn-1-yl, 1-methylbut-3-yn-1-yl. Multiple bonds may be placed at any position of each unsaturated group. The cycloalkyl is a carbocyclic saturated ring system having, for example, three to six carbon atoms, such as cyclopropyl, cyclobutyl, cyclopentyl or cyclohexyl. Similarly, the cycloalkenyl is monocycloalkenyl having, for example, three to six carbon ring members, such as cyclopropenyl, cyclobutenyl, cyclopentenyl, and cyclohexenyl, wherein double bond can be at any position. Halogen is fluorine, chlorine, bromine or iodine.

Unless otherwise specified, the “aryl” of the present invention includes, but is not limited to, phenyl, naphthyl,

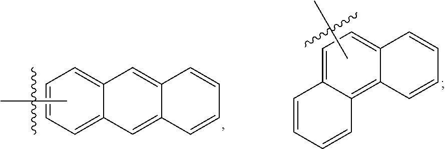

the heterocyclyl is a saturated, partially saturated or completely unsaturated cyclic group having 3 to 6 ring atoms and which may also be fused with a benzo ring, wherein 1 to 4 (for example, 1, 2, 3 or 4) heteroatoms of the ring are selected from the group consisting of oxygen, nitrogen and sulfur. For example, the heterocyclyl includes, but is not limited to,

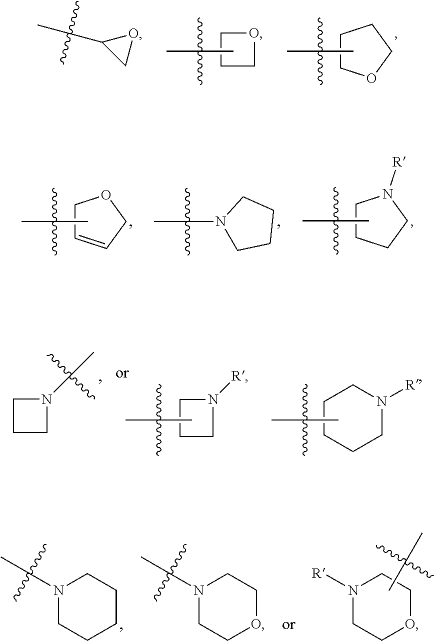

which has 0, 1 or 2 oxo groups. The heteroaryl is an aromatic cyclic group having, for example, 3 to 6 ring atoms and which may also be fused with a benzo ring, and 1 to 4 (for example, 1, 2, 3 or 4) heteroatoms of the ring are selected from the group consisting of oxygen, nitrogen and sulfur. For example, the heteroaryl group is

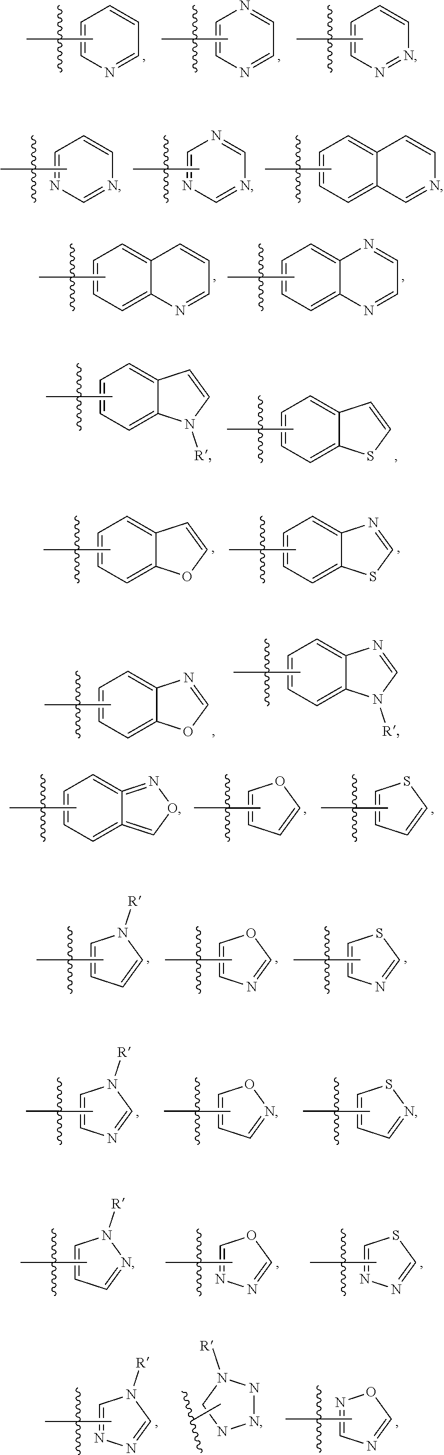

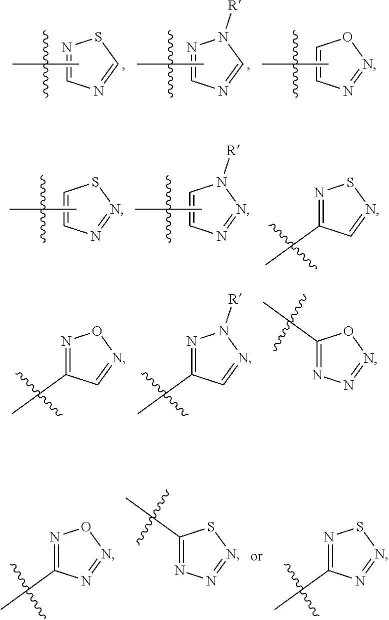

wherein R′ is as defined above.

If a group is substituted by a group, which should be understood to mean that the group is substituted by one or more groups, which are same or different groups, selected from the mentioned groups. In addition, the same or different substitution characters contained in the same or different substituents are independently selected, for example, the four R₂ groups in “N(R₂)₂” and “-alkyl-N(R₂)₂” are independently selected from the groups represented by R₂ and may be the same or different, and the three R₃ groups in “Si(R₃)₃” are independently selected from the groups represented by R₃ and may be the same or different.

In addition, unless specifically defined, the determinatives of the multiple parallel substituent (separated with “, “or” or”) of the present invention has limiting effect for each substituent, such as “alkyl, alkenyl, alkynyl or cycloalkyl, which is with or without halogen” means that “with or without halogen” has limiting effect on each group “alkyl”, “alkenyl”, “alkynyl”, “cycloalkyl”.

The salt derivative is a salt used in a common agricultural chemical, and for example, may be a metal salt, an amine salt, a sulfonium salt or a phosphonium salt, or when a molecule has a basic portion, it may be prepared into a salt such as a sulfate, a chloride, a nitrate, a phosphate. When these salts are used as herbicides for agriculture and horticulture, they are also included in the present invention. In the present invention, the “metal salt” may be, for example, an alkali metal salt, an alkaline earth metal salt, an aluminum salt or an iron salt. The “alkali metal salt” may be, for example, a sodium salt, a potassium salt or a lithium salt, preferably a sodium salt or a potassium salt. The “alkaline earth metal salt” may be, for example, a calcium salt or a magnesium salt, preferably a calcium salt. In the present invention, the “amine salt” may be, for example, an alkyl secondary amine salt, an alkyl tertiary amine salt or an alkyl quaternary ammonium salt; an alkanol primary amine salt, an alkanol secondary amine salt, an alkanol tertiary amine salt or an alkanol quaternary ammonium salt; an alkyl alkanol primary amine salt, an alkyl alkanol secondary amine salt, an alkyl alkanol tertiary amine salt or an alkyl alkanol tertiary amine salt; or an alkoxy alkanol primary amine salt, an alkoxy alkanol secondary salt, an alkoxy alkanol tertiary amine salt or an alkoxyalkanol quaternary ammonium salt, preferably wherein the alkyl, the alkanol and the alkoxy are independently saturated and independently contain 1 to 4 carbon atoms and may also be substituted by phenyl group and/or halogen. More preferably, the “amine salt” is a monoethanolamine salt, a dimethylethanolamine salt, a triethanolamine salt, a dimethylamine salt, a triethylamine salt, an isopropylamine salt, a choline salt, a diglycolamine salt,

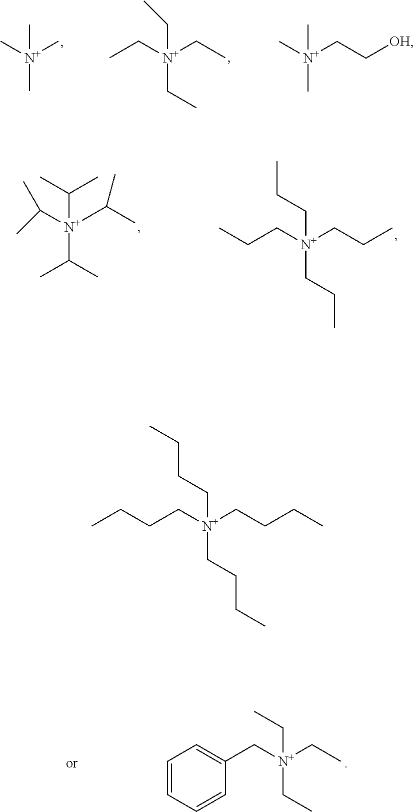

In the present invention, the “sulfonium salt” and “phosphonium salt” may, for example, be an alkyl sulfonium salt, an alkyl phosphonium salt or an alkanol phosphonium salt, preferably wherein the alkyl is independently saturated and independently contains 1 to 4 carbon atoms and may also be substituted by phenyl and/or halogen. More preferably, the “sulfonium salt” is

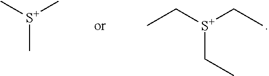

The solvates of the compounds of the present invention are also encompassed in the invention.

Depending on the property of substituents and the linkage manner thereof, the compound of Formula I may exist as a stereoisomer. For example, if a compound has one or more asymmetric carbon or sulfur atoms, it may has enantiomers and diastereomers. The stereoisomer can be obtained from the mixtures obtained in the preparation by conventional separation methods, for example by chromatographic separation. The stereoisomer may also be prepared selectively by using stereoselective reactions and using optically active starting materials and/or auxiliaries. The present invention also relates to all stereoisomers and mixtures thereof which are included in the general Formula I but are not specifically defined.

The present invention also provides a method for preparing the N-(1,3,4-oxadiazol-2-yl)arylcarboxamide compound, comprising the steps of:

a) when X represents Q in the Formula I, the method comprises the following steps:

(1) reacting a compound represented by Formula II with a compound represented by Formula III to prepare a compound represented by Formula IV;

(2) reacting the compound represented by Formula IV with carbon dioxide to obtain a compound represented by Formula V;

(3) reacting the compound represented by Formula V with a compound represented by VI in the presence of a halogenating agent to obtain a compound represented by Formula I-1;

wherein the reaction equation is as follows:

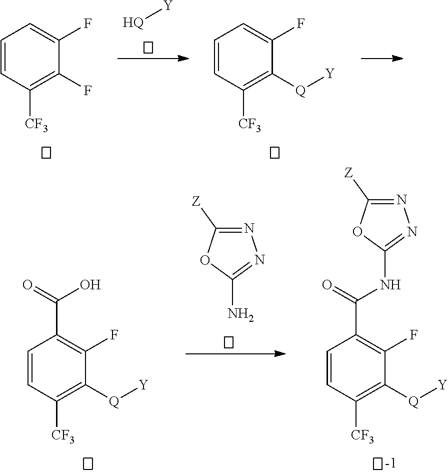

wherein, Q represents O, S or NR₁;

preferably, wherein the reaction of step (1) is carried out in the presence of a base and a solvent; more preferably, the base is one base or two bases in combination selected from the group consisting of NaOH and KOH, the solvent is one solvent or more solvents in combination selected from the group consisting of DMF, DCM, DCE, ACN, THF, and TFA; the reaction is conducted at a temperature of 0 to 25° C.;

the reaction of step (2) is carried out in the presence of a catalyst and a solvent;

preferably, the catalyst is n-butyllithium, the solvent is one solvent or more solvents in combination selected from THF, DMF, DCM, DCE, ACN, and TFA; the reaction is conducted at a temperature of −100 to −50° C.;

the reaction of step (3) is carried out in the presence of a halogenating agent, a catalyst and a solvent; preferably, the halogenating agent is SOCl₂, the catalyst is 4-dimethylaminopyridine, and the solvent is pyridine; the reaction is conducted at a temperature of 0 to 50° C.

Alternatively, b) when X represents S in the Formula I, the method comprises the following steps:

according to the above-mentioned method of the compound represented by Formula I-1, preparing the compound represented by Formula I-2, and then subjecting to a dealkylation reaction; preferably, wherein the reaction is carried out in the presence of a solvent; more preferably, the solvent is one solvent or more solvents in combination selected from DCM, DCE, ACN, DMF, THF, and TFA; the reaction is conducted at a temperature of 20 to 120° C.;

and then reacting the obtained compound represented by Formula VII with Hal-Y to prepare a compound represented by Formula I-3; preferably, wherein the reaction is carried out in the presence of a base and a solvent; more preferably, the base is one base or two bases in combination selected from the group consisting of potassium carbonate and sodium carbonate, the solvent is one solvent or more solvents in combination selected from the group consisting of DCM, DCE, ACN, THF, and DMF; the reaction is conducted at a temperature of 0 to 25° C.;

wherein the reaction equation is as follows:

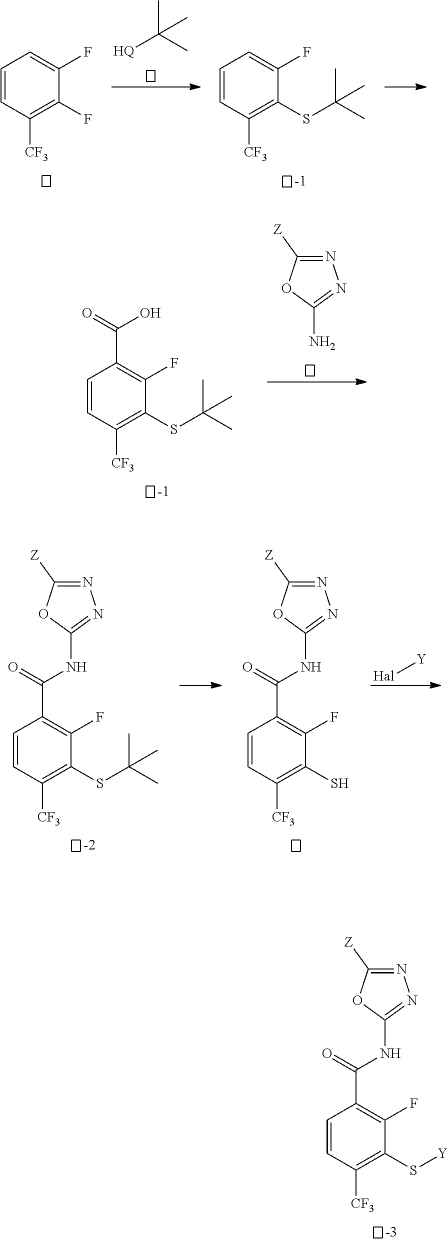

wherein, Hal represents halogen, preferably fluorine, chlorine or bromine.

Alternatively, c) when X represents SO, or SO₂ in the Formula I, the method comprises the following steps:

reacting a compound represented by Formula I-3 with a peroxide to prepare a compound represented by Formula I-4 and/or I-5;

wherein the reaction equation is as follows:

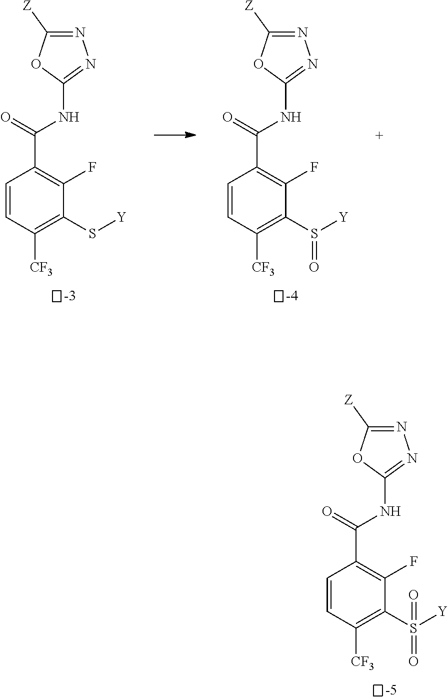

preferably, wherein the peroxide is

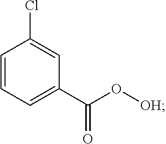 [SMILES: O=C(OO)c1cccc(Cl)c1]

the reaction is carried out in the presence of a solvent; more preferably, the solvent is one solvent or two solvents in combination selected from the group consisting of DCM and DCE; and the reaction is conducted at a temperature of 0 to 50° C.

Alternatively, d) when M is not hydrogen in Formula I, the method comprises the following steps:

reacting compounds represented by Formulas I-1, I-4 or I-5 with Hal-M to prepare compounds represented by Formulas I-6, I-7, or I-8, respectively;

wherein the reaction equation is as follows:

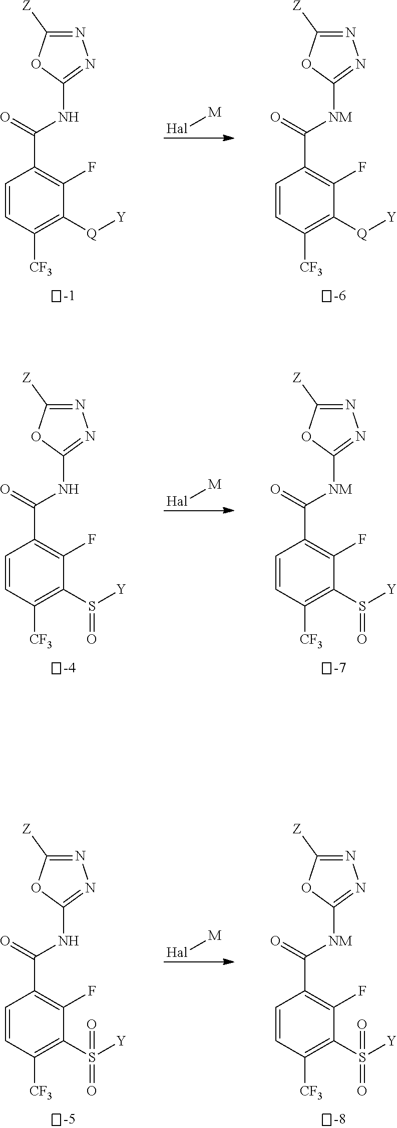

preferably, wherein the reaction is carried out in the presence of a base and a solvent; more preferably, the base is one base or two bases in combination selected from the group consisting of potassium carbonate and sodium carbonate, the solvent is one solvent or more solvents in combination selected from the group consisting of DCM, DCE, ACN, THF, and DMF, the reaction is conducted at a temperature of 0 to 100° C.;

alternatively, the reaction is carried out in the presence of a catalyst and a solvent;

preferably, the catalyst is 4-dimethylaminopyridine, the solvent is pyridine, and the reaction is conducted at a temperature of 0 to 80° C.

The present invention also provides a herbicidal composition, comprising (i) an N-(1,3,4-oxadiazol-2-yl)arylcarboxamide compound represented by Formula I; preferably, further comprising (ii) one or more additional herbicides and/or safeners; more preferably, further comprising (iii) an agrochemically acceptable formulation auxiliary.

The additional herbicides include the following compounds or salt or ester derivatives thereof;

a) ALS inhibitors: Pyrazosulfuron-ethyl (cas: 93697-74-6), Penoxsulam (cas: 219714-96-2), Bispyribac-sodium (cas: 125401-92-5), Pyriminobac-methyl (cas No.: 147411-69-6), Metazosulfuron (cas number: 868680-84-6), Propyrisulfuron (cas number: 570415-88-2), Triafamone (cas number: 874195-61-6);

b) ACCase inhibitors: Cyhalofop-butyl (cas number: 122008-85-9), Metamifop (cas number: 256412-89-2);

c) Hormones inhibitors: Quinclorac (cas number: 84087-01-4), MCPA (cas number: 94-74-6), 2,4-D (cas number: 94-75-7), 2,4-D butyric acid (cas number: 94-82-6), Fluroxypyr (cas number: 69377-81-7), Florpyrauxifen-benzyl (cas number: 1390661-72-9), MCPA butyric acid (cas number: 94-81-5), Dicamba (cas number: 1918-00-9), Quintrione (cas number: 130901-36-8), Clopyralid (cas number: 1702-17-6), Trichlopyr (cas number: 55335-06-3);

d) Cell division inhibitors: Pendimethalin (cas number: 40487-42-1), Butralin (cas number: 33629-47-9);

e) Lipid synthesis (non-ACC) inhibitors: Benthiocarb (cas number: 28249-77-6), Molinate (cas number: 2212-67-1);

f) HPPD inhibitors: Sanzuohuangcaotong ( [symbol] ) (cas number: 1911613-97-2), Shuangzuocaotong ( [symbol] ) (cas number: 1622908-18-2), Huanbifucaotong ( [symbol] ) (cas number: 1855929-45-1), Mesotrione (cas number: 104206-82-8), Benzobicylon (cas number: 156963-66-5), Tefuryltrione (cas number: 473278-76-1);

g) PDS inhibitors: Diflufenican (cas number: 83164-33-4), Fluorochloridone (cas number: 61213-25-0), Beflubutamid (cas number: 113614-08-7);

h) PPO inhibitors: Carfentrazone-ethyl (cas number: 128621-72-7), Pyraclonil (cas number: 158353-15-2), Oxadiazon (cas number: 19666-30-9), Oxadiargyl (cas number: 39807-15-3), Oxyfluorfen (cas number: 42874-03-3), Pentoxazone (cas number: 110956-75-7);

i) Long-chain fatty acid synthesis inhibitors: Butachlor (cas number: 23184-66-9), Pretilachlor (cas number: 51218-49-6), Mefenacet (cas number: 73250-68-7), Anilofos (cas number: 64249-01-0), Fentrazamide (cas number: 158237-07-1), Metolachlor (cas number: 51218-45-2), Piperophos (cas number: 24151-93-7), Pyroxasulfone (cas number: 447399-55-5);

j) PSII inhibitors: Simetryn (cas number: 1014-70-6), Prometryn (cas number: 7287-19-6), Amicarbazone (cas number: 129909-90-6), Isoproturon (cas number: 34123-59-6), Bromacil (cas number: 314-40-9), Pyridate (cas number: 55512-33-9), Chlortoluron (cas number: 15545-48-9), Bentazone (cas number: 25057-89-0), Propanil (cas number: 709-98-8), Metribuzin (cas number: 21087-64-9), Atrazine (cas No.: 1912-24-9), Bromoxynil (cas number: 1689-84-5), Bromoxynil octanoate (cas number: 1689-99-2), Terbuthylazine (CAS number: 5915-41-3);

k) DOXP inhibitors: Clomazone (cas number: 81777-89-1);

l) Others: Oxaziclomefone (cas number: 153197-14-9), Cinmethylin (cas number: 87818-31-3), Indanofan (cas number: 133220-30-1).

In the context of the present description, if an abbreviation of a generic name of active compound is used, it includes in each case all conventional derivatives thereof, such as esters and salts as well as isomers, in particular optical isomers, in particular one or more commercially available forms thereof. If the generic name denotes an ester or a salt, it also includes in each case all other conventional derivatives, such as other esters and salts, free acids and neutral compounds, as well as isomers, in particular optical isomers, in particular one or more commercially available forms thereof. The chemical name given to a compound means at least one compound encompassed by the generic name, and generally the preferred compound. In the case of sulfamide such as sulfonylurea, the salt thereof also includes a salt formed by exchanging a cation with a hydrogen atom in sulfonamide group. For example, 2,4-D or 2,4-D butyric acid derivatives include, but are not limited to, salts of 2,4-D or 2,4-D butyric acid, such as sodium salt, potassium salt, dimethylammonium salt, triethanol ammonium salt, isopropylamine salt, choline salt, etc., and esters of 2,4-D or 2,4-D butyric acid, such as methyl ester, ethyl ester, butyl ester, isooctyl ester, etc.; MCPA derivatives include, but are not limited to: MCPA sodium salt, potassium salt, dimethylammonium salt, isopropylamine salt, etc., and MCPA methyl ester, ethyl ester, isooctyl ester, ethyl thioester and the like.

The present invention also provides a method for controlling a weed, comprising: applying at least one of the N-(1,3,4-oxadiazol-2-yl)arylcarboxamide compound or the herbicidal composition in an herbicidally effective amount on a plant or in a weed area, preferably, wherein the plant is a rice (such as indica rice, japonica rice), the weed is a gramineous weed (such as Echinochloa phyllopogon, Leptochloa chinensis, Echinochloa crusgali, Digitaria sanguinalis, Setaria viridis), a broad-leaved weed (such as Monochoria vaginalis, Sagittaria trifolia, Abutilon theophrasti, Amaranthus retroflexus, Stellaria media), or a cyperaceae weed (such as Cyperus difformis, Scirpus juncoides).

The present invention also provides a use of at least one of the N-(1,3,4-oxadiazol-2-yl)arylcarboxamide compound or the herbicidal composition as above-described for controlling a weed, preferably, wherein the N-(1,3,4-oxadiazol-2-yl)arylcarboxamide compound is used for preventing and/or controlling a weed in a useful crop, wherein the useful crop is a transgenic crop or a crop treated by gene editing technique, the crop is a rice (such as indica rice, japonica rice), the weed is a gramineous weed (such as Echinochloa phyllopogon, Leptochloa chinensis, Echinochloa crusgali, Digitaria sanguinalis, Setaria viridis), a broad-leaved weed (such as Monochoria vaginalis, Sagittaria trifolia, Abutilon theophrasti, Amaranthus retroflexus, Stellaria media), a cyperaceae weed (such as Cyperus difformis, Scirpus juncoides).

The compounds of the formula I according to the invention have an outstanding herbicidal activity against a broad spectrum of economically important monocotyledonous and dicotyledonous harmful plants. The active compounds also act efficiently on perennial weeds which produce shoots from rhizomes, root stocks or other perennial organs and which are difficult to control. In this context, it is generally immaterial whether the substances are applied pre-sowing, pre-emergence or post-emergence. Specifically, examples may be mentioned of some representatives of the monocotyledonous and dicotyledonous weed flora which can be controlled by the compounds according to the invention, without these being a restriction to certain species. Examples of weed species on which the active compounds act efficiently are, from amongst the monocotyledons, Avena, Lolium, Alopecurus, Phalaris, Echinochloa, Digitaria, Setaria and also Cyperus species from the annual sector and from amongst the perennial species Agropyron, Cynodon, Imperata and Sorghum, and also perennial Cyperus species.

In the case of the dicotyledonous weed species, the spectrum of action extends to species such as, for example, Galium, Viola, Veronica, Lamium, Stellaria, Amaranthus, Sinapis, Ipomoea, Sida, Matricaria and Abutilon from amongst the annuals, and Convolvulus, Cirsium, Rumex and Artemisia in the case of the perennial weeds. The active compounds according to the invention also effect outstanding control of harmful plants which occur under the specific conditions of rice growing such as, for example, Echinochloa, Sagittaria, Alisma, Eleocharis, Scirpus and Cyperus. If the compounds according to the invention are applied to the soil surface prior to germination, then the weed seedlings are either prevented completely from emerging, or the weeds grow until they have reached the cotyledon stage but then their growth stops, and, eventually, after three to four weeks have elapsed, they die completely. If the compounds according to the invention are applied to the soil surface prior to germination, then the weed seedlings are either prevented completely from emerging, or the weeds grow until they have reached the cotyledon stage but then their growth stops, and, eventually, after three to four weeks have elapsed, they die completely. In particular, the compounds according to the invention exhibit excellent activity against Apera spica venti, Chenopodium album, Lamium purpureum, Polygonum convulvulus, Stellaria media, Veronica hederifolia, Veronica persica, Viola tricolor and against Amaranthus, Galium and Kochia species.

Although the compounds according to the invention have an excellent herbicidal activity against monocotyledonous and dicotyledonous weeds, crop plants of economically important crops such as, for example, wheat, barley, rye, rice, corn, sugarbeet, cotton and soya, are not damaged at all, or only to a negligible extent. In particular, they have excellent compatibility in cereals, such as wheat, barley and corn, in particular wheat. For these reasons, the present compounds are highly suitable for selectively controlling undesired plant growth in plantings for agricultural use or in plantings of ornamentals.

Owing to their herbicidal properties, these active compounds can also be employed for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested produce are known.

The use of the compounds of the formula I according to the invention or their salts in economically important transgenic crops of useful and ornamental plants, for example of cereal, such as wheat, barley, rye, oats, millet, rice, maniok and corn, or else in crops of sugarbeet, cotton, soya, rapeseed, potato, tomato, pea and other vegetable species is preferred. The compounds of the formula I can preferably be used as herbicides in crops of useful plants which are resistant or which have been made resistant by genetic engineering toward the phytotoxic effects of the herbicides.

Conventional ways for preparing novel plants which have modified properties compared to known plants comprise, for example, traditional breeding methods and the generation of mutants. Alternatively, novel plants having modified properties can be generated with the aid of genetic engineering methods (see, for example, EP-A 0 221 044, EP-A 0 131 624). For example, there have been described several cases of:
genetically engineered changes in crop plants in order to modify the starch synthesized in the plants (for example WO 92/11376, WO 92/14827, WO 91/19806),
transgenic crop plants which are resistant to certain herbicides of the glufosinate-(cf., for example, EP-A 0 242 236, EP-A 0 242 246) or glyphosate-type (WO 92/00377), or of the sulfonylurea-type (EP-A 0 257 993, U.S. Pat. No. 5,013,659A),
transgenic crop plants, for example cotton, having the ability to produce Bacillus thuringiensis toxins (Bt toxins) which impart resistance to certain pests to the plants (EP-A 0 142 924, EP-A 0 193 259),
transgenic crop plants having a modified fatty acid composition (WO 91/13972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene and Klone” [Genes and Clones], VCH Weinheim, 2nd edition 1996, or Christou, “Trends in Plant Science” 1 (1996) 423-431). In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the above-mentioned gene product.

To this end it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cells. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants. In this manner, it is possible to obtain transgenic plants which have modified properties by overexpression, suppression or inhibition of homologous (=natural) genes or gene sequences or by expression of heterologous (=foreign) genes or gene sequences.

When using the active compounds according to the invention in transgenic crops, in addition to the effects against harmful plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crops are resistant, and an effect on the growth and the yield of the transgenic crop plants. The invention therefore also provides for the use of the compounds according to the invention as herbicides for controlling harmful plants in transgenic crop plants.

In addition, the substances according to the invention have outstanding growth-regulating properties in crop plants. They engage in the plant metabolism in a regulating manner and can this be employed for the targeted control of plant constituents and for facilitating harvesting, for example by provoking desiccation and stunted growth. Furthermore, they are also suitable for generally regulating and inhibiting undesirable vegetative growth, without destroying the plants in the process. Inhibition of vegetative growth plays an important role in many monocotyledon and dicotyledon crops because lodging can be reduced hereby, or prevented completely.

The compounds according to the invention can be applied in the customary formulations in the form of wettable powders, emulsifiable concentrates, sprayable solutions, dusts or granules. The invention therefore also provides herbicidal compositions comprising compounds of the formula I. The compounds of the formula I can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil dispersions (OD), oil- or water-based dispersions, oil-miscible solutions, dusts (DP), capsule suspensions (CS), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules and waxes. These individual formulation types are known in principle and are described, for example, in Winnacker-Kühler, “Chemische Technologie” [Chemical Technology], Volume 7, C. Hauser Verlag Munich, 4th. Edition 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The necessary formulation auxiliaries, such as inert materials, surfactants, solvents and other additives, are likewise known and are described, for example, in Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflüchenaktive Äthylenoxidaddkte” [Surface-active ethylene oxide adducts], Wiss. Verlagagesell. Stuttgart 1976; Winnacker-Küchler, “Chemische Technologie” [Chemical Technology], Volume 7, C. Hauser Verlag Munich, 4th Edition 1986.

Wettable powders are preparations which are uniformly dispersible in water and which contain, in addition to the active compound and as well as a diluent or inert substance, surfactants of ionic and/or nonionic type (wetting agents, dispersants), for example polyethoxylated alkyl phenols, polyethoxylated fatty alcohols, polyethoxylated fatty amines, fatty alcohol polyglycol ethersulfates, alkanesulfonates, alkylbenzenesulfonates, sodium ligninsulfonate, sodium 2,2′-dinaphthylmethane-6,6′-disulfonate, sodium dibutyinaphthalenesulfona-te or else sodium oleoylmethyltaurinate. To prepare the wettable powders, the herbicidally active compounds are finely ground, for example in customary apparatus such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatic compounds or hydrocarbons or mixtures of the solvents, with the addition of one or more surfactants of ionic and/or nonionic type (emulsifiers). Examples of emulsifiers which can be used are calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth. Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers. Water-dispersible granules are generally prepared by the customary processes, such as spray-drying, fluidized-bed granulation, disk granulation, mixing using high-speed mixers, and extrusion without solid inert material.

For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th Ed., McGraw-Hill, New York 1973, pp. 8-57. For further details on the formulation of crop protection products, see for example G. C. Klingman, “Weed Control as a Science”, John Wiley and Sons Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical formulations generally contain from 0.1 to 99% by weight, in particular from 0.1 to 95% by weight, of active compound of the formula I. In wettable powders the concentration of active compound is, for example, from about 10 to 99% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, the formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

Based on these formulations it is also possible to produce combinations with other pesticidally active substances, for example insecticides, acaricides, herbicides and fungicides, and also with safeners, fertilizers and/or growth regulators, for example in the form of a ready-mix or tank mix.

Suitable active compounds which can be combined with the active compounds according to the invention in mixed formulations or in a tank mix are, for example, known active compounds as described in for example World Herbicide New Product Technology Handbook, China Agricultural Science and Farming Techniques Press, 2010.9 and in the literature cited therein. For example the following active compounds may be mentioned as herbicides which can be combined with the compounds of the formula I (note: the compounds are either named by the “common name” in accordance with the International Organization for Standardization (ISO) or by the chemical names, if appropriate together with a customary code number): acetochlor, butachlor, alachlor, propisochlor, metolachlor, s-metolachlor, pretilachlor, propachlor, ethachlor, napropamide, R-left handed napropamide, propanil, mefenacet, diphenamid, diflufenican, ethaprochlor, beflubutamid, bromobutide, dimethenamid, dimethenamid-P, etobenzanid, flufenacet, thenylchlor, metazachlor, isoxaben, flamprop-M-methyl, flamprop-M-propyl, allidochlor, pethoxamid, chloranocryl, cyprazine, mefluidide, monalide, delachlor, prynachlor, terbuchlor, xylachlor, dimethachlor, cisanilide, trimexachlor, clomeprop, propyzamide, pentanochlor, carbetamide, benzoylprop-ethyl, cyprazole, butenachlor, tebutam, benzipram, mogrton, dichlofluanid, naproanilide, diethatyl-ethyl, naptalam, flufenacet, benzadox, chlorthiamid, chlorophthalimide, isocarbamide, picolinafen, atrazine, simazine, prometryn, cyanatryn, simetryn, ametryn, propazine, dipropetryn, SSH-108, terbutryn, terbuthylazine, triaziflam, cyprazine, proglinazine, trietazine, prometon, simetone, aziprotryne, desmetryn, dimethametryn, procyazine, mesoprazine, sebuthylazine, secbumeton, terbumeton, methoprotryne, cyanatryn, ipazine, chlorazine, atraton, pendimethalin, eglinazine, cyanuric acid, indaziflam, chlorsulfuron, metsulfuron-methyl, bensulfuron methyl, chlorimuron-ethyl, tribenuron-methyl, thifensulfuron-methyl, pyrazosulfuron-ethyl, mesosulfuron, iodosulfuron-methyl sodium, foramsulfuron, cinosulfuron, triasulfuron, sulfometuron methyl, nicosulfuron, ethametsulfuron-methyl, amidosulfuron, ethoxysulfuron, cyclosulfamuron, rimsulfuron, azimsulfuron, flazasulfuron, monosulfuron, monosulfuron-ester, flucarbazone-sodium, flupyrsulfuron-methyl, halosulfuron-methyl, oxasulfuron, imazosulfuron, primisulfuron, propoxycarbazone, prosulfuron, sulfosulfuron, trifloxysulfuron, triflusulfuron-methyl, tritosulfuron, sodium metsulfuron methyl, flucetosulfuron, HNPC-C, orthosulfamuron, propyrisulfuron, metazosulfuron, acifluorfen, fomesafen, lactofen, fluoroglycofen, oxyfluorfen, chlornitrofen, aclonifen, ethoxyfen-ethyl, bifenox, nitrofluorfen, chlomethoxyfen, fluorodifen, fluoronitrofen, furyloxyfen, nitrofen, TOPE, DMNP, PPG1013, AKH-7088, halosafen, chlortoluron, isoproturon, linuron, diuron, dymron, fluometuron, benzthiazuron, methabenzthiazuron, cumyluron, ethidimuron, isouron, tebuthiuron, buturon, chlorbromuron, methyldymron, phenobenzuron, SK-85, metobromuron, metoxuron, afesin, monuron, siduron, fenuron, fluothiuron, neburon, chloroxuron, noruron, isonoruron, 3-cyclooctyl-1, thiazfluron, tebuthiuron, difenoxuron, parafluron, methylamine tribunil, karbutilate, trimeturon, dimefuron, monisouron, anisuron, methiuron, chloreturon, tetrafluron, phenmedipham, phenmedipham-ethyl, desmedipham, asulam, terbucarb, barban, propham, chlorpropham, rowmate, swep, chlorbufam, carboxazole, chlorprocarb, fenasulam, BCPC, CPPC, carbasulam, butylate, benthiocarb, vernolate, molinate, triallate, dimepiperate, esprocarb, pyributicarb, cycloate, avadex, EPTC, ethiolate, orbencarb, pebulate, prosulfocarb, tiocarbazil, CDEC, dimexano, isopolinate, methiobencarb, 2,4-D butyl ester, MCPA-Na, 2,4-D isooctyl ester, MCPA isooctyl ester, 2,4-D sodium salt, 2,4-D dimethyla mine salt, MCPA-thioethyl, MCPA, 2,4-D propionic acid, high 2,4-D propionic acid salt, 2,4-D butyric acid, MCPA propionic acid, MCPA propionic acid salt, MCPA butyric acid, 2,4,5-D, 2,4,5-D propionic acid, 2,4,5-D butyric acid, MCPA amine salt, dicamba, erbon, chlorfenac, saison, TBA, chloramben, methoxy-TBA, diclofop-methyl, fluazifop-butyl, fluazifop-p-butyl, haloxyfop-methyl, haloxyfop-P, quizalofop-ethyl, quizalofop-p-ethyl, fenoxaprop-ethy, fenoxaprop-p-ethyl, propaquizafop, cyhalofop-butyl, metamifop, clodinafop-propargyl, fenthiaprop-ethyl, chloroazifop-propynyl, poppenate-methyl, trifopsime, isoxapyrifop, paraquat, diquat, oryzalin, ethalfluralin, isopropalin, nitralin, profluralin, prodinamine, benfluralin, fluchloraline, dinitramina, dipropalin, chlornidine, methalpropalin, dinoprop, glyphosate, anilofos, glufosinate ammonium, amiprophos-methyl, sulphosate, piperophos, bialaphos-sodium, bensulide, butamifos, phocarb, 2,4-DEP, H-9201, zytron, imazapyr, imazethapyr, imazaquin, imazamox, imazamox ammonium salt, imazapic, imazamethabenz-methyl, fluroxypyr, fluroxypyr isooctyl ester, clopyralid, picloram, trichlopyr, dithiopyr, haloxydine, 3,5,6-trichloro-2-pyridinol, thiazopyr, fluridone, aminopyralid, diflufenzopyr, triclopyr-butotyl, Cliodinate, sethoxydim, clethodim, cycloxydim, alloxydim, clefoxydim, butroxydim, tralkoxydim, tepraloxydim, buthidazole, metribuzin, hexazinone, metamitron, ethiozin, ametridione, amibuzin, bromoxynil, bromoxynil octanoate, ioxynil octanoate, ioxynil, dichlobenil, diphenatrile, pyraclonil, chloroxynil, iodobonil, flumetsulam, florasulam, penoxsulam, metosulam, cloransulam-methyl, diclosulam, pyroxsulam, benfuresate, bispyribac-sodium, pyribenzoxim, pyriftalid, pyriminobac-methyl, pyrithiobac-sodium, benzobicylon, mesotrione, sulcotrione, tembotrione, tefuryltrione, bicyclopyrone, ketodpiradox, isoxaflutole, clomazone, fenoxasulfone, methiozolin, fluazolate, pyraflufen-ethyl, pyrazolynate, difenzoquat, pyrazoxyfen, benzofenap, nipyraclofen, pyrasulfotole, topramezone, pyroxasulfone, cafenstrole, flupoxam, aminotriazole, amicarbazone, azafenidin, carfentrazone-ethyl, sulfentrazone, bencarbazone, benzfendizone, butafenacil, bromacil, isocil, lenacil, terbacil, flupropacil, cinidon-ethyl, flumiclorac-pentyl, flumioxazin, propyzamide, MK-129, flumezin, pentachlorophenol, dinoseb, dinoterb, dinoterb acetate, dinosam, DNOC, chloronitrophene, medinoterb acetate, dinofenate, oxadiargyl, oxadiazon, pentoxazone, Flufenacet, fluthiacet-methyl, fentrazamide, flufenpyr-ethyl, pyrazon, brompyrazon, metflurazon, kusakira, dimidazon, oxapyrazon, norflurazon, pyridafol, quinclorac, quinmerac, bentazone, pyridate, oxaziclomefone, benazolin, clomazone, cinmethylin, ZJ0702, pyribambenz-propyl, indanofan, sodium chlorate, dalapon, trichloroacetic acid, monochloroacetic acid, hexachloroacetone, flupropanate, cyperquat, bromofenoxim, epronaz, methazole, flurtamone, benfuresate, ethofumesate, tioclorim, chlorthal, fluorochloridone, tavron, acrolein, bentranil, tridiphane, chlorfenpropmethyl, thidiarizonaimin, phenisopham, busoxinone, methoxyphenone, saflufenacil, clacyfos, chloropon, alorac, diethamquat, etnipromid, iprymidam, ipfencarbazone, thiencarbazone-methyl, pyrimisulfan, chlorflurazole, tripropindan, sulglycapin, prosulfalin, cambendichlor, aminocyclopyrachlor, rodethanil, benoxacor, fenclorim, flurazole, fenchlorazole-ethyl, cloquintocet-mexyl, oxabetrinil, MG/91, cyometrinil, DKA-24, mefenpyr-diethyl, furilazole, fluxofenim, isoxadifen-ethyl, dichlormid, halauxifen-methyl, DOW florpyrauxifen, UBH-509, D489, LS 82-556, KPP-300, NC-324, NC-330, KH-218, DPX-N8189, SC-0744, DOWC0535, DK-8910, V-53482, PP-600, MBH-001, KIH-9201, ET-751, KIH-6127 and KIH-2023.

For use, the formulations which are present in commercially available form are, if appropriate, diluted in the customary manner, for example using water in the case of wettable powders, emulsifiable concentrates, dispersions and water-dispersible granules. Products in the form of dusts, granules for soil application or broadcasting and sprayable solutions are usually not further diluted with other inert substances prior to use. The application rate of the compounds of the formula I required varies with the external conditions, such as temperature, humidity, the nature of the herbicide used and the like. It can vary within wide limits, for example between 0.001 and 1.0 kg a.i./ha or more of active substance, but it is preferably between 0.005 and 750 g a.i./ha, in particular between 0.005 and 500 g a.i./ha.

### SPECIFIC MODE FOR CARRYING OUT THE INVENTION

The following embodiments are used to illustrate the present invention in detail and should not be taken as any limit to the present invention. The scope of the invention would be explained through the Claims.

In view of economics, variety and biological activity of a compound, we preferably synthesized several compounds, part of which are listed in the following Table A1-A152. The structure and information of a certain compound are shown in Table A1 and Table 1. The compounds in Table A1-A152 are listed for further explication of the present invention, other than any limit therefor. The subject of the present invention should not be interpreted by those skilled in the art as being limited to the following compounds.

TABLE A1
Structures and 1H NMR data of compounds
I

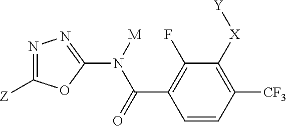

| Compound | | | | | |
|---|---|---|---|---|---|
| No. | X | Y | Z | M | ¹HNMR |
| 1-1 | S | Me | Me | H | ¹H NMR |
| | | | | | (500 MHz, |
| | | | | | DMSO-d6) |
| | | | | | 12.35 (s, |
| | | | | | 1H), 7.84 |
| | | | | | (dd, J = 8.0, |
| | | | | | 7.0 Hz, 1H), |
| | | | | | 7.75 (d, J = |
| | | | | | 8.0 Hz, 1H), |
| | | | | | 2.50 (s, 3H), |
| | | | | | 2.48 (s, 3H). |
| 1-2 | SO | Me | Me | H | ¹H NMR |
| | | | | | (500 MHz, |
| | | | | | DMSO-d₆) |
| | | | | | 12.37 (s, |
| | | | | | 1H), 8.04 |
| | | | | | (dd, J = 8.0, |
| | | | | | 7.0 Hz, 1H), |
| | | | | | 7.78 (d, J = |
| | | | | | 8.0 Hz, 1H), |
| | | | | | 3.35 (s, 3H), |
| | | | | | 2.50 (s, 3H). |
| 1-3 | SO₂ | Me | Me | H | ¹H NMR |
| | | | | | (500 MHz, |
| | | | | | DMSO-d₆) |
| | | | | | 12.39 (s, |
| | | | | | 1H), 8.24 |
| | | | | | (dd, J = 8.0, |
| | | | | | 7.0 Hz, 1H), |
| | | | | | 7.98 (d, J = |
| | | | | | 8.0 Hz, 1H), |
| | | | | | 3.45 (s, 3H), |
| | | | | | 2.50 (s, 3H). |
| 1-4 | S | Et | Me | H | ¹H NMR |
| | | | | | (500 MHz, |
| | | | | | DMSO-d6) |
| | | | | | 12.39 (s, |
| | | | | | 1H), 7.88 |
| | | | | | (dd, J = 8.0, |
| | | | | | 7.0 Hz, 1H), |
| | | | | | 7.78 (d, J = |
| | | | | | 8.0 Hz, 1H), |
| | | | | | 2.98 (q, J = |
| | | | | | 7.5 Hz, 2H) |
| | | | | | 2.51 (s, 3H), |
| | | | | | 1.15 (t, J = |
| | | | | | 7.5 Hz, 3H). |
| 1-5 | SO | Et | Me | H | ¹H NMR |
| | | | | | (500 MHz, |
| | | | | | DMSO-d6) |
| | | | | | 12.37 (s, |
| | | | | | 1H), 8.04 |
| | | | | | (dd, J = 8.0, |
| | | | | | 7.0 Hz, 1H), |
| | | | | | 7.78 (d, J = |
| | | | | | 8.0 Hz, 1H), |
| | | | | | 3.47 (q, J = |
| | | | | | 7.5 Hz, 2H), |
| | | | | | 2.50 (s, 3H), |
| | | | | | 1.12 (t, J = |
| | | | | | 7.5 Hz, 3H). |
| 1-6 | SO2 | Et | Me | H | ¹H NMR |
| | | | | | (500 MHz, |
| | | | | | DMSO-d6) |
| | | | | | 12.41 (s, |
| | | | | | 1H), 8.22 |
| | | | | | (dd, J = 8.0, |
| | | | | | 7.0 Hz, 1H), |
| | | | | | 7.96 (d, J = |
| | | | | | 8.0 Hz, 1H), |
| | | | | | 3.54 (q, J = |
| | | | | | 7.5 Hz, 2H), |
| | | | | | 2.49 (s, 3H), |
| | | | | | 1.20 (t, J = |
| | | | | | 7.5 Hz, 3H). |
| 1-7 | S | 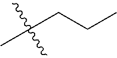 [SMILES: *CCC] | Me | H | ¹H NMR (500 MHz, DMSO-d₆) 12.38 (s, |
| | | | | | 1H), 7.86 |
| | | | | | (dd, J = 8.0, |
| | | | | | 7.0 Hz, 1H), |
| | | | | | 7.77 (d, J = |
| | | | | | 8.0 Hz, 1H), |
| | | | | | 2.95 (t, J = |
| | | | | | 7.5 Hz, 2H), |
| | | | | | 2.50 (s, 3H), |
| | | | | | 1.25-1.20 (m, |
| | | | | | 2H), 0.90 (t, |
| | | | | | J = 7.5 Hz, |
| | | | | | 3H). |
| 1-8 | SO | 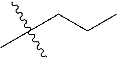 [SMILES: *CCC] | Me | H | ¹H NMR (500 MHz, DMSO-d₆) 12.57 (s, |
| | | | | | 1H), 8.07 |
| | | | | | (dd, J = 8.0, |
| | | | | | 7.0 Hz, 1H), |
| | | | | | 7.82 (d, J = |
| | | | | | 8.0 Hz, 1H), |
| | | | | | 3.57-3.47 (m, |
| | | | | | 2H), 2.48 (s, |
| | | | | | 3H), 1.70- |
| | | | | | 1.52 (m, 2H), |
| | | | | | 1.08-0.93 (m, |
| | | | | | 3H). |
| 1-9 | SO₂ | 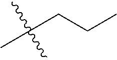 [SMILES: *CCC] | Me | H | ¹H NMR (500 MHz, DMSO-d6) 12.59 (s, |
| | | | | | 1H), 8.27 |
| | | | | | (dd, J = 8.0, |
| | | | | | 7.0 Hz, 1H), |
| | | | | | 3.75 (t, J = |
| | | | | | 7.5 Hz, 2H), |
| | | | | | 2.48 (s, 3H), |
| | | | | | 1.80-1.62 (m, |
| | | | | | 2H), 1.05 (t, |
| | | | | | J = 7.5 Hz, |
| | | | | | 3H). |
| 1-10 | S | 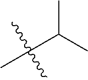 [SMILES: *C(C)C] | Me | H | ¹H NMR (500 MHz, DMSO-d6) 12.42 (s, 1H), 7.89 |
| | | | | | (dd, J = 8.0, |
| | | | | | 7.0 Hz, 1H), |
| | | | | | 7.80 (d, J = |
| | | | | | 8.0 Hz, |
| | | | | | 1H), 3.52 |
| | | | | | (hept., J = 7.5 |
| | | | | | Hz, 1H) 2.51 |
| | | | | | (s, 3H), 1.22 |
| | | | | | (d, J = 7.5 |
| | | | | | Hz, 6H). |
| 1-11 | SO | 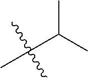 [SMILES: *C(C)C] | Me | H | ¹H NMR (500 MHz, DMSO-d6) 12.55 (s, 1H), 8.17 |
| | | | | | 7.0 Hz, 1H), |
| | | | | | 7.88 (d, J = |
| | | | | | 8.0 Hz, 1H), |
| | | | | | 3.80-3.73 (m, |
| | | | | | 1H), 2.50 (s, |
| | | | | | 3H), 1.47 (d, |
| | | | | | J = 7.0, 3H), |
| | | | | | 0.98 (d, J = |
| | | | | | 7.0 Hz, 3H). |
| 1-12 | SO2 | 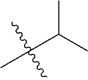 [SMILES: *C(C)C] | Me | H | ¹H NMR (500 MHz, DMSO-d6) 12.75 (s, 1H), 8.27 |
| | | | | | (dd, J = 8.0, |
| | | | | | 7.0 Hz, 1H), |
| | | | | | 8.08 (d, J = |
| | | | | | 8.0 Hz, 1H), |
| | | | | | 3.89 (hept., |
| | | | | | J = 7.5 Hz, |
| | | | | | 1H), 2.50 (s, |
| | | | | | 3H), 1.27 (d, |
| | | | | | J = 7.0, 6H). |
| 1-13 | S | 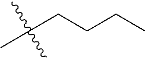 [SMILES: *CCCC] | Me | H | ¹H NMR (500 MHz, DMSO-d6) 12.25 (s, |
| | | | | | 1H), 7.86 |
| | | | | | (dd, J = 8.0, |
| | | | | | 7.0 Hz, 1H), |
| | | | | | 7.77 (d, J = |
| | | | | | 8.0 Hz, 1H), |
| | | | | | 2.99 |
| | | | | | 7.5 Hz, 2H), |
| | | | | | 2.50 (s, 3H), |
| | | | | | 1.48-1.42 (m, |
| | | | | | 2H), 1.39- |
| | | | | | 1.33 (m, 2H), |
| | | | | | 0.83 (t, J = |
| | | | | | 7.5 Hz, 3H). |
| 1-14 | SO | 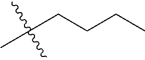 [SMILES: *CCCC] | Me | H | ¹H NMR (500 MHz, DMSO-d6) 12.27 (s, |
| | | | | | 1H), 8.06 |
| | | | | | (dd, J = 8.0, |
| | | | | | 7.0 Hz, 1H), |
| | | | | | 7.88 (d, J = |
| | | | | | 8.0 Hz, 1H), |
| | | | | | 3.57 (t, J = |
| | | | | | 7.5 Hz, 2H), |
| | | | | | 2.48 (s, 3H), |
| | | | | | 1.72-1.46 (m, |
| | | | | | 4H), 0.93 (t, |
| | | | | | J = 7.5 Hz, |
| | | | | | 3H). |
| 1-15 | SO₂ | 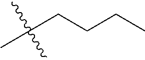 [SMILES: *CCCC] | Me | H | ¹H NMR (500 MHz, DMSO-d6) 12.47 (s, |
| | | | | | 1H), 8.26 |
| | | | | | (dd, J = 8.0, |
| | | | | | 7.0 Hz, 1H), |
| | | | | | 8.08 (d, J = |
| | | | | | 8.0 Hz, 1H), |
| | | | | | 3.77 (t, J = |
| | | | | | 7.5 Hz, 2H), |
| | | | | | 2.48 (s, 3H), |
| | | | | | 1.82-1.56 (m, |
| | | | | | 4H), 1.02 (t, |
| | | | | | J = 7.5 Hz, |
| | | | | | 3H). |
| 1-16 | S | 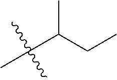 [SMILES: *C(C)CC] | Me | H | ¹H NMR (500 MHz, DMSO-d6) 12.40 (s, 1H), 7.93 |
| | | | | | (dd, J = 8.0, |
| | | | | | 7.0 Hz, 1H), |
| | | | | | 7.63 (d, J = |
| | | | | | 8.0 Hz, 1H), |
| | | | | | 3.68-3.60 |
| | | | | | (m, 1H), 2.50 |
| | | | | | (s, 3H), 1.75- |
| | | | | | 1.66 (m, |
| | | | | | 2H), 1.18 (d, |
| | | | | | J = 7.0 Hz, |
| | | | | | 3H), 1.07 (t, |
| | | | | | J = 7.5 Hz, |
| | | | | | 3H). |
| 1-17 | SO | 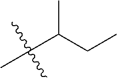 [SMILES: *C(C)CC] | Me | H | ¹H NMR (500 MHz, DMSO-d6) 12.41 (s, 1H), 8.12 |
| | | | | | (dd, J = 8.0, |
| | | | | | 7.0 Hz, 1H), |
| | | | | | 7.83 (d, J = |
| | | | | | 8.0 Hz, 1H), |
| | | | | | 3.88-3.80 |
| | | | | | (m, 1H), 2.50 |
| | | | | | (s, 3H), 1.85- |
| | | | | | 1.73 (m, |
| | | | | | 2H), 1.23 (d, |
| | | | | | J = 7.0 Hz, |
| | | | | | 3H), 1.10 (t, |
| | | | | | J = 7.5 Hz, |
| | | | | | 3H). |
| 1-18 | SO₂ | 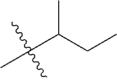 [SMILES: *C(C)CC] | Me | H | ¹H NMR (500 MHz, DMSO-d6) 12.43 (s, 1H), 8.32 |
| | | | | | (dd, J = 8.0, |
| | | | | | 7.0 Hz, 1H), |
| | | | | | 8.04 (d, J = |
| | | | | | 8.0 Hz, 1H), |
| | | | | | 4.08-4.00 |
| | | | | | (m, 1H), 2.50 |
| | | | | | (s, 3H), 1.95- |
| | | | | | 1.85 (m, |
| | | | | | 2H), 1.30 (d, |
| | | | | | J = 7.0 Hz, |
| | | | | | 3H), 1.14 (t, |
| | | | | | J = 7.5 Hz, |
| | | | | | 3H). |
| 1-19 | S | 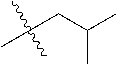 [SMILES: *CC(C)C] | Me | H | ¹H NMR (500 MHz, DMSO-d6) 12.38 (s, |
| | | | | | 1H), 7.85 |
| | | | | | (dd, J = 8.0, |
| | | | | | 7.0 Hz, 1H), |
| | | | | | 7.59 (d, J = |
| | | | | | 8.0 Hz, 1H), |
| | | | | | 3.46 (d, J = |
| | | | | | 7.0 Hz, 2H), |
| | | | | | 2.50 (s, 3H), |
| | | | | | 1.82-1.77 (m, |
| | | | | | 1H), 1.15 (d, |
| | | | | | J = 7.5 Hz, |
| | | | | | 6H). |
| 1-20 | SO | 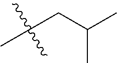 [SMILES: *CC(C)C] | Me | H | ¹H NMR (500 MHz, DMSO-d6) 12.41 (s, |
| | | | | | 1H), 8.05 |
| | | | | | (dd, J = 8.0, |
| | | | | | 7.0 Hz, 1H), |
| | | | | | 7.79 (d, J = |
| | | | | | 8.0 Hz, 1H), |
| | | | | | 3.55 (d, J = |
| | | | | | 7.0 Hz, 2H), |
| | | | | | 2.50 (s, 3H), |
| | | | | | 1.92-1.86 (m, |
| | | | | | 1H), 1.20 (d, |
| | | | | | J = 7.5 Hz, |
| | | | | | 6H). |
| 1-21 | SO₂ | 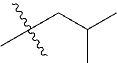 [SMILES: *CC(C)C] | Me | H | ¹H NMR (500 MHz, DMSO-d6) 1H), 8.26 |
| | | | | | (dd, J = 8.0, |
| | | | | | 7.0 Hz, 1H), |
| | | | | | 7.99 (d, J = |
| | | | | | 8.0 Hz, 1H), |
| | | | | | 3.75 (d, J = |
| | | | | | 7.0 Hz, 2H), |
| | | | | | 2.50 (s, 3H), |
| | | | | | 1.99-1.92 (m, |
| | | | | | 1H), 1.27 (d, |
| | | | | | J = 7.5 Hz, |
| | | | | | 6H). |
| 1-22 | S | 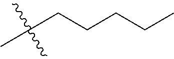 [SMILES: *CCCCC] | Me | H | ¹H NMR (500 MHz, DMSO-d6) 12.36 (s, |
| | | | | | 1H), 7.94 |
| | | | | | (dd, J = 8.0, |
| | | | | | 7.0 Hz, 1H), |
| | | | | | 7.68 (d J = |
| | | | | | 8.0 Hz, 1H), |
| | | | | | 3.25 (t, J = |
| | | | | | 7.5 Hz, 2H), |
| | | | | | 2.50 (s, 3H), |
| | | | | | 1.72-1.48 (m, |
| | | | | | 6H), 1.10 (t, |
| | | | | | J = 7.5 Hz, |
| | | | | | 3H). |
| 1-23 | SO | 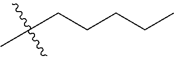 [SMILES: *CCCCC] | Me | H | ¹H NMR (500 MHz, DMSO-d6) 12.37 (s, |
| | | | | | 1H), 8.05 |
| | | | | | (dd, J = 8.0, |
| | | | | | 7.0 Hz, 1H), |
| | | | | | 7.75 (d J = |
| | | | | | 8.0 Hz, 1H), |
| | | | | | 3.45 (t, J = |
| | | | | | 7.5 Hz, 2H), |
| | | | | | 2.50 (s, 3H), |
| | | | | | 1.82-1.56 (m, |
| | | | | | 6H), 1.12 (t, |
| | | | | | J = 7.5 Hz, |
| | | | | | 3H). |
| 1-24 | SO₂ | 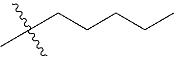 [SMILES: *CCCCC] | Me | H | ¹H NMR (500 MHz, DMSO-d6) 12.37 (s, |
| | | | | | 1H), 8.25 |
| | | | | | (dd, J = 8.0, |
| | | | | | 7.0 Hz, 1H), |
| | | | | | 7.96 (d, J = |
| | | | | | 8.0 Hz, 1H), |
| | | | | | 3.66 (t, J = |
| | | | | | 7.5 Hz, 2H), |
| | | | | | 2.50 (s, 3H), |
| | | | | | 1.99-1.56 (m, |
| | | | | | 6H), 1.15 (t, |
| | | | | | J = 7.5 Hz, |
| | | | | | 3H). |
| 1-25 | S | 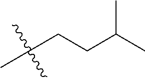 [SMILES: *CCC(C)C] | Me | H | ¹H NMR (500 MHz, DMSO-d6) 12.37 (s, 1H), 7.96 |
| | | | | | (dd, J = 8.0, |
| | | | | | 7.0 Hz, 1H), |
| | | | | | 7.65 (d, J = |
| | | | | | 8.0 Hz, 1H), |
| | | | | | 3.27 (t, J = |
| | | | | | 7.5 Hz, 2H), |
| | | | | | 2.50 (s, 3H), |
| | | | | | 1.75-1.66 (m, |
| | | | | | 3H), 1.18 (d, |
| | | | | | J = 7.5 Hz, |
| | | | | | 6H). |
| 1-26 | SO | 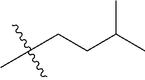 [SMILES: *CCC(C)C] | Me | H | ¹H NMR (500 MHz, DMSO-d6) 12.37 (s, 1H), 8.04 |
| | | | | | (dd, J = 8.0, |
| | | | | | 7.0 Hz, 1H), |
| | | | | | 3.49 (t, J = |
| | | | | | 7.5 Hz, 2H), |
| | | | | | 2.50 (s, 3H), |
| | | | | | 1.82-1.76 (m, |
| | | | | | 3H), 1.22 (d, |
| | | | | | J = 7.5 Hz, |
| | | | | | 6H). |
| 1-27 | SO₂ | 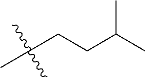 [SMILES: *CCC(C)C] | Me | H | ¹H NMR (500 MHz, DMSO-d6) 12.39 (s, 1H), 8.25 |
| | | | | | (dd, J = 8.0, |
| | | | | | 7.0 Hz, 1H), |
| | | | | | 3.68 (t, J = |
| | | | | | 7.5 Hz, 2H), |
| | | | | | 2.50 (s, 3H), |
| | | | | | 1.92-1.86 (m, |
| | | | | | 3H), 1.26 (d, |
| | | | | | J = 7.5 Hz, |
| | | | | | 6H). |
| 1-28 | S | 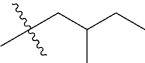 [SMILES: *CC(C)CC] | Me | H | ¹H NMR DMSO-d6) 12.40 (s, 1H), 7.88 |
| | | | | | (dd, J = 8.0, |
| | | | | | 7.0 Hz, 1H), |
| | | | | | 7.61 (d, J = |
| | | | | | 8.0 Hz, 1H), |
| | | | | | 3.32 (d, J = |
| | | | | | 7.0 Hz, 2H), |
| | | | | | 2.50 (s, 3H), |
| | | | | | 1.82-1.63 (m, |
| | | | | | 3H), 1.23 (d, |
| | | | | | J = 7.5 Hz, |
| | | | | | 3H), 1.08 (t, |
| | | | | | J = 7.5 Hz, |
| | | | | | 3H). |
| 1-29 | SO | 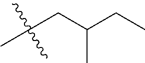 [SMILES: *CC(C)CC] | Me | H | ¹H NMR (500 MHz, DMSO-d6) 12.41 (s, |
| | | | | | 1H), 8.05 |
| | | | | | (dd, J = 8.0, |
| | | | | | 7.0 Hz, 1H), |
| | | | | | 7.79 (d, J = |
| | | | | | 8.0 Hz, 1H), |
| | | | | | 3.55 (d, J = |
| | | | | | 7.0 Hz, 2H), |
| | | | | | 2.50 (s, 3H), |
| | | | | | 1.92-1.66 (m, |
| | | | | | 3H), 1.29 (d, |
| | | | | | J = 7.5 Hz, |
| | | | | | 3H), 1.10 (t, |
| | | | | | J = 7.5 Hz, |
| | | | | | 3H). |
| 1-30 | SO₂ | 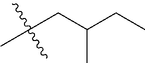 [SMILES: *CC(C)CC] | Me | H | ¹H NMR (500 MHz, DMSO-d6) 12.42 (s, |
| | | | | | 1H), 8.25 |
| | | | | | (dd, J = 8.0, |
| | | | | | 7.0 Hz, 1H), |
| | | | | | 7.96 (d, J = |
| | | | | | 8.0 Hz, 1H), |
| | | | | | 3.76 (d, J = |
| | | | | | 7.0 Hz, 2H), |
| | | | | | 2.50 (s, 3H), |
| | | | | | 1.99-1.69 (m, |
| | | | | | 3H), 1.36 (d, |
| | | | | | J = 7.5 Hz, |
| | | | | | 3H), 1.17 (t, |
| | | | | | J = 7.5 Hz, |
| | | | | | 3H). |
| 1-31 | S | 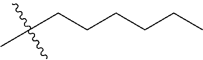 [SMILES: *CCCCCC] | Me | H | ¹H NMR (500 MHz, DMSO-d6) 12.37 (s, |
| | | | | | 1H), 7.98 |
| | | | | | (dd, J = 8.0, |
| | | | | | 7.0 Hz, 1H), |
| | | | | | 7.60 (d, J = |
| | | | | | 8.0 Hz, 1H), |
| | | | | | 3.27 (t, J = |
| | | | | | 7.5 Hz, 2H), |
| | | | | | 2.51 (s, 3H), |
| | | | | | 1.76-1.48 (m, |
| | | | | | 8H), 1.05 (t, |
| | | | | | J = 7.5 Hz, |
| | | | | | 3H). |
| 1-32 | SO | 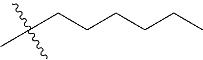 [SMILES: *CCCCCC] | Me | H | ¹H NMR (500 MHz, DMSO-d6) 12.38 (s, |
| | | | | | 1H), 8.05 |
| | | | | | (dd, J = 8.0, |
| | | | | | 7.0 Hz, 1H), |
| | | | | | 7.81 (d, J = |
| | | | | | 8.0 Hz, 1H), |
| | | | | | 3.47 (t, J = |
| | | | | | 7.5 Hz, 2H), |
| | | | | | 2.51 (s, 3H), |
| | | | | | 1.85-1.55 (m, |
| | | | | | 8H), 1.10 (t, |
| | | | | | J = 7.5 Hz, |
| | | | | | 3H). |
| 1-33 | SO₂ | 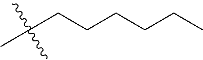 [SMILES: *CCCCCC] | Me | H | ¹H NMR (500 MHz, DMSO-d6) 12.39 (s, |
| | | | | | 1H), 8.25 |
| | | | | | (dd, J = 8.0, |
| | | | | | 7.0 Hz, 1H), |
| | | | | | 7.98 (d J = |
| | | | | | 8.0 Hz, 1H), |
| | | | | | 3.69 (t, J = |
| | | | | | 7.5 Hz, 2H), |
| | | | | | 2.51 (s, 3H), |
| | | | | | 1.95-1.57 (m, |
| | | | | | 8H), 1.19 (t, |
| | | | | | J = 7.5 Hz, |
| | | | | | 3H). |
| 1-34 | S | 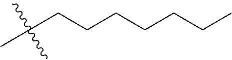 [SMILES: *CCCCCCC] | Me | H | ¹H NMR (500 MHz, DMSO-d6) 12.37 (s, |
| | | | | | 1H), 7.87 |
| | | | | | (dd, J = 8.0, |
| | | | | | 7.0 Hz, 1H), |
| | | | | | 7.60 (d, J = |
| | | | | | 8.0 Hz, 1H), |
| | | | | | 3.28 (t, J = |
| | | | | | 7.5 Hz, 2H), |
| | | | | | 2.50 (s, 3H), |
| | | | | | 1.76 -1.51 |
| | | | | | (m, 10H), |
| | | | | | 1.08 (t, J = |
| | | | | | 7.5 Hz, 3H). |
| 1-35 | SO | 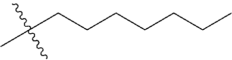 [SMILES: *CCCCCCC] | Me | H | ¹H NMR (500 MHz, DMSO-d6) 12.38 (s, |
| | | | | | 1H), 8.07 |
| | | | | | (dd, J = 8.0, |
| | | | | | 7.0 Hz, 1H), |
| | | | | | 3.48 (t, J = |
| | | | | | 7.5 Hz, 2H), |
| | | | | | 2.50 (s, 3H), |
| | | | | | 1.85-1.53 |
| | | | | | (m, 10H), |
| | | | | | 1.10 (t, J = |
| | | | | | 7.5 Hz, 3H). |
| 1-36 | SO₂ | 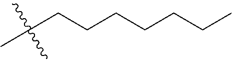 [SMILES: *CCCCCCC] | Me | H | ¹H NMR (500 MHz, DMSO-d6) 12.39 (s, |
| | | | | | 1H), 8.27 |
| | | | | | (dd, J = 8.0, |
| | | | | | 7.0 Hz, 1H), |
| | | | | | 3.68 (t, J = |
| | | | | | 7.5 Hz, 2H), |
| | | | | | 2.50 (s, 3H), |
| | | | | | 1.94-1.55 |
| | | | | | (m, 10H), |
| | | | | | 1.12 (t, J = |
| | | | | | 7.5 Hz, 3H). |
| 1-37 | S | 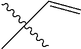 [SMILES: *C=C] | Me | H | ¹H NMR (500 MHz, DMSO-d6) |
| | | | | | 12.12 (s, |
| | | | | | 1H), 8.10 |
| | | | | | (dd, J = 8.0, |
| | | | | | 7.0 Hz, 1H), |
| | | | | | 7.99 (d, J = |
| | | | | | 8.0 Hz, 1H), |
| | | | | | 6.46-6.35 (m, |
| | | | | | 1H), 5.43- |
| | | | | | 5.32 (m, 2H), |
| | | | | | 2.50 (s, 3H). |
| 1-38 | SO | 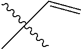 [SMILES: *C=C] | Me | H | |
| 1-39 | SO₂ | 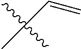 [SMILES: *C=C] | Me | H | |
| 1-40 | S | 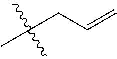 [SMILES: *CC=C] | Me | H | ¹H NMR (500 MHz, DMSO-d6) 12.40 (s, |
| | | | | | 1H), 7.95 |
| | | | | | (dd, J = 8.0, |
| | | | | | 7.0 Hz, 1H), |
| | | | | | 7.80 (d, J = |
| | | | | | 8.0 Hz, 1H), |
| | | | | | 6.01 ?+05.92 |
| | | | | | (m, 1H), 5.21 |
| | | | | | ?+05.12 (m, |
| | | | | | 2H), 3.82 ?+0 |
| | | | | | 3.76 (m, 2H), |
| | | | | | 2.50 (s, 3H) |
| 1-41 | S | 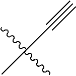 [SMILES: *C#C] | Me | H | ¹H NMR (500 MHz, DMSO-d6) 12.23 (s, 1H), 8.10 |
| | | | | | (dd, J = 8.0, |
| | | | | | 7.0 Hz, 1H), |
| | | | | | 7.99 (d, J = |
| | | | | | 8.0 Hz, 1H), |
| | | | | | 3.12 (s, 1H), |
| | | | | | 2.50 (s, 3H). |
| 1-42 | S | 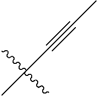 [SMILES: *C#CC] | Me | H | |
| 1-43 | S | 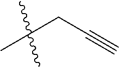 [SMILES: *CC#C] | Me | H | ¹H NMR (500 MHz, DMSO-d6) 12.40 (s, |
| | | | | | 1H), 8.02 |
| | | | | | (dd, J = 8.0, |
| | | | | | 7.0 Hz, 1H), |
| | | | | | 7.86 (d, J = |
| | | | | | 8.0 Hz, 1H), |
| | | | | | 3.35 (s, 2H), |
| | | | | | 2.95 (s, 1H), |
| | | | | | 2.49 (s, 3H) |
| 1-44 | S | 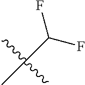 [SMILES: *C(F)F] | Me | H | ¹H NMR (500 MHz, DMSO-d6) 12.33 (s, 1H), 8.13 |
| | | | | | 7.0 Hz, 1H), |
| | | | | | 8.00 (d, J = |
| | | | | | 8.0 Hz, 1H), |
| | | | | | 6.65 (t, J = |
| | | | | | 73..5 Hz, |
| | | | | | 1H), 2.50 (s, |
| | | | | | 3H). |
| 1-45 | SO | 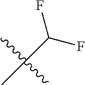 [SMILES: *C(F)F] | Me | H | |
| 1-46 | SO₂ | 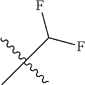 [SMILES: *C(F)F] | Me | H | |
| 1-47 | S | 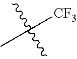 [SMILES: *C(F)(F)F] | Me | H | ¹H NMR (500 MHz, DMSO-d6) 12.48 (s, |
| | | | | | 1H), 8.18 |
| | | | | | (dd, J = 8.0, |
| | | | | | 7.0 Hz, 1H), |
| | | | | | 8.07 (d, J = |
| | | | | | 8.0 Hz, 1H), |
| | | | | | 2.51 (s, 3H) |
| 1-48 | S | 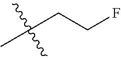 [SMILES: *CCF] | Me | H | ¹H NMR (500 MHz, DMSO-d6) 12.40 (s, |
| | | | | | 1H), 7.95 |
| | | | | | (dd, J = 8.0, |
| | | | | | 7.0 Hz, 1H), |
| | | | | | 8.0 Hz, 1H), |
| | | | | | 4.53-4.40 |
| | | | | | (m, 2H), 3.11- |
| | | | | | 3.00 (m, |
| | | | | | 2H), 2.50 (s, |
| | | | | | 3H) |
| 1-49 | S | 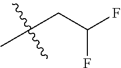 [SMILES: *CC(F)F] | Me | H | ¹H NMR (500 MHz, DMSO-d6) 12.41 (s, |
| | | | | | 1H), 7.97 |
| | | | | | (dd, J = 8.0, |
| | | | | | 7.0 Hz, 1H) |
| | | | | | 8.0 Hz, 1H), |
| | | | | | 5.59-5.41 |
| | | | | | (m, 1H), 3.21- |
| | | | | | 3.10 (m, |
| | | | | | 2H), 2.50 (s, |
| | | | | | 3H) |
| 1-50 | S | 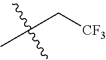 [SMILES: *CC(F)(F)F] | Me | H | ¹H NMR (500 MHz, DMSO-d6) 12.43 (s, |
| | | | | | 1H), 7.98 |
| | | | | | (dd, J = 8.0, |
| | | | | | 7.0 Hz, 1H), |
| | | | | | 7.86 (d, J = |
| | | | | | 8.0 Hz, 1H), |
| | | | | | 3.45-3, 38 |
| | | | | | (m, 2H), 2.51 |
| | | | | | (s, 3H) |
| 1-51 | S | 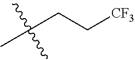 [SMILES: *CCC(F)(F)F] | Me | H | ¹H NMR (500 MHz, DMSO-d₆) 12.40 (s, |
| | | | | | 1H), 7.95 |
| | | | | | (dd, J = 8.0, |
| | | | | | 7.0 Hz, 1H), |
| | | | | | 7.83 (d, J = |
| | | | | | 8.0 Hz, 1H), |
| | | | | | 2.93-2.80 |
| | | | | | (m, 2H), 2.21- |
| | | | | | 2.10 (m, |
| | | | | | 2H), 2.50 (s, |
| | | | | | 3H) |
| 1-52 | SO | 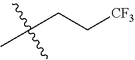 [SMILES: *CCC(F)(F)F] | Me | H | ¹H NMR (500 MHz, DMSO-d6) 12.42 (s, |
| | | | | | 1H), 8.15 |
| | | | | | (dd, J = 8.0, |
| | | | | | 7.0 Hz, 1H), |
| | | | | | 8.03 (d, J = |
| | | | | | 8.0 Hz, 1H), |
| | | | | | 3.13-2.96 |
| | | | | | (m, 2H), 2.28- |
| | | | | | 2.13 (m, |
| | | | | | 2H), 2.50 (s, |
| | | | | | 3H) |
| 1-53 | SO₂ | 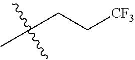 [SMILES: *CCC(F)(F)F] | Me | H | ¹H NMR (500 MHz, DMSO-d6) 12.43 (s, |
| | | | | | 1H), 8.34 |
| | | | | | (dd, J = 8.0, |
| | | | | | 7.0 Hz, 1H), |
| | | | | | 8.21 (d, J = |
| | | | | | 8.0 Hz, 1H), |
| | | | | | 3.13-3.00 |
| | | | | | (m, 2H), 2.31- |
| | | | | | 2.70 (m, |
| | | | | | 2H), 2.50 (s, |
| | | | | | 3H) |
| 1-54 | S | 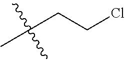 [SMILES: *CCCl] | Me | H | ¹H NMR (500 MHz, DMSO-d6) 12.41 (s, |
| | | | | | 1H), 7.93 |
| | | | | | (dd, J = 8.0, |
| | | | | | 7.0 Hz, 1H), |
| | | | | | 7.81 (d, J = |
| | | | | | 8.0 Hz, 1H), |
| | | | | | 3.73 (t, J = |
| | | | | | 8.0 Hz, 2H), |
| | | | | | 3.11 (t, J = |
| | | | | | 8.0 Hz, 2H), |
| | | | | | 2.50 (s, 3H) |
| 1-55 | S | 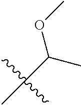 [SMILES: *C(C)OC] | Me | H | |
| 1-56 | S | 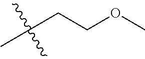 [SMILES: *CCOC] | Me | H | ¹H NMR (500 MHz, DMSO-d6) 12.41 (s, |
| | | | | | 1H), 7.93 |
| | | | | | (dd, J = 8.0, |
| | | | | | 7.0 Hz, 1H), |
| | | | | | 7.81 (d, J = |
| | | | | | 8.0 Hz, 1H), |
| | | | | | 3.78 (t, J = |
| | | | | | 8.0 Hz, 2H), |
| | | | | | 3.31 (t, J = |
| | | | | | 8.0 Hz, 2H), |
| | | | | | 3.21 (s, 3H), |
| | | | | | 2.50 (s, 3H) |
| 1-57 | SO | 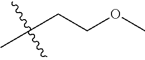 [SMILES: *CCOC] | Me | H | ¹H NMR (500 MHz, DMSO-d6) 12.42 (s, |
| | | | | | 1H), 8.13 |
| | | | | | (dd, J = 8.0, |
| | | | | | 7.0 Hz, 1H), |
| | | | | | 8.01 (d, J = |
| | | | | | 8.0 Hz, 1H), |
| | | | | | 3.98 (t, J = |
| | | | | | 8.0 Hz, 2H), |
| | | | | | 3.39 (t, J = |
| | | | | | 8.0 Hz, 2H), |
| | | | | | 3.31 (s, 3H), |
| | | | | | 2.50 (s, 3H) |
| 1-58 | SO₂ | 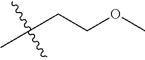 [SMILES: *CCOC] | Me | H | ¹H NMR (500 MHz, DMSO-d6) 12.44 (s, |
| | | | | | 1H), 8.33 |
| | | | | | (dd, J = 8.0, |
| | | | | | 7.0 Hz, 1H), |
| | | | | | 8.23 (d, J = |
| | | | | | 8.0 Hz, 1H), |
| | | | | | 4.17 (t, J = |
| | | | | | 8.0 Hz, 2H), |
| | | | | | 3.48 (t, J = |
| | | | | | 8.0 Hz, 2H), |
| | | | | | 3.33 (s, 3H), |
| | | | | | 2.50 (s, 3H) |
| 1-59 | S | 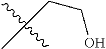 [SMILES: *CCO] | Me | H | ¹H NMR (500 MHz, DMSO-d6) |
| | | | | | 12.41 (s, |
| | | | | | 1H), 7.93 |
| | | | | | (dd, J = 8.0, |
| | | | | | 7.0 Hz, 1H), |
| | | | | | 7.81 (d, J = |
| | | | | | 8.0 Hz, 1H), |
| | | | | | 3.78 (t, J = |
| | | | | | 8.0 Hz, 2H), |
| | | | | | 3.31 (t, J = |
| | | | | | 8.0 Hz, 2H), |
| | | | | | 2.50 (s, 3H) |
| 1-60 | S | 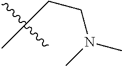 [SMILES: *CCN(C)C] | Me | H | ¹H NMR (500 MHz, DMSO-d6) 12.40 (s, |
| | | | | | 1H), 7.92 |
| | | | | | (dd, J = 8.0, |
| | | | | | 7.80 (d, J = |
| | | | | | 3.38 (t, J = |
| | | | | | 8.0 Hz, 2H), |
| | | | | | 2.61 (t, J = |
| | | | | | 8.0 Hz, 2H), |
| | | | | | 2.50 (s, 3H), |
| | | | | | 2, 21 (s, 6H) |
| 1-61 | SO | 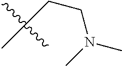 [SMILES: *CCN(C)C] | Me | H | ¹H NMR (500 MHz, DMSO-d6) 12.42 (s, |
| | | | | | 1H), 8.12 |
| | | | | | (dd, J = 8.0, |
| | | | | | 7.0 Hz, 1H), |
| | | | | | 8.00 (d, J = |
| | | | | | 8.0 Hz, 1H), |
| | | | | | 3.58 (t, J = |
| | | | | | 8.0 Hz, 2H), |
| | | | | | 2.72 (t, J = |
| | | | | | 8.0 Hz, 2H), |
| | | | | | 2.50 (s, 3H), |
| | | | | | 2.23 (s, 6H) |
| 1-62 | SO₂ | 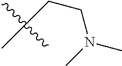 [SMILES: *CCN(C)C] | Me | H | ¹H NMR (500 MHz, DMSO-d6) 12.44 (s, |
| | | | | | 1H), 8.32 |
| | | | | | (dd, J = 8.0, |
| | | | | | 7.0 Hz, 1H), |
| | | | | | 8.20 (d, J = |
| | | | | | 8.0 Hz, 1H), |
| | | | | | 3.77 (t, J = |
| | | | | | 8.0 Hz, 2H), |
| | | | | | 2.83 (t, J = |
| | | | | | 8.0 Hz, 2H), |
| | | | | | 2.50 (s, 3H), |
| | | | | | 2, 24 (s, 6H) |
| 1-63 | S | 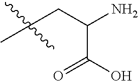 [SMILES: *CC(N)C(=O)O] | Me | H | ¹H NMR (500 MHz, DMSO-d6) 7.91 (dd, J = 8.0, 7.0 Hz, |
| | | | | | 1H), 7.80 (d, |
| | | | | | J = 8.0 Hz, |
| | | | | | 1H), 3.80- |
| | | | | | 3.69 (m, 1H), |
| | | | | | 3.11-3.03 |
| | | | | | (m, 2H), 2.50 |
| | | | | | (s, 3H) |
| 1-64 | S | 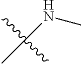 [SMILES: *NC] | Me | H | |
| 1-65 | SO₂ | 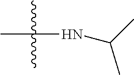 [SMILES: *NC(C)C] | Me | H | |
| 1-66 | SO | 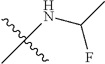 [SMILES: *NC(C)F] | Me | H | |
| 1-67 | S | 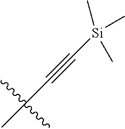 [SMILES: *C#C[Si](C)(C)C] | Me | H | |
| 1-68 | S | 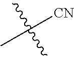 [SMILES: *C#N] | Me | H | ¹H NMR (500 MHz, DMSO-d6) 12.49 (s, |
| | | | | | 1H), 8.19 |
| | | | | | (dd, J = 8.0, |
| | | | | | 7.0 Hz, 1H), |
| | | | | | 8.09 (d, J = |
| | | | | | 8.0 Hz, 1H), |
| | | | | | 2.51 (s, 3H) |
| 1-69 | S | 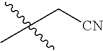 [SMILES: *CC#N] | Me | H | ¹H NMR (500 MHz, DMSO-d6) |
| | | | | | 12.43 (s, |
| | | | | | 1H), 8.05 |
| | | | | | (dd, J = 8.0, |
| | | | | | 7.0 Hz, 1H), |
| | | | | | 7.89 (d, J = |
| | | | | | 8.0 Hz, 1H), |
| | | | | | 4.22 (s, 2H), |
| | | | | | 2.50 (s, 3H) |
| 1-70 | S | 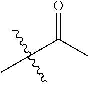 [SMILES: *C(C)=O] | H | | ¹H NMR (500 MHz, DMSO-d6) 12.43 (s, 1H), 8.12 |
| | | | | | (dd, J = 8.0, |
| | | | | | 7.0 Hz, 1H), |
| | | | | | 8.06 (d, J = |
| | | | | | 8.0 Hz, 1H), |
| | | | | | 2.51 (s, 3H), |
| | | | | | 2.34 (s, 3H) |
| 1-71 | S | 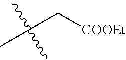 [SMILES: *CC(=O)OCC] | Me | H | ¹H NMR (500 MHz, DMSO-d6) 12.43 (s, |
| | | | | | 1H), 7.97 |
| | | | | | (dd, J = 8.0, |
| | | | | | 7.0 Hz, 1H), |
| | | | | | 7.85 (d, J = |
| | | | | | 8.0 Hz, 1H), |
| | | | | | 4.17 (s, 2H), |
| | | | | | 4.10 (q, J = |
| | | | | | 8.0 Hz, 2H), |
| | | | | | 2.51 (s, 3H), |
| | | | | | 1.29 (t, J = |
| | | | | | 8.0 Hz, 3H) |
| 1-72 | SO | 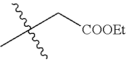 [SMILES: *CC(=O)OCC] | Me | H | ¹H NMR (500 MHz, DMSO-d6) 12.45 (s, |
| | | | | | 1H), 8.17 |
| | | | | | (dd, J = 8.0, |
| | | | | | 7.0 Hz, 1H), |
| | | | | | 8.05 (d, J = |
| | | | | | 8.0 Hz, 1H), |
| | | | | | 4.37 (s, 2H), |
| | | | | | 4.13 (q, J = |
| | | | | | 8.0 Hz, 2H), |
| | | | | | 2.51 (s, 3H), |
| | | | | | 1.30 (t, J = |
| | | | | | 8.0 Hz, 3H) |
| 1-73 | SO₂ | 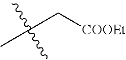 [SMILES: *CC(=O)OCC] | Me | H | ¹H NMR (500 MHz, DMSO-d6) 12.46 (s, |
| | | | | | 1H), 8.37 |
| | | | | | (dd, J = 8.0, |
| | | | | | 7.0 Hz, 1H), |
| | | | | | 8.25 (d, J = |
| | | | | | 8.0 Hz, 1H), |
| | | | | | 4.57 (s, 2H), |
| | | | | | 4.19 (q, J = |
| | | | | | 8.0 Hz, 2H), |
| | | | | | 2.51 (s, 3H), |
| | | | | | 1.33 (t, J = |
| | | | | | 8.0 Hz, 3H) |
| 1-74 | S | 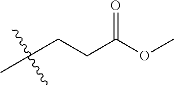 [SMILES: *CCC(=O)OC] | Me | H | ¹H NMR (500 MHz, DMSO-d6) 12.40 (s, 1H), 7.90 |
| | | | | | (dd, J = 8.0, |
| | | | | | 7.0 Hz, 1H), |
| | | | | | 7.80 (d, J = |
| | | | | | 8.0 Hz, |
| | | | | | 1H), 3.69 (s, |
| | | | | | 3H), 3.57 (t, |
| | | | | | J = 8.5 Hz, |
| | | | | | 2H), 2, 67 (t, |
| | | | | | J = 8.5 Hz, |
| | | | | | 2H), 2.50 (s, |
| | | | | | 3H) |
| | | | | | H |
| 1-75 | S | 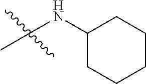 [SMILES: *NC1CCCCC1] | Me | H | |
| 1-76 | S | 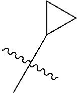 [SMILES: *C1CC1] | Me | H | ¹H NMR (500 MHz, DMSO-d6) 12.41 (s, 1H), 7.90 (dd, J = 8.0, |
| | | | | | 8.0 Hz, 1H), |
| | | | | | 2.50 (s, 3H), |
| | | | | | 1.67-1.58 |
| | | | | | (m, 1H), 1.17- |
| | | | | | 0.92 (m, |
| | | | | | 4H) |
| 1-77 | SO | 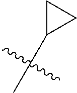 [SMILES: *C1CC1] | Me | H | ¹H NMR (500 MHz, DMSO-d6) 12.51 (s, 1H), 8.10 (dd, J = 8.0, |
| | | | | | 8.0 Hz, 1H), |
| | | | | | 2.50 (s, 3H), |
| | | | | | 1.78-1.69 |
| | | | | | (m, 1H), 1.20- |
| | | | | | 0.95 (m, |
| | | | | | 4H) |
| 1-78 | SO₂ | 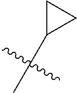 [SMILES: *C1CC1] | Me | H | ¹H NMR (500 MHz, DMSO-d6) 12.55 (s, 1H), 8.29 (dd, J = 8.0, |
| | | | | | 7.0 Hz, 1H), |
| | | | | | 8.17 (d, J = |
| | | | | | 8.0 Hz, 1H), |
| | | | | | 2.50 (s, 3H), |
| | | | | | 1.98-1.89 |
| | | | | | (m, 1H), 1.25- |
| | | | | | 0.99 (m, |
| | | | | | 4H) |
| 1-79 | S | 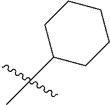 [SMILES: *C1CCCCC1] | Me | H | ¹H NMR (500 MHz, DMSO-d₆) δ 12.37 (s, 1H), 7.86- 7.83 (m, |
| | | | | | 1H), 7.76 (d, |
| | | | | | J = 8.0 Hz, |
| | | | | | 1H), 2.46 (s, |
| | | | | | 3H), 1.79 (d, |
| | | | | | J = 11.5 Hz, |
| | | | | | 2H), 1.70- |
| | | | | | 1.63 (m, |
| | | | | | 2H), 1.56- |
| | | | | | 1.49 (m, |
| | | | | | 1H), 1.35- |
| | | | | | 1.15(m, 6H) |
| 1-80 | SO₂ | 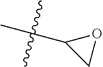 [SMILES: *C1CO1] | Me | H | ¹H NMR (500 MHz, DMSO-d6) 12.43 (s, |
| | | | | | 1H), 8.19 |
| | | | | | (dd, J = 8.0, |
| | | | | | 7.0 Hz, 1H), |
| | | | | | 8.07 (d, J = |
| | | | | | 8.0 Hz, 1H), |
| | | | | | 3.92-3.83 (m, |
| | | | | | 1H), 3.11- |
| | | | | | 2.99 (m, 2H), |
| | | | | | 2.50 (s, 3H). |
| 1-81 | SO₂ | 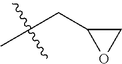 [SMILES: *CC1CO1] | Me | H | ¹H NMR (500 MHz, DMSO-d6) 12.47 (s, |
| | | | | | 1H), 8.22 |
| | | | | | (dd, J = 8.0, |
| | | | | | 7.0 Hz, 1H), |
| | | | | | 7.96 (d, J = |
| | | | | | 8.0 Hz, 1H), |
| | | | | | 3.55-3.48 |
| | | | | | (m, 2H), 2.49 |
| | | | | | (s, 3H), 2.41- |
| | | | | | 2.32 (m, |
| | | | | | 3H) |
| 1-82 | SO | 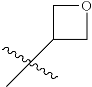 [SMILES: *C1COC1] | Me | H | ¹H NMR (500 MHz, DMSO-d6) 12.41 (s, 1H), 8.13 |
| | | | | | (dd, J = 8.0, |
| | | | | | 7.0 Hz, 1H), |
| | | | | | 8.01 (d, J = |
| | | | | | 8.0 Hz, 1H), |
| | | | | | 5.11-.5.01 |
| | | | | | (m, 4H), 4.25- |
| | | | | | 4.16 (m, |
| | | | | | 1H), 2.50 (s, |
| | | | | | 3H) |
| 1-83 | S | 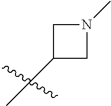 [SMILES: *C1CN(C)C1] | Me | H | |
| 1-84 | S | 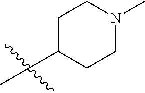 [SMILES: *C1CCN(C)CC1] | Me | H | |
| 1-85 | SO₂ | 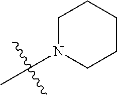 [SMILES: *N1CCCCC1] | Me | H | ¹H NMR (500 MHz, DMSO-d6) 12.47 (s, 1H), 8.22 (dd, J = 8.0, |
| | | | | | 7.0 Hz 1H), |
| | | | | | 7.96 (d, J = |
| | | | | | 8.0 Hz, 1H), |
| | | | | | 3.15-3.01 |
| | | | | | (m, 4H), 2.49 |
| | | | | | (s, 3H), 1.49- |
| | | | | | 1.42 (m, |
| | | | | | 6H) |
| 1-86 | S | 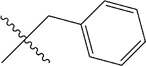 [SMILES: *Cc1ccccc1] | Me | H | ¹H NMR (500 MHz, DMSO-d₆) δ 12.40 (s, |
| | | | | | 1H), 7.85 (t, |
| | | | | | d, J = |
| | | | | | 7.5 Hz, 1H), |
| | | | | | 7.69 (d, J = |
| | | | | | 8.0 Hz, |
| | | | | | 1H), 7.25- |
| | | | | | 7.16 (m, |
| | | | | | 5H), 4.18 (s, |
| | | | | | 2H), 2.47 (s, |
| | | | | | 3H). |
| 1-87 | S | 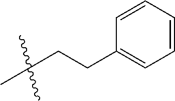 [SMILES: *CCc1ccccc1] | Me | H | ¹H NMR (500 MHz, DMSO-d6) 12.40 (s, 1H), 7.90 7.0 Hz, 1H), |
| | | | | | 7.80 (d, J = |
| | | | | | 8.0 Hz, 1H), |
| | | | | | 7.27-7.17 |
| | | | | | (m, 5H), 3.17 |
| | | | | | (t, J = 8.5 Hz, |
| | | | | | 2H), 3.07 (t, |
| | | | | | J = 8.5 Hz, |
| | | | | | 2H), 2.50 (s, |
| | | | | | 3H) |
| 1-88 | S | 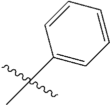 [SMILES: *c1ccccc1] | Me | H | ¹H NMR (500 MHz, DMSO-d6) 12.33 (s, 1H), 8.03 (dd, J = 8.0, 7.0 Hz, 1H), |
| | | | | | 7.93 (d, J = |
| | | | | | 8.0 Hz, 1H), |
| | | | | | 7.47-7.32 (m, |
| | | | | | 5H), 2.51 (s, |
| | | | | | 3H). |
| 1-89 | S | 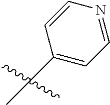 [SMILES: *c1ccncc1] | Me | H | ¹H NMR (500 MHz, DMSO-d6) 12.39 (s, 1H), 8.82 (d, J = 5.5 Hz, 2H), 8.09 (dd, |
| | | | | | J = 8.0, 7.0 |
| | | | | | Hz, 1H), 7.96 |
| | | | | | (d, J = 8.0 |
| | | | | | Hz, 1H), 7.45 |
| | | | | | (d, J = 5.5 |
| | | | | | Hz, 2H), 2.51 |
| | | | | | (s, 3H). |
| 1-90 | S | 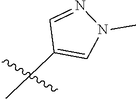 [SMILES: *c1cnn(C)c1] | Me | H | ¹H NMR (500 MHz, DMSO-d6) 12.31 (s, 1H), 8.12 (s, 1H), 8.09 (dd, |
| | | | | | J = 8.0, 7.0 |
| | | | | | Hz, 1H), 7.96 |
| | | | | | (d, J = 8.0 |
| | | | | | Hz, 1H), 7.85 |
| | | | | | (s, 1H), 3.81 |
| | | | | | (s, 3H), 2.51 |
| | | | | | (s, 3H). |
| 1-91 | S | 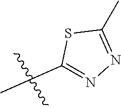 [SMILES: *c1nnc(C)s1] | Me | H | ¹H NMR (500 MHz, DMSO-d6) 12.47 (s, 1H), 8.22 (dd, J = 8.0, 7.0 Hz, 1H), |
| | | | | | 7.96 (d, J = |
| | | | | | 8.0 Hz, 1H), |
| | | | | | 2.65 (s, 3H), |
| | | | | | 2.49 (s, 3H) |
| 1-92 | S | 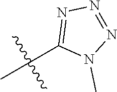 [SMILES: *c1nnnn1C] | Me | H | ¹H NMR (500 MHz, DMSO-d6) 12.41 (s, 1H), 8.13 (dd, J = 8.0, 7.0 |
| | | | | | Hz, 1H), 7.99 |
| | | | | | (d, J = 8.0 |
| | | | | | Hz, 1H), 3.91 |
| | | | | | (s, 3H), 2.51 |
| | | | | | (s, 3H). |
| 1-93 | S | 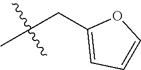 [SMILES: *Cc1ccco1] | Me | H | ¹H NMR (500 MHz, DMSO-d6) 12.41 (s, 1H), 7.95 |
| | | | | | (dd, J = 8.0, |
| | | | | | 7.0 Hz, 1H), |
| | | | | | 7.83 (d, J = |
| | | | | | 8.0 Hz, 1H), |
| | | | | | 7.50 (d, J = |
| | | | | | 2.0 Hz, 1H), |
| | | | | | 6.31 (dd, J = |
| | | | | | 2.5, 2.0 Hz, |
| | | | | | 1H), 6.15 (d, |
| | | | | | J = 2.5 Hz, |
| | | | | | 1H), 3.97 (s, |
| | | | | | 2H), 2.50 (s, |
| | | | | | 3H) |
| 1-94 | S | Et | Me | Me | |
| 1-95 | S | Et | Me | Et | |
| 1-96 | S | Et | Me | 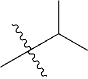 [SMILES: *C(C)C] | |
| 1-97 | S | Et | Me | 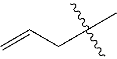 [SMILES: *CC=C] | |
| 1-98 | S | Et | Me | 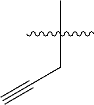 [SMILES: *CC#C] | |
| 1-99 | S | Et | Me | 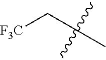 [SMILES: *CC(F)(F)F] | |
| 1-100 | S | Et | Me | 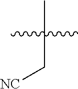 [SMILES: *CC#N] | |
| 1-101 | S | Et | Me | 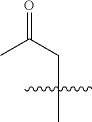 [SMILES: *CC(C)=O] | |
| 1-102 | S | Et | Me | 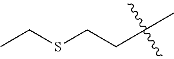 [SMILES: *CCSCC] | |
| 1-103 | S | Et | Me | 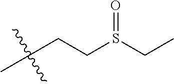 [SMILES: *CCS(=O)CC] | |
| 1-104 | S | Et | Me | 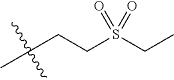 [SMILES: *CCS(=O)(=O)CC] | |
| 1-105 | S | Et | Me | 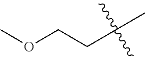 [SMILES: *CCOC] | |
| 1-106 | S | Et | Me | 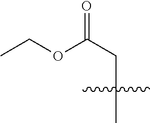 [SMILES: *CC(=O)OCC] | |
| 1-107 | S | Et | Me | 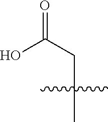 [SMILES: *CC(=O)O] | |
| 1-108 | S | Et | Me | 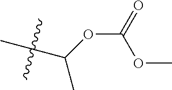 [SMILES: *C(C)OC(=O)OC] | |
| 1-109 | S | Et | Me | 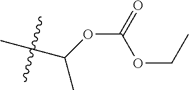 [SMILES: *C(C)OC(=O)OCC] | ¹H NMR (500 MHz, DMSO-d6) 7.98 (dd, J = 8.0, 7.0 Hz, 1H), 7.88 (d, |
| | | | | | J = 8.0 Hz, |
| | | | | | 1H), 6, 32 (q, |
| | | | | | J = 7.5 Hz, |
| | | | | | 1H), 4.11 (q, |
| | | | | | J = 8.5, 2H), |
| | | | | | 2.98 (q, J = |
| | | | | | 7.5 Hz, 2H) |
| | | | | | 2.51 (s, 3H), |
| | | | | | 1.56 (d, J = |
| | | | | | 7.5 Hz, 3H), |
| | | | | | 1.32 (t, J = |
| | | | | | 8.5 Hz, 3H), |
| | | | | | 1.15 (t, J = |
| | | | | | 7.5 Hz, 3H). |
| 1-110 | S | Et | Me | 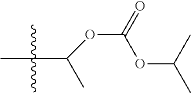 [SMILES: *C(C)OC(=O)OC(C)C] | |
| 1-111 | S | Et | Me | 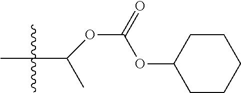 [SMILES: *C(C)OC(=O)OC1CCCCC1] | |
| 1-112 | S | Et | Me | 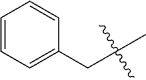 [SMILES: *Cc1ccccc1] | ¹H NMR (500 MHz, DMSO-d6) 7.99 (dd, J = 8.0, 7.0 Hz, |
| | | | | | 1H), 7.85 (d, |
| | | | | | J = 8.0 Hz, |
| | | | | | 1H), 7.31- |
| | | | | | 7.20 (m, 5H), |
| | | | | | 4.97 (s, 2H), |
| | | | | | 2.99 (q, J = |
| | | | | | 7.5 Hz, 2H) |
| | | | | | 2.51 (s, 3H), |
| | | | | | 1.15 (t, J = |
| | | | | | 7.5 Hz, 3H). |
| 1-113 | S | Et | Me | 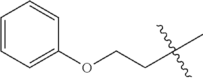 [SMILES: *CCOc1ccccc1] | |
| 1-114 | S | Et | Me | 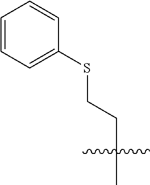 [SMILES: *CCSc1ccccc1] | |
| 1-115 | S | Et | Me | 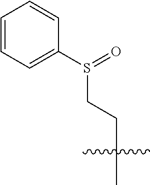 [SMILES: *CCS(=O)c1ccccc1] | |
| 1-116 | S | Et | Me | 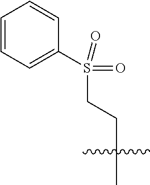 [SMILES: *CCS(=O)(=O)c1ccccc1] | |
| 1-117 | S | Et | Me | 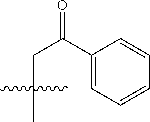 [SMILES: *CC(=O)c1ccccc1] | |
| 1-118 | S | Et | Me | 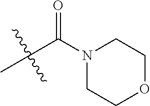 [SMILES: *C(=O)N1CCOCC1] | ¹H NMR (500 MHz, DMSO-d6) 8.07 (dd, J = 8.0, 7.0 Hz, 1H), 7.89 (d, J = 8.0 Hz, |
| | | | | | 1H), 3.61- |
| | | | | | 3.52 (m, 4H), |
| | | | | | 3.31-3.23 |
| | | | | | (m, 4H), 2.98 |
| | | | | | (q, J = 7.5 |
| | | | | | Hz, 2H) 2.53 |
| | | | | | (s, 3H), 1.15 |
| | | | | | (t, J = 7.5 Hz, |
| | | | | | 3H). |
| 1-119 | S | Et | Me | 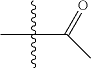 [SMILES: *C(C)=O] | ¹H NMR (500 MHz, DMSO-d6) 8.08 (dd, J = |
| | | | | | 8.0, 7.0 Hz, |
| | | | | | 1H), 7.89 (d, |
| | | | | | J = 8.0 Hz, |
| | | | | | 1H), 2.98 (q, |
| | | | | | J = 7.5 Hz, |
| | | | | | 2H) 2.51 (s, |
| | | | | | 3H), 2.27 (s, |
| | | | | | 3H), 1.15 (t, |
| | | | | | J = 7.5 Hz, |
| | | | | | 3H). |
| 1-120 | S | Et | Me | 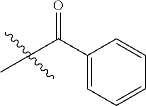 [SMILES: *C(=O)c1ccccc1] | |
| 1-121 | S | Et | Me | 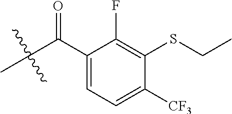 [SMILES: *C(=O)c1ccc(C(F)(F)F)c(SCC)c1F] | |
| 1-122 | S | Et | Me | 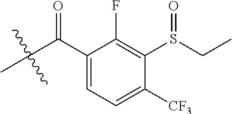 [SMILES: *C(=O)c1ccc(C(F)(F)F)c(S(=O)CC)c1F] | |
| 1-123 | S | Et | Me | 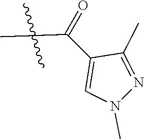 [SMILES: *C(=O)c1cn(C)nc1C] | |
| 1-124 | S | Et | Me | 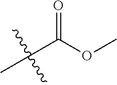 [SMILES: *C(=O)OC] | |
| 1-125 | S | Et | Me | 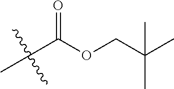 [SMILES: *C(=O)OCC(C)(C)C] | |
| 1-126 | O | Me | Me | | ¹H NMR |
| | | | | | (500 MHz, |
| | | | | | DMSO-d6) |
| | | | | | 7.60-7.48 (m, |
| | | | | | 2H), 3.96 (s, |
| | | | | | 3H), 2.50 (s, |
| | | | | | 3H). |
| 1-127 | O | Et | Me | H | ¹H NMR |
| | | | | | (500 MHz, |
| | | | | | DMSO-d6) |
| | | | | | 12.12 (s, |
| | | | | | 1H), 7.61- |
| | | | | | 7.50 (m, 2H), |
| | | | | | 4.18 (q, J = |
| | | | | | 7.5 Hz, 2H), |
| | | | | | 2.50 (s, 3H), |
| | | | | | 1.20 (t, J = |
| | | | | | 7.5 Hz, 3H). |
| 1-128 | O | 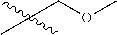 [SMILES: *COC] | Me | H | |
| 1-129 | O | 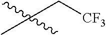 [SMILES: *CC(F)(F)F] | Me | H | ¹H NMR (500 MHz, |
| | | | | | DMSO-d6) |
| | | | | | 12.41 (s, |
| | | | | | 1H), 7.99- |
| | | | | | 7.88 (m, 2H), |
| | | | | | 4.51 (q, J = |
| | | | | | 13.5 Hz, 2H), |
| | | | | | 2.50 (s, 3H) |
| 1-130 | O | 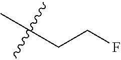 [SMILES: *CCF] | Me | H | ¹H NMR (500 MHz, DMSO-d6) 12.12 (s, |
| | | | | | 1H), 7.61- |
| | | | | | 7.50 (m, 2H), |
| | | | | | 4.53-4.20 |
| | | | | | (m, 4H), 2.50 |
| | | | | | (s, 3H) |
| 1-131 | NH | Me | Me | H | |
| 1-132 | NMe | Me | Me | H | ¹H NMR |
| | | | | | (500 MHz, |
| | | | | | DMSO-d6) |
| | | | | | 12.12 (s, |
| | | | | | 1H), 7.61- |
| | | | | | 7.50 (m, 2H), |
| | | | | | 3.08 (s, 6H), |
| | | | | | 2.50 (s, 3H). |
| 1-133 | NMe | Et | Me | H | |
| 1-134 | -X-Y represents 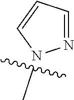 | | Me | H | ¹H NMR (500 MHz, DMSO-d6) 12.32 (s, 1H), 8.10 (dd, J = 8.0, 7.0 Hz, 1H), 7.99 (d, J = |
| | | | | | 8.0 Hz, 1H), |
| | | | | | 3.73-3.66 (m, |
| | | | | | 2H), 2.50 (s, |
| | | | | | 3H), 2.45- |
| | | | | | 2.38 (m, 2H), |
| | | | | | 1.98-1.89 (m, |
| | | | | | 2H). |
| 1-135 | -X-Y represents 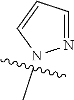 | | Me | H | ¹H NMR (500 MHz, DMSO-d6) 12.43 (s, 1H), 7.99- 7.82 (m, 2H), 7.52-7.40 (m, 3H), 2.50 |
| | | | | | (s, 3H) |
| 1-136 | S | 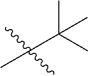 [SMILES: *C(C)(C)C] | Me | H | ¹H NMR (500 MHz, DMSO-d6) 12.35 (s, 1H), 7.94 |
| | | | | | (dd, J = 8.0, |
| | | | | | 7.0 Hz, 1H), |
| | | | | | 7.59 (d, J = |
| | | | | | 8.0 Hz, 1H), |
| | | | | | 2.50 (s, 3H), |
| | | | | | 1.28 (s, 9H) |
| 1-137 | SO | 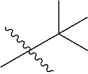 [SMILES: *C(C)(C)C] | Me | H | ¹H NMR (500 MHz, DMSO-d6) 12.37 (s, 1H), 8.08 |
| | | | | | (dd, J = 8.0, |
| | | | | | 7.0 Hz, 1H), |
| | | | | | 7.72 (d, J = |
| | | | | | 8.0 Hz, 1H), |
| | | | | | 2.50 (s, 3H), |
| | | | | | 1.36 (s, 9H). |
| 1-138 | SO₂ | 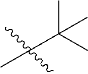 [SMILES: *C(C)(C)C] | Me | H | ¹H NMR (500 MHz, DMSO-d6) 12.40 (s, 1H), 8.24 |
| | | | | | (dd, J = 8.0, |
| | | | | | 7.0 Hz, 1H), |
| | | | | | 7.98 (d, J = |
| | | | | | 8.0 Hz, 1H), |
| | | | | | 2.50 (s, 3H), |
| | | | | | 1.37 (s, 9H) |
| 1-139 | S | CF₂CF₃ | Me | H | ¹H NMR |
| | | | | | (500 MHz, |
| | | | | | DMSO-d6) |
| | | | | | 12.41 (s, |
| | | | | | 1H), 8.14 |
| | | | | | (dd, J = 8.0, |
| | | | | | 7.0 Hz, 1H), |
| | | | | | 7.99 (d, J = |
| | | | | | 8.0 Hz, 1H), |
| | | | | | 2.51 (s, 3H). |
| 1-140 | S | CF₂CF₂CF₃ | Me | H | ¹H NMR |
| | | | | | (500 MHz, |
| | | | | | DMSO-d6) |
| | | | | | 12.42 (s, |
| | | | | | 1H), 8.20 |
| | | | | | (dd, J = 8.0, |
| | | | | | 7.0 Hz, 1H), |
| | | | | | 8.07 (d, J = |
| | | | | | 8.0 Hz, 1H), |
| | | | | | 2.51 (s, 3H). |
| 1-141 | S | 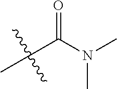 [SMILES: *C(=O)N(C)C] | Me | H | ¹H NMR (500 MHz, DMSO-d₆) 12.41 (s, 1H), 8.22 (dd, J = 8.0, |
| | | | | | 8.09 (d, J = |
| | | | | | 8.0 Hz, 1H), |
| | | | | | 3.27 (s, 6H), |
| | | | | | 2.51 (s, 3H). |
| 1-142 | S | 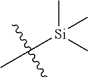 [SMILES: *[Si](C)(C)C] | Me | H | ¹H NMR (500 MHz, DMSO-d6) 12.40 (s, 1H), 8.00 |
| | | | | | (dd, J = 8.0, |
| | | | | | 7.0 Hz, 1H), |
| | | | | | 7.89 (d, J = |
| | | | | | 8.0 Hz, 1H), |
| | | | | | 2.51 (s, 3H), |
| | | | | | 0.02 (s, 9H). |
| 1-143 | S | 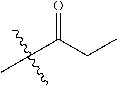 [SMILES: *C(=O)CC] | Me | H | ¹H NMR (500 MHz, DMSO-d6) 12.41 (s, 1H), 8.05 |
| | | | | | (dd, J = 8.0, |
| | | | | | 7.0 Hz, 1H), |
| | | | | | 7.93 (d, J = |
| | | | | | 8.0 Hz, 1H), |
| | | | | | 2.50 (s, 3H), |
| | | | | | 2.31 (q, J = |
| | | | | | 7.5 Hz, 2H), |
| | | | | | 1.16 (t, J = |
| | | | | | 7.5 Hz, 3H). |
| 1-144 | S | 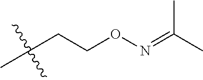 [SMILES: *CCON=C(C)C] | Me | H | ¹H NMR (500 MHz, DMSO-d6) 12.40 (s, 1H), 8.00 |
| | | | | | (dd, J = 8.0, |
| | | | | | 7.0 Hz, 1H), |
| | | | | | 7.89 (d, J = |
| | | | | | 8.0 Hz, 1H), |
| | | | | | 3.97 (t, J = |
| | | | | | 8.0 Hz, 2H), |
| | | | | | 3.36 (t, J = |
| | | | | | 8.0 Hz, 2H), |
| | | | | | 3.01 (s, 6H), |
| | | | | | 2.51 (s, 3H), |
| 1-145 | S | 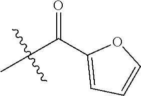 [SMILES: *C(=O)c1ccco1] | Me | H | ¹H NMR (500 MHz, DMSO-d6) 12.41 (s, 1H), 8.20 (d, J = 2.0 Hz, |
| | | | | | 1H), 8.02 |
| | | | | | (dd, J = 8.0, |
| | | | | | 7.0 Hz, 1H), |
| | | | | | 7.93 (d, J = |
| | | | | | 8.0 Hz, 1H), |
| | | | | | 7.56 (d, J = |
| | | | | | 2.5 Hz, 1H), |
| | | | | | 6.89 (dd, J = |
| | | | | | 2.5, 2.0 Hz, |
| | | | | | 1H), 2.50 (s, |
| | | | | | 3H) |
| 1-146 | S | 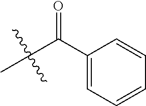 [SMILES: *C(=O)c1ccccc1] | Me | H | ¹H NMR (500 MHz, DMSO-d6) 12.41 (s, 1H), 8.08 (dd, J = 8.0, |
| | | | | | 7.0 Hz, 1H), |
| | | | | | 7.98-7.73 |
| | | | | | (m, 6H), |
| | | | | | 2.50 (s, 3H) |
| 1-147 | S | 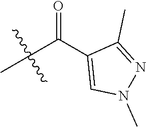 [SMILES: *C(=O)c1cn(C)nc1C] | Me | H | ¹H NMR (500 MHz, DMSO-d6) 12.40 (s, 1H), 8.77 (s, 1H), 8.06 (dd, J = 8.0, 7.0 Hz, 1H), |
| | | | | | 7.92 (d, J = |
| | | | | | 8.0 Hz, 1H), |
| | | | | | 3.77 (s, 3H), |
| | | | | | 2.50 (s, 3H), |
| | | | | | 2.47 (s, 3H). |
| 1-148 | S | 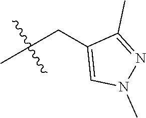 [SMILES: *Cc1cn(C)nc1C] | Me | H | ¹H NMR (500 MHz, DMSO-d6) 12.41 (s, 1H), 8.37 (s, 1H), 8.00 (dd, J = 8.0, |
| | | | | | 7.88 (d, J = |
| | | | | | 8.0 Hz, 1H), |
| | | | | | 4.35 (s, 2H), |
| | | | | | 3.75 (s, 3H), |
| | | | | | 2.50 (s, 3H), |
| | | | | | 2.10 (s, 3H). |
| 1-149 | NCOMe | 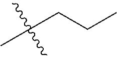 [SMILES: *CCC] | Me | H | |
| 1-150 | NSO₂Me | Me | Me | H | |
| 1-151 | NPh | Et | Me | H | |
| 1-152 | NH | CH₂Ph | Me | H | |
| 1-153 | NH | 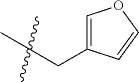 [SMILES: *Cc1ccoc1] | Me | H | |
| 1-154 | 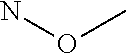 | Me | Me | H | |
| 1-155 | 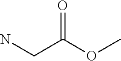 | Me | Me | H | |
| 1-156 | 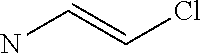 | Et | Me | H | |
| 1-157 | S | F | Me | H | |
| 1-158 | O | Cl | Me | H | |
| 1-159 | SO | Br | Me | H | |
| 1-160 | SO₂ | I | Me | H | |
| 1-161 | SO | 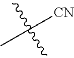 [SMILES: *C#N] | Me | H | ¹H NMR (500 MHz, DMSO-d₆) δ 12.38 (s, |
| | | | | | 1H), 8.03 (dd, |
| | | | | | J = 7.5. 6.0 |
| | | | | | Hz, 1H), 7.62 |
| | | | | | (d, J = 7.5 |
| | | | | | Hz, 1H), 2.43 |
| | | | | | (s, 3H). |
| 1-162 | SO₂ | 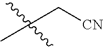 [SMILES: *CC#N] | Me | H | |
| 1-163 | O | 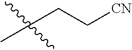 [SMILES: *CCC#N] | Me | H | |
| 1-164 | S | COOH | Me | H | |
| 1-165 | S | NO₂ | Me | H | |
| 1-166 | S | NH₂ | Me | H | |
| 1-167 | S | 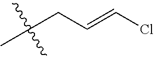 [SMILES: *C/C=C/Cl] | Me | H | |
| 1-168 | O | 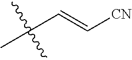 [SMILES: */C=C/C#N] | Me | H | |
| 1-169 | NH | 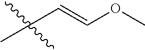 [SMILES: */C=C/OC] | Me | H | |
| 1-170 | SO | 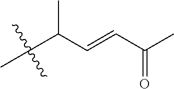 [SMILES: *C(C)/C=C/C(C)=O] | Me | H | |
| 1-171 | SO₂ | 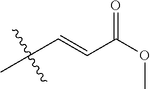 [SMILES: */C=C/C(=O)OC] | Me | H | |
| 1-172 | S | 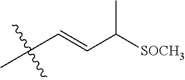 | Me | H | |
| 1-173 | S | 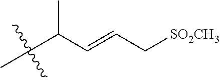 [SMILES: *C(C)/C=C/CS(C)(=O)=O] | Me | H | |
| 1-174 | S | 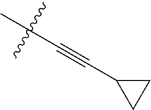 [SMILES: *C#CC1CC1] | Me | H | |
| 1-175 | S | 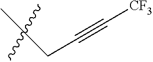 [SMILES: *CC#CC(F)(F)F] | Me | H | |
| 1-176 | S | 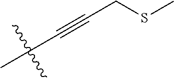 [SMILES: *C#CCSC] | Me | H | |
| 1-177 | O | 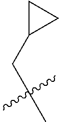 [SMILES: *CC1CC1] | Me | H | |
| 1-178 | NMe | 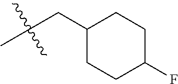 [SMILES: *CC1CCC(F)CC1] | Me | H | |
| 1-179 | NH | 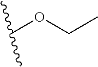 [SMILES: *OCC] | Me | H | |
| 1-180 | NH | 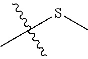 [SMILES: *SC] | Me | H | |
| 1-181 | S | 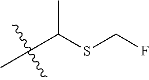 [SMILES: *C(C)SCF] | Me | H | |
| 1-182 | S | 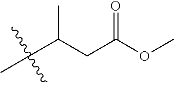 [SMILES: *C(C)CC(=O)OC] | Me | H | |
| 1-183 | NH | 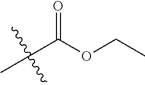 [SMILES: *C(=O)OCC] | Me | H | |
| 1-184 | O | 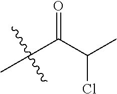 [SMILES: *C(=O)C(C)Cl] | Me | H | |
| 1-185 | S | 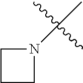 [SMILES: *N1CCC1] | Me | H | |
| 1-186 | S | 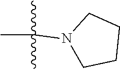 [SMILES: *N1CCCC1] | Me | H | |
| 1-187 | S | 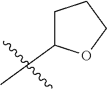 [SMILES: *C1CCCO1] | Me | H | |
| 1-188 | O | 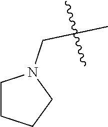 [SMILES: *CN1CCCC1] | Me | H | |
| 1-189 | NH | 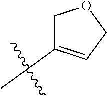 [SMILES: *C1=CCOC1] | Me | H | |
| 1-190 | SO | 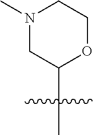 [SMILES: *C1CN(C)CCO1] | Me | H | |
| 1-191 | O | 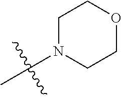 [SMILES: *N1CCOCC1] | Me | H | |
| 1-192 | S | 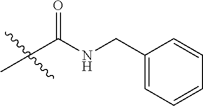 [SMILES: *C(=O)NCc1ccccc1] | Me | H | |
| 1-193 | S | 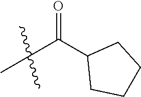 [SMILES: *C(=O)C1CCCC1] | Me | H | |
| 1-194 | S | 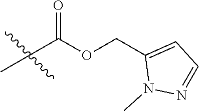 [SMILES: *C(=O)OCc1ccnn1C] | Me | H | |
| 1-195 | S | 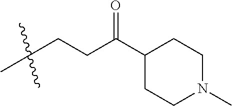 [SMILES: *CCC(=O)C1CCN(C)CC1] | Me | H | |
| 1-196 | NH | 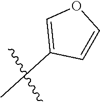 [SMILES: *c1ccoc1] | Me | H | |
| 1-197 | NH | 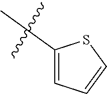 [SMILES: *c1cccs1] | Me | H | |
| 1-198 | S | 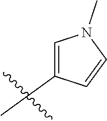 [SMILES: *c1ccn(C)c1] | Me | H | |
| 1-199 | SO | 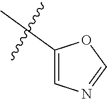 [SMILES: *c1cnco1] | Me | H | |
| 1-200 | SO₂ | 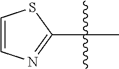 [SMILES: *c1nccs1] | Me | H | |
| 1-201 | O | 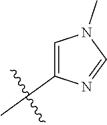 [SMILES: *c1cn(C)cn1] | Me | H | |
| 1-202 | NEt | 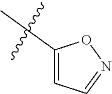 [SMILES: *c1ccno1] | Me | H | |
| 1-203 | NH | 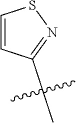 [SMILES: *c1ccsn1] | Me | H | |
| 1-204 | S | 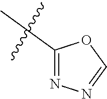 [SMILES: *c1nnco1] | Me | H | |
| 1-205 | SO | 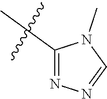 [SMILES: *c1nncn1C] | Me | H | |
| 1-206 | SO₂ | 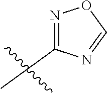 [SMILES: *c1ncon1] | Me | H | |
| 1-207 | S | 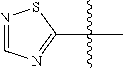 [SMILES: *c1ncns1] | Me | H | |
| 1-208 | SO | 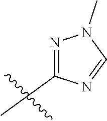 [SMILES: *c1ncn(C)n1] | Me | H | |
| 1-209 | SO₂ | 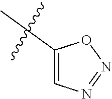 [SMILES: *c1cnno1] | Me | H | |
| 1-210 | O | 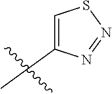 [SMILES: *c1csnn1] | Me | H | |
| 1-211 | S | 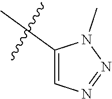 [SMILES: *c1cnnn1C] | Me | H | |
| 1-212 | O | 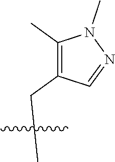 [SMILES: *Cc1cnn(C)c1C] | Me | H | |
| 1-213 | S | 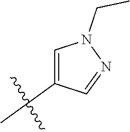 [SMILES: *c1cnn(CC)c1] | Me | H | |
| 1-214 | S | 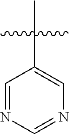 [SMILES: *c1cncnc1] | Me | H | |
| 1-215 | O | 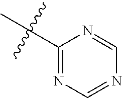 [SMILES: *c1ncncn1] | Me | H | |
| 1-216 | S | 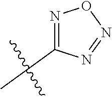 [SMILES: *c1nnon1] | Me | H | |
| 1-217 | O | 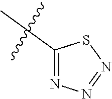 [SMILES: *c1nnns1] | Me | H | |
| 1-218 | SO | 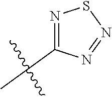 [SMILES: *c1nnsn1] | Me | H | |
| 1-219 | SO₂ | 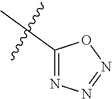 [SMILES: *c1nnno1] | Me | H | |
| 1-220 | S | 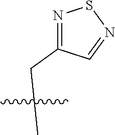 [SMILES: *Cc1cnsn1] | Me | H | |
| 1-221 | S | 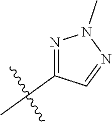 [SMILES: *c1cnn(C)n1] | Me | H | |
| 1-222 | S | 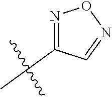 [SMILES: *c1cnon1] | Me | H | |
| 1-223 | S | 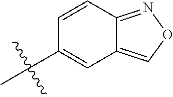 [SMILES: *c1ccc2nocc2c1] | Me | H | |
| 1-224 | NH | 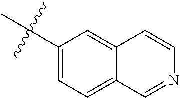 [SMILES: *c1ccc2cnccc2c1] | Me | H | |
| 1-225 | S | 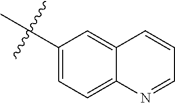 [SMILES: *c1ccc2ncccc2c1] | Me | H | |
| 1-226 | S | 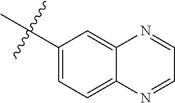 [SMILES: *c1ccc2nccnc2c1] | Me | H | |
| 1-227 | S | 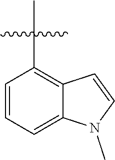 [SMILES: *c1cccc2c1ccn2C] | Me | H | |
| 1-228 | SO | 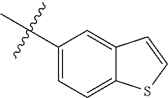 [SMILES: *c1ccc2sccc2c1] | Me | H | |
| 1-229 | SO₂ | 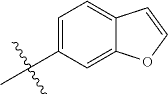 [SMILES: *c1ccc2ccoc2c1] | Me | H | |
| 1-230 | SO | 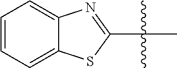 [SMILES: *c1nc2ccccc2s1] | Me | H | |
| 1-231 | SO₂ | 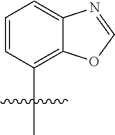 [SMILES: *c1cccc2ncoc12] | Me | H | |
| 1-232 | NH | 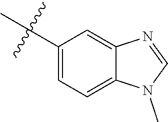 [SMILES: *c1ccc2c(c1)ncn2C] | Me | H | |
| 1-233 | S | 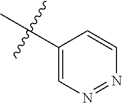 [SMILES: *c1ccnnc1] | Me | H | |
| 1-234 | O | 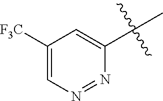 [SMILES: *c1cc(C(F)(F)F)cnn1] | Me | H | |
| 1-235 | S | 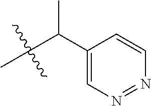 [SMILES: *C(C)c1ccnnc1] | Me | H | |
| 1-236 | S | 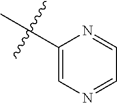 [SMILES: *c1cnccn1] | Me | H | |
| 1-237 | S | 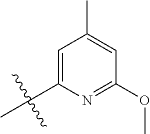 [SMILES: *c1cc(C)cc(OC)n1] | Me | H | |
| 1-238 | O | 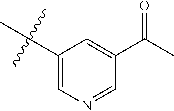 [SMILES: *c1cncc(C(C)=O)c1] | Me | H | |
| 1-239 | S | 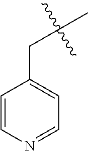 [SMILES: *Cc1ccncc1] | Me | H | |
| 1-240 | SO | 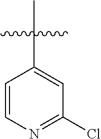 [SMILES: *c1ccnc(Cl)c1] | Me | H | |
| 1-241 | S | 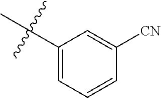 [SMILES: *c1cccc(C#N)c1] | Me | H | |
| 1-242 | SO | 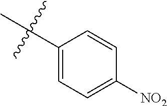 [SMILES: *c1ccc([N+](=O)[O-])cc1] | Me | H | |
| 1-243 | SO₂ | 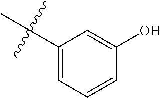 [SMILES: *c1cccc(O)c1] | Me | H | |
| 1-244 | NH | 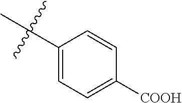 [SMILES: *c1ccc(C(=O)O)cc1] | Me | H | |
| 1-245 | O | 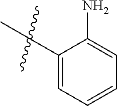 [SMILES: *c1ccccc1N] | Me | H | |
| 1-246 | S | 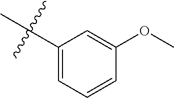 [SMILES: *c1cccc(OC)c1] | Me | H | |
| 1-247 | S | 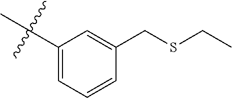 [SMILES: *c1cccc(CSCC)c1] | Me | H | |
| 1-248 | S | 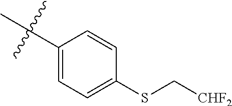 [SMILES: *c1ccc(SCC(F)F)cc1] | Me | H | |
| 1-249 | S | 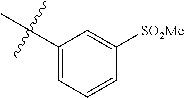 [SMILES: *c1cccc(S(C)(=O)=O)c1] | Me | H | |
| 1-250 | S | 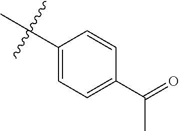 [SMILES: *c1ccc(C(C)=O)cc1] | Me | H | |
| 1-251 | S | 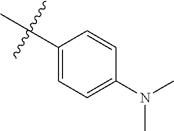 [SMILES: *c1ccc(N(C)C)cc1] | Me | H | |
| 1-252 | S | 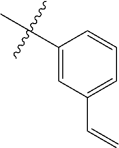 [SMILES: *c1cccc(C=C)c1] | Me | H | |
| 1-253 | S | 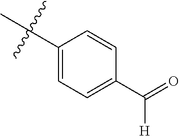 [SMILES: *c1ccc(C=O)cc1] | Me | H | |
| 1-254 | SO | 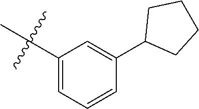 [SMILES: *c1cccc(C2CCCC2)c1] | Me | H | |
| 1-255 | SO₂ | 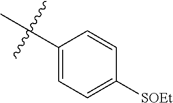 | Me | H | |
| 1-256 | SO | 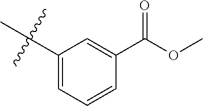 [SMILES: *c1cccc(C(=O)OC)c1] | Me | H | |
| 1-257 | SO₂ | 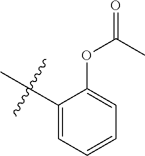 [SMILES: *c1ccccc1OC(C)=O] | Me | H | |
| 1-258 | SO | 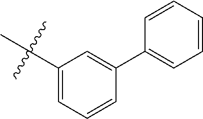 [SMILES: *c1cccc(-c2ccccc2)c1] | Me | H | |
| 1-259 | SO₂ | 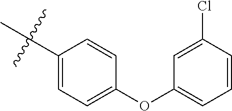 [SMILES: *c1ccc(Oc2cccc(Cl)c2)cc1] | Me | H | |
| 1-260 | S | 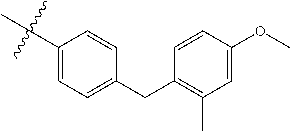 [SMILES: *c1ccc(Cc2ccc(OC)cc2C)cc1] | Me | H | |
| 1-261 | S | 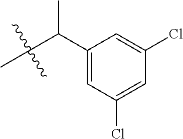 [SMILES: *C(C)c1cc(Cl)cc(Cl)c1] | Me | H | |
| 1-262 | S | 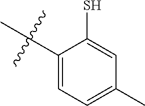 [SMILES: *c1ccc(C)cc1S] | Me | H | |
| 1-263 | S | 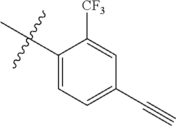 [SMILES: *c1ccc(C#C)cc1C(F)(F)F] | Me | H | |
| 1-264 | O | 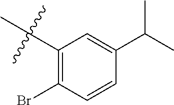 [SMILES: *c1cc(C(C)C)ccc1Br] | Me | H | |
| 1-265 | S | 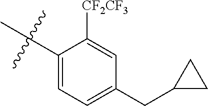 | Me | H | |
| 1-266 | SO | 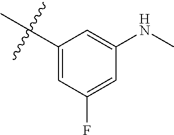 [SMILES: *c1cc(F)cc(NC)c1] | Me | H | |
| 1-267 | S | 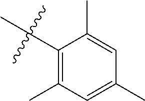 [SMILES: *c1c(C)cc(C)cc1C] | Me | H | |
| 1-268 | S | 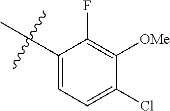 [SMILES: *c1ccc(Cl)c(OC)c1F] | Me | H | |
| 1-269 | S | 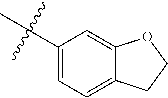 [SMILES: *c1ccc2c(c1)OCC2] | Me | H | |
| 1-270 | O | 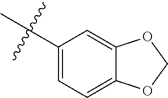 [SMILES: *c1ccc2c(c1)OCO2] | Me | H | |
| 1-271 | NH | 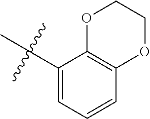 [SMILES: *c1cccc2c1OCCO2] | Me | H | |
| 1-272 | S | 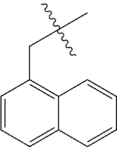 [SMILES: *Cc1cccc2ccccc12] | Me | H | |
| 1-273 | S | 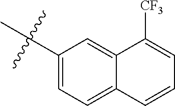 [SMILES: *c1ccc2cccc(C(F)(F)F)c2c1] | Me | H | |
| 1-274 | S | 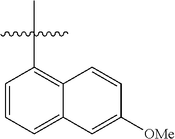 [SMILES: *c1cccc2cc(OC)ccc12] | Me | H | |
| 1-275 | S | 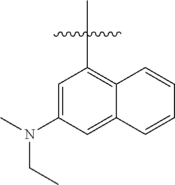 [SMILES: *c1cc(N(C)CC)cc2ccccc12] | Me | H | |
| 1-276 | S | 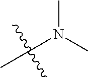 [SMILES: *N(C)C] | Me | H | |
| 1-277 | SO₂ | 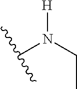 [SMILES: *NCC] | Me | H | |
| 1-278 | SO | 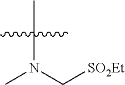 | Me | H | |
| 1-279 | S | 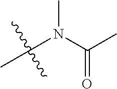 [SMILES: *N(C)C(C)=O] | Me | H | |
| 1-280 | O | 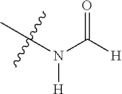 [SMILES: *NC=O] | Me | H | |
| 1-281 | NH | 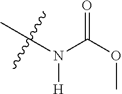 [SMILES: *NC(=O)OC] | Me | H | |
| 1-282 | S | 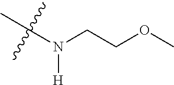 [SMILES: *NCCOC] | Me | H | |
| 1-283 | S | 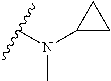 [SMILES: *N(C)C1CC1] | Me | H | |
| 1-284 | S | 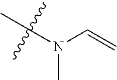 [SMILES: *N(C)C=C] | Me | H | |
| 1-285 | S | 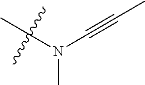 [SMILES: *N(C)C#CC] | Me | H | |
| 1-286 | S | 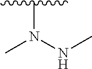 | Me | H | |
| 1-287 | S | 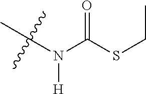 [SMILES: *NC(=O)SCC] | Me | H | |
| 1-288 | S | 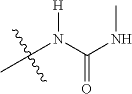 [SMILES: *NC(=O)NC] | Me | H | |
| 1-289 | S | 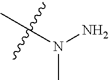 [SMILES: *N(C)N] | Me | H | |
| 1-290 | S | 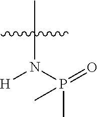 [SMILES: *NP(C)(C)=O] | Me | H | |
| 1-291 | S | 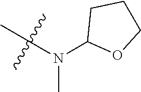 [SMILES: *N(C)C1CCCO1] | Me | H | |
| 1-292 | S | 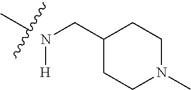 | Me | H | |
| 1-293 | SO₂ | 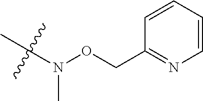 [SMILES: *N(C)OCc1ccccn1] | Me | H | |
| 1-294 | SO | 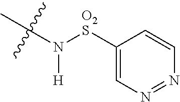 [SMILES: *NS(=O)(=O)c1ccnnc1] | Me | H | |
| 1-295 | S | 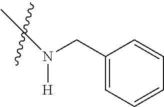 [SMILES: *NCc1ccccc1] | Me | H | |
| 1-296 | S | 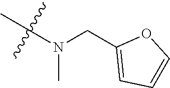 [SMILES: *N(C)Cc1ccco1] | Me | H | |
| 1-297 | S | 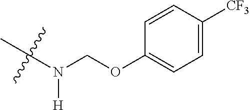 [SMILES: *NCOc1ccc(C(F)(F)F)cc1] | Me | H | |
| 1-298 | S | 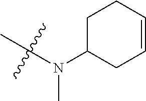 [SMILES: *N(C)C1CC=CCC1] | Me | H | |
| 1-299 | S | 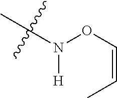 [SMILES: *NO/C=C\C] | Me | H | |
| 1-300 | S | 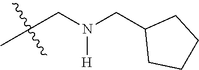 [SMILES: *CNCC1CCCC1] | Me | H | |
| 1-301 | S | 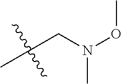 [SMILES: *CN(C)OC] | Me | H | |
| 1-302 | O | 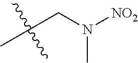 [SMILES: *CN(C)[N+](=O)[O-]] | Me | H | |
| 1-303 | NH | 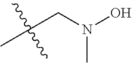 [SMILES: *CN(C)O] | Me | H | |
| 1-304 | S | 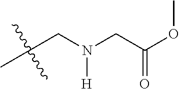 [SMILES: *CNCC(=O)OC] | Me | H | |
| 1-305 | S | 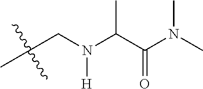 [SMILES: *CNC(C)C(=O)N(C)C] | Me | H | |
| 1-306 | SO₂ | 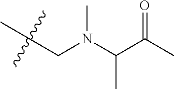 [SMILES: *CN(C)C(C)C(C)=O] | Me | H | |
| 1-307 | SO | 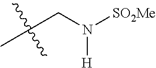 [SMILES: *CNS(C)(=O)=O] | Me | H | |
| 1-308 | SO₂ | 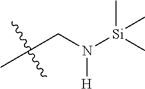 [SMILES: *CN[Si](C)(C)C] | Me | H | |
| 1-309 | SO | 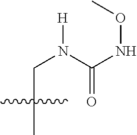 [SMILES: *CNC(=O)NOC] | Me | H | |
| 1-310 | S | 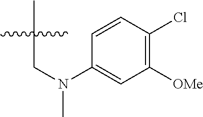 [SMILES: *CN(C)c1ccc(Cl)c(OC)c1] | Me | H | |
| 1-311 | S | 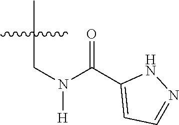 [SMILES: *CNC(=O)c1ccn[nH]1] | Me | H | |
| 1-312 | 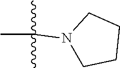 [SMILES: *N1CCCC1] | | Me | H | |
| 1-313 | 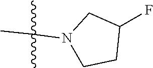 [SMILES: *N1CCC(F)C1] | | Me | H | |
| 1-314 | 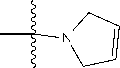 [SMILES: *N1CC=CC1] | | Me | H | |
| 1-315 | 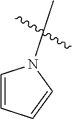 [SMILES: *n1cccc1] | | Me | H | |
| 1-316 | 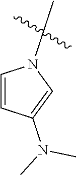 [SMILES: *n1ccc(N(C)C)c1] | | Me | H | |
| 1-317 | 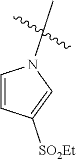 | | Me | H | |
| 1-318 | 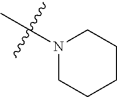 [SMILES: *N1CCCCC1] | | Me | H | ¹H NMR (500 MHz, DMSO-d₆) δ 12.36 (s, 1H), 7.72- 7.65 (m, |
| | | | | | 2H), 3.12- |
| | | | | | 3.09 (m, |
| | | | | | 4H), 2.47 (s, |
| | | | | | 3H), 1.74- |
| | | | | | 1.64 (m, 6H). |
| 1-319 | 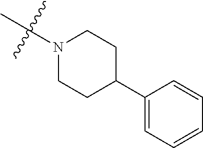 [SMILES: *N1CCC(c2ccccc2)CC1] | | Me | H | |
| 1-320 | 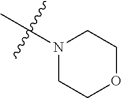 [SMILES: *N1CCOCC1] | | Me | H | |
| 1-321 | 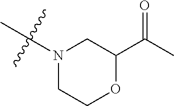 [SMILES: *N1CCOC(C(C)=O)C1] | | Me | H | |
| 1-322 | 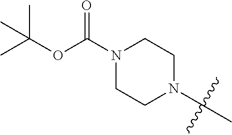 [SMILES: *N1CCN(C(=O)OC(C)(C)C)CC1] | | Me | H | |
| 1-323 | 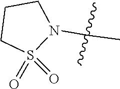 [SMILES: *N1CCCS1(=O)=O] | | Me | H | |
| 1-324 | 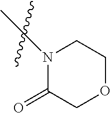 [SMILES: *N1CCOCC1=O] | | Me | H | |
| 1-325 | 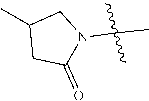 [SMILES: *N1CC(C)CC1=O] | | Me | H | |
| 1-326 | 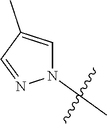 [SMILES: *n1cc(C)cn1] | | Me | H | ¹H NMR (500 MHz, DMSO-d6) δ 12.27 (s, 1H), 7.96- 7.82 (m, 2H), 7.65 (s, 1H), 7.42 (s, |
| | | | | | 1H), 2.47 (s, |
| | | | | | 3H), 2.08 (s, |
| | | | | | 3H). |
| 1-327 | 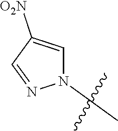 [SMILES: *n1cc([N+](=O)[O-])cn1] | | Me | H | |
| 1-328 | 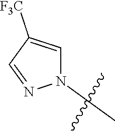 [SMILES: *n1cc(C(F)(F)F)cn1] | | Me | H | |
| 1-329 | 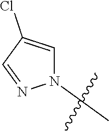 [SMILES: *n1cc(Cl)cn1] | | Me | H | |
| 1-330 | 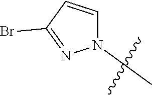 [SMILES: *n1ccc(Br)n1] | | Me | H | |
| 1-331 | 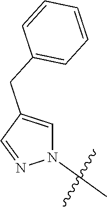 [SMILES: *n1cc(Cc2ccccc2)cn1] | | Me | H | |
| 1-332 | 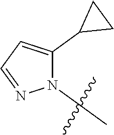 [SMILES: *n1nccc1C1CC1] | | Me | H | |
| 1-333 | 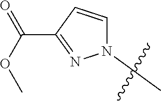 [SMILES: *n1ccc(C(=O)OC)n1] | | Me | H | |
| 1-334 | 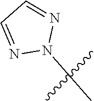 [SMILES: *n1nccn1] | | Me | H | |
| 1-335 | 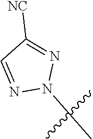 [SMILES: *n1ncc(C#N)n1] | | Me | H | |
| 1-336 | 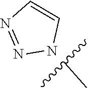 | | Me | H | |
| 1-337 | 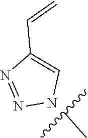 | | Me | H | |
| 1-338 | 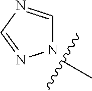 | | Me | H | |
| 1-339 | 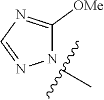 | | Me | H | |
| 1-340 | S | Et | Me | OMe | |
| 1-341 | O | Me | Me | 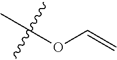 [SMILES: *OC=C] | |
| 1-342 | NMe | Me | Me | 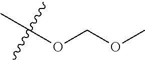 [SMILES: *OCOC] | |
| 1-343 | S | Et | Me | 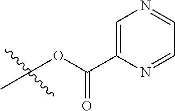 [SMILES: *OC(=O)c1cnccn1] | |
| 1-344 | S | Et | Me | 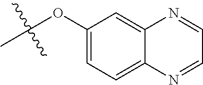 [SMILES: *Oc1ccc2nccnc2c1] | |
| 1-345 | O | Me | Me | 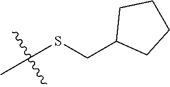 [SMILES: *SCC1CCCC1] | |
| 1-346 | NMe | Me | Me | 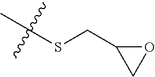 [SMILES: *SCC1CO1] | |
| 1-347 | S | Et | Me | 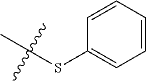 [SMILES: *Sc1ccccc1] | |
| 1-348 | O | Me | Me | 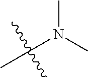 [SMILES: *N(C)C] | |
| 1-349 | S | Et | Me | 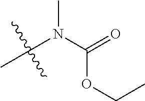 [SMILES: *N(C)C(=O)OCC] | |
| 1-350 | NMe | Me | Me | 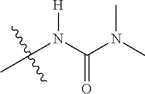 [SMILES: *NC(=O)N(C)C] | |
| 1-351 | S | Et | Me | 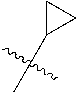 [SMILES: *C1CC1] | |
| 1-352 | S | Et | Me | 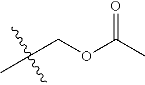 [SMILES: *COC(C)=O] | |
| 1-353 | O | Me | Me | 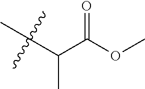 [SMILES: *C(C)C(=O)OC] | |
| 1-354 | S | Et | Me | 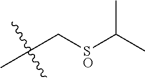 | |
| 1-355 | NMe | Me | Me | 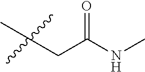 [SMILES: *CC(=O)NC] | |
| 1-356 | S | Et | Me | 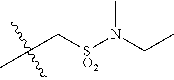 [SMILES: *CS(=O)(=O)N(C)CC] | |
| 1-357 | S | Et | Me | 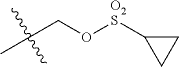 [SMILES: *COS(=O)(=O)C1CC1] | |
| 1-358 | S | Et | Me | 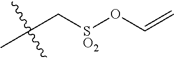 [SMILES: *CS(=O)(=O)OC=C] | |
| 1-359 | O | Me | Me | 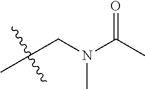 [SMILES: *CN(C)C(C)=O] | |
| 1-360 | S | Et | Me | 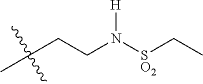 [SMILES: *CCNS(=O)(=O)CC] | |
| 1-361 | S | Et | Me | 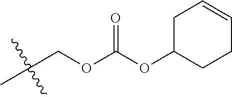 [SMILES: *COC(=O)OC1CC=CCC1] | |
| 1-362 | S | Et | Me | 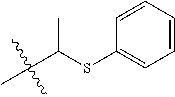 [SMILES: *C(C)Sc1ccccc1] | |
| 1-363 | O | Me | Me | 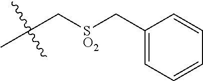 [SMILES: *CS(=O)(=O)Cc1ccccc1] | |
| 1-364 | NMe | Me | Me | 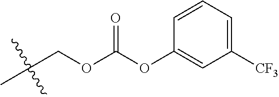 [SMILES: *COC(=O)Oc1cccc(C(F)(F)F)c1] | |
| 1-365 | S | Et | Me | 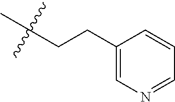 [SMILES: *CCc1cccnc1] | |
| 1-366 | S | Et | Me | 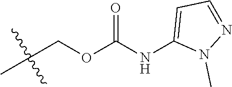 [SMILES: *COC(=O)Nc1ccnn1C] | |
| 1-367 | NMe | Me | Me | 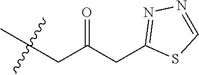 [SMILES: *CC(=O)Cc1nncs1] | |
| 1-368 | S | Et | Me | 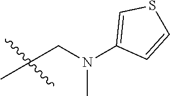 [SMILES: *CN(C)c1ccsc1] | |
| 1-369 | SO₂ | Et | Me | 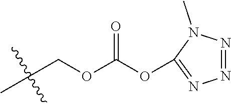 [SMILES: *COC(=O)Oc1nnnn1C] | |
| 1-370 | O | Me | Me | 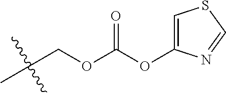 [SMILES: *COC(=O)Oc1cscn1] | |
| 1-371 | S | Et | Me | 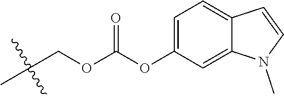 [SMILES: *COC(=O)Oc1ccc2ccn(C)c2c1] | |
| 1-372 | S | Et | Me | 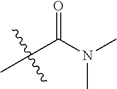 [SMILES: *C(=O)N(C)C] | |
| 1-373 | SO | Et | Me | 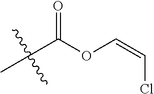 [SMILES: *C(=O)O/C=C\Cl] | |
| 1-374 | NMe | Me | Me | 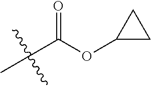 [SMILES: *C(=O)OC1CC1] | |
| 1-375 | S | Et | Me | 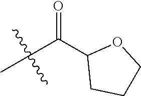 [SMILES: *C(=O)C1CCCO1] | |
| 1-376 | O | Me | Me | 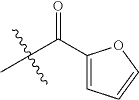 [SMILES: *C(=O)c1ccco1] | |
| 1-377 | NMe | Me | Me | 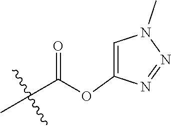 [SMILES: *C(=O)Oc1cn(C)nn1] | |
| 1-378 | S | Et | Me | 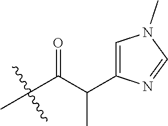 [SMILES: *C(=O)C(C)c1cn(C)cn1] | |
| 1-379 | O | Me | Me | 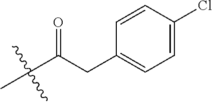 [SMILES: *C(=O)Cc1ccc(Cl)cc1] | |
| 1-380 | S | Et | Me | 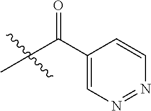 [SMILES: *C(=O)c1ccnnc1] | |
| 1-381 | S | Et | Me | 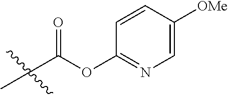 [SMILES: *C(=O)Oc1ccc(OC)cn1] | |
| 1-382 | SO₂ | Et | Me | 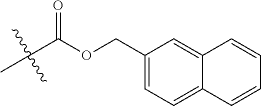 [SMILES: *C(=O)OCc1ccc2ccccc2c1] | |
| 1-383 | NMe | Me | Me | 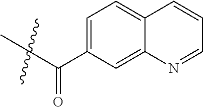 [SMILES: *C(=O)c1ccc2cccnc2c1] | |
| 1-384 | O | Me | Me | 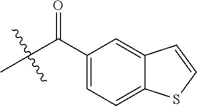 [SMILES: *C(=O)c1ccc2sccc2c1] | |
| 1-385 | S | Et | Me | 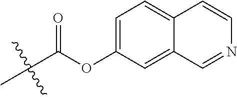 [SMILES: *C(=O)Oc1ccc2ccncc2c1] | |
| 1-386 | SO | Et | Me | 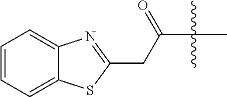 [SMILES: *C(=O)Cc1nc2ccccc2s1] | |
| 1-387 | SO₂ | Et | Me | 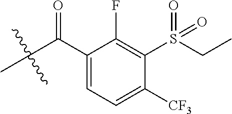 [SMILES: *C(=O)c1ccc(C(F)(F)F)c(S(=O)(=O)CC)c1F] | |
| 1-388 | S | Et | Me | 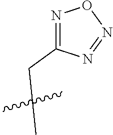 [SMILES: *Cc1nnon1] | |
| 1-389 | S | Et | Me | 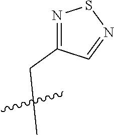 [SMILES: *Cc1cnsn1] | |
| 1-390 | S | Et | Me | 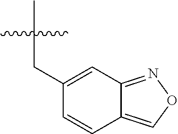 [SMILES: *Cc1ccc2conc2c1] | |
| 1-391 | S | Et | Me | 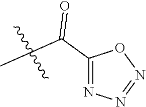 [SMILES: *C(=O)c1nnno1] | |
| 1-392 | SO | 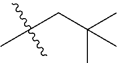 [SMILES: *CC(C)(C)C] | Me | H | ¹H NMR (500 MHz, DMSO-d₆) δ 12.38 (s, |
| | | | | | 1H), 8.33 (dd, |
| | | | | | J = 8.0, 7.0 |
| | | | | | Hz, 1H), 7.59 |
| | | | | | (d, J = 8.0 |
| | | | | | Hz, 1H), 2.64 |
| | | | | | (s, 2H), 2.46 |
| | | | | | (s, 3H), 0.94 |
| | | | | | (s, 9H). |
| 1-393 | SO | 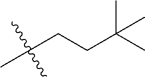 [SMILES: *CCC(C)(C)C] | Me | H | ¹H NMR (500 MHz, DMSO-d₆) δ 12.38 (s, 1H), 8.34 |
| | | | | | (dd, J = |
| | | | | | 7.5, 6.0 Hz, |
| | | | | | 1H), 7.61 (d, |
| | | | | | J = 7.5 Hz, |
| | | | | | 1H), 2.76 (t, |
| | | | | | J = 8.0 Hz, |
| | | | | | 2H), 2.46 (s, |
| | | | | | 3H), 1.49 (t, |
| | | | | | J = 8.0 Hz, |
| | | | | | 2H), 0.86 (s, |
| | | | | | 9H). |
| 1-394 | SO | 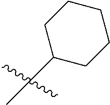 [SMILES: *C1CCCCC1] | Me | H | ¹H NMR (500 |
| | | | | | MHz, |
| | | | | | DMSO-d₆) δ |
| | | | | | 12.55 (s, |
| | | | | | 1H), 8.10 (d, |
| | | | | | J = 7.5 Hz, |
| | | | | | 1H), 7.89- |
| | | | | | 7.86 (m, |
| | | | | | 1H), 2.47 |
| | | | | | (s, 3H), |
| | | | | | 2.21-2.19 |
| | | | | | (m, 1H), |
| | | | | | 1.90-1.79 |
| | | | | | (m, 1H), |
| | | | | | 1.71-1.63 |
| | | | | | (m, 1H), |
| | | | | | 1.62-1.56 |
| | | | | | (m, 1H), |
| | | | | | 1.49-1.41 |
| | | | | | (m, 1H), |
| | | | | | 1.38-1.10 |
| | | | | | (m, 6H). |
| 1-395 | SO | 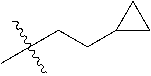 [SMILES: *CCC1CC1] | Me | H | ¹H NMR (500 MHz, DMSO-d6) δ 12.38 (s, 1H), 8.34 (dd, |
| | | | | | Hz, 1H), 7.59 |
| | | | | | (d J= 7.5 Hz, |
| | | | | | 1H), 2.76 (t, |
| | | | | | J = 8.1 Hz, |
| | | | | | 2H), 2.46 (s, |
| | | | | | 3H), 1.55- |
| | | | | | 1.46 (m, 2H), |
| | | | | | 0.98-0.87 (m, |
| | | | | | 1H), 0.42- |
| | | | | | 0.31 (m, 2H), |
| | | | | | 0.24-0.18 (m, |
| | | | | | 2H). |
| 1-396 | SO | 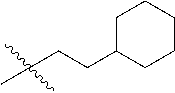 [SMILES: *CCC1CCCCC1] | Me | H | ¹H NMR (500 MHz, DMSO-d₆) δ 12.38 (s, 1H), 8.33 (dd, J = 7.5, 6.0 |
| | | | | | Hz, 1H), 7.59 |
| | | | | | (d, J = 7.5, |
| | | | | | 1H), 2.76 (t, |
| | | | | | J = 5.2 Hz, |
| | | | | | 2H), 2.46 (s, |
| | | | | | 3H), 1.72- |
| | | | | | 1.43 (m, 7H), |
| | | | | | 1.40-1.29 |
| | | | | | (m, 3H), 1.29- |
| | | | | | 1.20 (m, |
| | | | | | 2H), 1.20- |
| | | | | | 1.15 (m, 1H). |
| 1-397 | SO | 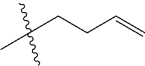 [SMILES: *CCC=C] | Me | H | ¹H NMR |
| | | | | | (500 MHz, |
| | | | | | DMSO-d₆) δ |
| | | | | | 12.38 (s, |
| | | | | | 1H), 8.33 (dd, |
| | | | | | J = 7.5, 6.0 |
| | | | | | Hz, 1H), 7.59 |
| | | | | | (d, J = 7.5 |
| | | | | | Hz, 1H), |
| | | | | | 5.87-5.76 (m, |
| | | | | | 1H), 5.08- |
| | | | | | 4.93 (m, 2H), |
| | | | | | 2.82 (t, J = |
| | | | | | 8.0 Hz, 2H), |
| | | | | | 2.46 (s, 3H), |
| | | | | | 2.34-2.26 |
| | | | | | (m, 2H). |
| 1-398 | SO | 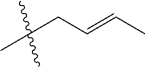 [SMILES: *C/C=C/C] | Me | H | ¹H NMR (500 MHz, DMSO-d6) δ 12.38 (s, |
| | | | | | 1H), 8.38 (dd, |
| | | | | | J = 7.5, 6.0 |
| | | | | | Hz, 1H), 7.60 |
| | | | | | (d, J = 7.5 |
| | | | | | Hz, 1H), 5.55 |
| | | | | | 2H), 3.45- |
| | | | | | 3.34 (m, 2H), |
| | | | | | 2.46 (s, 3H), |
| | | | | | 1.65-1.60 |
| | | | | | (m, 3H). |
| 1-399 | SO | 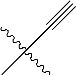 [SMILES: *C#C] | Me | H | |
| 1-400 | SO | 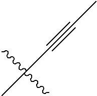 [SMILES: *C#CC] | Me | H | |
| 1-401 | SO | 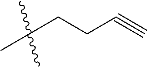 [SMILES: *CCC#C] | Me | H | ¹H NMR (500 MHz, DMSO-d6) δ 12.38 (s, |
| | | | | | 1H), 8.34 (dd, |
| | | | | | J = 7.5, 6.0 |
| | | | | | Hz, 1H), 7.59 |
| | | | | | (d, J = 7.5 |
| | | | | | Hz, 1H), 3.05 |
| | | | | | (t, J = 3.0 Hz, |
| | | | | | 1H), 2.95 (t, |
| | | | | | J = 8.0 Hz, |
| | | | | | 2H), 2.46 (s, |
| | | | | | 3H), 2.37- |
| | | | | | 2.26 (m, 2H). |
| 1-402 | SO | 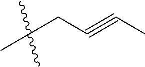 [SMILES: *CC#CC] | Me | H | ¹H NMR (500 MHz, DMSO-d6) δ 12.38 (s, |
| | | | | | 1H), 8.32 (dd, |
| | | | | | J = 7.5, 6.0 |
| | | | | | Hz, 1H), 7.59 |
| | | | | | (d, J = 7.5 |
| | | | | | Hz, 1H), 3.47 |
| | | | | | (q, J = 2.0 |
| | | | | | Hz, 2H), 2.46 |
| | | | | | (s, 3H), 1.77 |
| | | | | | (t, J = 2.0 Hz, |
| | | | | | 3H). |
| 1-403 | SO | 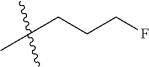 [SMILES: *CCCF] | Me | H | ¹H NMR (500 MHz, DMSO-d6) δ 12.38 (s, |
| | | | | | 1H), 8.33 (dd, |
| | | | | | J = 7.5, 6.0 |
| | | | | | Hz, 1H), 7.59 |
| | | | | | (d, J = 7.5 |
| | | | | | Hz, 1H), 4.35 |
| | | | | | (t, J = 7.5 Hz, |
| | | | | | 1H), 4.25 (t, |
| | | | | | J = 7.5 Hz, |
| | | | | | 1H), 2.76 (t, |
| | | | | | J = 5.5 Hz, |
| | | | | | 2H), 2.46 (s, |
| | | | | | 3H), 1.90- |
| | | | | | 1.76 (m, 2H). |
| 1-404 | SO | 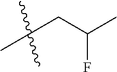 [SMILES: *CC(C)F] | Me | H | ¹H NMR (500 MHz, DMSO-d6) δ 12.38 (s, |
| | | | | | 1H), 8.28 |
| | | | | | (dd, J = 7.5, |
| | | | | | 6.0 Hz, 1H), |
| | | | | | 7.59 (d, J = |
| | | | | | 7.5 Hz, 1H), |
| | | | | | 5.20-5.11 |
| | | | | | (m, 1H), |
| | | | | | 3.04-2.93 (m, |
| | | | | | 1H), 2.79- |
| | | | | | 2.68 (m, 1H), |
| | | | | | 2.40 (s, 3H), |
| | | | | | 1.41 (dd, J = |
| | | | | | 25.0, 7.0 Hz, |
| | | | | | 3H). |
| 1-405 | SO | 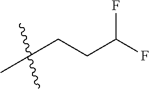 [SMILES: *CCC(F)F] | Me | H | ¹H NMR (500 MHz, DMSO-d6) δ 12.38 (s, 1H), 8.34 (dd, J = 7.5, 6.0 |
| | | | | | Hz, 1H), 7.59 |
| | | | | | (d, J = 7.5 |
| | | | | | Hz, 1H), 5.37 |
| | | | | | (t, J = 7.0 Hz, |
| | | | | | 1H), 2.76 (t, |
| | | | | | J = 8.5 Hz, |
| | | | | | 2H), 2.46 (s, |
| | | | | | 3H), 1.99- |
| | | | | | 1.88 (m, 2H). |
| 1-406 | SO | 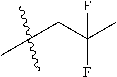 [SMILES: *CC(C)(F)F] | Me | H | ¹H NMR (500 MHz, DMSO-d₆) δ 12.38 (s, 1H), 8.33 (dd, |
| | | | | | J = 7.5, 6.0 |
| | | | | | Hz, 1H), 7.59 |
| | | | | | (d, J = 7.5 |
| | | | | | Hz, |
| | | | | | 1H), 3.45- |
| | | | | | 3.32 (m, 2H), |
| | | | | | 2.46 (s, 3H), |
| | | | | | 1.63-1.51 (m, |
| | | | | | 3H). |
| 1-407 | SO | 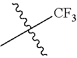 [SMILES: *C(F)(F)F] | Me | H | ¹H NMR (500 MHz, DMSO-d6) δ 12.38 (s, |
| | | | | | 1H), 8.32 (dd, |
| | | | | | J = 7.5, 6.0 |
| | | | | | Hz, 1H), 7.57 |
| | | | | | (d, J = 7.5 |
| | | | | | Hz, 1H), 2.45 |
| | | | | | (s, 3H). |
| 1-408 | SO | 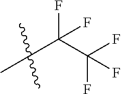 [SMILES: *C(F)(F)C(F)(F)F] | Me | H | ¹H NMR (500 MHz, DMSO-d6) δ 12.38 (s, 1H), 8.31 (dd, Hz, 1H), 7.59 |
| | | | | | (d, J = 7.5 |
| | | | | | Hz, 1H), 2.45 |
| | | | | | (s, 3H). |
| 1-409 | SO | 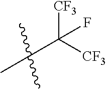 [SMILES: *C(F)(C(F)(F)F)C(F)(F)F] | Me | H | ¹H NMR (500 MHz, DMSO-d6) δ 12.38 (s, 1H), 8.36 (dd, J = 7.5, 6.0 |
| | | | | | Hz, 1H), 7.61 |
| | | | | | (d, J = 7.5 |
| | | | | | Hz, 1H), 2.46 |
| | | | | | (s, 3H). |
| 1-410 | SO | 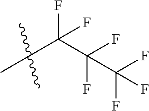 [SMILES: *C(F)(F)C(F)(F)C(F)(F)F] | Me | H | ¹H NMR (500 MHz, DMSO-d6) δ 12.38 (s, 1H), 8.35 (dd, J = 7.5, 6.0 Hz, 1H), 7.59 |
| | | | | | (d, J = 7.5 |
| | | | | | Hz, 1H), 2.45 |
| | | | | | (s, 3H). |
| 1-411 | SO | 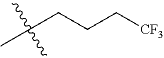 [SMILES: *CCCC(F)(F)F] | Me | H | ¹H NMR (500 MHz, DMSO-d6) δ 12.38 (s, |
| | | | | | 1H), 8.34 (dd, |
| | | | | | J = 7.5, 6.0 |
| | | | | | Hz, 1H), 7.61 |
| | | | | | (d, J = 7.5 |
| | | | | | Hz, 1H), 2.76 |
| | | | | | (t, J = 8.0 Hz, |
| | | | | | 2H), 2.52- |
| | | | | | 2.40 (m, 5H), |
| | | | | | 1.38-1.27 (m, |
| | | | | | 2H). |
| 1-412 | SO | 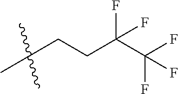 [SMILES: *CCC(F)(F)C(F)(F)F] | Me | H | ¹H NMR (500 MHz, DMSO-d6) δ 12.38 (s, 1H), 8.42 (dd, J = 7.5, 6.0 |
| | | | | | Hz, 1H), 7.60 |
| | | | | | (d, J = 7.5 |
| | | | | | Hz, 1H), 2.76 |
| | | | | | (t, J = 8.5 Hz, |
| | | | | | 2H), 2.46 (s, |
| | | | | | 3H), 1.93- |
| | | | | | 1.82 (m, 2H). |
| 1-413 | SO | 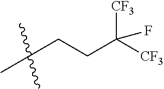 [SMILES: *CCC(F)(C(F)(F)F)C(F)(F)F] | Me | H | ¹H NMR (500 MHz, DMSO-d6) δ 12.38 (s, 1H), 8.37 (dd, J = 7.5, 6.0 |
| | | | | | Hz, 1H), 7.59 |
| | | | | | (d, J = 7.5 |
| | | | | | Hz, 1H), 2.76 |
| | | | | | (t, J = 8.0 Hz, |
| | | | | | 2H), 2.46 (s, |
| | | | | | 3H), 1.71 (dt, |
| | | | | | J = 25.0, 8.0 |
| | | | | | Hz, 2H). |
| 1-414 | SO | 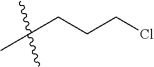 [SMILES: *CCCCl] | Me | H | ¹H NMR (500 MHz, DMSO-d₆) δ 12.38 (s, |
| | | | | | 1H), 8.33 (dd, |
| | | | | | J = 7.5, 6.0 |
| | | | | | Hz, 1H), 7.59 |
| | | | | | (d, J = 7.5 |
| | | | | | Hz, 1H), 3.57 |
| | | | | | (t, J = 7.5 |
| | | | | | Hz, 2H), 2.76 |
| | | | | | (t, J = 5.5 |
| | | | | | Hz, 2H), 2.46 |
| | | | | | (s, 3H), 1.91- |
| | | | | | 1.80 (m, 2H). |
| 1-415 | SO | 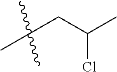 [SMILES: *CC(C)Cl] | Me | H | 1H NMR (500 MHz, DMSO-d6) δ 12.38 (s, 1H), 8.33 (dd, |
| | | | | | J = 7.5, 6.0 |
| | | | | | Hz, 1H), 7.59 |
| | | | | | (d, J = 7.5 |
| | | | | | Hz, 1H), 4.76- |
| | | | | | 4.64 (m, |
| | | | | | 1H), 3.13 |
| | | | | | (dd, J = 12.5, |
| | | | | | 7.0 Hz, 1H), |
| | | | | | 2.88 (dd, J = |
| | | | | | 12.5, 7.0 Hz, |
| | | | | | 1H), 2.46 (s, |
| | | | | | 3H), 1.55 (d, |
| | | | | | J = 7.0 Hz, |
| | | | | | 3H). |
| 1-416 | SO | 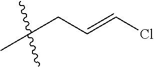 [SMILES: *C/C=C/Cl] | Me | H | |
| 1-417 | SO | 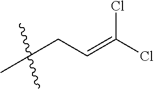 [SMILES: *CC=C(Cl)Cl] | Me | H | ¹H NMR (500 MHz, DMSO-d6) δ 12.38 (s, 1H), 8.33 (dd, J = 7.5, 6.0 |
| | | | | | Hz, 1H), 7.56 |
| | | | | | (d, J = 7.5 |
| | | | | | Hz, 1H), 6.01 |
| | | | | | (t, J = 6.0 Hz, |
| | | | | | 1H), 3.45 (d, |
| | | | | | J = 6.0 Hz, |
| | | | | | 2H), 2.46 (s, |
| | | | | | 3H). |
| 1-418 | SO | 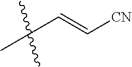 [SMILES: */C=C/C#N] | Me | H | |
| 1-419 | SO | 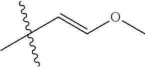 [SMILES: */C=C/OC] | Me | H | |
| 1-420 | SO | 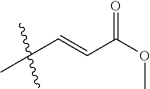 [SMILES: */C=C/C(=O)OC] | Me | H | |
| 1-421 | SO | 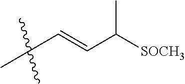 | Me | H | |
| 1-422 | SO | 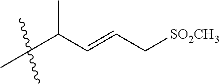 [SMILES: *C(C)/C=C/CS(C)(=O)=O] | Me | H | |
| 1-423 | SO | 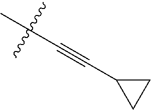 [SMILES: *C#CC1CC1] | Me | H | |
| 1-424 | SO | 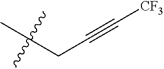 [SMILES: *CC#CC(F)(F)F] | Me | H | |
| 1-425 | SO | 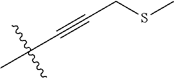 [SMILES: *C#CCSC] | Me | H | |
| 1-426 | SO | 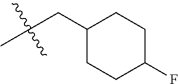 [SMILES: *CC1CCC(F)CC1] | Me | H | |
| 1-427 | SO | 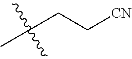 [SMILES: *CCC#N] | Me | H | ¹H NMR (500 MHz, DMSO-d6) δ 12.38 (s, |
| | | | | | 1H), 8.35 (dd, |
| | | | | | J = 7.5, 6.0 |
| | | | | | Hz, 1H), 7.60 |
| | | | | | (d, J = 7.5 |
| | | | | | Hz, 1H), 3.15 |
| | | | | | (t, J = 8.0 |
| | | | | | Hz, 2H), 2.55 |
| | | | | | (t, J = 8.0 |
| | | | | | Hz, 2H), 2.46 |
| | | | | | (s, 3H). |
| 1-428 | SO | 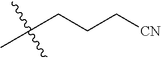 [SMILES: *CCCC#N] | Me | H | ¹H NMR (500 MHz, DMSO-d₆) δ 12.38 (s, |
| | | | | | 1H), 8.34 (dd, |
| | | | | | J = 7.5, 6.0 |
| | | | | | Hz, 1H), 7.59 |
| | | | | | (d, J = 7.5 |
| | | | | | Hz, 1H), 2.76 |
| | | | | | (t, J = 5.0 |
| | | | | | Hz, 2H), 2.46 |
| | | | | | (s, 3H), 2.14 |
| | | | | | (t, J = 5.5 |
| | | | | | Hz, 2H), 2.00 |
| | | | | | (1), J = 5.5 |
| | | | | | Hz, 2H). |
| 1-429 | SO | 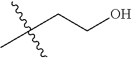 [SMILES: *CCO] | Me | H | ¹H NMR (500 MHz, DMSO-d6) δ 12.38 (s, |
| | | | | | 1H), 8.33 (dd, |
| | | | | | J = 7.5, 6.0 |
| | | | | | Hz, 1H), 7.60 |
| | | | | | (d, J = 7.5 |
| | | | | | Hz, 1H), 4.27 |
| | | | | | (t, J = 5.0 Hz, |
| | | | | | 1H), 3.87- |
| | | | | | 3.72 (m, 2H), |
| | | | | | 2.97 (t, J = |
| | | | | | 4.0 Hz, 2H), |
| | | | | | 2.46 (s, 3H). |
| 1-430 | SO | 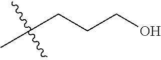 [SMILES: *CCCO] | Me | H | ¹H NMR (500 MHz, DMSO-d6) δ 12.38 (s, |
| | | | | | 1H), 8.34 (dd, |
| | | | | | J = 7.5, 6.0 |
| | | | | | Hz, 1H), 7.59 |
| | | | | | (d, J = 7.5 |
| | | | | | Hz, 1H), 4.34 |
| | | | | | (t, J = 5.0 Hz, |
| | | | | | 1H), 3.50 (q, |
| | | | | | J = 5.0 Hz, |
| | | | | | 2H), 2.76 (t, |
| | | | | | J = 8.0 Hz, |
| | | | | | 2H), 2.46 (s, |
| | | | | | 3H), 1.82- |
| | | | | | 1.71 (m, 2H). |
| 1-431 | SO | 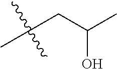 [SMILES: *CC(C)O] | Me | H | ¹H NMR (500 MHz, DMSO-d6) δ 12.38 (s, 1H), 8.34 (dd, |
| | | | | | J = 7.5, 6.0 |
| | | | | | Hz, 1H), 7.60 |
| | | | | | (d, J = 7.5 |
| | | | | | Hz, 1H), |
| | | | | | 5.24-5.15 (m, |
| | | | | | 1H), 4.34 (d, |
| | | | | | J = 5.0 Hz, |
| | | | | | 1H), 4.41 (d, |
| | | | | | J = 5.0 Hz, |
| | | | | | 2H), 3.12 (d, |
| | | | | | J = 7.0 Hz, |
| | | | | | 2H), 2.46 (s, |
| | | | | | 3H). |
| 1-432 | SO | 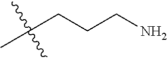 [SMILES: *CCCN] | Me | H | ¹H NMR (500 MHz, DMSO-d6) δ 12.38 (s, |
| | | | | | 1H), 8.33 (dd, |
| | | | | | J = 7.5, 6.0 |
| | | | | | Hz, 1H), 7.61 |
| | | | | | (d, J = 7.5 |
| | | | | | Hz, 1H), 3.08- |
| | | | | | 2.93 (m, |
| | | | | | 4H), 3.01- |
| | | | | | 2.95 (m, 2H), |
| | | | | | 2.60 (s, 2H), |
| | | | | | 2.46 (s, 3H). |
| 1-433 | SO | 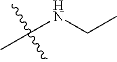 [SMILES: *NCC] | Me | H | ¹H NMR (500 MHz, DMSO-d6) δ 12.38 (s, |
| | | | | | 1H), 8.35 (dd, |
| | | | | | J = 7.5, 6.0 |
| | | | | | Hz, 1H), 7.61 |
| | | | | | (d, J = 7.5 |
| | | | | | Hz, 1H), 4.96 |
| | | | | | (s, 1H), 2.81 |
| | | | | | (q, J = 8.0 |
| | | | | | Hz, 2H), 2.46 |
| | | | | | (s, 3H), 0.87 |
| | | | | | (t, J = 8.0 |
| | | | | | Hz, 3H). |
| 1-434 | SO | 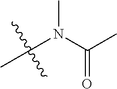 [SMILES: *N(C)C(C)=O] | Me | H | |
| 1-435 | SO | 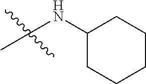 [SMILES: *NC1CCCCC1] | Me | H | |
| 1-436 | SO | 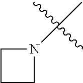 [SMILES: *N1CCC1] | Me | H | |
| 1-437 | SO | 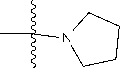 [SMILES: *N1CCCC1] | Me | H | |
| 1-438 | SO | 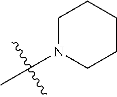 [SMILES: *N1CCCCC1] | Me | H | |
| 1-439 | SO | 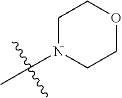 [SMILES: *N1CCOCC1] | Me | H | |
| 1-440 | SO | 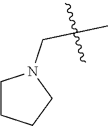 [SMILES: *CN1CCCC1] | Me | H | |
| 1-441 | SO | 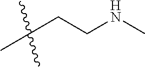 [SMILES: *CCNC] | Me | H | ¹H NMR (500 MHz, DMSO-d6) δ 12.38 (s, |
| | | | | | 1H), 8.34 (dd, |
| | | | | | J = 7.5, 6.0 |
| | | | | | Hz, 1H), 7.59 |
| | | | | | (d, J = 7.5 |
| | | | | | Hz, 1H), |
| | | | | | 4.04-3.92 (m, |
| | | | | | 2H), 3.85- |
| | | | | | 3.72 (m, 2H), |
| | | | | | 2.46 (s, 3H), |
| | | | | | 2.32 (s, 3H), |
| | | | | | 1.97 (s, 1H). |
| 1-442 | SO | 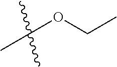 [SMILES: *OCC] | Me | H | ¹H NMR (500 MHz, DMSO-d6) δ 12.38 (s, |
| | | | | | 1H), 8.34 (dd, |
| | | | | | J = 7.5, 6.0 |
| | | | | | Hz, 1H), 7.60 |
| | | | | | (d, J = 7.5 |
| | | | | | Hz, 1H), 3.57 |
| | | | | | (q, J = 8.0 |
| | | | | | Hz, 2H), 2.46 |
| | | | | | (s, 3H), 1.15 |
| | | | | | (t, J = 8.0 Hz, |
| | | | | | 3H). |
| 1-443 | SO | 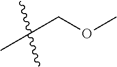 [SMILES: *COC] | Me | H | ¹H NMR (500 MHz, DMSO-d6) δ 12.38 (s, |
| | | | | | 1H), 8.32 (dd, |
| | | | | | J = 7.5, 6.0 |
| | | | | | Hz, 1H), 7.61- |
| | | | | | 7.55 (m, |
| | | | | | 1H), 4.86- |
| | | | | | 4.73 (m, 2H), |
| | | | | | 3.21 (s, 3H), |
| | | | | | 2.46 (s, 3H). |
| 1-444 | SO | 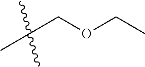 [SMILES: *COCC] | Me | H | ¹H NMR (500 MHz, DMSO-d₆) δ 12.38 (s, |
| | | | | | 1H), 8.34 (dd, |
| | | | | | J = 7.5, 6.0 |
| | | | | | Hz, 1H), 7.61 |
| | | | | | (d, J = 7.5 |
| | | | | | Hz, 1H), 3.71 |
| | | | | | (t, J = 7.5 |
| | | | | | Hz, 1H), 3.43 |
| | | | | | (q, J = 8.0 |
| | | | | | Hz, 2H), 2.95 |
| | | | | | (t, J = 7.5 |
| | | | | | Hz, 1H), 2.45 |
| | | | | | (s, 3H), 1.08 |
| | | | | | (t, J = 8.0 |
| | | | | | Hz, 3H). |
| 1-445 | SO | 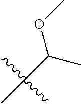 [SMILES: *C(C)OC] | Me | H | ¹H NMR (500 MHz, DMSO-d6) 12.41 (s, 1H), 8.12 (dd, J = 8.0, |
| | | | | | 7.0 Hz, 1H), |
| | | | | | 7.83 (d, J = |
| | | | | | 8.0 Hz, 1H), |
| | | | | | 4.35-4.24 (m, |
| | | | | | 1H), 3.32 (s, |
| | | | | | 3H), 2.50 (s, |
| | | | | | 3H), 1.45 (d, |
| | | | | | J = 7.5 Hz, |
| | | | | | 3H) |
| 1-446 | SO | 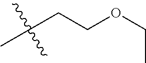 [SMILES: *CCOCC] | Me | H | ¹H NMR (500 MHz, DMSO-d₆) δ 12.38 (s, |
| | | | | | 1H), 8.32 (dd, |
| | | | | | J = 7.5, 6.0 |
| | | | | | Hz, 1H), 7.58 |
| | | | | | (d, J = 7.5 |
| | | | | | Hz, 1H), |
| | | | | | 4.86-4.73 (m, |
| | | | | | 2H), 4.56- |
| | | | | | 4.43 (m, 2H), |
| | | | | | 3.46 (q, J = |
| | | | | | 8.0 Hz, 2H), |
| | | | | | 2.46 (s, 3H), |
| | | | | | 1.03 (t, J = |
| | | | | | 8.0 Hz, 3H). |
| 1-447 | SO | 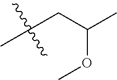 [SMILES: *CC(C)OC] | Me | H | ¹H NMR (500 MHz, DMSO-d₆) δ 12.38 (s, 1H), 8.34 (dd, |
| | | | | | J = 7.5, 6.0 |
| | | | | | Hz, 1H), 7.64- |
| | | | | | 7.58 (m, |
| | | | | | 1H), 3.70- |
| | | | | | 3.61 (m, 1H), |
| | | | | | 3.22 (s, 3H), |
| | | | | | 3.02 (dd, J = |
| | | | | | 12.0, 7.0 Hz, |
| | | | | | 1H), 2.77 |
| | | | | | (dd, J = 12.0, |
| | | | | | 7.0 Hz, 1H), |
| | | | | | 2.46 (s, 3H), |
| | | | | | 1.18 (d, J = |
| | | | | | 7.0 Hz, 3H). |
| 1-448 | SO | 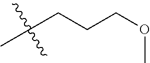 [SMILES: *CCCOC] | Me | H | ¹H NMR (500 MHz, DMSO-d₆) δ 12.38 (s, |
| | | | | | 1H), 8.33 (dd, |
| | | | | | J = 7.5, 6.0 |
| | | | | | Hz, 1H), 7.59 |
| | | | | | (d, J = 7.5 |
| | | | | | Hz, 1H), 3.34 |
| | | | | | (t, J = 7.5 |
| | | | | | Hz, 2H), 3.18 |
| | | | | | (s, 3H), 2.76 |
| | | | | | (t, J = 5.5 |
| | | | | | Hz, 2H), 2.46 |
| | | | | | (s, 3H), 1.80- |
| | | | | | 1.71 (m, 2H). |
| 1-449 | SO | 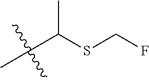 [SMILES: *C(C)SCF] | Me | H | |
| 1-450 | SO | 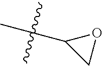 [SMILES: *C1CO1] | Me | H | |
| 1-451 | SO | 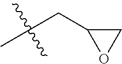 [SMILES: *CC1CO1] | Me | H | ¹H NMR (500 MHz, DMSO-d₆) δ 12.34 (s, |
| | | | | | 1H), 8.07- |
| | | | | | 8.95 (m, |
| | | | | | 1H), 7.59 (d, |
| | | | | | J = 8.0 Hz, |
| | | | | | 1H), 3.89- |
| | | | | | 3.80 (m, |
| | | | | | 1H), 3.02- |
| | | | | | 2.95 (m, |
| | | | | | 1H), 2.81- |
| | | | | | 2.72 (m, |
| | | | | | 2H), 2.47 (s, |
| | | | | | 3H), 2.44- |
| | | | | | 2.39 (m, |
| | | | | | 1H). |
| 1-452 | SO | 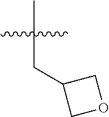 [SMILES: *CC1COC1] | Me | H | ¹H NMR (500 MHz, DMSO-d₆) δ 12.38 (s, 1H), 7.78 (dd, J = 7.5, 6.0 Hz, 1H), 7.60 |
| | | | | | (d, J = 7.5 |
| | | | | | Hz, 1H), 4.75 |
| | | | | | (dd, J = 7.0 |
| | | | | | 6.0 Hz, 2H), |
| | | | | | 4.51 (dd, J = |
| | | | | | 7.0, 6.0 Hz, |
| | | | | | 2H), 3.28- |
| | | | | | 3.17 (m, 1H), |
| | | | | | 2.70 (d, J = |
| | | | | | 7.0 Hz, 2H), |
| | | | | | 2.45 (s, 3H). |
| 1-453 | SO | 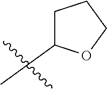 [SMILES: *C1CCCO1] | Me | H | |
| 1-454 | SO | 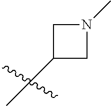 [SMILES: *C1CN(C)C1] | Me | H | |
| 1-455 | SO | 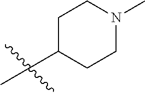 [SMILES: *C1CCN(C)CC1] | Me | H | |
| 1-456 | SO | 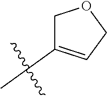 [SMILES: *C1=CCOC1] | Me | H | |
| 1-457 | SO | 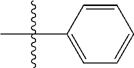 [SMILES: *c1ccccc1] | Me | H | ¹H NMR (500 MHz, DMSO-d₆) δ 12.01 (s, |
| | | | | | 1H), 8.23- |
| | | | | | 8.10 (m, |
| | | | | | 1H), 7.93 (d, |
| | | | | | J = 8.0 Hz, |
| | | | | | 1H), 7.65- |
| | | | | | 7.46 (m, |
| | | | | | 5H), 2.48 (s, |
| | | | | | 3H). |
| 1-458 | SO | 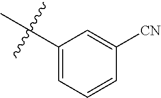 [SMILES: *c1cccc(C#N)c1] | Me | H | |
| 1-459 | SO | 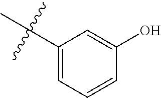 [SMILES: *c1cccc(O)c1] | Me | H | |
| 1-460 | SO | 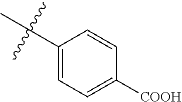 [SMILES: *c1ccc(C(=O)O)cc1] | Me | H | |
| 1-461 | SO | 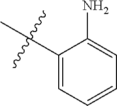 [SMILES: *c1ccccc1N] | Me | H | |
| 1-462 | SO | 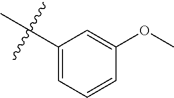 [SMILES: *c1cccc(OC)c1] | Me | H | |
| 1-463 | SO | 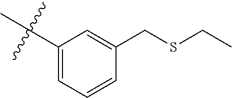 [SMILES: *c1cccc(CSCC)c1] | Me | H | |
| 1-464 | SO | 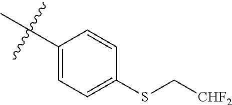 [SMILES: *c1ccc(SCC(F)F)cc1] | Me | H | |
| 1-465 | SO | 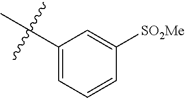 [SMILES: *c1cccc(S(C)(=O)=O)c1] | Me | H | |
| 1-466 | SO | 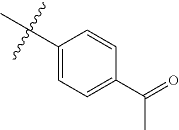 [SMILES: *c1ccc(C(C)=O)cc1] | Me | H | |
| 1-467 | SO | 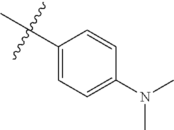 [SMILES: *c1ccc(N(C)C)cc1] | Me | H | |
| 1-468 | SO | 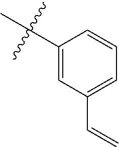 [SMILES: *c1cccc(C=C)c1] | Me | H | |
| 1-469 | SO | 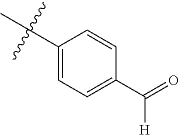 [SMILES: *c1ccc(C=O)cc1] | Me | H | |
| 1-470 | SO | 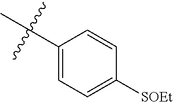 | Me | H | |
| 1-471 | SO | 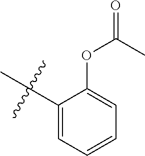 [SMILES: *c1ccccc1OC(C)=O] | Me | H | |
| 1-472 | SO | 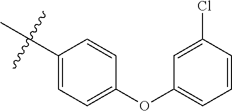 [SMILES: *c1ccc(Oc2cccc(Cl)c2)cc1] | Me | H | |
| 1-473 | SO | 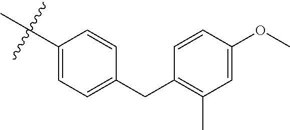 [SMILES: *c1ccc(Cc2ccc(OC)cc2C)cc1] | Me | H | |
| 1-474 | SO | 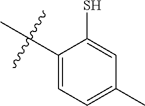 [SMILES: *c1ccc(C)cc1S] | Me | H | |
| 1-475 | SO | 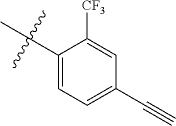 [SMILES: *c1ccc(C#C)cc1C(F)(F)F] | Me | H | |
| 1-476 | SO | 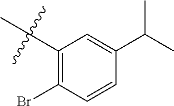 [SMILES: *c1cc(C(C)C)ccc1Br] | Me | H | |
| 1-477 | SO | 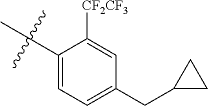 | Me | H | |
| 1-478 | SO | 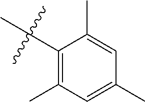 [SMILES: *c1c(C)cc(C)cc1C] | Me | H | |
| 1-479 | SO | 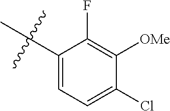 [SMILES: *c1ccc(Cl)c(OC)c1F] | Me | H | |
| 1-480 | SO | 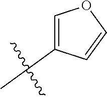 [SMILES: *c1ccoc1] | Me | H | |
| 1-481 | SO | 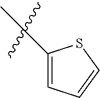 [SMILES: *c1cccs1] | Me | H | |
| 1-482 | SO | 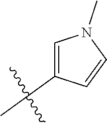 [SMILES: *c1ccn(C)c1] | Me | H | |
| 1-483 | SO | 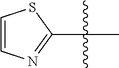 [SMILES: *c1nccs1] | Me | H | |
| 1-484 | SO | 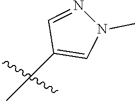 [SMILES: *c1cnn(C)c1] | Me | H | |
| 1-485 | SO | 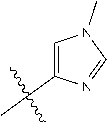 [SMILES: *c1cn(C)cn1] | Me | H | |
| 1-486 | SO | 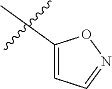 [SMILES: *c1ccno1] | Me | H | |
| 1-487 | SO | 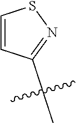 [SMILES: *c1ccsn1] | Me | H | |
| 1-488 | SO | 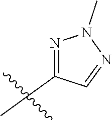 [SMILES: *c1cnn(C)n1] | Me | H | |
| 1-489 | SO | 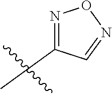 [SMILES: *c1cnon1] | Me | H | |
| 1-490 | SO | 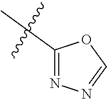 [SMILES: *c1nnco1] | Me | H | |
| 1-491 | SO | 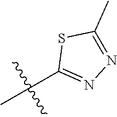 [SMILES: *c1nnc(C)s1] | Me | H | |
| 1-492 | SO | 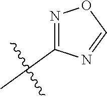 [SMILES: *c1ncon1] | Me | H | |
| 1-493 | SO | 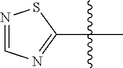 [SMILES: *c1ncns1] | Me | H | |
| 1-494 | SO | 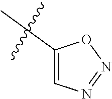 [SMILES: *c1cnno1] | Me | H | |
| 1-495 | SO | 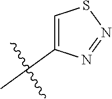 [SMILES: *c1csnn1] | Me | H | |
| 1-496 | SO | 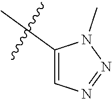 [SMILES: *c1cnnn1C] | Me | H | |
| 1-497 | SO | 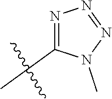 [SMILES: *c1nnnn1C] | Me | H | |
| 1-498 | SO | 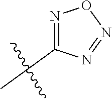 [SMILES: *c1nnon1] | Me | H | |
| 1-499 | SO | 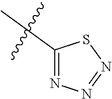 [SMILES: *c1nnns1] | Me | H | |
| 1-500 | SO | 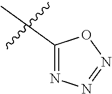 [SMILES: *c1nnno1] | Me | H | |
| 1-501 | SO | 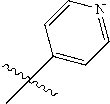 [SMILES: *c1ccncc1] | Me | H | ¹H NMR (500 MHz, DMSO-d₆) δ 12.59 (s, 1H) 8.10- 7.91 (m, |
| | | | | | 4H), 7.17 (d, |
| | | | | | J = 6.5 Hz, |
| | | | | | 2H), 2.47 (s, |
| | | | | | 3H). |
| 1-502 | SO | 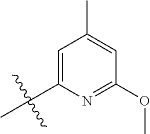 [SMILES: *c1cc(C)cc(OC)n1] | Me | H | |
| 1-503 | SO | 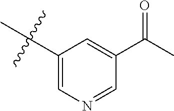 [SMILES: *c1cncc(C(C)=O)c1] | Me | H | |
| 1-504 | SO | 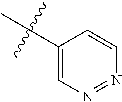 [SMILES: *c1ccnnc1] | Me | H | |
| 1-505 | SO | 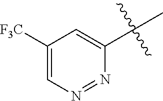 [SMILES: *c1cc(C(F)(F)F)cnn1] | Me | H | |
| 1-506 | SO | 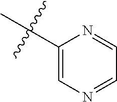 [SMILES: *c1cnccn1] | Me | H | |
| 1-507 | SO | 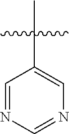 [SMILES: *c1cncnc1] | Me | H | |
| 1-508 | SO | 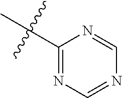 [SMILES: *c1ncncn1] | Me | H | |
| 1-509 | SO | 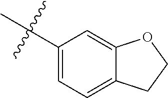 [SMILES: *c1ccc2c(c1)OCC2] | Me | H | |
| 1-510 | SO | 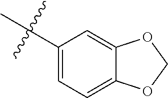 [SMILES: *c1ccc2c(c1)OCO2] | Me | H | |
| 1-511 | SO | 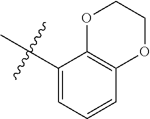 [SMILES: *c1cccc2c1OCCO2] | Me | H | |
| 1-512 | SO | 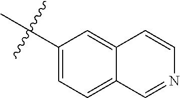 [SMILES: *c1ccc2cnccc2c1] | Me | H | |
| 1-513 | SO | 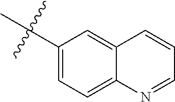 [SMILES: *c1ccc2ncccc2c1] | Me | H | |
| 1-514 | SO | 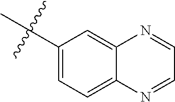 [SMILES: *c1ccc2nccnc2c1] | Me | H | |
| 1-515 | SO | 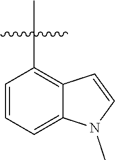 [SMILES: *c1cccc2c1ccn2C] | Me | H | |
| 1-516 | SO | 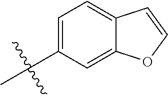 [SMILES: *c1ccc2ccoc2c1] | Me | H | |
| 1-517 | SO | 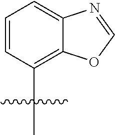 [SMILES: *c1cccc2ncoc12] | Me | H | |
| 1-518 | SO | 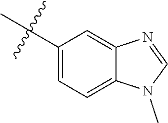 [SMILES: *c1ccc2c(c1)ncn2C] | Me | H | |
| 1-519 | SO | 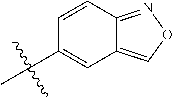 [SMILES: *c1ccc2nocc2c1] | Me | H | |
| 1-520 | SO | 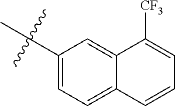 [SMILES: *c1ccc2cccc(C(F)(F)F)c2c1] | Me | H | |
| 1-521 | SO | 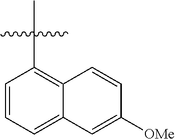 [SMILES: *c1cccc2cc(OC)ccc12] | Me | H | |
| 1-522 | SO | 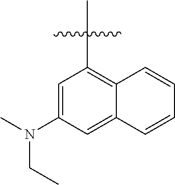 [SMILES: *c1cc(N(C)CC)cc2ccccc12] | Me | H | |
| 1-523 | SO | 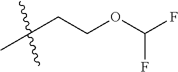 [SMILES: *CCOC(F)F] | Me | H | ¹H NMR (500 MHz, DMSO-d₆) δ 12.38 (s, 1H), 8.27 |
| | | | | | (dd, J = 7.5, |
| | | | | | 6.0 Hz, 1H), |
| | | | | | 7.58 (d, J = |
| | | | | | 7.5 Hz, 1H), |
| | | | | | 7.15 (t, J = |
| | | | | | 50.0 Hz, 1H), |
| | | | | | 3.71 (t, J = |
| | | | | | 8.0 Hz, 2H), |
| | | | | | 2.95 (t, J = |
| | | | | | 8.0 Hz, 2H), |
| | | | | | 2.40 (s, 3H). |
| 1-524 | SO | 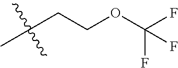 [SMILES: *CCOC(F)(F)F] | Me | H | ¹H NMR (500 MHz, DMSO-d₆) δ 12.38 (s, |
| | | | | | 1H), 8.34 (dd, |
| | | | | | J = 7.5, 6.0 |
| | | | | | Hz, 1H), 7.59 |
| | | | | | (d, J = 7.5 |
| | | | | | Hz, 1H), |
| | | | | | 3.71.-3.62 |
| | | | | | (m, 2H), 2.95 |
| | | | | | (t, J = 8.0 |
| | | | | | Hz, 2H), 2.46 |
| | | | | | (s, 3H). |
| 1 -525 | SO | 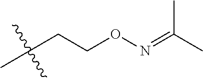 [SMILES: *CCON=C(C)C] | Me | H | ¹H NMR (500 MHz, DMSO-d6) δ 12.38 (s, 1H), 8.33 (dd, |
| | | | | | J = 7.5, 6.0 |
| | | | | | Hz, 1H), 7.59 |
| | | | | | (d, J = 7.5 |
| | | | | | Hz, 1H), |
| | | | | | 3.87-3.75 (m, |
| | | | | | 2H), 2.97 (t, |
| | | | | | J = 4.5 Hz, |
| | | | | | 2H), 2.46 (s, |
| | | | | | 3H), 2.42 (s, |
| | | | | | 6H). |
| 1-526 | SO | 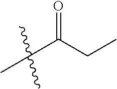 [SMILES: *C(=O)CC] | Me | H | ¹H NMR (500 MHz, DMSO-d6) δ 12.38 (s, 1H), 8.34 (dd, J = 7.5, 6.0 |
| | | | | | Hz, 1H), 7.59 |
| | | | | | (d, J = 7.5 |
| | | | | | Hz, 1H), 2.48- |
| | | | | | 2.40 (m, |
| | | | | | 2H), 1.26 (t, |
| | | | | | J = 8.0 Hz, |
| | | | | | 3H). |
| 1-527 | SO | 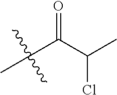 [SMILES: *C(=O)C(C)Cl] | Me | H | |
| 1-528 | SO | 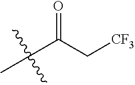 [SMILES: *C(=O)CC(F)(F)F] | Me | H | ¹H NMR (500 MHz, DMSO-d6) δ 12.38 (s, 1H), 8.34 (dd, |
| | | | | | J = 7.5, 6.0 |
| | | | | | Hz, 1H), 7.59 |
| | | | | | (d, J = 7.5 |
| | | | | | Hz, 1H), 2.92 |
| | | | | | (q, J = 45.0 |
| | | | | | Hz, 2H), 2.45 |
| | | | | | (s, 3H). |
| 1-529 | SO | 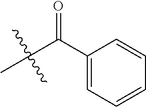 [SMILES: *C(=O)c1ccccc1] | Me | H | ¹H NMR (500 MHz, DMSO-d6) δ 12.38 (s, 1H), 8.10- 8.03 (m, 2H), 7.79 (dd, J = |
| | | | | | 7.5, 6.0 Hz, |
| | | | | | 1H), 7.67- |
| | | | | | 7.54 (m, 1H), |
| | | | | | 7.61-7.51 |
| | | | | | (m, 3H), 2.45 |
| | | | | | (s, 3H). |
| 1-530 | SO | 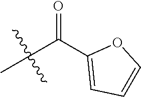 [SMILES: *C(=O)c1ccco1] | Me | H | ¹H NMR (500 MHz, DMSO-d6) δ 12.38 (s, 1H), 8.35 (dd, J = 7.5, 6.0 |
| | | | | | Hz, 1H), 7.84 |
| | | | | | (d, J = 7.5 |
| | | | | | Hz, 1H), 7.60 |
| | | | | | (dd, J = 7.5 |
| | | | | | Hz, 1H), 7.52 |
| | | | | | (dd, J = 7.5, |
| | | | | | 1.5 Hz, 1H), |
| | | | | | 6.74 (t, J = |
| | | | | | 7.5 Hz, 1H), |
| | | | | | 2.46 (s, 3H). |
| 1-531 | SO | 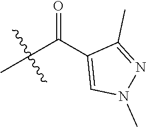 [SMILES: *C(=O)c1cn(C)nc1C] | Me | H | ¹H NMR (500 MHz, DMSO-d6) δ 12.38 (s, 1H), 8.35 (dd, J = 7.5, 6.0 Hz, 1H), 8.24 (s, 1H), 7.60 |
| | | | | | (d, J = 7.5 |
| | | | | | Hz, 1H), 3.81 |
| | | | | | (s, 3H), 2.60 |
| | | | | | (s, 3H), 2.46 |
| | | | | | (s, 3H). |
| 1-532 | SO | 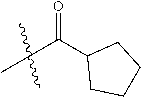 [SMILES: *C(=O)C1CCCC1] | Me | H | |
| 1-533 | SO | 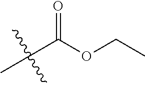 [SMILES: *C(=O)OCC] | Me | H | |
| 1-534 | SO | 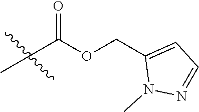 [SMILES: *C(=O)OCc1ccnn1C] | Me | H | |
| 1-535 | SO | 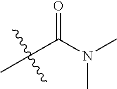 [SMILES: *C(=O)N(C)C] | Me | H | ¹H NMR (500 MHz, DMSO-d6) δ 12.38 (s, 1H), 8.34 (dd, J = 7.5, 6.0 |
| | | | | | Hz, 1H), 7.59 |
| | | | | | (d, J = 7.5 |
| | | | | | Hz, 1H), 3.06 |
| | | | | | (s, 6H), 2.45 |
| | | | | | (s, 3H). |
| 1-536 | SO | 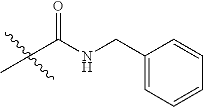 [SMILES: *C(=O)NCc1ccccc1] | Me | H | |
| 1-537 | SO | 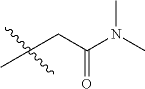 [SMILES: *CC(=O)N(C)C] | Me | H | ¹H NMR (500 MHz, DMSO-d6) δ 12.38 (s, 1H), 8.06 (dd, J = 7.5, 6.0 |
| | | | | | Hz, 1H), 7.59 |
| | | | | | (d, J = 7.5 |
| | | | | | Hz, 1H), 3.67 |
| | | | | | (s, 2H), 2.91 |
| | | | | | (s, 6H), 2.46 |
| | | | | | (s, 3H). |
| 1-538 | SO | 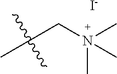 | Me | H | ¹H NMR (500 MHz, DMSO-d6) δ 12.38 (s, 1H), 7.77 (dd, |
| | | | | | J = 7.5, 6.0 |
| | | | | | Hz, 1H), 7.60- |
| | | | | | 7.55 (m, |
| | | | | | 1H), 4.73 (s, |
| | | | | | 2H), 3.06 (s, |
| | | | | | 9H), 2.45 (s, |
| | | | | | 3H). |
| 1-539 | SO | 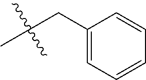 [SMILES: *Cc1ccccc1] | Me | H | ¹H NMR (500 MHz, DMSO-d₆) δ 12.67 (s, 1H), 8.16- |
| | | | | | 8.05 (m, |
| | | | | | 1H), 7.78 (d, |
| | | | | | J = 8.0 Hz, |
| | | | | | 1H), 7.30- |
| | | | | | 7.16 (m, |
| | | | | | 5H), 4.78- |
| | | | | | 4.63 (m, |
| | | | | | 2H), 2.47 (s, |
| | | | | | 3H). |
| 1-540 | SO | 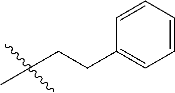 [SMILES: *CCc1ccccc1] | Me | H | ¹H NMR (500 MHz, DMSO-d₆) δ 12.55 (s, 1H), 8.10- 8.01 (m, |
| | | | | | 1H), 7.87 (d, |
| | | | | | J = 8.0 Hz, |
| | | | | | 1H), 7.35- |
| | | | | | 7.26 (m, |
| | | | | | 4H), 7.27- |
| | | | | | 7.20 (m, |
| | | | | | 1H), 3.88- |
| | | | | | 3.77 (m, |
| | | | | | 1H), 3.48- |
| | | | | | 3.40 (m, |
| | | | | | 1H), 3.18- |
| | | | | | 3.08 (m, |
| | | | | | 1H), 3.02- |
| | | | | | 2.92 (m, |
| | | | | | 1H), 2.48 (s, |
| | | | | | 3H). |
| 1-541 | SO | 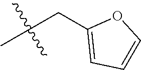 [SMILES: *Cc1ccco1] | Me | H | ¹H NMR (500 MHz, DMSO-d₆) δ 12.59 (s, 1H), 8.14- |
| | | | | | 8.02 (m, |
| | | | | | 1H), 7.87- |
| | | | | | 7.82 (m, |
| | | | | | 1H), 7.62- |
| | | | | | 7.59 (m, |
| | | | | | 1H), 6.39- |
| | | | | | 6.34 (m, |
| | | | | | 2H), 4.97- |
| | | | | | 4.86 (m, |
| | | | | | 1H), 4.84- |
| | | | | | 4.75 (m, |
| | | | | | 1H), 2.47 (s, |
| | | | | | 3H). |
| 1-542 | SO | 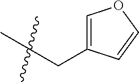 [SMILES: *Cc1ccoc1] | Me | H | |
| 1-543 | SO | 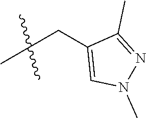 [SMILES: *Cc1cn(C)nc1C] | Me | H | ¹H NMR (500 MHz, DMSO-d6) δ 12.38 (s, 1H), 8.34 (dd, J = 7.5, 6.0 Hz, 1H), 7.60 |
| | | | | | (d, J = 7.5 |
| | | | | | Hz, 1H), 6.88 |
| | | | | | (s, 1H), 4.70 |
| | | | | | (s, 2H), 3.72 |
| | | | | | (s, 3H), 2.46 |
| | | | | | (s, 3H), 2.29 |
| | | | | | (s, 3H). |
| 1-544 | SO | 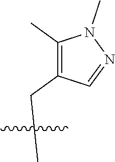 [SMILES: *Cc1cnn(C)c1C] | Me | H | |
| 1-545 | SO | 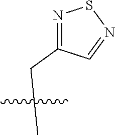 [SMILES: *Cc1cnsn1] | Me | H | |
| 1-546 | SO | 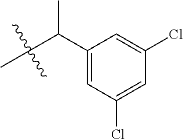 [SMILES: *C(C)c1cc(Cl)cc(Cl)c1] | Me | H | |
| 1-547 | SO | 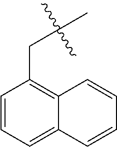 [SMILES: *Cc1cccc2ccccc12] | Me | H | |
| 1-548 | SO | 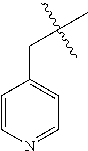 [SMILES: *Cc1ccncc1] | Me | H | |
| 1-549 | SO | 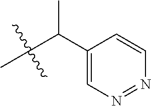 [SMILES: *C(C)c1ccnnc1] | Me | H | |
| 1-550 | SO | 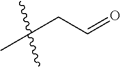 [SMILES: *CC=O] | Me | H | ¹H NMR (500 MHz, DMSO-d6) δ 12.38 (s, |
| | | | | | 1H), 9.72 (t, |
| | | | | | J = 6.0 Hz, |
| | | | | | 1H), 8.39 |
| | | | | | (dd, J = 7.5, |
| | | | | | 6.0 Hz, 1H), |
| | | | | | 7.60 (d, J = |
| | | | | | 7.5 Hz, 1H), |
| | | | | | 3.89-3.78 (m, |
| | | | | | 2H), 2.46 (s, |
| | | | | | 3H). |
| 1-551 | SO | 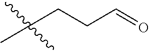 [SMILES: *CCC=O] | Me | H | ¹H NMR (500 MHz, DMSO-d6) δ |
| | | | | | 12.38 (s, |
| | | | | | 1H), 9.90 (t, |
| | | | | | J = 6.0 Hz, |
| | | | | | 1H), 8.34 |
| | | | | | (dd, J = 7.5, |
| | | | | | 6.0 Hz, 1H), |
| | | | | | 7.61 (d, J = |
| | | | | | 7.5 Hz, 1H), |
| | | | | | 3.11-3.01 (m, |
| | | | | | 2H), 2.74- |
| | | | | | 2.61 (m, 2H), |
| | | | | | 2.46 (s, 3H). |
| 1-552 | SO | 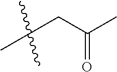 [SMILES: *CC(C)=O] | Me | H | ¹H NMR (500 MHz, DMSO-d₆) δ 12.38 (s, 1H), 8.34 |
| | | | | | (dd, J = 7.5, |
| | | | | | 6.0 Hz, 1H), |
| | | | | | 7.57 (d, J = |
| | | | | | 7.5 Hz, 1H), |
| | | | | | 3.94-3.83 (m, |
| | | | | | 2H), 2.45 (s, |
| | | | | | 3H), 2.16 (s, |
| | | | | | 3H). |
| 1-553 | SO | 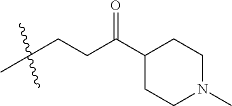 [SMILES: *CCC(=O)C1CCN(C)CC1] | Me | H | |
| 1-554 | SO | 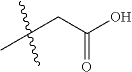 [SMILES: *CC(=O)O] | Me | H | ¹H NMR (500 MHz, DMSO-d₆) δ 12.41 (s, 1H), 12.38 (s, |
| | | | | | 1H), 8.18 |
| | | | | | (dd, J = 7.5, |
| | | | | | 6.0 Hz, 1H), |
| | | | | | 7.61 (d, J = |
| | | | | | 7.5 Hz, 1H), |
| | | | | | 3.72-3.63 (m, |
| | | | | | 2H), 2.46 (s, |
| | | | | | 3H). |
| 1-555 | SO | 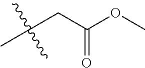 [SMILES: *CC(=O)OC] | Me | H | ¹H NMR (500 MHz, DMSO-d₆) δ 12.38 (s, |
| | | | | | 1H), 8.33 (dd, |
| | | | | | J = 7.5, 6.0 |
| | | | | | Hz, 1H), 7.61 |
| | | | | | (d, J = 7.5 |
| | | | | | Hz, 1H), |
| | | | | | 4.31-4.23 (m, |
| | | | | | 2H), 3.73 (s, |
| | | | | | 3H), 2.47 (s, |
| | | | | | 3H). |
| 1-556 | SO | 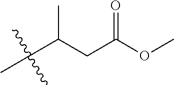 [SMILES: *C(C)CC(=O)OC] | Me | H | |
| 1-557 | SO | 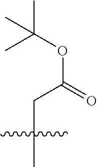 [SMILES: *CC(=O)OC(C)(C)C] | Me | H | ¹H NMR (500 MHz, DMSO-d₆) δ 12.38 (s, 1H), 8.35 (dd, J = 7.5, 6.0 Hz, 1H), 7.60 (d, J = 7.5 Hz, 1H), 4.31-4.22 (m, |
| | | | | | 2H), 2.46 (s, |
| | | | | | 3H), 1.39 (s, |
| | | | | | 9H). |
| 1-558 | SO | 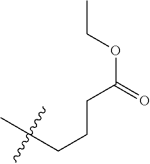 [SMILES: *CCCC(=O)OCC] | Me | H | ¹H NMR (500 MHz, DMSO-d₆) δ 12.38 (s, 1H), 8.33 (dd, J = 7.5, 6.0 Hz, 1H), 7.63-7.57 (m, 1H), 4.31-4.22 (m, |
| | | | | | 2H), 4.02- |
| | | | | | 3.86 (m, 4H), |
| | | | | | 4.17 (q, J = |
| | | | | | 8.0 Hz, 2H), |
| | | | | | 2.46 (s, 3H), |
| | | | | | 1.12 (t, J = |
| | | | | | 8.0 Hz, 3H). |
| 1-559 | SO | 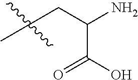 [SMILES: *CC(N)C(=O)O] | Me | H | |
| 1-560 | SO | 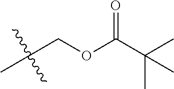 [SMILES: *COC(=O)C(C)(C)C] | Me | H | ¹H NMR (500 MHz, DMSO-d₆) δ 12.38 (s, 1H), 8.35 (dd, J = 7.5, |
| | | | | | 6.0 Hz, 1H), |
| | | | | | 7.60 (d, J = |
| | | | | | 7.5 Hz, 1H), |
| | | | | | 4.31-4.22 (m, |
| | | | | | 2H), 2.46 (s, |
| | | | | | 3H), 1.39 (s, |
| | | | | | 9H). |
| 1-561 | SO | 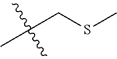 [SMILES: *CSC] | Me | H | ¹H NMR (500 MHz, DMSO-d₆) δ 12.38 (s, |
| | | | | | 1H), 8.29 |
| | | | | | (dd, J = 7.5, |
| | | | | | 6.0 Hz, 1H), |
| | | | | | 7.59 (d, J = |
| | | | | | 7.5 Hz, 1H), |
| | | | | | 3.93-3.82 (m, |
| | | | | | 2H), 2.45 (s, |
| | | | | | 3H), 2.21 (s, |
| | | | | | 3H). |
| 1-562 | SO | 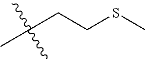 [SMILES: *CCSC] | Me | H | ¹H NMR (500 MHz, DMSO-d₆) δ 12.38 (s, |
| | | | | | 1H), 8.34 |
| | | | | | (dd, J = 7.5, |
| | | | | | 6.0 Hz, 1H), |
| | | | | | 7.60 (d, J = |
| | | | | | 7.5 Hz, 1H), |
| | | | | | 3.15 (t, J = |
| | | | | | 8.0 Hz, 2H), |
| | | | | | 2.67 (t, J = |
| | | | | | 8.0 Hz, 2H), |
| | | | | | 2.46 (s, 3H), |
| | | | | | 2.07 (s, 3H) |
| 1-563 | SO | 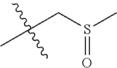 [SMILES: *CS(C)=O] | Me | H | ¹H NMR (500 MHz, DMSO-d₆) δ 12.38 (s, 1H), 8.24 |
| | | | | | (dd, J = 7.5, |
| | | | | | 6.0 Hz, 1H), |
| | | | | | 7.56 (d, J = |
| | | | | | 7.5 Hz, 1H), |
| | | | | | 4.06-3.91 (m, |
| | | | | | 2H), 2.53 (s, |
| | | | | | 3H), 2.45 (s, |
| | | | | | 3H). |
| 1-564 | SO | 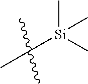 [SMILES: *[Si](C)(C)C] | Me | H | ¹H NMR (500 MHz, DMSO-d₆) δ 12.38 (s, 1H), 8.03 |
| | | | | | (dd, J = |
| | | | | | 7.5, 6.0 Hz, |
| | | | | | 1H), 7.62 (d, |
| | | | | | J = 7.5 Hz, |
| | | | | | 1H), 2.63 (s, |
| | | | | | 3H), 0.08 (s, |
| | | | | | 9H). |
| 1-565 | SO | 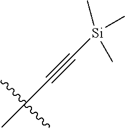 [SMILES: *C#C[Si](C)(C)C] | Me | H | |
| 1-566 | SO | 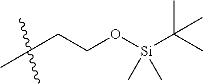 [SMILES: *CCO[Si](C)(C)C(C)(C)C] | Me | H | ¹H NMR (500 MHz, DMSO-d₆) δ 12.38 (s, 1H), 8.32 |
| | | | | | (dd, J = 7.5, |
| | | | | | 6.0 Hz, 1H), |
| | | | | | 7.59 (d, J = |
| | | | | | 7.5 Hz, 1H), |
| | | | | | 4.13-4.01 (m, |
| | | | | | 2H), 3.02 (t, |
| | | | | | J = 3.5 Hz, |
| | | | | | 2H), 2.46 (s, |
| | | | | | 3H), 1.03 (s, |
| | | | | | 9H), 0.21 (s, |
| | | | | | 6H). |
| 1-567 | SO | 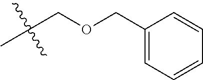 [SMILES: *COCc1ccccc1] | Me | H | ¹H NMR (500 MHz, DMSO-d₆) δ 12.38 (s, 1H), 8.33 |
| | | | | | (dd, J = 7.5, |
| | | | | | 6.0 Hz, 1H), |
| | | | | | 7.59 (dd, J = |
| | | | | | 7.5 Hz, 1H), |
| | | | | | 7.36-7.25 |
| | | | | | (m, 5H), 4.86 |
| | | | | | (s, 2H), 4.68 |
| | | | | | (s, 2H), 2.48 |
| | | | | | (s, 3H). |
| 1-568 | SO | Et | Me | Me | ¹H NMR |
| | | | | | (500 MHz, |
| | | | | | DMSO-d₆) δ |
| | | | | | 7.96 (dd, J = |
| | | | | | 7.5, 6.0 Hz, |
| | | | | | 1H), 7.57 (d, |
| | | | | | J = 7.5 Hz, |
| | | | | | 1H), 3.25 (s, |
| | | | | | 3H), 2.82 (q, |
| | | | | | J = 8.0 Hz, |
| | | | | | 2H), 2.45 (s, |
| | | | | | 3H), 1.33 (t, |
| | | | | | J = 8.0 Hz, |
| | | | | | 3H). |
| 1-569 | SO | Et | Me | Et | |
| 1-570 | SO | Et | Me | 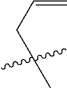 [SMILES: *CC=C] | |
| 1-571 | SO | Et | Me | 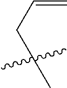 [SMILES: *CC=C] | ¹H NMR (500 MHz, DMSO-d₆) δ 7.79 (dd, J = 7.5, 6.0 Hz, 1H), 7.54 (d, |
| | | | | | J = 7.5 Hz, |
| | | | | | 1H), 5.93- |
| | | | | | 5.85 (m, |
| | | | | | 1H), 5.10- |
| | | | | | 5.01 (m, |
| | | | | | 1H), 4.89- |
| | | | | | 4.79 (m, |
| | | | | | 1H), 4.47- |
| | | | | | 4.43 (m, |
| | | | | | 2H), 2.85 (q, |
| | | | | | J = 8.0 Hz, |
| | | | | | 2H), 2.46 (s, |
| | | | | | 3H), 1.31 (t, |
| | | | | | J = 8.0 Hz, |
| | | | | | 3H). |
| 1-572 | SO | Et | Me | 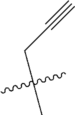 [SMILES: *CC#C] | ¹H NMR (500 MHz, DMSO-d₆) δ 7.81 (dd, J = 7.5, 6.0 Hz, 1H), 7.57 (d, J = 7.5 Hz, |
| | | | | | 1H), 3.94 (d, |
| | | | | | J = 3.0 Hz, |
| | | | | | 2H), 3.29 (t, |
| | | | | | J = 3.0 Hz, |
| | | | | | 1H), 2.84 (q, |
| | | | | | J = 8.0 Hz, |
| | | | | | 2H), 2.46 (s, |
| | | | | | 3H), 1.32 (t, |
| | | | | | J = 8.0 Hz, |
| | | | | | 3H). |
| 1-573 | SO | Et | Me | 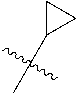 [SMILES: *C1CC1] | |
| 1-574 | SO | Et | Me | 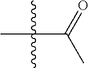 [SMILES: *C(C)=O] | ¹H NMR (500 MHz, DMSO-d₆) δ 7.66 (dd, J = |
| | | | | | 7.5, 6.0 Hz, |
| | | | | | 1H), 7.55- |
| | | | | | 7.49 (m, 1H), |
| | | | | | 2.88 (q, J = |
| | | | | | 8.0 Hz, 2H), |
| | | | | | 2.46 (s, 3H), |
| | | | | | 2.22 (s, 3H), |
| | | | | | 1.34 (t, J = |
| | | | | | 8.0 Hz, 3H). |
| 1-575 | SO | Et | Me | 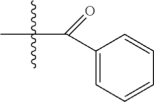 [SMILES: *C(=O)c1ccccc1] | ¹H NMR (500 MHz, DMSO-d₆) δ 8.10 (dd, J = 7.5, 6.0 Hz, 1H), 7.91- |
| | | | | | 7.84 (m, 2H), |
| | | | | | 7.63-7.56 |
| | | | | | (m, 2H), 7.52 |
| | | | | | (t, J = 7.5 |
| | | | | | Hz, 2H), 2.81 |
| | | | | | (q, J = 8.0 |
| | | | | | Hz, 2H), 2.33 |
| | | | | | (s, 3H), 1.32 |
| | | | | | (t, J = 8.0 |
| | | | | | Hz, 3H). |
| 1-576 | SO | Et | Me | 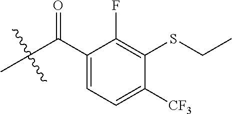 [SMILES: *C(=O)c1ccc(C(F)(F)F)c(SCC)c1F] | |
| 1-577 | SO | Et | Me | 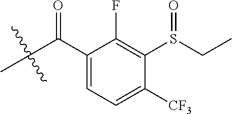 [SMILES: *C(=O)c1ccc(C(F)(F)F)c(S(=O)CC)c1F] | |
| 1-578 | SO | Et | Me | 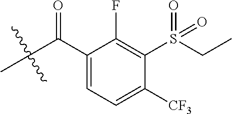 [SMILES: *C(=O)c1ccc(C(F)(F)F)c(S(=O)(=O)CC)c1F] | |
| 1-579 | SO | Et | Me | 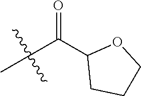 [SMILES: *C(=O)C1CCCO1] | |
| 1-580 | SO | Et | Me | 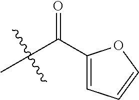 [SMILES: *C(=O)c1ccco1] | |
| 1-581 | SO | Et | Me | 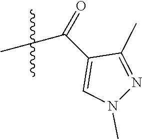 [SMILES: *C(=O)c1cn(C)nc1C] | ¹H NMR (500 MHz, DMSO-d₆) δ 8.03 (dd, J = 7.5, 6.0 Hz, 1H), 7.62 (d, J = 7.5 Hz, 1H), 7.50 (s, 1H), 3.64 (s, |
| | | | | | 3H), 2.83 (q, |
| | | | | | J = 8.0 Hz, |
| | | | | | 2H), 2.63 (s, |
| | | | | | 3H), 2.46 (s, |
| | | | | | 3H), 1.17 (t, |
| | | | | | J = 8.0 Hz, |
| | | | | | 3H). |
| 1-582 | SO | Et | Me | 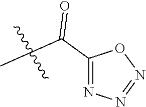 [SMILES: *C(=O)c1nnno1] | |
| 1-583 | SO | Et | Me | 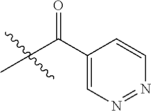 [SMILES: *C(=O)c1ccnnc1] | |
| 1-584 | SO | Et | Me | 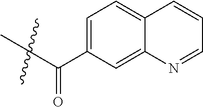 [SMILES: *C(=O)c1ccc2cccnc2c1] | |
| 1-585 | SO | Et | Me | 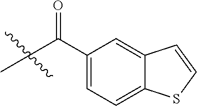 [SMILES: *C(=O)c1ccc2sccc2c1] | |
| 1-586 | SO | Et | Me | 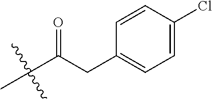 [SMILES: *C(=O)Cc1ccc(Cl)cc1] | |
| 1-587 | SO | Et | Me | 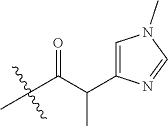 [SMILES: *C(=O)C(C)c1cn(C)cn1] | |
| 1-588 | SO | Et | Me | 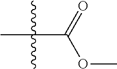 [SMILES: *C(=O)OC] | ¹H NMR (500 MHz, DMSO-d₆) δ 8.11 (dd, J = |
| | | | | | 7.5, 6.0 Hz, |
| | | | | | 1H), 7.61 (d, |
| | | | | | J = 7.5 Hz, |
| | | | | | 1H), 3.71 (s, |
| | | | | | 3H), 2.84 (q, |
| | | | | | J = 8.0 Hz, |
| | | | | | 2H), 2.46 (s, |
| | | | | | 3H), 1.33 (t, |
| | | | | | J = 8.0 Hz, |
| | | | | | 3H). |
| 1-589 | SO | Et | Me | 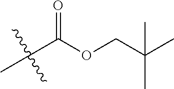 [SMILES: *C(=O)OCC(C)(C)C] | |
| 1-590 | SO | Et | Me | 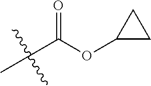 [SMILES: *C(=O)OC1CC1] | |
| 1-591 | SO | Et | Me | 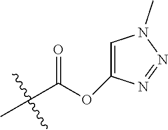 [SMILES: *C(=O)Oc1cn(C)nn1] | |
| 1-592 | SO | Et | Me | 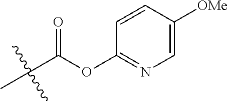 [SMILES: *C(=O)Oc1ccc(OC)cn1] | |
| 1-593 | SO | Et | Me | 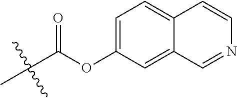 [SMILES: *C(=O)Oc1ccc2ccncc2c1] | |
| 1-594 | SO | Et | Me | 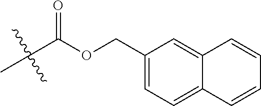 [SMILES: *C(=O)OCc1ccc2ccccc2c1] | |
| 1-595 | SO | Et | Me | 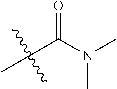 [SMILES: *C(=O)N(C)C] | |
| 1-596 | SO | Et | Me | 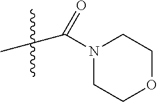 [SMILES: *C(=O)N1CCOCC1] | ¹H NMR (500 MHz, DMSO-d₆) δ 8.11 (dd, J = 7.5, 6.0 Hz, 1H), 7.62 (d, |
| | | | | | J = 7.5 Hz, |
| | | | | | 1H), 3.52 (t, |
| | | | | | J = 4.5 Hz, |
| | | | | | 4H), 3.12 (t, |
| | | | | | J = 4.5 Hz, |
| | | | | | 4H), 2.85 (q, |
| | | | | | J = 8.0 Hz, |
| | | | | | 2H), 2.44 (s, |
| | | | | | 3H), 1.30 (t, |
| | | | | | J = 8.0 Hz, |
| | | | | | 3H). |
| 1-597 | SO | Et | Me | 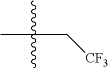 [SMILES: *CC(F)(F)F] | ¹H NMR (500 MHz, DMSO-d₆) δ 7.94 (dd, J = |
| | | | | | 7.5, 6.0 Hz, |
| | | | | | 1H), 7.57 (d, |
| | | | | | J = 7.5 Hz, |
| | | | | | 1H), 3.72 (q, |
| | | | | | J = 9.0 Hz, |
| | | | | | 2H), 2.82 (q, |
| | | | | | J = 8.0 Hz, |
| | | | | | 2H), 2.46 (s, |
| | | | | | 3H), 1.31 (t, |
| | | | | | J = 8.0 Hz, |
| | | | | | 3H). |
| 1-598 | SO | Et | Me | 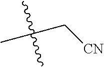 [SMILES: *CC#N] | ¹H NMR (500 MHz, DMSO-d₆) δ 7.90 (dd, J = |
| | | | | | 7.5, 6.0 Hz, |
| | | | | | 1H), 7.61 (d, |
| | | | | | J = 7.5 Hz, |
| | | | | | 1H), 4.32 (s, |
| | | | | | 2H), 2.83 (q, |
| | | | | | J = 8.0 Hz, |
| | | | | | 2H), 2.47 (s, |
| | | | | | 3H), 1.31 (t, |
| | | | | | J = 8.0 Hz, |
| | | | | | 3H). |
| 1-599 | SO | Et | Me | 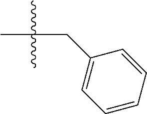 [SMILES: *Cc1ccccc1] | ¹H NMR (500 MHz, DMSO-d₆) δ 7.67 (dd, J = 7.5, 6.0 Hz, 1H), 7.40 (d, J = 7.5 Hz, |
| | | | | | 1H), 7.28- |
| | | | | | 7.19 (m, 5H), |
| | | | | | 5.03 (s, 2H), |
| | | | | | 2.85 (q, J = |
| | | | | | 8.0 Hz, 2H), |
| | | | | | 2.47 (s, 3H), |
| | | | | | 1.27 (t, J = |
| | | | | | 8.0 Hz, 3H). |
| 1-600 | SO | Et | Me | 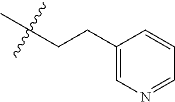 [SMILES: *CCc1cccnc1] | |
| 1-601 | SO | Et | Me | 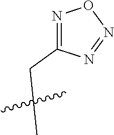 [SMILES: *Cc1nnon1] | |
| 1-602 | SO | Et | Me | 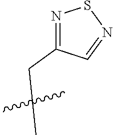 [SMILES: *Cc1cnsn1] | |
| 1-603 | SO | Et | Me | 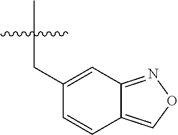 [SMILES: *Cc1ccc2conc2c1] | |
| 1-604 | SO | Et | Me | 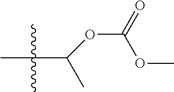 [SMILES: *C(C)OC(=O)OC] | ¹H NMR (500 MHz, DMSO-d₆) δ 7.87 (dd, J = 7.5, 6.0 Hz, 1H), 7.66 (d, |
| | | | | | J = 7.5 Hz, |
| | | | | | 1H), 6.28 (q, |
| | | | | | J = 7.5 Hz, |
| | | | | | 1H), 3.81 (s, |
| | | | | | 3H), 2.83 (q, |
| | | | | | J = 8.0 Hz, |
| | | | | | 2H), 2.46 (s, |
| | | | | | 3H), 1.64 (d, |
| | | | | | J = 7.0 Hz, |
| | | | | | 3H), 1.31 (t, |
| | | | | | J = 8.0 Hz, |
| | | | | | 3H). |
| 1-605 | SO | 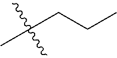 [SMILES: *CCC] | Me | Me | |
| 1-606 | SO | 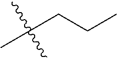 [SMILES: *CCC] | Me | 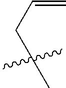 [SMILES: *CC=C] | |
| 1-607 | SO | 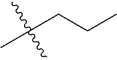 [SMILES: *CCC] | Me | 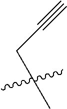 [SMILES: *CC#C] | |
| 1-608 | SO | 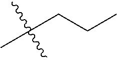 [SMILES: *CCC] | Me | 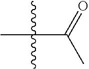 [SMILES: *C(C)=O] | |
| 1-609 | SO | 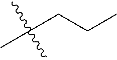 [SMILES: *CCC] | Me | 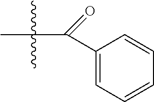 [SMILES: *C(=O)c1ccccc1] | |
| 1-610 | SO | 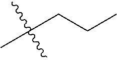 [SMILES: *CCC] | Me | 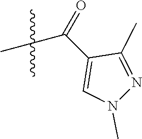 [SMILES: *C(=O)c1cn(C)nc1C] | |
| 1-611 | SO | 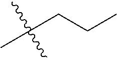 [SMILES: *CCC] | Me | 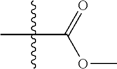 [SMILES: *C(=O)OC] | |
| 1-612 | SO | 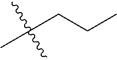 [SMILES: *CCC] | Me | 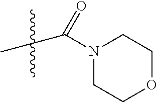 [SMILES: *C(=O)N1CCOCC1] | |
| 1-613 | SO | 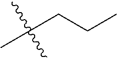 [SMILES: *CCC] | Me | 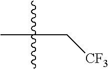 [SMILES: *CC(F)(F)F] | |
| 1-614 | SO | 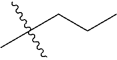 [SMILES: *CCC] | Me | 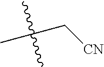 [SMILES: *CC#N] | |
| 1-615 | SO | 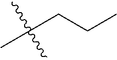 [SMILES: *CCC] | Me | 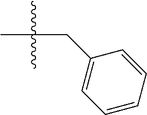 [SMILES: *Cc1ccccc1] | |
| 1-616 | SO | 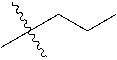 [SMILES: *CCC] | Me | 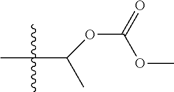 [SMILES: *C(C)OC(=O)OC] | |
| 1-617 | SO | 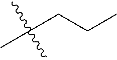 [SMILES: *CCC] | Me | 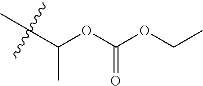 [SMILES: *C(C)OC(=O)OCC] | ¹H NMR (500 MHz, DMSO-d₆) δ 7.82 (dd, J = 7.5, 6.0 Hz, |
| | | | | | 1H), 7.56 (d, |
| | | | | | J = 7.5 Hz, |
| | | | | | 1H), 6.31 (q, |
| | | | | | J = 7.0 Hz, |
| | | | | | 1H), 4.70- |
| | | | | | 4.53 (m, 2H), |
| | | | | | 3.50 (td, J = |
| | | | | | 12.5, 3.0 Hz, |
| | | | | | 1H), 3.24 (td, |
| | | | | | J = 12.5, 3.0 |
| | | | | | Hz, 1H), 2.56- |
| | | | | | 2.42 (m, |
| | | | | | 1H), 2.36 (s, |
| | | | | | 3H), 1.67- |
| | | | | | 1.52 (m, 4H), |
| | | | | | 1.32 (t, J = |
| | | | | | 8.0 Hz, 3H), |
| | | | | | 0.88 (t, J = |
| | | | | | 8.0 Hz, 3H). |
| 1-618 | SO | 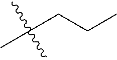 [SMILES: *CCC] | Me | 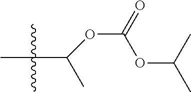 [SMILES: *C(C)OC(=O)OC(C)C] | |
| 1-619 | SO | 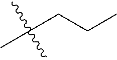 [SMILES: *CCC] | Me | 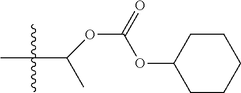 [SMILES: *C(C)OC(=O)OC1CCCCC1] | |
| 1-620 | SO | 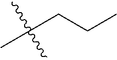 [SMILES: *CCC] | Me | 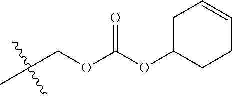 [SMILES: *COC(=O)OC1CC=CCC1] | |
| 1-621 | SO | 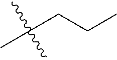 [SMILES: *CCC] | Me | 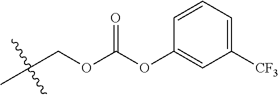 [SMILES: *COC(=O)Oc1cccc(C(F)(F)F)c1] | |
| 1-622 | SO | 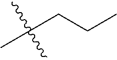 [SMILES: *CCC] | Me | 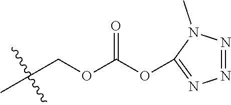 [SMILES: *COC(=O)Oc1nnnn1C] | |
| 1-623 | SO | 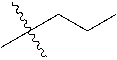 [SMILES: *CCC] | Me | 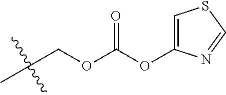 [SMILES: *COC(=O)Oc1cscn1] | |
| 1-624 | SO | 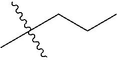 [SMILES: *CCC] | Me | 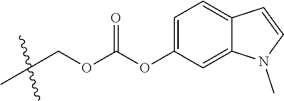 [SMILES: *COC(=O)Oc1ccc2ccn(C)c2c1] | |
| 1-625 | SO | 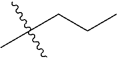 [SMILES: *CCC] | Me | 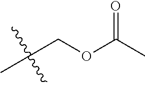 [SMILES: *COC(C)=O] | |
| 1-626 | SO | 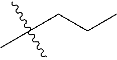 [SMILES: *CCC] | Me | 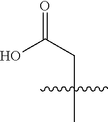 [SMILES: *CC(=O)O] | |
| 1-627 | SO | 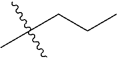 [SMILES: *CCC] | Me | 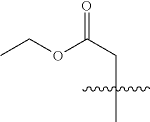 [SMILES: *CC(=O)OCC] | |
| 1-628 | SO | 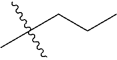 [SMILES: *CCC] | Me | 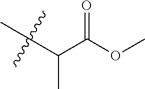 [SMILES: *C(C)C(=O)OC] | |
| 1-629 | SO | 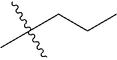 [SMILES: *CCC] | Me | 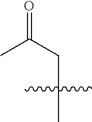 [SMILES: *CC(C)=O] | |
| 1-630 | SO | 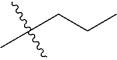 [SMILES: *CCC] | Me | 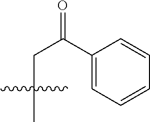 [SMILES: *CC(=O)c1ccccc1] | |
| 1-631 | SO | 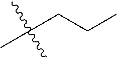 [SMILES: *CCC] | Me | 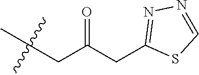 [SMILES: *CC(=O)Cc1nncs1] | |
| 1-632 | SO | 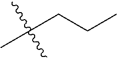 [SMILES: *CCC] | Me | 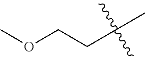 [SMILES: *CCOC] | |
| 1-633 | SO | 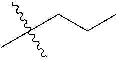 [SMILES: *CCC] | Me | 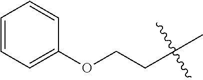 [SMILES: *CCOc1ccccc1] | |
| 1-634 | SO | 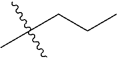 [SMILES: *CCC] | Me | 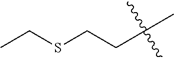 [SMILES: *CCSCC] | |
| 1-635 | SO | 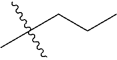 [SMILES: *CCC] | Me | 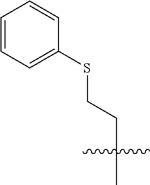 [SMILES: *CCSc1ccccc1] | |
| 1-636 | SO | 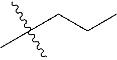 [SMILES: *CCC] | Me | 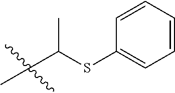 [SMILES: *C(C)Sc1ccccc1] | |
| 1-637 | SO | 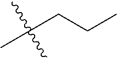 [SMILES: *CCC] | Me | 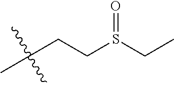 [SMILES: *CCS(=O)CC] | |
| 1-638 | SO | 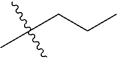 [SMILES: *CCC] | Me | 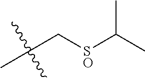 | |
| 1-639 | SO | 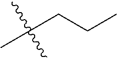 [SMILES: *CCC] | Me | 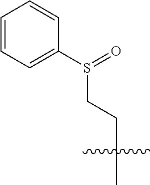 [SMILES: *CCS(=O)c1ccccc1] | |
| 1-640 | SO | 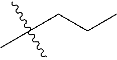 [SMILES: *CCC] | Me | 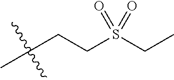 [SMILES: *CCS(=O)(=O)CC] | |
| 1-641 | SO | 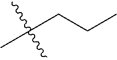 [SMILES: *CCC] | Me | 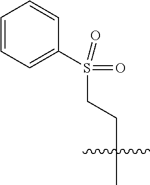 [SMILES: *CCS(=O)(=O)c1ccccc1] | |
| 1-642 | SO | 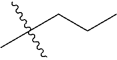 [SMILES: *CCC] | Me | 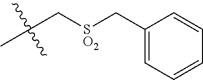 [SMILES: *CS(=O)(=O)Cc1ccccc1] | |
| 1-643 | SO | 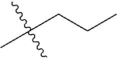 [SMILES: *CCC] | Me | 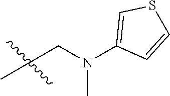 [SMILES: *CN(C)c1ccsc1] | |
| 1-644 | SO | 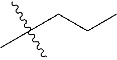 [SMILES: *CCC] | Me | 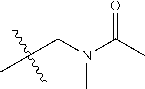 [SMILES: *CN(C)C(C)=O] | |
| 1-645 | SO | 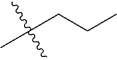 [SMILES: *CCC] | Me | 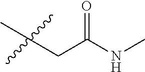 [SMILES: *CC(=O)NC] | |
| 1-646 | SO | 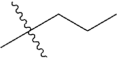 [SMILES: *CCC] | Me | 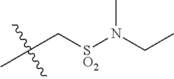 [SMILES: *CS(=O)(=O)N(C)CC] | |
| 1-647 | SO | 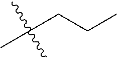 [SMILES: *CCC] | Me | 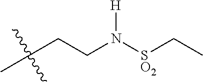 [SMILES: *CCNS(=O)(=O)CC] | |
| 1-648 | SO | 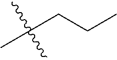 [SMILES: *CCC] | Me | 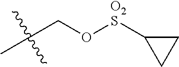 [SMILES: *COS(=O)(=O)C1CC1] | |
| 1-649 | SO | 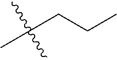 [SMILES: *CCC] | Me | 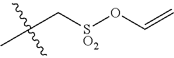 [SMILES: *CS(=O)(=O)OC=C] | |
| 1-650 | SO | 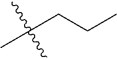 [SMILES: *CCC] | Me | 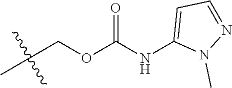 [SMILES: *COC(=O)Nc1ccnn1C] | |
| 1-651 | SO | 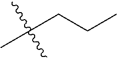 [SMILES: *CCC] | Me | OMe | |
| 1-652 | SO | 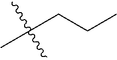 [SMILES: *CCC] | Me | 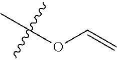 [SMILES: *OC=C] | |
| 1-653 | SO | 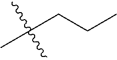 [SMILES: *CCC] | Me | 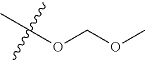 [SMILES: *OCOC] | |
| 1-654 | SO | 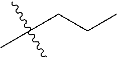 [SMILES: *CCC] | Me | 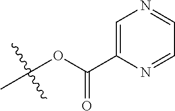 [SMILES: *OC(=O)c1cnccn1] | |
| 1-655 | SO | 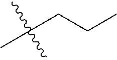 [SMILES: *CCC] | Me | 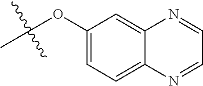 [SMILES: *Oc1ccc2nccnc2c1] | |
| 1-656 | SO | 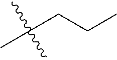 [SMILES: *CCC] | Me | 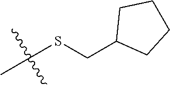 [SMILES: *SCC1CCCC1] | |
| 1-657 | SO | 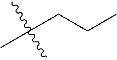 [SMILES: *CCC] | Me | 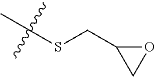 [SMILES: *SCC1CO1] | |
| 1-658 | SO | 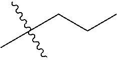 [SMILES: *CCC] | Me | 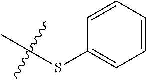 [SMILES: *Sc1ccccc1] | |
| 1-659 | SO | 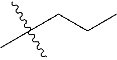 [SMILES: *CCC] | Me | 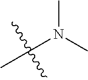 [SMILES: *N(C)C] | |
| 1-660 | SO | 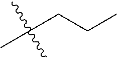 [SMILES: *CCC] | Me | 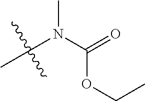 [SMILES: *N(C)C(=O)OCC] | |
| 1-661 | SO | 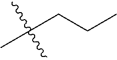 [SMILES: *CCC] | Me | 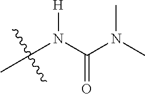 [SMILES: *NC(=O)N(C)C] | |

Table A2 is constructed in the same manner as Table A1 except that the fourth column (i.e., “Z” column) are replaced by the corresponding “Z” column shown below. Accordingly, the first column in Table A2 is the number of compound (Compound No.), i.e., Compound 2-1 (wherein, X is S, Y is Me, Z is Et, and M is H), followed by Compound 2-2, Compound 2-3, and Compound 2-4, the last number of compound is Compound 2-661.

| Table | “Z” |
|---|---|
| A2 | Et |
| A3 | H |
| A4 | 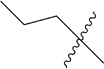 [SMILES: *CCC] |
| A5 | I |
| A6 | 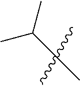 [SMILES: *C(C)C] |
| A7 | Br |
| A8 | 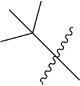 [SMILES: *C(C)(C)C] |
| A9 | F |
| A10 | 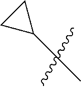 [SMILES: *C1CC1] |
| A11 | 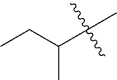 [SMILES: *C(C)CC] |
| A12 | 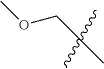 [SMILES: *COC] |
| A13 | 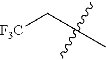 [SMILES: *CC(F)(F)F] |
| A14 | 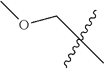 [SMILES: *COC] |
| A15 | 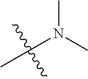 [SMILES: *N(C)C] |
| A16 | Cl |
| A17 | 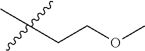 [SMILES: *CCOC] |
| A18 | CN |
| A19 | 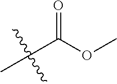 [SMILES: *C(=O)OC] |
| A20 | CF₃ |
| A21 | 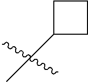 [SMILES: *C1CCC1] |
| A22 | 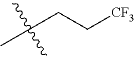 [SMILES: *CCC(F)(F)F] |
| A23 | 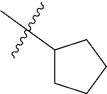 [SMILES: *C1CCCC1] |
| A24 | 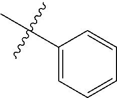 [SMILES: *c1ccccc1] |
| A25 | 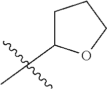 [SMILES: *C1CCCO1] |
| A26 | 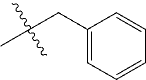 [SMILES: *Cc1ccccc1] |
| A27 | 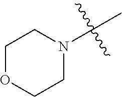 [SMILES: *N1CCOCC1] |
| A28 | 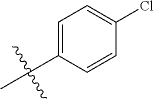 [SMILES: *c1ccc(Cl)cc1] |
| A29 | 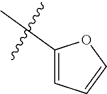 [SMILES: *c1ccco1] |
| A30 | 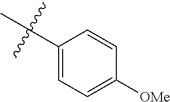 [SMILES: *c1ccc(OC)cc1] |
| A31 | 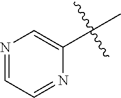 [SMILES: *c1cnccn1] |
| A32 | 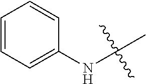 [SMILES: *Nc1ccccc1] |
| A33 | 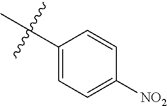 [SMILES: *c1ccc([N+](=O)[O-])cc1] |
| A34 | 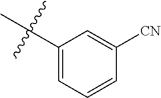 [SMILES: *c1cccc(C#N)c1] |
| A35 | 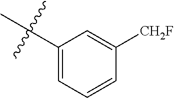 |
| A36 | 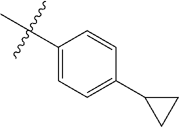 [SMILES: *c1ccc(C2CC2)cc1] |
| A37 | 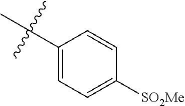 [SMILES: *c1ccc(S(C)(=O)=O)cc1] |
| A38 | 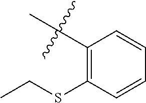 [SMILES: *c1ccccc1SCC] |
| A39 | 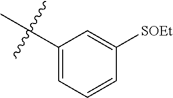 |
| A40 | 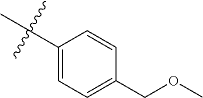 [SMILES: *c1ccc(COC)cc1] |
| A41 | 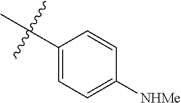 [SMILES: *c1ccc(NC)cc1] |
| A42 | 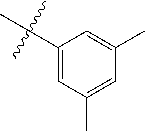 [SMILES: *c1cc(C)cc(C)c1] |
| A43 | 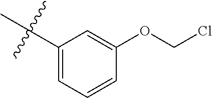 [SMILES: *c1cccc(OCCl)c1] |
| A44 | 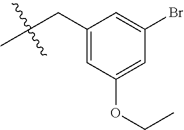 [SMILES: *Cc1cc(Br)cc(OCC)c1] |
| A45 | 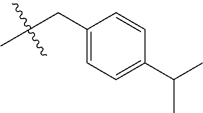 [SMILES: *Cc1ccc(C(C)C)cc1] |
| A46 | 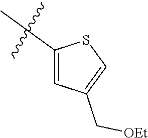 [SMILES: *c1cc(COCC)cs1] |
| A47 | 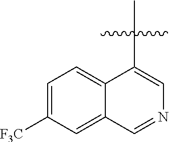 [SMILES: *c1cncc2cc(C(F)(F)F)ccc12] |
| A48 | 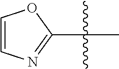 [SMILES: *c1ncco1] |
| A49 | 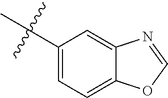 [SMILES: *c1ccc2ocnc2c1] |
| A50 | 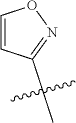 [SMILES: *c1ccon1] |
| A51 | 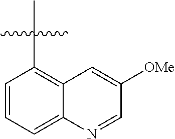 [SMILES: *c1cccc2ncc(OC)cc12] |
| A52 | 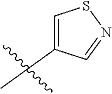 [SMILES: *c1cnsc1] |
| A53 | 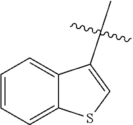 [SMILES: *c1csc2ccccc12] |
| A54 | 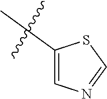 [SMILES: *c1cncs1] |
| A55 | 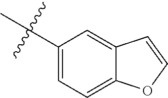 [SMILES: *c1ccc2occc2c1] |
| A56 | 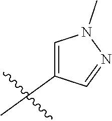 [SMILES: *c1cnn(C)c1] |
| A57 | 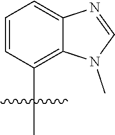 [SMILES: *c1cccc2ncn(C)c12] |
| A58 | 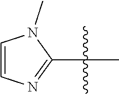 [SMILES: *c1nccn1C] |
| A59 | 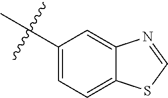 [SMILES: *c1ccc2scnc2c1] |
| A60 | 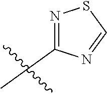 [SMILES: *c1ncsn1] |
| A61 | 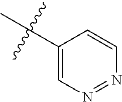 [SMILES: *c1ccnnc1] |
| A62 | 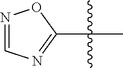 [SMILES: *c1ncno1] |
| A63 | 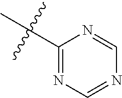 [SMILES: *c1ncncn1] |
| A64 | 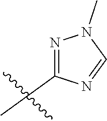 [SMILES: *c1ncn(C)n1] |
| A65 | 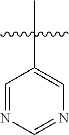 [SMILES: *c1cncnc1] |
| A66 | 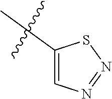 [SMILES: *c1cnns1] |
| A67 | 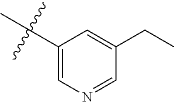 [SMILES: *c1cncc(CC)c1] |
| A68 | 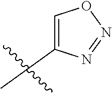 [SMILES: *c1conn1] |
| A69 | 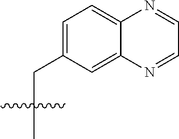 [SMILES: *Cc1ccc2nccnc2c1] |
| A70 | 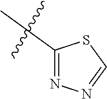 [SMILES: *c1nncs1] |
| A71 | 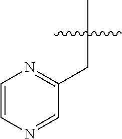 [SMILES: *Cc1cnccn1] |
| A72 | 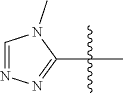 [SMILES: *c1nncn1C] |
| A73 | 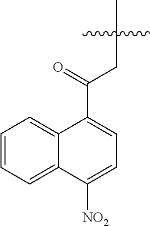 [SMILES: *CC(=O)c1ccc([N+](=O)[O-])c2ccccc12] |
| A74 | 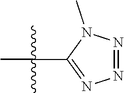 [SMILES: *c1nnnn1C] |
| A75 | 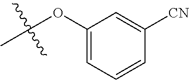 [SMILES: *Oc1cccc(C#N)c1] |
| A76 | 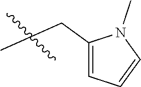 [SMILES: *Cc1cccn1C] |
| A77 | 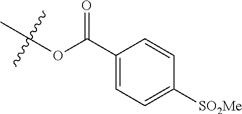 [SMILES: *OC(=O)c1ccc(S(C)(=O)=O)cc1] |
| A78 | 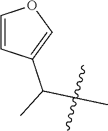 [SMILES: *C(C)c1ccoc1] |
| A79 | 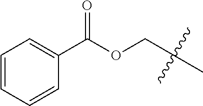 [SMILES: *COC(=O)c1ccccc1] |
| A80 | 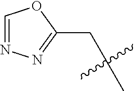 [SMILES: *Cc1nnco1] |
| A81 | 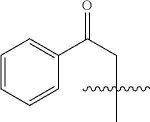 [SMILES: *CC(=O)c1ccccc1] |
| A82 | 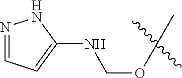 [SMILES: *OCNc1ccn[nH]1] |
| A83 | 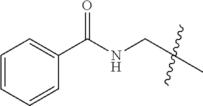 [SMILES: *CNC(=O)c1ccccc1] |
| A84 | 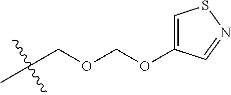 [SMILES: *COCOc1cnsc1] |
| A85 | 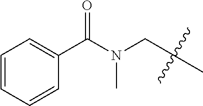 [SMILES: *CN(C)C(=O)c1ccccc1] |
| A86 | 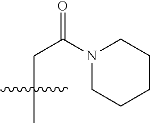 [SMILES: *CC(=O)N1CCCCC1] |
| A87 | 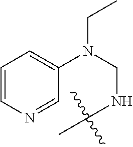 [SMILES: *NCN(CC)c1cccnc1] |
| A88 | 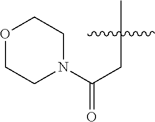 [SMILES: *CC(=O)N1CCOCC1] |
| A89 | 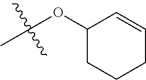 [SMILES: *OC1C=CCCC1] |
| A90 | 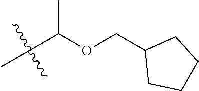 [SMILES: *C(C)OCC1CCCC1] |
| A91 | 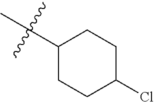 [SMILES: *C1CCC(Cl)CC1] |
| A92 | 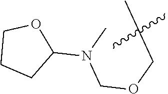 [SMILES: *COCN(C)C1CCCO1] |
| A93 | 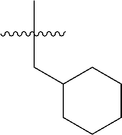 [SMILES: *CC1CCCCC1] |
| A94 | 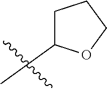 [SMILES: *C1CCCO1] |
| A95 | 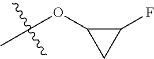 [SMILES: *OC1CC1F] |
| A96 | CHF₂ |
| A97 | 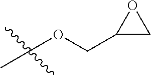 [SMILES: *OCC1CO1] |
| A98 | 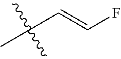 [SMILES: */C=C/F] |
| A99 | CH₂F |
| A100 | 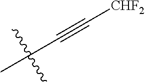 [SMILES: *C#CC(F)F] |
| A101 | 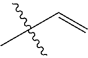 [SMILES: *C=C] |
| A102 | 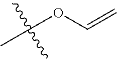 [SMILES: *OC=C] |
| A103 | 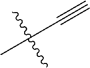 [SMILES: *C#C] |
| A104 | SMe |
| A105 | 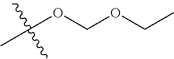 [SMILES: *OCOCC] |
| A106 | SO₂Me |
| A107 | SOMe |
| A108 | 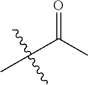 [SMILES: *C(C)=O] |
| A109 | 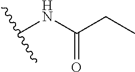 [SMILES: *NC(=O)CC] |
| A110 | COOEt |
| A111 | 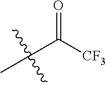 [SMILES: *C(=O)C(F)(F)F] |
| A112 | 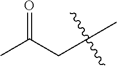 [SMILES: *CC(C)=O] |
| A113 | 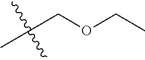 [SMILES: *COCC] |
| A114 | 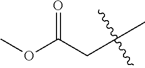 [SMILES: *CC(=O)OC] |
| A115 | 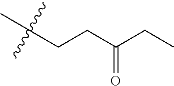 [SMILES: *CCC(=O)CC] |
| A116 | 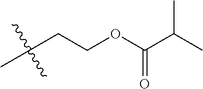 [SMILES: *CCOC(=O)C(C)C] |
| A117 | 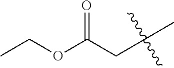 [SMILES: *CC(=O)OCC] |
| A118 | 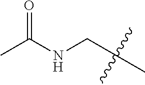 [SMILES: *CNC(C)=O] |
| A119 | 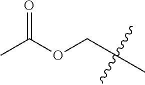 [SMILES: *COC(C)=O] |
| A120 | 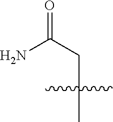 [SMILES: *CC(N)=O] |
| A121 | 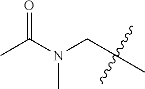 [SMILES: *CN(C)C(C)=O] |
| A122 | 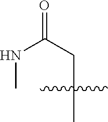 [SMILES: *CC(=O)NC] |
| A123 | 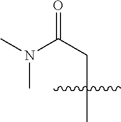 [SMILES: *CC(=O)N(C)C] |
| A124 | 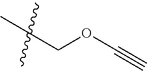 [SMILES: *COC#C] |
| A125 | 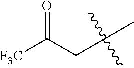 [SMILES: *CC(=O)C(F)(F)F] |
| A126 | 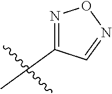 [SMILES: *c1cnon1] |
| A127 | 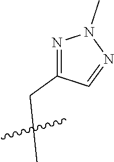 [SMILES: *Cc1cnn(C)n1] |
| A128 | 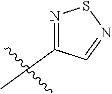 [SMILES: *c1cnsn1] |
| A129 | 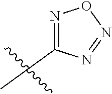 [SMILES: *c1nnon1] |
| A130 | 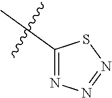 [SMILES: *c1nnns1] |
| A131 | 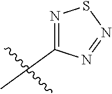 [SMILES: *c1nnsn1] |
| A132 | 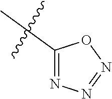 [SMILES: *c1nnno1] |
| A133 | 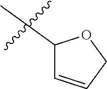 [SMILES: *C1C=CCO1] |
| A134 | 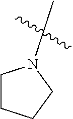 [SMILES: *N1CCCC1] |
| A135 | 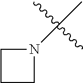 [SMILES: *N1CCC1] |
| A136 | 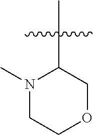 [SMILES: *C1COCCN1C] |
| A137 | 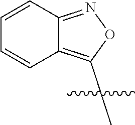 [SMILES: *c1onc2ccccc12] |
| A138 | NH₂ |
| A139 | 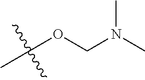 [SMILES: *OCN(C)C] |
| A140 | 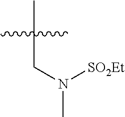 |
| A141 | 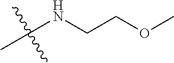 [SMILES: *NCCOC] |
| A142 | 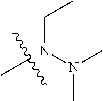 |
| A143 | 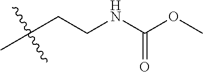 [SMILES: *CCNC(=O)OC] |
| A144 | 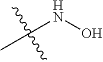 [SMILES: *NO] |
| A145 | 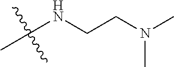 [SMILES: *NCCN(C)C] |
| A146 | 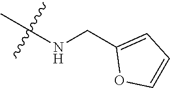 [SMILES: *NCc1ccco1] |
| A147 | 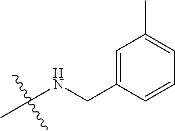 [SMILES: *NCc1cccc(C)c1] |
| A148 | 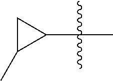 [SMILES: *C1CC1C] |
| A149 | 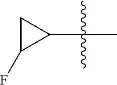 [SMILES: *C1CC1F] |
| A150 | 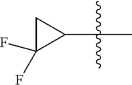 [SMILES: *C1CC1(F)F] |
| A151 | 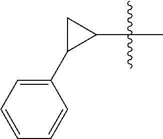 [SMILES: *C1CC1c1ccccc1] |
| A152 | 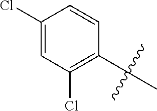 [SMILES: *c1ccc(Cl)cc1Cl] |

The ¹H NMR data of some of the compounds in the above tables are shown in Table 1:

TABLE 1
¹H NMR data of compounds

| Compound | |
|---|---|
| No. | ¹HNMR |
| 2-3 | ¹HNMR (500 MHz, DMSO-d6) 12.37 (s, 1 H), 8.23 (dd, J = 8.0, 7.0 Hz, 1 H), 7.98 (d, J = 8.0 Hz, |
| | 1 H), 3.45 (s, 3 H), 2.63 (q, J = 8.0 Hz, 2 H), 1.32 (t, J = 8.0 Hz, 3 H) |
| 2-4 | ¹H NMR (500 MHz, DMSO-d₆) 12.38 (s, 1 H), 7.88 (dd, J = 8.0, 7.0 Hz, 1 H), 7.78 (d, J = 8.0 Hz, |
| | 1 H), 2.98 (q, J = 7.5 Hz, 2 H), 2.71 (q, J = 7.0 Hz, 2 H), 1.15 (t, J = 7.5 Hz, 3 H), 1.10 (t, J = 7.5 Hz, |
| | 3 H). |
| 2-8 | ¹H NMR (500 MHz, DMSO-d₆) δ 12.38 (s, 1 H),8.24 (dd, J = 7.5, 6.0 Hz, 1 H), 7.65 (d, J = 7.5 Hz, |
| | 1 H), 2.76 (t, J = 5.0 Hz, 2 H), 2.59 (q, J = 8.0 Hz, 2 H), 1.47-1.38 (m, 2 H), 1.13 (t, J = 8.0 Hz, 3 H), |
| | 0.94 (t, J = 8.0 Hz, 3 H). |
| 3-4 | ¹H NMR (500 MHz, DMSO-d₆) δ 12.68 (s, 1 H), 9.06 (s, 1 H), 7.89-7.83 (m, 1 H), 7.76 (d, J = |
| | 8.0 Hz, 1 H), 3.00-2.92 (m, 2 H), 1.13 (t, J = 7.5 Hz, 3 H). |
| 3-5 | ¹H NMR (500 MHz, DMSO-d₆) δ 12.72 (s, 1 H), 9.06 (s, 1 H), 8.18-8.04 (m, 1 H), 7.86 (d, J = |
| | 8.0 Hz, 1 H), 3.51-3.44 (m, 1 H), 3.29-3.22 (m, 1 H), 1.17 (t, J = 7.5 Hz, 3 H). |
| 3-8 | ¹H NMR (500 MHz, DMSO-d₆) δ 12.38 (s, 1 H),8.24 (dd, J = 7.5, 6.0 Hz, 1 H), 7.71 (s, 1 H), 7.65 |
| | (d, J = 7.5 Hz, 1 H), 2.76 (t, J = 5.0 Hz, 2 H), 1.47-1.44 (m, 2 H), 0.95 (t, J = 8.0 Hz, 3 H). |
| 3-11 | ¹H NMR (500 MHz, DMSO-d₆) δ 12.58 (s, 1 H), 9.03 (s, 1 H), 8.19-8.08 (m, 1 H), 7.59 (d, J = |
| | 8.0 Hz, 1 H), 3.86-3.74 (m, 1 H), 1.43 -1.09 (m, 6 H). |
| 3-14 | ¹H NMR (500 MHz, DMSO-d₆) δ 12.78 (s, 1 H), 9.07 (s, 1 H), 8.14-8.06 (m, 1 H), 7.76 (d, J = |
| | 8.0 Hz, 1 H), 3.58-3.47 (m, 1 H), 3.20 ? 3.10 (m, 1 H), 1.73-1.51 (m, 2 H), 1.50-1.35(m, 2 H), |
| | 0.88 (t, J = 7.5 Hz, 3 H). |
| 3-17 | ¹H NMR (500 MHz, DMSO-d₆) δ 12.61 (s, 1 H), 9.08 (s, 1 H), 8.01-7.92 (m, 1 H), 7.59 (d, J = |
| | 8.0 Hz, 1 H), 2.75-2.68 (m, 1 H), 1.78-1.69 (m, 2 H), 1.40 (d, J = 7.0 Hz, 3 H), 0.94 (t, J = 8.0 |
| | Hz, 3 H). |
| 3-20 | ¹H NMR (500 MHz, DMSO-d₆) δ 12.77 (s, 1 H), 9.02 (s, 1 H), 8.13-8.03 (m, 1 H), 7.86 (d, J |
| | 8.0 Hz, 1 H), 3.59-3.48 (m, 1 H), 2.90-2.81 (m, 1 H), 2.19-2.04 (m, 1 H), 1.11-0.99 (m, 6 H). |
| 3-52 | ¹H NMR (500 MHz, DMSO-d₆) δ 12.67 (s, 1 H), 9.05 (s, 1 H), 8.27-8.18 (m, 1 H), 7.92 (d J = |
| | 8.0 Hz, 1 H), 2.76-2.62 (m, 2 H), 2.15-2.05 (m, 2 H). |
| 3-54 | ¹H NMR (500 MHz, DMSO-d₆) δ 12.71 (s, 1 H), 9.01 (s, 1 H), 8.03-7.93 (m, 1 H), 7.82 (d, J |
| | 8.0 Hz, 1 H), 3.78 (t, J = 8.0 Hz, 2 H), 3.07 (t, J = 8.0 Hz, 2 H). |
| 3-57 | ¹H NMR (500 MHz, DMSO-d₆) δ 8.67 (s, 1 H), 8.08-7.97 (m, 1 H), 7.74 (d, J = 8.0 Hz, 1 H), |
| | 3.81-3.71 (m, 1 H), 3.69-3.60 (m, 2 H), 3.42-3.35 (m, 1 H), 3.23 (s, 3 H). |
| 4-4 | ¹H NMR (500 MHz, DMSO-d₆) 12.40 (s, 1 H), 7.89 (dd, J = 8.0, 7.0 Hz, 1 H), 7.79 (d, J = 8.0 Hz, |
| | 1 H), 2.99 (q, J = 7.5 Hz, 2 H), 2.77 (t, J = 7.5 Hz, 2 H), 1.37-1.32 (m, 2 H), 1.15 (t, J = 7.5 Hz, 3 H), |
| | 1.06 (t, J = 7.0 Hz, 3 H). |
| 4-8 | ¹H NMR (500 MHz, DMSO-d₆) δ 12.38 (s, 1 H),8.32 (dd, J = 7.5, 6.0 Hz, 1 H), 7.67 (d, J = 7.5 Hz, |
| | 1 H), 2.76 (t, J = 8.0 Hz, 2 H), 2.53 (t, J = 8.0 Hz, 2 H), 1.61-1.55 (m, 2 H), 1.47-1.33 (m, 2 H), 0.87- |
| | 0.83 (m, 6 H). |
| 6-4 | ¹H NMR (500 MHz, DMSO-d₆) 12.42 (s, 1 H), 7.91 (dd, J = 8.0, 7.0 Hz, 1 H), 7.80 (d, J = 8.0 Hz, |
| | 1 H), 3.01 (q, J = 7.5 Hz, 2 H), 2.97 (hept., J = 7.0 Hz, 1 H), 1.15 (t, J = 7.5 Hz, 3 H), 1.13 (d, J = 7.0 |
| | Hz, 6 H). |
| 6-8 | ¹H NMR (500 MHz, DMSO-d₆) δ 12.38 (s, 1 H), 8.30 (dd, J = 7.5, 6.0 Hz, 1 H), 7.68 (d, J = 7.5 Hz, |
| | 1 H), 3.08 (p,J = 6.8 Hz, 1 H), 2.76 (t,J = 8.1 Hz, 2 H), 1.47-.33(m, 2 H), 1.26 (d,J = 6.8 Hz, 6 H), |
| | 0.89 (t,J = 8.0 Hz, 3 H). |
| 8-4 | ¹H NMR (500 MHz, DMSO-d₆) 12.40 (s, 1 H), 7.92 (dd, J = 8.0, 7.0 Hz, 1 H), 7.81 (d, J = 8.0 Hz, |
| | 1 H), 3.00 (q, J = 7.5 Hz, 2 H), 1.16 (t, J = 7.5 Hz, 3 H), 1.14 (s, 9 H). |
| 8-8 | ¹H NMR (500 MHz, DMSO-d₆) δ 12.38 (s, 1 H),8.24 (dd, J = 7.5, 6.0 Hz, 1 H), 7.65 (d, J = 7.5 Hz, |
| | 1 H), 2.76 (t, J = 5.0 Hz, 2 H), 1.47-1.43 (m, 2 H), 1.25 (s, 9H), 0.94 (t, J = 8.0 Hz, 3 H). |
| 10-4 | ¹H NMR (500 MHz, DMSO-d₆) 12.40 (s, 1 H), 7.91 (dd, J = 8.0, 7.0 Hz, 1 H), 7.80 (d, J = 8.0 Hz, |
| | 1 H), 3.01 (q, J = 7.5 Hz, 2 H), 2.30- 2.20 (m, 1 H), 1.15 (t, J = 7.5 Hz, 3 H), 1.10 - 0.94 (m, 4H) |
| 10-8 | ¹H NMR (500 MHz, DMSO-d₆) δ 12.38 (s, 1 H),8.11 (dd, J = 7.5, 6.0 Hz, 1 H), 7.53 (d, J = 7.5 Hz, |
| | 1 H), 2.76 (t, J = 7.0 Hz, 2 H), 1.55 - 1.41 (m, 3 H), 0.97 (t, J = 8.0 Hz, 3 H), 0.89 - 0.78 (m, 2 H), |
| | 0.71-0.61 (m, 2 H). |
| 12-8 | ¹H NMR (500 MHz, Chloroform-a) δ 8.19 (s, 1 H), 7.79 (dd, J = 7.5, 6.0 Hz, 1 H), 7.68 (d, J = 7.5 |
| | Hz, 1 H), 3.77 (s, 3 H), 2.76 (t, J = 8.0 Hz, 2 H), 1.67-1.58 (m, 2 H), 0.91 (t, J = 8.0 Hz, 3 H). |
| 14-4 | ¹HNMR (500 MHz, DMSO-d₆) 12.36 (s, 1 H), 7.90 (dd, J = 8.0, 7.0 Hz, 1 H), 7.80 (d, J = 8.0 Hz, |
| | 1 H),4.78 (s, 2 H), 3.21 (s, 3 H), 2.97 (q, J = 7.5 Hz, 2 H), 1.14 (t, J = 7.5 Hz, 3 H). |
| 14-8 | ¹H NMR (500 MHz, Chloroform-a) δ 8.20 (s, 1 H), 7.79 (dd, J = 7.5, 6.0 Hz, 1 H), 7.67 (d, J = 7.5 |
| | Hz, 1 H), 4.63 (s, 2 H), 3.35 (s, 3 H), 2.76 (t, J = 5.0 Hz, 2 H), 1.67 (qt, J = 8.0, 5.0 Hz, 2 H), 0.93 (t, J = |
| | 8.0 Hz, 3 H). |
| 16-8 | ¹H NMR (500 MHz, Chloroform-a) δ 8.23 (s, 1 H), 8.19 (dd, J = 7.5, 6.0 Hz, 1 H), 7.42 (d, J = 7.5 |
| | Hz, 1 H), 2.76 (t, J = 5.0 Hz, 2 H), 1.67 (qt, J = 8.0, 5.0 Hz, 2 H), 0.95 (t, J = 8.0 Hz, 3 H). |
| 17-4 | ¹H NMR (500 MHz, DMSO-d₆) 12.40 (s, 1 H), 7.92 (dd, J = 8.0, 7.0 Hz, 1 H), 7.81 (d, J = 8.0 Hz, |
| | 1 H), 3.59 (t, J = 8.5 Hz, 2 H), 3.32 (s, 3 H), 3.00 (q, J = 7.5 Hz, 2 H), 2.77 (t, J = 8.5 Hz, 2 H), 1.16 (t, |
| | J = 7.5 Hz, 3 H). |
| 17-5 | ¹H NMR (500 MHz, DMSO-d₆) 12.40 (s, 1 H), 8.12 (dd, J = 8.0, 7.0 Hz, 1 H), 8.01 (d, J = 8.0 Hz, |
| | 1 H), 3.59 (t, J = 8.5 Hz, 2 H), 3.32 (s, 3 H), 3.20 (q, J = 7.5 Hz, 2 H), 2.77 (t, J = 8.5 Hz, 2 H), 1.23 (t, |
| | J = 7.5 Hz, 3 H). |
| 17-8 | ¹H NMR (500 MHz, Chloroform-a) δ 8.21 (s, 1 H), 7.80 (dd, J = 7.5, 6.0 Hz, 1 H), 7.67 (d, J = 7.5 |
| | Hz, 1 H),3.55 (t, J = 8.0 Hz, 2 H), 3.25 (s, 3 H), 2.74-2.65 (m, 4H), 1.67-1.58 (m, 2 H), 0.91 (t, J = |
| | 8.0 Hz, 3 H). |
| 18-4 | ¹H NMR (500 MHz, DMSO-d₆) 12.59 (s, 1 H), 8.03 (dd, J = 8.0, 7.0 Hz, 1 H), 7.95 (d, J = 8.0 Hz, |
| | 1 H), 3.09 (q, J = 7.5 Hz, 2 H), 1.19 (t, J = 7.5 Hz, 3 H) |
| 18-8 | ¹H NMR (500 MHz, Chloroform-a) δ 8.77 (s, 1 H), 8.32 (dd, J = 7.5, 6.0 Hz, 1 H), 7.68 (d, J = 7.5 |
| | Hz, 1 H), 2.76 (t, J = 5.0 Hz, 2 H), 1.67 (qt, J = 8.0, 5.0 Hz, 2 H), 0.97 (t, J = 8.0 Hz, 3 H). |
| 20-2 | ¹H NMR (500 MHz, DMSO-d₆) 12.53 (s, 1 H), 8.09 (dd, J = 8.0, 7.0 Hz, 1 H), 7.82 (d, J = 8.0 Hz, |
| | 1 H), 3.37 (s, 3 H) |
| 20-4 | ¹H NMR (500 MHz, DMSO-d₆) 12.51 (s, 1 H), 7.96 (dd, J = 8.0, 7.0 Hz, 1 H), 7.88 (d, J = 8.0 Hz, |
| | 1 H), 3.07 (q, J = 7.5 Hz, 2 H), 1.17 (t, J = 7.5 Hz, 3 H) |
| 20-8 | ¹H NMR (500 MHz, DMSO-d₆) δ 12.38 (s, 1 H), 8.33 (dd, J = 7.5, 6.0 Hz, 1 H), 7.68 - 7.63 (m, |
| | 1 H), 2.76 (t, J = 5.5 Hz, 2 H), 1.47-1.38 (m, 2 H), 0.95 (t, J = 8.0 Hz, 3 H). |
| 21-4 | ¹H NMR (500 MHz, DMSO-d₆) 12.41 (s, 1 H), 7.95 (dd, J = 8.0, 7.0 Hz, 1 H), 7.84 (d, J = 8.0 Hz, |
| | 1 H), 3.21 -3.17 (m, 1 H), 3.01 (q, J = 7.5 Hz, 2 H), 2.05 - 1.75 (m, 6 H), 1.17 (t, J = 7.5 Hz, 3 H). |
| 21-5 | ¹H NMR (500 MHz, DMSO-d₆) 12.41 (s, 1 H), 8.15 (dd, J = 8.0, 7.0 Hz, 1 H), 8.04 (d, J = 8.0 Hz, |
| | 1 H), 3.21 -3.17 (m, 1 H), 3.21 (q, J = 7.5 Hz, 2 H), 2.05 - 1.75 (m, 6 H), 1.24 (t, J = 7.5 Hz, 3 H). |
| 21-8 | ¹H NMR (500 MHz, DMSO-d₆) δ 12.38 (s, 1 H), 8.11 (dd, J = 7.5, 6.0 Hz, 1 H), 7.53 (dd, J = 7.5, |
| | 1.0 Hz, 1 H), 3.24 (p, J = 7.0 Hz, 1 H), 2.76 (t, J = 7.0 Hz, 2 H), 2.38-2.29 (m, 2 H), 2.18 -2.08 (m, |
| | 2 H), 2.04 - 1.86 (m, 2 H), 1.47-1.38 (m, 2 H), 0.97 (t, J = 8.0 Hz, 3 H). |
| 22-4 | ¹H NMR (500 MHz, DMSO-d₆) 12.43 (s, 1 H), 7.93 (dd, J = 8.0, 7.0 Hz, 1 H), 7.79 (d, J = 8.0 Hz, |
| | 1 H), 3.03 (q, J = 7.5 Hz, 2 H), 2.51 (t, J = 7.5 Hz, 2 H), 2.21 -2.10 (m, 2 H), 1.15 (t, J = 7.5 Hz, 3 H) |
| 22-8 | ¹H NMR (500 MHz, Chloroform-a) δ 8.23 (s, 1 H), 7.80 (dd, J = 7.5, 6.0 Hz, 1 H), 7.68 (d, J = 7.5 |
| | Hz, 1 H), 2.76 (t, J = 8.0 Hz, 2 H), 2.53 (t, J = 8.5 Hz, 2 H), 2.14-2.05 (m, 2 H), 1.67-1.55 (m, 2 H), |
| | 0.92 (t, J = 8.0 Hz, 3 H). |
| 23-8 | ¹H NMR (500 MHz, DMSO-d₆) δ 12.38 (s, 1 H), 8.32 (dd, J = 7.5, 6.0 Hz, 1 H), 7.66 (d, J = 7.5 Hz, |
| | 1 H), 3.02 (p, J = 7.0 Hz, 1 H), 2.76 (t, J = 8.0 Hz, 2 H), 1.87- 1.76 (m, 2 H), 1.72- 1.61 (m, 2 H), |
| | 1.59 - 1.36 (m, 6 H), 0.90 (t, J = 8.0 Hz, 3 H). |
| 24-4 | ¹H NMR (500 MHz, DMSO-d₆) 12.46 (s, 1 H), 7.90 (dd, J = 8.0, 7.0 Hz, 1 H), 7.88 - 7,81 (m, 2 H), |
| | 7.78 (d, J = 8.0 Hz, 1 H), 7.27 - 7.19 (m, 3 H), 3.03 (q, J = 7.5 Hz, 2 H), 1.14 (t, J = 7.5 Hz, 3 H) |
| 26-4 | ¹H NMR (500 MHz, DMSO-d₆) δ 12.45 (s, 1 H), 7.90-7.81 (m, 1 H), 7.79-7.72 (m, 1 H), 7.40- |
| | 7.24 (m, 5H), 4.26 (s, 2 H) , 3.01 (q, J = 7.5 Hz, 2 H), 1.15 (t, J = 7.5 Hz, 3 H). |
| 29-4 | ¹H NMR (500 MHz, DMSO-d₆) δ 12.73 (s, 1 H), 8.06-7.99 (m, 1 H), 7.94-7.87 (m, 1 H), 7.83- |
| | 7.78 (m, 1 H), 7.31-7.26 (m, 1 H), 6.83-6.77 (m, 1 H), 3.00 (q, J = 7.5 Hz, 2 H), 1.16 (t, J = 7.5 |
| | Hz, 3 H). |
| 96-4 | ¹H NMR (500 MHz, DMSO-d₆) 12.42 (s, 1 H), 7.99 (dd, J = 8.0, 7.0 Hz, 1 H), 7.88 (d, J = 8.0 Hz, |
| | 1 H), 6.50 (t, J = 72.5 Hz, 1 H), 3.03 (q, J = 7.5 Hz, 2 H), 1.18 (t, J = 7.5 Hz, 3 H). |
| 96-5 | ¹H NMR (500 MHz, DMSO-d₆) 12.42 (s, 1 H), 8.19 (dd, J = 8.0, 7.0 Hz, 1 H), 8.08 (d, J = 8.0 Hz, |
| | 1 H), 6.50 (t, J = 72.5 Hz, 1 H), 3.23 (q, J = 7.5 Hz, 2 H), 1.23 (t, J = 7.5 Hz, 3 H). |
| 96-8 | ¹H NMR (500 MHz, DMSO-d₆) δ 12.38 (s, 1 H), 7.84 (dd, J = 7.5, 6.0 Hz, 1 H), 7.64 (d, J = 7.5 Hz, |
| | 1 H), 6.61 (s, 1 H), 2.76 (t,J = 5.5 Hz, 2 H), 1.47-1.38 (m, 2 H), 0.94 (t, J = 8.0 Hz, 6 H). |
| 101-4 | ¹HNMR (500 MHz, DMSO-d₆) 12.41 (s, 1 H), 7.94 (dd, J = 8.0, 7.0 Hz, 1 H), 7.86 (d, J = 8.0 Hz, |
| | 1 H), 6.71-6.60 (m, 1 H), 5.81-5.72 (m, 1 H), 5.53-5.45 (m, 1 H), 3.02 (q, J - 7.5 hz, 2 H), 1.17 |
| | (t, J = 7.5 Hz, 3 H). |
| 101-5 | ¹H NMR (500 MHz, DMSO-d₆) 12.41 (s, 1 H), 8.14 (dd, J = 8.0, 7.0 Hz, 1 H), 8.06 (d, J = 8.0 Hz, |
| | 1 H), 6.71-6.60 (m, 1 H), 5.81-5.72 (m, 1 H), 5.53-5.45 (m, 1 H), 3.22 (q, J - 7.5 hz, 2 H), 1.23 |
| | (t, J = 7.5 Hz, 3 H). |
| 101-8 | ¹HNMR (500 MHz, DMSO-d₆) δ 12.38 (s, 1 H), 7.84 (dd, J = 7.5, 6.0 Hz, 1 H), 7.64 (d, J = 7.5 Hz, |
| | 1 H), 6.37 (dd, J = 16.5, 10.0 Hz, 1 H), 5.73 (dd, J = 14.0, 10.0 Hz, 1 H), 5.48 (dd, J = 16.5, 14.0 Hz, |
| | 1 H), 2.76 (t, J = 5.5 Hz, 2 H), 1.47-1.38 (m, 2 H), 0.94 (t, J = 8.0 Hz, 3 H). |
| 103-4 | ¹HNMR (500 MHz, DMSO-d₆) 12.41 (s, 1 H), 7.94 (dd, J = 8.0, 7.0 Hz, 1 H), 7.86 (d, J = 8.0 Hz, |
| | 1 H), 4.13 (s, 1 H), 3.02 (q, J = 7.5 Hz, 2 H), 1.17 (t, J = 7.5 Hz, 3 H). |
| 103-5 | ¹H NMR (500 MHz, DMSO-d₆) 12.41 (s, 1 H), 8.14 (dd, J = 8.0, 7.0 Hz, 1 H), 8.05 (d, J = 8.0 Hz, |
| | 1 H), 4.13 (s, 1 H), 3.22 (q, J = 7.5 Hz, 2 H), 1.22 (t, J = 7.5 Hz, 3 H). |
| 103-8 | ¹H NMR (500 MHz, DMSO-d₆) δ 12.38 (s, 1 H), 7.82 (dd, J = 7.5, 6.0 Hz, 1 H), 7.66 (dd, J = 7.5, |
| | 1.0 Hz, 1 H), 4.28 (s, 1 H), 2.76 (t, J = 5.5 Hz, 2 H), 1.47-1.42 (m, 2 H), 0.94 (t,J = 8.0 Hz, 3 H). |
| 148-8 | ¹HNMR (500 MHz, DMSO-d₆) δ 12.38 (s, 1 H), 8.11 (dd, J = 7.5, 6.0 Hz, 1 H), 7.53 (d, J+327.5, 1.0 |
| | Hz, 1 H), 2.76 (t, J = 7.0 Hz, 2 H), 1.56 ? 1.41 (m, 4H), 0.97 (t, J = 8.0 Hz, 3 H), 0.87 (d, J = 6.6 Hz, |
| | 3 H), 0.63-0.54 (m, 1 H), 0.38-0.29 (m, 1 H). |
| 149-8 | ¹HNMR (500 MHz, Chloroform-a) δ 8.23 (s, 1 H),8.08 (dd, J = 7.5, 6.0 Hz, 1 H), 7.52 (d, J = 7.5 |
| | Hz, 1 H), 6.60 (q, J = 7.0 Hz, 1 H), 6.50 (q, J = 7.0 Hz, 1 H), 2.76 (t, J = 7.0 Hz, 2 H), 2.05-1.91 (m, |
| | 1 H), 1.73 ? 1.61 (m, 1 H), 1.08-0.92 (m, 1 H), 0.95 (t, J = 8.0 Hz, 3 H), 0.82-0.73 (m, 1 H). |
| 150-8 | ¹HNMR (500 MHz, Chloroform-a) δ 8.23 (s, 1 H),8.08 (dd, J = 7.5, 6.0 Hz, 1 H), 7.52 (d, J = 7.5 |
| | Hz, 1 H), 2.76 (t, J = 7.0 Hz, 2 H), 2.60 -2.51(m, 1 H), 1.73 ? 1.61 (m, 2 H), 1.51-1.40 (m, 1 H), 1.26- |
| | 1.13 (m, 1 H), 0.95 (t, J = 8.0 Hz, 3 H). |
| 151-8 | ¹HNMR (500 MHz, Chloroform-a) δ 8.23 (s, 1 H), 8.08 (dd, J = 7.5, 6.0 Hz, 1 H), 7.52 (d,J = 7.5 |
| | Hz, 1 H), 7.37-7.32 (m, 2 H), 7.25-7.14 (m, 3 H), 3.51 (t, J = 7.0 Hz, 1 H), 2.76 (t, J = 7.0 Hz, 2 H), |
| | 2.45 (q, J = 7.0 Hz, 1 H), 1.73 ? 1.61 (m, 2 H), 1.50-1.41 (m, 1 H), 1.25-1.16 (m, 1 H), 0.95 (t, J = 8.0 |
| | Hz, 3 H). |
| 152-4 | ¹H NMR (500 MHz, DMSO-d₆) δ 12.60 (s, 1 H), 8.18-7.95 (m, 3 H), 7.52-7.35 (m, 2 H), 3.02 |
| | (q, J = 7.5 Hz, 2 H), 1.19 (t, J = 7.5 Hz, 3 H). |

The method for preparing the compound of the invention will be explained in detail in the following program and embodiment. The material is commercial available or prepared through known method reported in the literature or shown in the route. Those skilled in the art should understand that the compound of the invention can also be synthesized by other synthetic route. Although the detailed material and reaction condition in the synthetic route have been explicated in the following text, it is still easy to be replaced by other similar material and condition. Isomer of the compound, for example, that produced with the variation of the preparation method of the present invention is included in the scope of the present invention. In addition, the following preparation method can be further modified according to the disclosures of the present invention by using common chemical method known to those skilled in the art, for example, protection of suitable group in the process of the reaction, etc.

The following method of application can be used to improve further understanding of the preparation method of the present invention. The specific material, class and condition have been determined to be further explication of the present invention, not to be any limit of the reasonable scope thereof. Reagents of the following synthetic compound showed in the table can either be purchased from the market or easily prepared by those skilled in the art.

Examples of representative compounds are as follows:

1. Synthesis of Compound 1-4

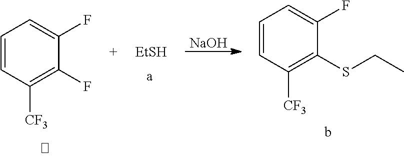

(1) Compound II (3 g, 16 mmol, 1.0 eq), NaOH (0.72 g, 18 mmol, 1.1 eq) were added sequentially into 30 ml of DMF, and then compound a (1.07 g, 1.2 mmol, 1.05 eq) was added dropwise at 0° C., and the reaction solution was stirred at 0° C. for 1 hour. When LCMS test showed that the reaction of starting materials was basically completed, there was one major new peak. The reaction solution was poured into 30 ml of water, and the mixture was separated, and the aqueous phase was extracted once with 50 ml of ethyl acetate, and the resultant organic phase was washed three times with saturated saline solution (50 ml), dried, evaporated to dryness under reduced pressure and separated by column chromatography to obtain compound b (3.4 g, 92% yield) (colorless oil).

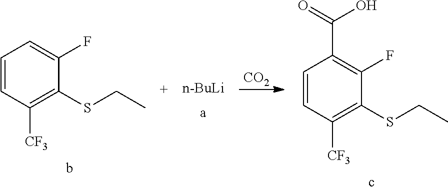

(2) Compound b (3 g, 13 mmol, 1.0 eq) was added to 30 ml of THF, then n-BuLi (6.42 ml, 2.5 M, 16 mmol, 1.2 eq) was slowly added at −78° C., then the reaction solution was stirred at −78° C. for 0.5 hour, and slowly fed with CO₂ for 10 minutes, then the reaction solution was slowly warmed to room temperature. The product was detected by LCMS. 20 ml of water was poured into the reaction solution, the mixture was separated, the aqueous phase was extracted once with 30 ml of ethyl acetate, and the resultant aqueous phase was gradually adjusted to pH=4-5 with concentrated hydrochloric acid, filtered and dried to give compound c (3 g, 83.6% yield) (white solid).

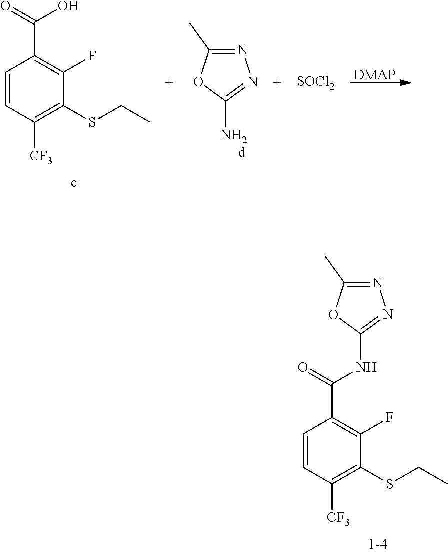

(3) Compound c (3 g, 11 mmol, 1.0 eq), compound d (1.66 g, 16.8 mmol, 1.5 eq), DMAP (0.13 g, 1.1 mmol, 0.1 eq) were sequentially added to 30 ml of pyridine. Then, SOCl₂ (2.0 g, 16.8 mmol, 1.5 eq) was slowly added at 0° C., and the reaction solution was stirred at room temperature for 3 hours. The product was detected by LCMS. Pyridine was removed by concentration, then 30 ml of water was poured into the reaction solution, and the mixture was separated. The aqueous phase was extracted three times with 30 ml of ethyl acetate, and the resultant organic phase was washed three times with saturated saline solution (50 ml), dried, and evaporated to dryness under reduced pressure and separated by column chromatography to to obtain Compound 1-4 (2.5 g, 64% yield) (white solid).

2. Synthesis of Compound 1-5

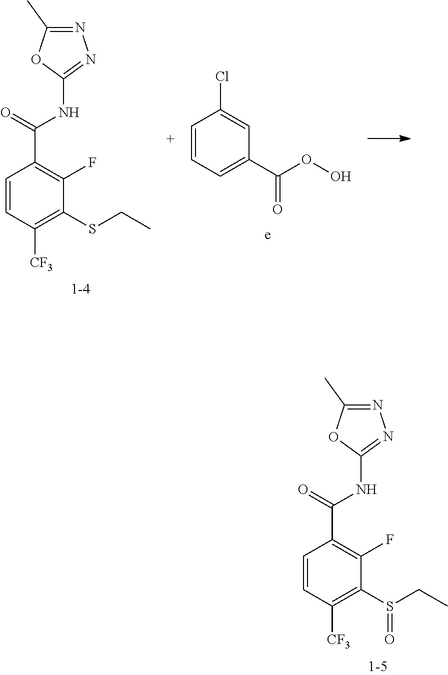 [SMILES: CCSc1c(C(F)(F)F)ccc(C(=O)Nc2nnc(C)o2)c1F ; O=C(OO)c1cccc(Cl)c1 ; CCS(=O)c1c(C(F)(F)F)ccc(C(=O)Nc2nnc(C)o2)c1F]

Compound 1-4 (1 g, 2.8 mmol, 1.0 eq) and compound e (0.54 g, 3.1 mmol, 1.1 eq) were added sequentially in 10 mL of dichloromethane. The reaction solution was then stirred at room temperature for 1 hour. The product was detected by LCMS, and the reaction of raw materials was basically completed. The reaction solution was poured into 10 ml of water, the reaction was quenched with sodium hydrogen sulfite, and the mixture was separated. The aqueous phase was extracted three times with 30 ml of dichloromethane, and the resultant organic phase was washed once with saturated saline solution (30 ml), dried, and evaporated to dryness under reduced pressure, and separated by column chromatography to give Compound 1-5 (0.8 g, 76.5% yield) (greyish white solid).

3. Synthesis of Compound 1-6

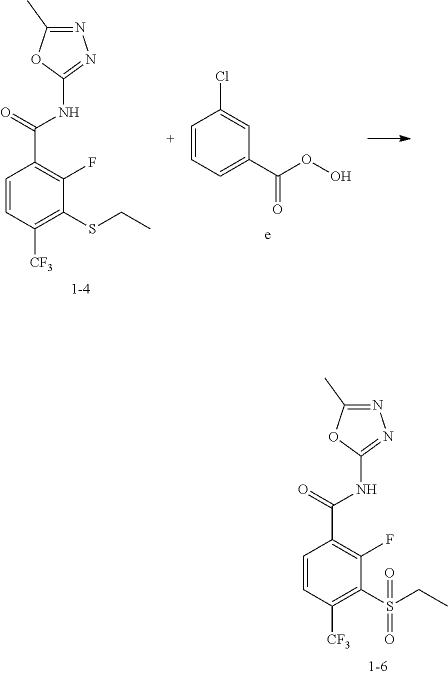 [SMILES: CCSc1c(C(F)(F)F)ccc(C(=O)Nc2nnc(C)o2)c1F ; O=C(OO)c1cccc(Cl)c1 ; CCS(=O)(=O)c1c(C(F)(F)F)ccc(C(=O)Nc2nnc(C)o2)c1F]

Compound 1-4 (1 g, 2.8 mmol, 1.0 eq) and compound e (1.24 g, 7.2 mmol, 2.5 eq) were added sequentially in 10 mL of dichloromethane. The reaction solution was then stirred at room temperature for 24 hours. The product was detected by LCMS and the reaction of raw materials was basically completed. The reaction solution was filtrated, the filtrate was quenched with sodium hydrogen sulfite, concentrated and separated by column chromatography to give Compound 1-6 (0.5 g, 45.8% yield) (greyish white solid).

4. Synthesis of Compound 1-69

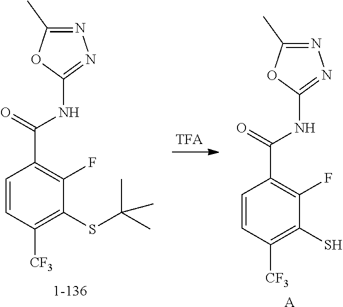

(1) Compound 1-136 was prepared by using the above method for synthesis of Compound 1-4, and then Compound 1-136 (0.34 g, 1.0 mmol, 1.0 eq) was added to 5 ml of TFA, and then the reaction solution was stirred at 90° C. for 16 hours. The product was detected by LCMS, and evaporated to dryness under reduced pressure to give compound A (0.29 g) (yellow oil, crude).

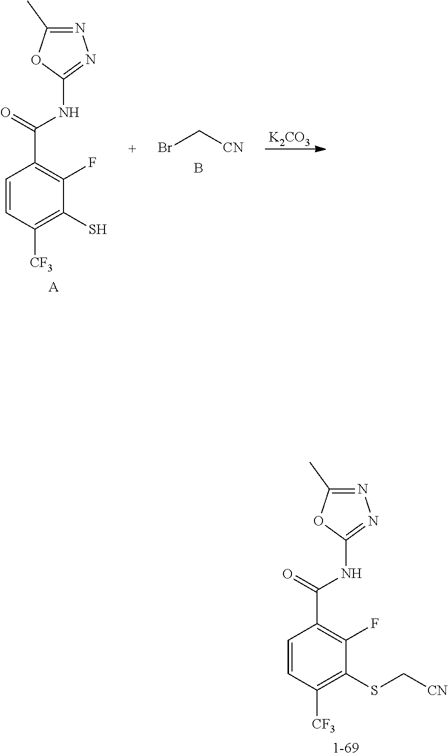

(2) Compound A (0.32 g, 1.0 mmol, 1.0 eq), K₂CO₃ (0.28 g, 2 mmol, 2.0 eq) and compound B (0.13 g, 1.1 mmol, 1.1 eq) were sequentially added in 10 ml of DMF, then the reaction solution was stirred at 25° C. for 2 hours. The product was detected by LCMS. The reaction solution was poured into 20 ml of water, and the mixture was separated, the aqueous phase was extracted three times with 20 ml of ethyl acetate, and the resultant organic phase was washed three times with saturated saline solution (20 ml), dried, and evaporated to dryness under reduced pressure, and separated by column chromatography to give Compound 1-69 (0.25 g, 69% yield) (white solid).

5. Synthesis of Compound 1-112

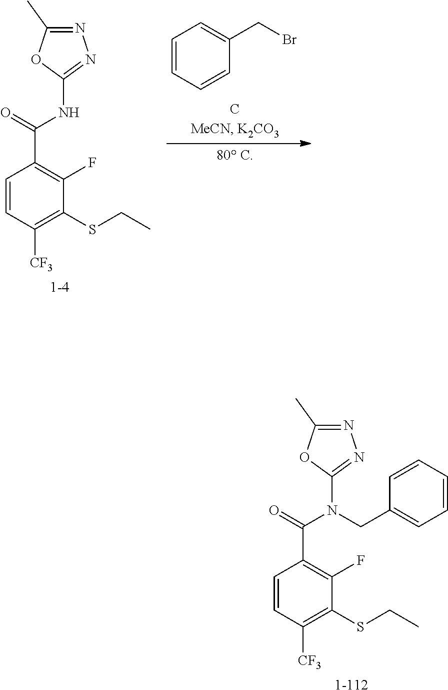

Compound 1-4 (500 mg, 1.4 mmol), Compound C (367 mg, 2.1 mmol), potassium carbonate (595 mg, 2.8 mmol) and acetonitrile 10 mL were placed in a round bottom flask, and stirred at 80° C. for 12 hours. The completion of the reaction was detected by high performance liquid chromatography, then the temperature of the reaction solution was lowered to 20° C., and the remaining potassium carbonate solid was filtered off, and the mother liquid was directly separated by prep-HPLC to obtain Compound 1-112 (160 mg, 25% yield, white solid).

6. Synthesis of Compound 1-118

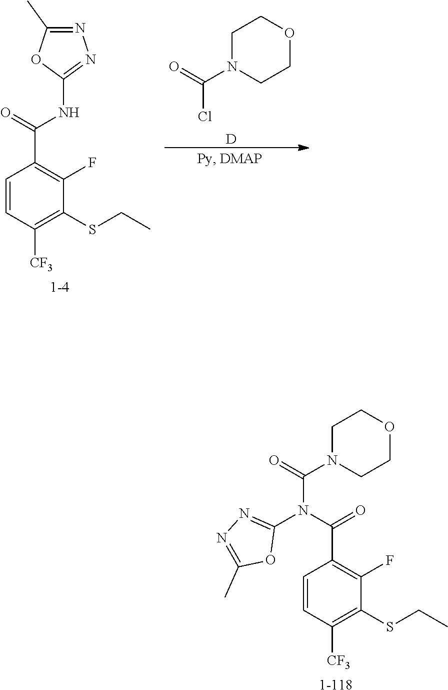

Compound 1-4 (500 mg, 1.4 mmol), a catalytic amount of 4-dimethylaminopyridine (10 mg) and 5 mL of pyridine were placed in a round bottom flask, heated to 60° C., and Compound D (1.1 g, 7.0 mmol) was slowly added dropwise under the protection of nitrogen atmosphere. The completion of the reaction was detected by high performance liquid chromatography, the temperature of the reaction solution was lowered to 20° C., and the reaction system was concentrated and separated by prep-HPLC to give Compound 1-118 (150 mg, 22% yield, yellow solid).

7. Synthesis of Compound 1-127

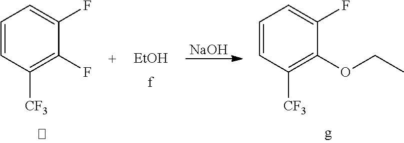

(1) Compound II (3 g, 16 mmol, 1.0 eq), NaOH (0.72 g, 18 mmol, 1.1 eq) were sequentially added to 30 ml of DMF, then compound f (0.8 g, 17 mmol, 1.05 eq) was added dropwise at 0° C., then the reaction solution was stirred at 0° C. for 1 hour. When LCMS detection showed the reaction of starting materials were basically completed, there was one major new peak. The reaction solution was poured into 30 ml of water, and the mixture was separated, and the aqueous phase was extracted once with 50 ml of ethyl acetate, and the resultant organic phase was washed three times with saturated saline solution (50 ml), dried, evaporated to dryness under reduced pressure, and separated by column chromatography to give compound g (3.0 g, 90% yield) (colorless oil).

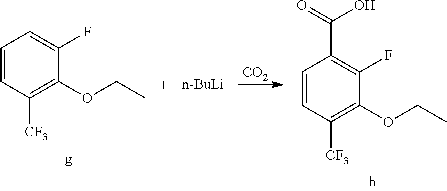

(2) Compound g (2.8 g, 13 mmol, 1.0 eq) was added to 30 ml of THF, then n-BuLi (6.42 ml, 2.5 M, 16 mmol, 1.2 eq) was slowly added at −78° C., then the reaction solution was stirred at −78° C. for 0.5 hour, and then slowly fed with CO₂ for 10 minutes, the reaction solution was slowly warmed to room temperature. The product was detected by LCMS. 20 ml of water was poured into the reaction solution, and the mixture was separated. The aqueous phase was extracted once with 30 ml of ethyl acetate, and the resultant aqueous phase was gradually adjusted to pH=4 to 5 with concentrated hydrochloric acid, filtered and dried to give compound h (2.7 g, 85% yield) (white solid).

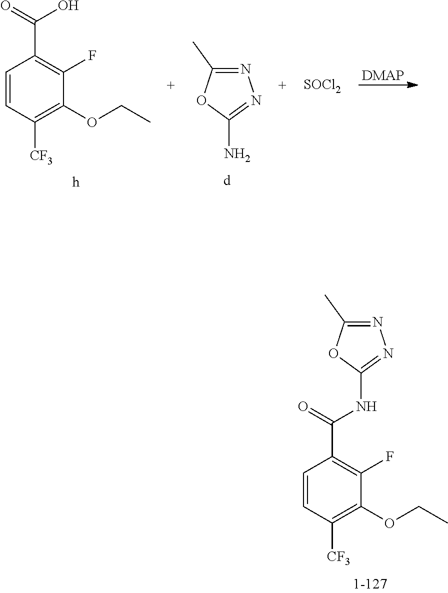

(3) Compound h (2.7 g, 11 mmol, 1.0 eq), compound d (1.66 g, 16.8 mmol, 1.5 eq), DMAP (0.13 g, 1.1 mmol, 0.1 eq) were added sequentially to 30 ml of pyridine. Then, SOCl₂ (2.0 g, 16.8 mmol, 1.5 eq) was slowly added at 0° C., and then the reaction solution was stirred at room temperature for 3 hours. The product was detected by LCMS. Pyridine was removed by concentration, then 30 ml of water was poured into the reaction solution, and the mixture was separated. The aqueous phase was extracted three times with 30 ml of ethyl acetate, and the resultant organic phase was washed three times with saturated saline solution (50 ml), dried, and evaporated to dryness under reduced pressure, and separated by column chromatography to give Compound 1-127 (2.3 g, 63% yield) (white solid).

8. Synthesis of Compound 1-132

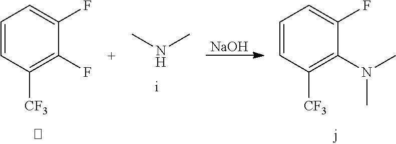

(1) Compound II (3 g, 16 mmol, 1.0 eq), NaOH (0.72 g, 18 mmol, 1.1 eq) were added to 30 ml of DMF, then compound i (17 ml, 1M in THE, 17 mmol, 1.05 eq) was added dropwise at 0° C., then the reaction solution was stirred at 0° C. for 1 hour. When LCMS detection showed that the reaction of starting materials was basically completed, there was one major new peak. The reaction solution was poured into 30 ml of water, and the mixture was separated. The aqueous phase was extracted once with 50 ml of ethyl acetate, and the resultant organic phase was washed three times with saturated saline solution (50 ml), dried, evaporated to dryness under reduced pressure, and separated by column chromatography to give compound j (3.1 g, 92% yield) (colorless oil).

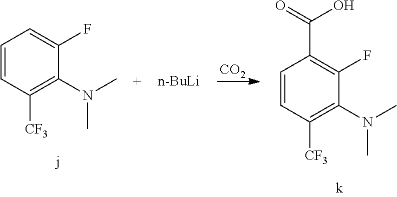

(2) Compound j (2.75 g, 13 mmol, 1.0 eq) was added to 30 ml of THF, then n-BuLi (6.42 ml, 2.5 M, 16 mmol, 1.2 eq) was slowly added at −78° C., then the reaction solution was stirred at −78° C. for 0.5 hour, and then slowly fed with CO₂ for 10 minutes, the reaction solution was slowly warmed to room temperature. The product was detected by LCMS. 20 ml of water was poured into the reaction solution, the mixture was separated. The aqueous phase was extracted once with 30 ml of ethyl acetate, and the resultant aqueous phase was gradually adjusted with concentrated hydrochloric acid to pH=4 to 5, filtered and dried to obtain compound k (2.5 g, 80% yield) (white solid).

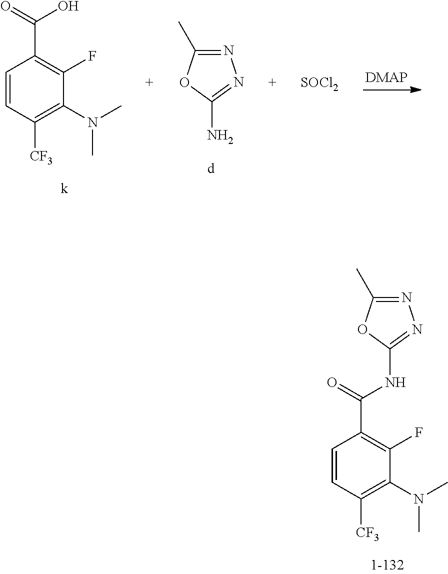

(3) Compound k (2.5 g, 11 mmol, 1.0 eq), compound d (1.66 g, 16.8 mmol, 1.5 eq), DMAP (0.13 g, 1.1 mmol, 0.1 eq) were sequentially added to 30 ml of pyridine. Then, SOCl₂ (2.0 g, 16.8 mmol, 1.5 eq) was slowly added at 0° C., and then the reaction solution was stirred at room temperature for 3 hours. The product was detected by LCMS. Pyridine was removed by concentration, then 30 ml of water was poured into the reaction solution, and the mixture was separated. The aqueous phase was extracted three times with 30 ml of ethyl acetate, and the resultant organic phase was washed three times with saturated saline solution (50 ml), dried, evaporated to dryness under reduced pressure, and separated by column chromatography to give Compound 1-132 (2.1 g, 61% yield) was obtained (white solid).

9. Synthesis of Compound 1-8 (R-Configuration)

Compound 1-8 was prepared according to the method for the preparation of the above Compound 1-5, and then Compound 1-8 (0.5 g, 98% purity) was passed through chiral HPLC (Column: CHIRALPAK IG; Column Size: 3 cm×25 cm, 5 um; Injection: 3.0 ml; Mobile phase: Hex (0.2% FA): IPA=50:50; Flow rate: 28 ml/min; Wavelength: UV 254 nm; Temperature: 25° C.; Sample solution: 70 mg/ml in EtOH/DCM; Run time=60 minutes) for separation, and then concentrated to obtain Compound 1-8 (R-configuration) (0.16 g, Rt=10.51 min, 100% ee, purity 98%) and Compound 1-8 (S-configuration) (0.13 g, Rt=30.81 min, 99.8% ee, purity 96%) in white solids, which were confirmed by single crystal diffraction.

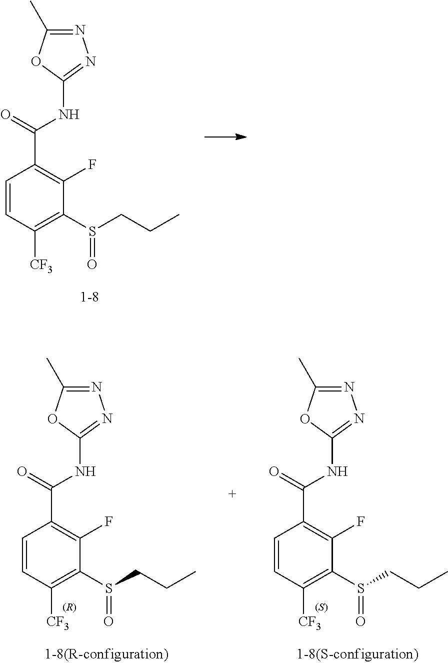

Biological Activity Evaluation:

The activity level criteria for plant damage (i.e., growth control rate) are as follows:

Level 5: growth control rate is above 85%;

Level 4: growth control rate is greater than or equal to 60% and less than 85%;

Level 3: growth control rate is greater than or equal to 40% and less than 60%;

Level 2: growth control rate is greater than or equal to 20% and less than 40%;

Level 1: growth control rate is greater than or equal to 5% and less than 20%;

Level 0: growth control rate is less than 5%.

The above growth control rates are fresh weight control rates.

Experiment on weeding effect in post-emergence stage: monocotyledonous and dicotyledonous weed seeds (Descurainia sophia, Capsella bursa-pastoris, Abutilon theophrasti, Galium aparine, Stellaria media, Lithospermum arvense, rorippa indica, Alopecurus aequalis, Beckmannia syzigachne, Sclerochloa dura, Phleum paniculatum, Bromus japonicus, Aegilops tauschii, Phalaris arundinacea, Amaranthus retroflexus, Chenopodiaceae, Commelina communis, Sonchus arvensis, convolvulus arvensis, Cirsium setosum, Solanum nigrum, Acalypha australis, Digitaria sanguinalis, Echinochloa crusgalli, Setaria viridis, Setaria glauca, Leptochloa chinensis, Monochoria vaginalis, Sagittaria trifolia, Scirpus juncoides, Cyperus rotundus, Cyperus iria, Cyperus difformis, Fimbristylis, Portulaca oleracea, Xanthium sibiricum, Pharbitis nil) and major crop seeds (wheat, corn, rice, soybean, cotton, oilseed rape, millet, sorghum, potato, sesame, ricinus) were placed in plastic pots filled with soil, then covered with 0.5-2 cm of soil, allowed to grow in a good greenhouse environment. After 2 weeks of sowing, the test plants were treated in the 2 to 3 leaf stage. The tested compounds of the present invention were respectively dissolved in acetone, then added with Tween 80 and 1.5 liter/ha of emulsifiable concentrate of methyl oleate as synergist, diluted with a certain amount of water to obtain a solution with a certain concentration, and sprayed with a spray tower onto the plants. After the application, the plants were cultured for 3 weeks in the greenhouse, and then the experimental results of the weeding were counted. The doses of the used compounds were 500, 250, 125, 60, 15 g a.i./ha, and the averages were obtained by repeating for three times. Representative data are listed in Table 2.

TABLE 2
Results on weeding effect in post-emergence stage

| Compound | Echinochloa | Digitaria | Abutilion | Amaranthus | Stellaria | Dose (g |
|---|---|---|---|---|---|---|
| No. | crusgah | sanguinalis | theophrasti | retroflexus | media | a.i./ha) |
| 1-1 | 4 | 4 | 4 | 4 | 4 | 500 |
| 1-1 | 4 | 3 | 4 | 4 | 4 | 250 |
| 1-1 | 4 | 2 | 4 | 4 | 4 | 125 |
| 1-1 | 2 | 1 | 2 | 4 | 3 | 60 |
| 1-2 | 4 | 4 | 4 | 4 | 4 | 500 |
| 1-2 | 4 | 4 | 4 | 4 | 4 | 250 |
| 1-2 | 4 | 3 | 4 | 4 | 3 | 125 |
| 1-2 | 3 | 3 | 4 | 4 | 3 | 60 |
| 1-4 | 5 | 5 | 5 | 5 | 5 | 250 |
| 1-4 | 5 | 5 | 5 | 5 | 5 | 125 |
| 1-5 | 5 | 5 | 5 | 5 | 5 | 125 |
| 1-5 | 5 | 5 | 5 | 5 | 5 | 60 |
| 1-6 | 5 | 5 | 5 | 5 | 5 | 250 |
| 1-6 | 5 | 5 | 5 | 5 | 5 | 125 |
| 1-7 | 5 | 5 | 5 | 5 | 5 | 500 |
| 1-7 | 5 | 5 | 5 | 5 | 5 | 250 |
| 1-8 | 5 | 5 | 5 | 5 | 5 | 125 |
| 1-8 | 5 | 5 | 5 | 5 | 5 | 60 |
| 1-9 | 5 | 5 | 5 | 5 | 5 | 250 |
| 1-9 | 5 | 5 | 5 | 5 | 5 | 125 |
| 1-10 | 5 | N | 5 | 5 | 5 | 250 |
| 1-11 | 5 | 5 | 5 | 5 | 5 | 250 |
| 1-12 | 5 | 5 | 5 | 5 | 5 | 250 |
| 1-13 | 5 | 5 | 5 | 5 | 5 | 250 |
| 1-14 | 5 | 5 | 5 | 5 | 5 | 125 |
| 1-15 | N | N | 5 | 5 | 5 | 250 |
| 1-20 | 5 | 5 | 5 | 5 | 5 | 250 |
| 1-29 | 5 | 5 | 5 | 5 | 5 | 500 |
| 1-40 | 5 | 5 | N | 5 | N | 500 |
| 1-43 | 5 | N | N | N | N | 500 |
| 1-48 | 5 | 5 | N | N | N | 500 |
| 1-49 | 5 | 5 | 5 | 5 | 5 | 500 |
| 1-50 | 5 | 5 | 5 | 5 | 5 | 500 |
| 1-51 | 5 | 5 | 5 | 5 | 5 | 500 |
| 1-52 | 5 | 5 | 5 | 5 | 5 | 500 |
| 1-53 | 5 | 5 | 5 | 5 | 5 | 500 |
| 1-54 | 5 | 5 | N | N | N | 500 |
| 1-57 | 5 | 5 | 5 | 5 | 5 | 500 |
| 1-59 | 5 | 5 | 5 | 5 | 5 | 500 |
| 1-68 | 5 | N | N | N | N | 500 |
| 1-69 | 5 | N | N | N | N | 500 |
| 1-70 | 5 | N | N | N | N | 500 |
| 1-82 | 5 | 5 | 5 | 5 | 5 | 500 |
| 1-137 | 5 | 5 | 5 | 5 | 5 | 500 |
| 1-138 | 5 | 5 | N | N | N | 500 |
| 1-143 | 5 | N | 5 | 5 | 5 | 500 |
| 1-144 | 5 | N | 5 | 5 | 5 | 500 |
| 1-392 | 5 | 5 | 5 | 5 | 5 | 500 |
| 1-394 | 5 | N | N | N | N | 500 |
| 1-395 | 5 | 5 | 5 | 5 | N | 500 |
| 1-397 | 5 | 5 | 5 | 5 | 5 | 500 |
| 1-398 | 5 | 5 | 5 | 5 | 5 | 500 |
| 1-401 | 5 | 5 | 5 | 5 | N | 250 |
| 1-402 | 5 | 5 | 5 | 5 | N | 250 |
| 1-403 | 5 | 5 | 5 | 5 | N | 125 |
| 1-404 | 5 | 5 | 5 | 5 | N | 125 |
| 1-411 | 5 | 5 | 5 | 5 | N | 250 |
| 1-412 | 5 | 5 | 5 | 5 | N | 250 |
| 1-413 | 5 | 5 | N | 5 | N | 500 |
| 1-414 | 5 | 5 | 5 | 5 | N | 250 |
| 1-427 | 5 | 5 | 5 | 5 | N | 500 |
| 1-428 | 5 | 5 | 5 | 5 | N | 500 |
| 1-429 | 5 | 5 | 5 | N | N | 500 |
| 1-430 | 5 | 5 | 5 | N | N | 500 |
| 1-441 | 5 | 5 | 5 | 5 | 5 | 500 |
| 1-443 | 5 | 5 | 5 | 5 | 5 | 500 |
| 1-444 | 5 | N | N | N | N | 500 |
| 1-446 | 5 | 5 | 5 | 5 | 5 | 250 |
| 1-447 | 5 | 5 | 5 | 5 | 5 | 500 |
| 1-448 | 5 | 5 | 5 | 5 | 5 | 500 |
| 1-452 | 5 | 5 | 5 | 5 | 5 | 250 |
| 1-457 | 5 | 5 | N | N | N | 500 |
| 1-525 | 5 | N | N | N | N | 500 |
| 1-537 | 5 | N | N | N | N | 500 |
| 1-541 | 5 | N | 5 | 5 | N | 500 |
| 1-543 | 5 | 5 | 5 | 5 | 5 | 500 |
| 1-552 | 5 | 5 | 5 | N | 5 | 250 |
| 1-558 | 5 | N | N | N | N | 500 |
| 1-560 | 5 | N | 5 | N | N | 500 |
| 1-563 | 5 | 5 | 5 | 5 | 5 | 500 |
| 1-566 | 5 | N | N | N | N | 500 |
| 1-617 | 5 | 5 | N | N | N | 500 |
| 2-4 | 5 | 5 | 5 | 5 | 5 | 500 |
| 3-4 | 5 | 5 | 5 | 5 | 5 | 500 |
| 3-5 | 5 | 5 | 5 | 5 | 5 | 500 |
| 3-8 | 5 | 5 | 5 | 5 | 5 | 500 |
| 3-11 | 5 | 5 | 5 | 5 | 5 | 500 |
| 3-14 | 5 | 5 | 5 | 5 | 5 | 500 |
| 3-17 | 5 | 5 | 5 | 5 | 5 | 500 |
| 3-20 | 5 | N | N | N | N | 500 |
| 3-52 | 5 | 5 | 5 | 5 | 5 | 500 |
| 3-54 | 5 | 5 | 5 | 5 | 5 | 500 |
| 3-57 | 5 | 5 | 5 | 5 | 5 | 500 |
| Control | 0 | 1 | N | 1 | 0 | 500 |
| Compound A | | | | | | |

Note:
Control compound A: 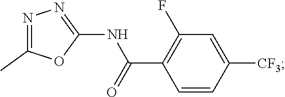 [SMILES: Cc1nnc(NC(=O)c2ccc(C(F)(F)F)cc2F)o1] N indicates partial data were unavailable.

TABLE 3
Results on weeding effect in post-emergence stage

| | Echinochloa | Abutilon | Digitaria | | |
|---|---|---|---|---|---|
| Compound No. | crusgali | theophrasti | sanguinahs | Setaria viridis | Dose (g a.i./ha) |
| 1-1 | N | 4 | 2 | 2 | 120 |
| 1-1 | 2 | 2 | 1 | 1 | 60 |
| 1-1 | 2 | N | 1 | 1 | 30 |
| 1-1 | 1 | N | 0 | 0 | 15 |
| 1-1 | 0 | N | 0 | 0 | 7.5 |
| 1-2 (R- | 4 | N | 4 | 3 | 60 |
| configuration) | | | | | |
| 1-2 (R- | 3 | N | 3 | 3 | 30 |
| configuration) | | | | | |
| 1-2 (R- | 1 | N | 1 | 2 | 15 |
| configuration) | | | | | |
| 1-2 (R- | 0 | N | 0 | 0 | 7.5 |
| configuration) | | | | | |
| 1-2 | 3 | 4 | 3 | 3 | 60 |
| 1-4 | N | 5 | 5 | 5 | 60 |
| 1-5 (R- | 5 | N | 5 | 5 | 60 |
| configuration) | | | | | |
| 1-5 (R- | 5 | N | 5 | 5 | 30 |
| configuration) | | | | | |
| 1-5 (R- | 5 | N | 5 | 5 | 15 |
| configuration) | | | | | |
| 1-5 (R- | 5 | N | 4 | 4 | 7.5 |
| configuration) | | | | | |
| 1-5 (R- | 4 | N | 3 | 3 | 3.75 |
| configuration) | | | | | |
| 1-5 | 5 | 5 | 5 | 5 | 60 |
| 1-8 (R- | 5 | N | 5 | 5 | 60 |
| configuration) | | | | | |
| 1-8 (R- | 5 | N | 5 | 5 | 30 |
| configuration) | | | | | |
| 1-8 (R- | 5 | N | 5 | 5 | 15 |
| configuration) | | | | | |
| 1-8 (R- | 5 | N | 5 | 4 | 7.5 |
| configuration) | | | | | |
| 1-8 (R- | 5 | N | 4 | 2 | 3.75 |
| configuration) | | | | | |
| 1-8 | 5 | 5 | 5 | 5 | 60 |
| 1-14 (R-configuration) | 5 | N | 5 | 5 | 60 |
| 1-14 (R-configuration) | 5 | N | 5 | 5 | 30 |
| 1-14 (R-configuration) | 5 | N | 5 | 4 | 15 |
| 1-14 (R-configuration) | 5 | N | 4 | 4 | 7.5 |
| 1-14 | N | 5 | 5 | 5 | 60 |
| 1-17 | N | 5 | 5 | 5 | 60 |
| 1-20 | N | 5 | 5 | 5 | 60 |
| 1-52 | N | 5 | 5 | 5 | 60 |
| 1-57 (R-configuration) | 5 | N | 5 | 5 | 125 |
| 1-82 (R-configuration) | 5 | N | 5 | 5 | 125 |
| 1-392 (R-configuration) | 5 | N | 5 | 5 | 500 |
| 1-395 (R-configuration) | 5 | N | 5 | 5 | 500 |
| 1-397 (R-configuration) | 5 | N | 5 | 5 | 125 |
| 1-398 (R-configuration) | 5 | N | 5 | 5 | 250 |
| 1-401 (R-configuration) | 5 | N | 5 | 5 | 500 |
| 1-402 (R-configuration) | 5 | N | 5 | 5 | 500 |
| 1-403 (R-configuration) | 5 | N | 5 | 5 | 60 |
| 1-404 (R-configuration) | 5 | N | 5 | 5 | 60 |
| 1-405 (R-configuration) | 5 | N | 5 | 5 | 125 |
| 1-411 (R-configuration) | 5 | N | 5 | 5 | 250 |
| 1-412 (R-configuration) | 5 | N | 5 | 5 | 500 |
| 1-413 (R-configuration) | 5 | N | 5 | 5 | 500 |
| 1-414 (R-configuration) | 5 | N | 5 | 5 | 500 |
| 1-427 (R-configuration) | 5 | N | 5 | 5 | 500 |
| 1-428 (R-configuration) | 5 | N | 5 | 5 | 500 |
| 1-429 (R-configuration) | 5 | N | 5 | 5 | 500 |
| 1-430 (R-configuration) | 5 | N | 5 | 5 | 500 |
| 1-431 (R-configuration) | 5 | N | 5 | 5 | 500 |
| 1-432 (R-configuration) | 5 | N | 5 | 5 | 500 |
| 1-441 (R-configuration) | 5 | N | 5 | 5 | 500 |
| 1-443 (R-configuration) | 5 | N | 5 | 5 | 500 |
| 1-444 (R-configuration) | 5 | N | 5 | 5 | 500 |
| 1-446 (R-configuration) | 5 | N | 5 | 5 | 500 |
| 1-447 (R-configuration) | 5 | N | 5 | 5 | 500 |
| 1-448 (R-configuration) | 5 | N | 5 | 5 | 500 |
| 1-523 (R-configuration) | 5 | N | 5 | 5 | 500 |
| 1-525 (R-configuration) | 5 | N | 5 | 5 | 500 |
| 1-552 (R-configuration) | 5 | N | 5 | 5 | 125 |
| 1-563 (R-configuration) | 5 | N | N | N | 125 |
| 1-617 (R-configuration) | 5 | N | 5 | 5 | 60 |
| 2-8 (R- | 5 | N | 5 | 5 | 250 |
| configuration) | | | | | |
| 3-4 | 5 | N | 5 | 5 | 250 |
| 3-5 (R- | 5 | N | 5 | 5 | 120 |
| configuration) | | | | | |
| 3-8 (R- | 5 | N | 5 | 5 | 60 |
| configuration) | | | | | |
| 3-11 (R-configuration) | 5 | N | 5 | 5 | 125 |
| 3-14 (R-configuration) | 5 | N | 5 | 5 | 60 |
| 3-17 (R-configuration) | 5 | N | 5 | 5 | 125 |
| 3-20 (R-configuration) | 5 | N | 5 | 5 | 125 |
| 3-52 (R-configuration) | 5 | N | 5 | 5 | 125 |
| 3-54 | 5 | N | 5 | 5 | 250 |
| 3-57 (R-configuration) | 5 | N | 5 | 5 | 125 |
| 6-8 (R- | 5 | N | 5 | 5 | 250 |
| configuration) | | | | | |
| Control | N | 3 | 1 | 1 | 120 |
| Compound A | | | | | |

It was found that when the group Y in the compound of the formula of the present invention was changed from methyl to ethyl or C₃-C₈ alkyl, the herbicidal activity and herbicidal spectrum thereof were significally improved. Especially at low doses, the activity on the gramineous weed (such as the main gramineous weed Echinochloa crusgali, Leptochloa chinensis or Digitaria sanguinalis) of Compound 1-4 was higher by more than 2 times than Compound 1-1, the activity of Compound 1-5 was higher by more than 2 times than and Compound 1-2, Compound 1-8 was much more higher than Compound 1-2; when the carbon number exceeded 8, the activity was significantly decreased; while the change of carbon atom number in other groups in the formula had no significant effect on the activity.

Comparative Experiments:

TABLE 4
Safety test results of control experiments (the herbicide selection index
between rice and Echinochloa crusgali was calculated by dividing the average dose of
causing 10% phytotoxicity to rice by the average dose of causing 90% control efficiency to
Echinochloa crusgali, which was an important index for safety evaluation of rice field
herbicides. When a herbicide had a selection index of less than 2, its risk was relatively
higher and it had no commercial value.)

| | | | | Selection index |
|---|---|---|---|---|
| | Indica rice | Japonica rice | | between rice and |
| Compound No. | (Huanghuazhan) | (Huaidao No. 5) | Dose | Echinochloa crusgali |
| 1-5 | 0 | 0 | 250 g a.i./ha | >5 |
| 1-6 | 0 | 0 | 250 g a.i./ha | >5 |
| 1-7 | 0 | 0 | 250 g a.i./ha | >5 |
| 1-8 | 0 | 0 | 250 g a.i./ha | >5 |
| 1-9 | 0 | 0 | 250 g a.i./ha | >5 |
| 1-13 | 0 | 0 | 250 g a.i./ha | >5 |
| 1-14 | 0 | 0 | 250 g a.i./ha | >5 |
| 1-20 | 0 | 0 | 250 g a.i./ha | >5 |
| 1-52 | 0 | 0 | 250 g a.i./ha | >5 |
| 1-57 | 0 | 0 | 250 g a.i./ha | >5 |
| 1-82 | 0 | 0 | 250 g a.i./ha | >5 |
| 1-134 | 0 | 0 | 250 g a.i./ha | >5 |
| Control | 4 | 3 | 250 g a.i./ha | 0.8 |
| Compound B | | | | |
| Control | 3 | 3 | 125 g a.i./ha | 0.8 |
| Compound B | | | | |
| Control | 5 | 4 | 125 g a.i./ha | 1.1 |
| Compound C | | | | |
| Control | 5 | 5 | 60 g a.i./ha | 1 |
| Compound D | | | | |

Note:
Control Compound B: 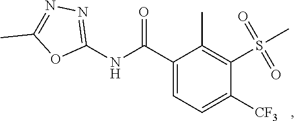 Control Compound C: 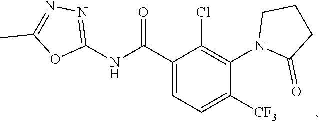 [SMILES: Cc1nnc(NC(=O)c2ccc(C(F)(F)F)c(N3CCCC3=O)c2Cl)o1] Control compound D:

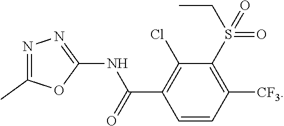 [SMILES: CCS(=O)(=O)c1c(C(F)(F)F)ccc(C(=O)Nc2nnc(C)o2)c1Cl]

From Table 4, it was understood that the compounds of the present invention had very high safety and selectivity for rice, while the control compounds were unsafe for rice, which produced unacceptable phytotoxicity for both indica rice and japonica rice.

In summary, the compounds of the present invention were very safe to rice, and their selection indexes were much greater than 2, and could effectively prevent gramineous weeds such as Echinochloa crusgali, Digitaria sanguinalis, Setaria viridis, and various broad-leaved weeds, and thus were ideal herbicides for rice fields.

Experiment on weeding effect in post-emergence stage: after 3-4 weeks of sowing, the test plants were treated in the 1.5-2 leaf stage of the directly sowed rice Longyang 16, and the 4-5 leaf stage of weeds. After the application of the compounds of the present invention, the plants were cultured for 25 days in the greenhouse, and then the experimental results of the weeding were counted. The test results were shown in Tables 5-6.

TABLE 5
Control experiment results of R configuration compound activity
(inhibition rate, %)

| | Digitaria | | Leptochloa | Ablation |
|---|---|---|---|---|
| | sanguinalis (30 g | Green Setaria | chinensis | theophrasti (30 |
| Compound No. | a.i./ha) | viridis (60 g a.i./ha) | (60 g a.i./ha) | g a.i./ha) |
| 1-8 (R-configuration) | 100% | 100% | 85% | 100% |
| 1-8 (S-configuration) | 0% | 15% | 0% | 0% |
| 1-8 | 80% | 90% | 30% | 90% |
| 1-2 | 0% | 20% | 0% | 60% |

TABLE 6
Control experiment results of R configuration compound activity
(inhibition rate, %)

| | Green | Leptochloa | | |
|---|---|---|---|---|
| | Setaria | chinensis | Rice | Rice |
| | viridis (30 | (125 | (Longyang 16) | (Longyang 16) |
| Compound No. | g a.i./ha) | g a.i./ha) | (250 g a.i./ha) | (500 g a.i./ha) |
| 1-8 (R- | 75% | 100% | 0% | 0% |
| configuration) | | | | |
| 1-8 (S- | 0% | 10% | 15% | 25% |
| configuration) | | | | |
| 1-8 | 10% | 80% | 5% | 15% |

Longyang 16 is a representative and important long-grain fragrant rice in Northeast China. Long-grain fragrant rice is more sensitive to herbicides, especially to HPPD herbicides such as Mesotrione, Benzobicylon and the like, which are prone to causing phytotoxicity. Unexpectedly, compound 1-8 (R-configuration) had high safety on rice (Longyang 16) and had better activity against key weed Leptochloa chinensis, while the S-configuration was not safe for rice, i.e., had severe phytotoxicity on crops, and had lower activity on key weeds, even had no activity at low dose. It was inconsistent with our well-known structure-activity relationships about aryloxyphenoxypropionic acid herbicides and phenoxypropionic acid herbicides that their active isomers had high activity and heavy phytotoxicity while their inactive isomers had almost no activity.

Experiment on weed effect in pre-emergence stage:

Seeds of monocotyledonous and dicotyledonous weeds and main crops (e.g. wheat, corn, rice, soybean, cotton, oilseed rape, millet and sorghum) were put into a plastic pot loaded with soil and covered with 0.5-2 cm soil. The test compounds of the present invention was dissolved with acetone, then added with tween 80, diluted by a certain amount of water to reach a certain concentration, and sprayed immediately after sowing. The obtained seeds were incubated for 4 weeks in the greenhouse after spraying. The test results were observed 4 weeks later. It was observed that the herbicide mostly had excellent effect at the application rate of 250 g a.i./ha, especially to weeds such as Echinochloa crusgali, Digitaria sanguinalis and Abutilon theophrasti, etc. And many compounds had good selectivity for corn, wheat, rice, soybean, oilseed rape, etc.

It is indicated from the experiment that the compound of the present invention generally have good weed control efficacy, especially for major grass weeds such as echinochloa crusgali, Digitaria sanguinalis and Setaria viridis, etc. and major broad-leaved weeds such as Abutilon theophrasti, rorippa indica and Bidens pilosa, which are widely occurred in corn, rice and wheat fields, and have excellent commercial value. Above all, it is noted that the compound of the invention have extremely high activity to broad-leaved weeds, which are resistant to ALS inhibitor, like rorippa indica, Descurainia sophia, Capsella bursa-pastoris, Lithospermum arvense, Galium aparine and Stellaria media, etc.

Transplanted rice safety evaluation and weed control effect evaluation in rice field:

Rice field soil was loaded into a 1/1,000,000 ha pot. The seeds of echinochloa crusgali, scirpus juncoides, Bidens tripartita, Sagittaria trifolia, Monochoria vaginalis and Leptochloa chinensis were sowed and gently covered with soil, then left to stand still in greenhouse in the state of 0.5-1 cm of water storage. The tuber of Sagittaria trifolia was planted in the next day or 2 days later. It was kept at 3-4 cm of water storage thereafter. The weeds were treated by dripping the WP or SC water diluents prepared according to the common preparation method of the compounds of the present invention with pipette homogeneously to achieve specified effective amount when echinochloa crusgali, scirpus juncoides, Bidens tripartita, Monochoria vaginalis and Leptochloa chinensis reached 0.5 leaf stage and Sagittaria trifolia reached the time point of primary leaf stage.

In addition, the rice field soil that loaded into the 1/1,000,000 ha pot was leveled to keep water storage at 3-4 cm depth. The 3 leaf stage rice (japonica rice/Indica rice) was transplanted at 3 cm of transplanting depth the next day. The compound of the present invention was treated by the same way after 5 days of transplantation.

The fertility condition of echinochloa crusgali, scirpus juncoides, Bidens tripartita, Leptochloa chinensis, Sagittaria trifolia and Monochoria vaginalis 14 days after the treatment of the compound of the invention and the fertility condition of rice 21 days after the treatment of the compound of the invention respectively with the naked eye. Evaluate the weed control effect with 0-5 activity standard level. Many compounds show excellent activity and selectivity.

TABLE 7
Experimental results of activity and safety (dose: 125 g a.i./ha)

| | Echinochloa | | Monochoria | |
|---|---|---|---|---|
| Compound No. | crusgali | Scirpus juncoides | vaginalis | Rice |
| 1-4 | 5 | 5 | 5 | 0 |
| 1-5 | 5 | 5 | 5 | 0 |
| 1-6 | 5 | 5 | 5 | 0 |
| 1-8 | 5 | 5 | 5 | 0 |
| 1-11 | 5 | 5 | 5 | 0 |
| 1-14 | 5 | 5 | 5 | 0 |
| 1-57 | 5 | 5 | 5 | 0 |
| 1-76 | 5 | 5 | 5 | 0 |

TABLE 8
Herbicidal effects of compounds (the dose of the compounds of the present invention: 60 g a.i./ha)

| | Echinochloa | Leptochloa | Sagittaria | Monochoria | |
|---|---|---|---|---|---|
| Compound No. | crusgali | chinensis | trifolia | vaginalis | Rice |
| 1-8 (R-configuration) | 5 | 5 | 5 | 5 | 0 |
| 1-14 (R-configuration) | 5 | 5 | 5 | 5 | 0 |
| 1-20 (R-configuration) | 5 | 5 | 5 | 5 | 0 |
| 1-82 (R-configuration) | 5 | 5 | 5 | 5 | 0 |
| 1-403 (R-configuration) | 5 | 5 | 5 | 5 | 0 |
| 1-414 (R-configuration) | 5 | 5 | 5 | 5 | 0 |
| Penoxsulam (30 g a.i./ha) | 0 | 1 | 0 | 1 | 1 |

Note:
The seeds of Echinochloa crusgali, Scirpus juncoides, Bidens tripartita, Sagittaria trifolia and Monochoria vaginalis were collected from Heilongjiang Province of China. The tests indicated that the weeds were resistant to the common doses of Pyrazosulfuron-ethyland Penoxsulam.

It could be seen from the present experiment that the compound of the present invention had excellent activity against weeds having an anti-ALS inhibiting mechanism and being a serious challenge in production, and thus could solve the increasingly serious resistance problem.

In addition, the present invention further relates to a herbicidal composition comprising herbicidally effective amounts of active ingredient (i) (such as the compound represented by Formula I) and active ingredient (ii), wherein the herbicidal composition may further comprise one or more safeners and/or agrochemically acceptable formulation auxiliaries, and the like.

Wherein, the actual weed control effects of the mixtures of the above active ingredients (i) and (ii) were determined by stem-leaf treatment method, i.e., the weeds in the 3-4 leaf stage were sprayed evenly on stems and leaves by a hand sprayer, in which the added-water amount was 30 kg/667 m². The investigation was carried out after 20 days of treatment, and the theoretical fresh weight inhibition rate (calculation formula: E0=X+Y−X*Y/100) of each mixture in the treatment was calculated by Gowing method, and then compared with the measured inhibition rate (E), and the type of combined effect on weeds for the two ingredients in the mixture was evaluated, in which if E-E0 value is greater than 10%, the type of combined effect was synergism; if E-E0 value is less than −10%, the type of combined effect was antagonism; if E-E0 value is between −10% and 10%, the type of combined effect was additive effect. According to the actual control effect and the property of the herbicides, the balance of the formula and other factors, the optimal ratio was determined. In the calculation formula: X represents the fresh weight inhibition rate when the active ingredient (i) is used in an amount of P; and Y represents the fresh weight inhibition rate when the active ingredient (ii) is used in an amount of Q. The statistical results are shown in Tables 9-11 below.

TABLE 9
Evaluation of actual control effects and combined effects of the mixtures
of active ingredients (i) and (ii) in weeds (Gowing method)

| Active | | | | Control effect | Control effect | | | |
|---|---|---|---|---|---|---|---|---|
| Ingredient | Active | (i) + (ii) | | of (i) alone | of (ii) alone | | | |
| (i)/Comp. | Ingredient | g | | at corresponding | at corresponding | E | E0 | E − E0 |
| No. | (ii) | a.i./mu | Weed type | dose (%) | dose (%) | (%) | (%) | (%) |
| 1-1 | Florpyrauxifen- | 3 + 0.6 | Echinochloa | 43.1 | 21.5 | 78.1 | 55.33 | 22.77 |
| | benzyl | | phyllopogon | | | | | |
| 1-1 | Florpyrauxifen- | 1 + 3 | Echinochloa | 8.3 | 70.5 | 87.9 | 72.95 | 14.95 |
| | benzyl | | phyllopogon | | | | | |
| 1-1 | Fluroxypyr | 15 + 3 | Monochoria | 23.9 | 60.3 | 83.2 | 69.79 | 13.41 |
| | | | vaginalis | | | | | |
| 1-1 | Fluroxypyr | 5 + 5 | Monochoria | 9.6 | 73.4 | 90.5 | 75.95 | 14.55 |
| | | | vaginalis | | | | | |
| 1-1 | MCPA | 15 + 3 | Monochoria | 23.9 | 28.2 | 62.1 | 45.36 | 16.74 |
| | | | vaginalis | | | | | |
| 1-1 | MCPA | 5 + 3 | Monochoria | 9.6 | 28.2 | 51.2 | 35.09 | 16.11 |
| | | | vaginalis | | | | | |
| 1-1 | 2,4-D butyric | 15 + 3 | Monochoria | 23.9 | 23.5 | 56.7 | 41.78 | 14.92 |
| | acid | | vaginalis | | | | | |
| 1-1 | 2,4-D butyric | 5 + 15 | Monochoria | 9.6 | 65.3 | 80.8 | 68.63 | 12.17 |
| | acid | | vaginalis | | | | | |
| 1-1 | Dicamba | 5 + 3 | Monochoria | 9.6 | 50.9 | 70.5 | 55.61 | 14.89 |
| | | | vaginalis | | | | | |
| 1-1 | Dicamba | 5 + 5 | Monochoria | 9.6 | 70.2 | 86.2 | 73.06 | 13.14 |
| | | | vaginalis | | | | | |
| 1-1 | Quinclorac | 2 + 3 | Echinochloa | 18.7 | 35.2 | 62.5 | 47.32 | 15.18 |
| | | | phyllopogon | | | | | |
| 1-1 | Quinclorac | 3 + 15 | Echinochloa | 43.1 | 52.9 | 92.2 | 73.20 | 19.00 |
| | | | phyllopogon | | | | | |
| 1-1 | Quintrione | 1 + 3 | Echinochloa | 8.3 | 15.8 | 45.5 | 22.79 | 22.71 |
| | | | phyllopogon | | | | | |
| 1-1 | Quintrione | 3 + 30 | Echinochloa | 43.1 | 66.7 | 94.6 | 81.05 | 13.55 |
| | | | phyllopogon | | | | | |
| 1-1 | Clopyralid | 5 + 1 | Monochoria | 9.6 | 49.6 | 77.3 | 54.44 | 22.86 |
| | | | vaginalis | | | | | |
| 1-1 | Simetryn | 3 + 0.6 | Echinochloa | 43.1 | 20.8 | 72.6 | 54.94 | 17.66 |
| | | | phyllopogon | | | | | |
| 1-1 | Simetryn | 1 + 15 | Echinochloa | 8.3 | 45.3 | 68.2 | 49.84 | 18.36 |
| | | | phyllopogon | | | | | |
| 1-1 | Prometryn | 3 + 0.6 | Echinochloa | 43.1 | 21.5 | 72.6 | 55.33 | 17.27 |
| | | | phyllopogon | | | | | |
| 1-1 | Prometryn | 1 + 20 | Echinochloa | 8.3 | 68.2 | 85.6 | 70.84 | 14.76 |
| | | | phyllopogon | | | | | |
| 1-1 | Pyraclonil | 5 + 15 | Sagittaria | 33.5 | 72.5 | 99.6 | 81.71 | 17.89 |
| | | | trifolia | | | | | |
| 1-1 | Bromoxynil | 1 + 3 | Sagittaria | 10.9 | 12.5 | 38.3 | 22.04 | 16.26 |
| | octanoate | | trifolia | | | | | |
| 1-1 | Carfentrazone- | 1 + 0.2 | Echinochloa | 8.3 | 5.3 | 30.2 | 13.16 | 17.04 |
| | ethyl | | phyllopogon | | | | | |
| 1-1 | Carfentrazone- | 2 + 8 | Echinochloa | 18.7 | 73.6 | 93.6 | 78.54 | 15.06 |
| | ethyl | | phyllopogon | | | | | |
| 1-1 | Amicarbazone | 15 + 3 | Monochoria | 23.9 | 23.3 | 67.2 | 41.63 | 25.57 |
| | | | vaginalis | | | | | |
| 1-1 | Amicarbazone | 5 + 10 | Monochoria | 9.6 | 51.6 | 76.1 | 56.25 | 19.85 |
| | | | vaginalis | | | | | |
| 1-1 | Atrazine | 3 + 15 | Echinochloa | 43.1 | 30.6 | 78.6 | 60.51 | 18.09 |
| | | | phyllopogon | | | | | |
| 1-1 | Isoproturon | 3 + 0.6 | Echinochloa | 43.1 | 20.3 | 70.6 | 54.65 | 15.95 |
| | | | phyllopogon | | | | | |
| 1-1 | Isoproturon | 3 + 3 | Echinochloa | 43.1 | 40.2 | 84.1 | 65.97 | 18.13 |
| | | | phyllopogon | | | | | |
| 1-1 | Bromacil | 3 + 3 | Echinochloa | 43.1 | 12.8 | 70.2 | 50.38 | 19.82 |
| | | | phyllopogon | | | | | |
| 1-1 | Pyridate | 5 + 15 | Cyperus | 30.6 | 53.2 | 86.7 | 67.52 | 19.18 |
| | | | difformis | | | | | |
| 1-1 | Propanil | 5 + 15 | Cyperus | 30.6 | 46.2 | 80.1 | 62.66 | 17.44 |
| | | | difformis | | | | | |
| 1-1 | Oxadiazon | 3 + 3 | Echinochloa | 43.1 | 14.3 | 72.5 | 51.24 | 21.26 |
| | | | phyllopogon | | | | | |
| 1-1 | Oxadiazon | 3 + 15 | Echinochloa | 43.1 | 41.3 | 79.6 | 66.60 | 13.00 |
| | | | phyllopogon | | | | | |
| 1-1 | Oxadiargyl | 3 + 0.6 | Echinochloa | 43.1 | 3.4 | 60.3 | 45.03 | 15.27 |
| | | | phyllopogon | | | | | |
| 1-1 | Oxadiargyl | 3 + 15 | Echinochloa | 43.1 | 35.3 | 78.6 | 63.19 | 15.41 |
| | | | phyllopogon | | | | | |
| 1-1 | Bentazone | 5 + 9 | Cyperus | 30.6 | 40.4 | 76.5 | 58.64 | 17.86 |
| | | | difformis | | | | | |
| 1-1 | Oxyfluorfen | 2 + 0.2 | Echinochloa | 18.7 | 6.7 | 46.7 | 24.15 | 22.55 |
| | | | phyllopogon | | | | | |
| 1-1 | Oxyfluorfen | 2 + 8 | Echinochloa | 18.7 | 76.5 | 98.6 | 80.89 | 17.71 |
| | | | phyllopogon | | | | | |
| 1-1 | Pentoxazone | 7.5 + 10 | Monochoria | 15.1 | 43.6 | 66.5 | 52.12 | 14.38 |
| | | | vaginalis | | | | | |
| 1-1 | Metribuzin | 2 + 3 | Echinochloa | 18.7 | 32.3 | 60.6 | 44.96 | 15.64 |
| | | | phyllopogon | | | | | |
| 1-1 | Terbuthylazine | 3 + 15 | Echinochloa | 43.1 | 18.6 | 71.6 | 53.68 | 17.92 |
| | | | phyllopogon | | | | | |
| 1-1 | Chlortoluron | 3 + 0.6 | Echinochloa | 43.1 | 18.3 | 70.6 | 53.51 | 17.09 |
| | | | phyllopogon | | | | | |
| 1-1 | Chlortoluron | 3 + 15 | Echinochloa | 43.1 | 57.6 | 93.8 | 75.87 | 17.93 |
| | | | phyllopogon | | | | | |
| 1-1 | Fluorochloridone | 3 + 3 | Echinochloa | 43.1 | 9.1 | 60.3 | 48.28 | 12.02 |
| | | | phyllopogon | | | | | |
| 1-1 | Beflubutamid | 7.5 + 5 | Monochoria | 15.1 | 40.3 | 65.2 | 49.31 | 15.89 |
| | | | vaginalis | | | | | |
| 1-1 | Pyrazosulfuron- | 7.5 + 1 | Monochoria | 15.1 | 75.6 | 93.6 | 79.28 | 14.32 |
| | ethyl | | vaginalis | | | | | |
| 1-1 | Penoxsulam | 1 + 1 | Cyperus | 6.2 | 66.4 | 83.2 | 68.48 | 14.72 |
| | | | difformis | | | | | |
| 1-1 | Bispyribac- | 5 + 1 | Cyperus | 30.6 | 70.4 | 93.7 | 79.46 | 14.24 |
| | sodium | | difformis | | | | | |
| 1-1 | Metazosulfuron | 7.5 + 1 | Monochoria | 15.1 | 25.6 | 50.5 | 36.83 | 13.67 |
| | | | vaginalis | | | | | |
| 1-1 | Propyrisulfuron | 1 + 1.5 | Cyperus | 6.2 | 46.3 | 63.8 | 49.63 | 14.17 |
| | | | difformis | | | | | |
| 1-1 | Triafamone | 15 + 0.5 | Monochoria | 23.9 | 16.3 | 70.2 | 36.30 | 33.90 |
| | | | vaginalis | | | | | |
| 1-1 | Triafamone | 15 + 1 | Monochoria | 23.9 | 29.6 | 69.5 | 46.43 | 23.07 |
| | | | vaginalis | | | | | |
| 1-1 | Butralin | 1 + 3 | Cyperus | 6.2 | 16.4 | 38.5 | 21.58 | 16.92 |
| | | | difformis | | | | | |
| 1-1 | Pendimethalin | 5 + 30 | Cyperus | 30.6 | 40.3 | 79.5 | 58.57 | 20.93 |
| | | | difformis | | | | | |
| 1-1 | Sanzuohuangcaotong | 5 + 3 | Monochoria | 9.6 | 21.5 | 46.6 | 29.04 | 17.56 |
| | ( [symbol] ) | | vaginalis | | | | | |
| 1-1 | Shuangzuocaotong | 3 + 0.6 | Echinochloa | 43.1 | 7.8 | 70.5 | 47.54 | 22.96 |
| | ( [symbol] ) | | phyllopogon | | | | | |
| 1-1 | Shuangzuocaotong | 2 + 8 | Echinochloa | 18.7 | 70.9 | 95.6 | 76.34 | 19.26 |
| | ( [symbol] ) | | phyllopogon | | | | | |
| 1-1 | Huanbifucaotong | 15 + 0.5 | Leptochloa | 20.3 | 9.2 | 46.7 | 27.63 | 19.07 |
| | ( [symbol] [symbol] ) | | chinensis | | | | | |
| 1-1 | Mesotrione | 5 + 1 | Cyperus | 30.6 | 50.4 | 85.7 | 65.58 | 20.12 |
| | | | difformis | | | | | |
| 1-1 | Benzobicylon | 5 + 3 | Leptochloa | 3.8 | 26.8 | 46.3 | 29.58 | 16.72 |
| | | | chinensis | | | | | |
| 1-1 | Benzobicylon | 5 + 15 | Leptochloa | 3.8 | 68.2 | 88.5 | 69.41 | 19.09 |
| | | | chinensis | | | | | |
| 1-1 | Tefuryltrione | 1 + 1 | Cyperus | 6.2 | 37.4 | 55.9 | 41.28 | 14.62 |
| | | | difformis | | | | | |
| 1-1 | Molinate | 3 + 3 | Echinochloa | 43.1 | 7.8 | 63.5 | 47.54 | 15.96 |
| | | | phyllopogon | | | | | |
| 1-1 | Benthiocarb | 5 + 3 | Cyperus | 30.6 | 15.2 | 60.9 | 41.15 | 19.75 |
| | | | difformis | | | | | |
| 1-1 | Butachlor | 3 + 15 | Echinochloa | 43.1 | 19.3 | 72.3 | 54.08 | 18.22 |
| | | | phyllopogon | | | | | |
| 1-1 | Pretilachlor | 3 + 3 | Echinochloa | 43.1 | 15.2 | 70.5 | 51.75 | 18.75 |
| | | | phyllopogon | | | | | |
| 1-1 | Pretilachlor | 3 + 15 | Echinochloa | 43.1 | 43.2 | 85.5 | 67.68 | 17.82 |
| | | | phyllopogon | | | | | |
| 1-1 | Mefenacet | 2 + 3 | Echinochloa | 18.7 | 19.2 | 56.3 | 34.31 | 21.99 |
| | | | phyllopogon | | | | | |
| 1-1 | Mefenacet | 3 + 15 | Echinochloa | 43.1 | 46.7 | 90.1 | 69.67 | 20.43 |
| | | | phyllopogon | | | | | |
| 1-1 | Fentrazamide | 15 + 3 | Monochoria | 23.9 | 30.2 | 62.5 | 46.88 | 15.62 |
| | | | vaginalis | | | | | |
| 1-1 | Metolachlor | 3 + 3 | Echinochloa | 43.1 | 7.8 | 67.5 | 47.54 | 19.96 |
| | | | phyllopogon | | | | | |
| 1-1 | Anilofos | 15 + 3 | Leptochloa | 20.3 | 30.8 | 70.2 | 44.85 | 25.35 |
| | | | chinensis | | | | | |
| 1-1 | Piperophos | 15 + 3 | Monochoria | 23.9 | 9.9 | 50.6 | 31.43 | 19.17 |
| | | | vaginalis | | | | | |
| 1-1 | Pyroxasulfone | 6 + 9 | Monochoria | 12.3 | 34.5 | 60.5 | 42.56 | 17.94 |
| | | | vaginalis | | | | | |
| 1-1 | Cyhalofop- | 3 + 3 | Echinochloa | 43.1 | 12.2 | 70.3 | 50.04 | 20.26 |
| | butyl | | phyllopogon | | | | | |
| 1-1 | Metamifop | 3 + 0.6 | Echinochloa | 43.1 | 6.8 | 70.5 | 46.97 | 23.53 |
| | | | phyllopogon | | | | | |
| 1-1 | Metamifop | 2 + 8 | Echinochloa | 18.7 | 60.9 | 82.6 | 68.21 | 14.39 |
| | | | phyllopogon | | | | | |
| 1-1 | Clomazone | 15 + 15 | Leptochloa | 20.3 | 53.6 | 82.6 | 63.02 | 19.58 |
| | | | chinensis | | | | | |
| 1-1 | Cinmethylin | 3 + 3 | Echinochloa | 43.1 | 13.2 | 68.3 | 50.61 | 17.69 |
| | | | phyllopogon | | | | | |
| 1-1 | Indanofan | 5 + 5 | Monochoria | 9.6 | 20.8 | 50.9 | 28.40 | 22.50 |
| | | | vaginalis | | | | | |
| 1-1 | Oxaziclomefone | 3 + 0.6 | Echinochloa | 43.1 | 12.5 | 70.1 | 50.21 | 19.89 |
| | | | phyllopogon | | | | | |
| 1-1 | Oxaziclomefone | 1 + 1 | Echinochloa | 8.3 | 18.7 | 46.2 | 25.45 | 20.75 |
| | | | phyllopogon | | | | | |

Note:
mu is a unit of land area in China, fifteen mus equal one hectare, i.e., 1 mu = 1/15 ha.

TABLE 10
Evaluation of actual control effects and combined effects of the mixtures
of active ingredients (i) and (ii) in weeds (Gowing method)

| Active | | | | Control effect | Control effect | | | |
|---|---|---|---|---|---|---|---|---|
| Ingredient | Active | (i) + (ii) | | of (i)alone | of (ii) alone | | | |
| (i)/Comp. | Ingredient | g | | at corresponding | at corresponding | E | E0 | E − E0 |
| No. | (ii) | a.i./mu | Weed type | dose (%) | dose (%) | (%) | (%) | (%) |
| 1-2 | Florpyrauxifen- | 3 + 0.6 | Echinochloa | 53.7 | 21.5 | 80.1 | 63.65 | 16.45 |
| | benzyl | | phyllopogon | | | | | |
| 1-2 | Florpyrauxifen- | 1 + 3 | Echinochloa | 12.6 | 70.5 | 88.5 | 74.22 | 14.28 |
| | benzyl | | phyllopogon | | | | | |
| 1-2 | Fluroxypyr | 15 + 3 | Monochoria | 30.9 | 60.3 | 85.3 | 72.57 | 12.73 |
| | | | vaginalis | | | | | |
| 1-2 | Fluroxypyr | 5 + 5 | Monochoria | 7.8 | 73.4 | 93.5 | 75.47 | 18.03 |
| | | | vaginalis | | | | | |
| 1-2 | MCPA | 15 + 3 | Monochoria | 30.9 | 28.2 | 70.3 | 50.39 | 19.91 |
| | | | vaginalis | | | | | |
| 1-2 | MCPA | 5 + 3 | Monochoria | 7.8 | 28.2 | 51.2 | 33.80 | 17.40 |
| | | | vaginalis | | | | | |
| 1-2 | 2,4-D butyric | 15 + 3 | Monochoria | 30.9 | 23.5 | 66.7 | 47.14 | 19.56 |
| | acid | | vaginalis | | | | | |
| 1-2 | 2,4-D butyric | 3 + 15 | Monochoria | 7.8 | 65.3 | 83.8 | 68.01 | 15.79 |
| | acid | | vaginalis | | | | | |
| 1-2 | Dicamba | 5 + 3 | Monochoria | 7.8 | 50.9 | 71.3 | 54.73 | 16.57 |
| | | | vaginalis | | | | | |
| 1-2 | Dicamba | 5 + 5 | Monochoria | 7.8 | 70.2 | 87.9 | 72.52 | 15.38 |
| | | | vaginalis | | | | | |
| 1-2 | Quinclorac | 2 + 3 | Echinochloa | 26.8 | 35.2 | 69.5 | 52.57 | 16.93 |
| | | | phyllopogon | | | | | |
| 1-2 | Quinclorac | 3 + 15 | Echinochloa | 53.7 | 52.9 | 93.6 | 78.19 | 15.41 |
| | | | phyllopogon | | | | | |
| 1-2 | Quintrione | 1 + 3 | Digitaria | 18.2 | 15.2 | 46.9 | 30.63 | 16.27 |
| | | | sanguinalis | | | | | |
| 1-2 | Clopyralid | 5 + 1 | Monochoria | 7.8 | 49.6 | 70.5 | 53.53 | 16.97 |
| | | | vaginalis | | | | | |
| 1-2 | Simetryn | 3 + 0.6 | Echinochloa | 53.7 | 20.8 | 77.6 | 63.33 | 14.27 |
| | | | phyllopogon | | | | | |
| 1-2 | Simetryn | 1 + 15 | Echinochloa | 12.6 | 45.3 | 65.2 | 52.19 | 13.01 |
| | | | phyllopogon | | | | | |
| 1-2 | Prometryn | 3 + 0.6 | Echinochloa | 53.7 | 21.5 | 78.6 | 63.65 | 14.95 |
| | | | phyllopogon | | | | | |
| 1-2 | Prometryn | 1 + 20 | Echinochloa | 12.6 | 68.2 | 87.3 | 72.21 | 15.09 |
| | | | phyllopogon | | | | | |
| 1-2 | Pyraclonil | 3 + 15 | Sagittaria | 50.3 | 72.5 | 99.6 | 86.33 | 13.27 |
| | | | trifolia L. | | | | | |
| 1-2 | Bromoxynil | 1 + 3 | Sagittaria | 19.7 | 12.5 | 49.3 | 29.74 | 19.56 |
| | octanoate | | trifolia L. | | | | | |
| 1-2 | Carfentrazone- | 1 + 0.2 | Echinochloa | 12.6 | 5.3 | 34.2 | 17.23 | 16.97 |
| | ethyl | | phyllopogon | | | | | |
| 1-2 | Carfentrazone- | 2 + 8 | Echinochloa | 26.8 | 73.6 | 95.6 | 80.68 | 14.92 |
| | ethyl | | phyllopogon | | | | | |
| 1-2 | Amicarbazone | 15 + 3 | Monochoria | 30.9 | 23.3 | 65.2 | 47.00 | 18.20 |
| | | | vaginalis | | | | | |
| 1-2 | Amicarbazone | 5 + 10 | Monochoria | 7.8 | 51.6 | 75.1 | 55.38 | 19.72 |
| | | | vaginalis | | | | | |
| 1-2 | Atrazine | 3 + 15 | Echinochloa | 53.7 | 30.6 | 83.6 | 67.87 | 15.73 |
| | | | phyllopogon | | | | | |
| 1-2 | Isoproturon | 3 + 0.6 | Echinochloa | 53.7 | 20.3 | 79.6 | 63.10 | 16.50 |
| | | | phyllopogon | | | | | |
| 1-2 | Isoproturon | 3 + 3 | Echinochloa | 53.7 | 40.2 | 86.1 | 72.31 | 13.79 |
| | | | phyllopogon | | | | | |
| 1-2 | Bromacil | 3 + 3 | Echinochloa | 53.7 | 12.8 | 75.1 | 59.63 | 15.47 |
| | | | phyllopogon | | | | | |
| 1-2 | Pyridate | 5 + 15 | Cyperus | 36.5 | 53.2 | 88.2 | 70.28 | 17.92 |
| | | | difformis | | | | | |
| 1-2 | Propanil | 5 + 15 | Cyperus | 36.5 | 46.2 | 81.1 | 65.84 | 15.26 |
| | | | difformis | | | | | |
| 1-2 | Oxadiazon | 3 + 3 | Echinochloa | 53.7 | 14.3 | 76.5 | 60.32 | 16.18 |
| | | | phyllopogon | | | | | |
| 1-2 | Oxadiazon | 3 + 15 | Echinochloa | 53.7 | 41.3 | 87.6 | 72.82 | 14.78 |
| | | | phyllopogon | | | | | |
| 1-2 | Oxadiargyl | 3 + 0.6 | Echinochloa | 53.7 | 3.4 | 72.3 | 55.27 | 17.03 |
| | | | phyllopogon | | | | | |
| 1-2 | Bentazone | 5 + 9 | Cyperus | 36.5 | 40.4 | 76.5 | 62.15 | 14.35 |
| | | | difformis | | | | | |
| 1-2 | Oxyfluorfen | 2 + 8 | Echinochloa | 26.8 | 76.5 | 96.6 | 82.80 | 13.80 |
| | | | phyllopogon | | | | | |
| 1-2 | Pentoxazone | 5 + 10 | Monochoria | 7.8 | 43.6 | 63.5 | 48.00 | 15.50 |
| | | | vaginalis | | | | | |
| 1-2 | Metribuzin | 2 + 3 | Echinochloa | 26.8 | 32.3 | 67.6 | 50.44 | 17.16 |
| | | | phyllopogon | | | | | |
| 1-2 | Terbuthylazine | 3 + 15 | Echinochloa | 53.7 | 18.6 | 78.6 | 62.31 | 16.29 |
| | | | phyllopogon | | | | | |
| 1-2 | Chlortoluron | 3 + 0.6 | Echinochloa | 53.7 | 18.3 | 79.6 | 62.17 | 17.43 |
| | | | phyllopogon | | | | | |
| 1-2 | Chlortoluron | 3 + 15 | Echinochloa | 53.7 | 57.6 | 92.8 | 80.37 | 12.43 |
| | | | phyllopogon | | | | | |
| 1-2 | Fluorochloridone | 3 + 3 | Echinochloa | 53.7 | 9.1 | 72.3 | 57.91 | 14.39 |
| | | | phyllopogon | | | | | |
| 1-2 | Beflubutamid | 5 + 5 | Monochoria | 7.8 | 40.3 | 60.2 | 44.96 | 15.24 |
| | | | vaginalis | | | | | |
| 1-2 | Pyrazosulfuron- | 5 + 1 | Monochoria | 7.8 | 75.6 | 90.6 | 77.50 | 13.10 |
| | ethyl | | vaginalis | | | | | |
| 1-2 | Penoxsulam | 1 + 1 | Cyperus | 7.8 | 66.4 | 85.2 | 69.02 | 16.18 |
| | | | difformis | | | | | |
| 1-2 | Bispyribac- | 5 + 1 | Cyperus | 36.5 | 70.4 | 95.6 | 81.20 | 14.40 |
| | sodium | | difformis | | | | | |
| 1-2 | Metazosulfuron | 5 + 1 | Monochoria | 7.8 | 25.6 | 49.5 | 31.40 | 18.10 |
| | | | vaginalis | | | | | |
| 1-2 | Propyrisulfuron | 1 + 1.5 | Cyperus | 7.8 | 46.3 | 65.8 | 50.49 | 15.31 |
| | | | difformis | | | | | |
| 1-2 | Triafamone | 15 + 1 | Monochoria | 30.9 | 29.6 | 67.5 | 51.35 | 16.15 |
| | | | vaginalis | | | | | |
| 1-2 | Butralin | 1 + 3 | Cyperus | 7.8 | 16.4 | 44.5 | 22.92 | 21.58 |
| | | | difformis | | | | | |
| 1-2 | Pendimethalin | 5 + 30 | Cyperus | 36.5 | 40.3 | 78.5 | 62.09 | 16.41 |
| | | | difformis | | | | | |
| 1-2 | Sanzuohuangcaotong | 5 + 3 | Monochoria | 7.8 | 21.5 | 43.6 | 27.62 | 15.98 |
| | ( [symbol] ) | | vaginalis | | | | | |
| 1-2 | Shuangzuocaotong | 2 + 8 | Digitaria | 40.6 | 75.1 | 98.6 | 85.21 | 13.39 |
| | ( [symbol] ) | | sanguinalis | | | | | |
| 1-2 | Huanbifucaotong | 1 + 3 | Digitaria | 18.2 | 6.2 | 40.7 | 23.27 | 17.43 |
| | ( [symbol] ) | | sanguinalis | | | | | |
| 1-2 | Mesotrione | 5 + 1 | Cyperus | 36.5 | 50.4 | 82.7 | 68.50 | 14.20 |
| | | | difformis | | | | | |
| 1-2 | Benzobicylon | 5 + 3 | Leptochloa | 6.5 | 26.8 | 44.3 | 31.56 | 12.74 |
| | | | chinensis | | | | | |
| 1-2 | Benzobicylon | 5 + 15 | Leptochloa | 6.5 | 68.2 | 87.2 | 70.27 | 16.93 |
| | | | chinensis | | | | | |
| 1-2 | Tefuryltrione | 1 + 1 | Cyperus | 7.8 | 37.4 | 58.9 | 42.28 | 16.62 |
| | | | difformis | | | | | |
| 1-2 | Molinate | 3 + 3 | Echinochloa | 53.7 | 7.8 | 73.8 | 57.31 | 16.49 |
| | | | phyllopogon | | | | | |
| 1-2 | Benthiocarb | 5 + 3 | Cyperus | 36.5 | 15.2 | 63.9 | 46.15 | 17.75 |
| | | | difformis | | | | | |
| 1-2 | Butachlor | 3 + 15 | Echinochloa | 53.7 | 19.3 | 77.3 | 62.64 | 14.66 |
| | | | phyllopogon | | | | | |
| 1-2 | Pretilachlor | 3 + 3 | Echinochloa | 53.7 | 15.2 | 76.2 | 60.74 | 15.46 |
| | | | phyllopogon | | | | | |
| 1-2 | Pretilachlor | 3 + 15 | Echinochloa | 53.7 | 43.2 | 87.6 | 73.70 | 13.90 |
| | | | phyllopogon | | | | | |
| 1-2 | Mefenacet | 2 + 3 | Echinochloa | 26.8 | 19.2 | 59.3 | 40.85 | 18.45 |
| | | | phyllopogon | | | | | |
| 1-2 | Mefenacet | 3 + 15 | Echinochloa | 53.7 | 46.7 | 95.1 | 75.32 | 19.78 |
| | | | phyllopogon | | | | | |
| 1-2 | Fentrazamide | 15 + 3 | Monochoria | 30.9 | 30.2 | 69.5 | 51.77 | 17.73 |
| | | | vaginalis | | | | | |
| 1-2 | Metolachlor | 3 + 3 | Digitaria | 65.3 | 15.3 | 89.5 | 70.61 | 18.89 |
| | | | sanguinalis | | | | | |
| 1-2 | Anilofos | 15 + 3 | Leptochloa | 30.6 | 30.8 | 79.2 | 51.98 | 27.22 |
| | | | chinensis | | | | | |
| 1-2 | Piperophos | 15 + 3 | Monochoria | 30.9 | 9.9 | 53.6 | 37.74 | 15.86 |
| | | | vaginalis | | | | | |
| 1-2 | Cyhalofop- | 3 + 3 | Echinochloa | 53.7 | 12.2 | 76.3 | 59.35 | 16.95 |
| | butyl | | phyllopogon | | | | | |
| 1-2 | Metamifop | 3 + 0.6 | Echinochloa | 53.7 | 6.8 | 75.9 | 56.85 | 19.05 |
| | | | phyllopogon | | | | | |
| 1-2 | Metamifop | 2 + 8 | Echinochloa | 26.8 | 60.9 | 87.6 | 71.38 | 16.22 |
| | | | phyllopogon | | | | | |
| 1-2 | Clomazone | 15 + 15 | Leptochloa | 30.6 | 53.6 | 85.8 | 67.80 | 18.00 |
| | | | chinensis | | | | | |
| 1-2 | Cinmethylin | 3 + 3 | Echinochloa | 53.7 | 13.2 | 75.3 | 59.81 | 15.49 |
| | | | phyllopogon | | | | | |
| 1-2 | Indanofan | 5 + 5 | Monochoria | 7.8 | 20.8 | 48.9 | 26.98 | 21.92 |
| | | | vaginalis | | | | | |
| 1-2 | Oxaziclomefone | 3 + 0.6 | Echinochloa | 53.7 | 12.5 | 79.1 | 59.49 | 19.61 |
| | | | phyllopogon | | | | | |
| 1-2 | Oxaziclomefone | 1 + 1 | Echinochloa | 12.6 | 18.7 | 50.2 | 28.94 | 21.26 |
| | | | phyllopogon | | | | | |

Note:
mu is a unit of land area in China, fifteen mus equal one hectare, i.e., 1 mu = 1/15 ha.

TABLE 11
Evaluation of actual control effects and combined effects of the mixtures
of activity ingredient (i) and (ii) in weeds (Gowing method)

| Activity | | | | Control effect | Control effect | | | |
|---|---|---|---|---|---|---|---|---|
| Ingredient | Activity | (i) + (ii) | | of (i)alone | of (ii) alone | | | |
| (i)/Comp. | Ingredient | g | | at corresponding | at corresponding | E | E0 | E − E0 |
| No. | (ii) | a.i./mu | Weed type | dose (%) | dose (%) | (%) | (%) | (%) |
| 1-4 | Florpyrauxifen- | 3 + 0.6 | Echinochloa | 60.2 | 21.5 | 89.1 | 68.8 | 20.3 |
| | benzyl | | phyllopogon | | | | | |
| 1-4 | Florpyrauxifen- | 0.6 + 3 | Echinochloa | 20.5 | 70.5 | 100 | 76.5 | 23.5 |
| | benzyl | | phyllopogon | | | | | |
| 1-4 | Fluroxypyr | 15 + 3 | Monochoria | 40.5 | 60.3 | 98 | 76.4 | 21.6 |
| | | | vaginalis | | | | | |
| 1-4 | Fluroxypyr | 0.8 + 4 | Monochoria | 5.8 | 70.4 | 100 | 72.1 | 27.9 |
| | | | vaginalis | | | | | |
| 1-4 | MCPA | 15 + 3 | Monochoria | 40.5 | 28.2 | 80.6 | 57.3 | 23.3 |
| | | | vaginalis | | | | | |
| 1-4 | MCPA | 3 + 3 | Monochoria | 12.7 | 28.2 | 64.5 | 37.3 | 27.2 |
| | | | vaginalis | | | | | |
| 1-4 | 2,4-D butyric | 15 + 3 | Monochoria | 40.5 | 23.5 | 75 | 54.5 | 20.5 |
| | acid | | vaginalis | | | | | |
| 1-4 | 2,4-D butyric | 3 + 15 | Monochoria | 12.7 | 65.3 | 86 | 69.7 | 16.3 |
| | acid | | vaginalis | | | | | |
| 1-4 | Dicamba | 3 + 3 | Monochoria | 12.7 | 50.9 | 84.5 | 57.1 | 27.4 |
| | | | vaginalis | | | | | |
| 1-4 | Dicamba | 1 + 5 | Monochoria | 6.2 | 70.2 | 98 | 72.0 | 26.0 |
| | | | vaginalis | | | | | |
| 1-4 | Quinclorac | 3 + 3 | Echinochloa | 60.2 | 35.2 | 96.3 | 74.2 | 22.1 |
| | | | phyllopogon | | | | | |
| 1-4 | Quinclorac | 3 + 15 | Echinochloa | 60.2 | 52.9 | 100 | 81.3 | 18.7 |
| | | | phyllopogon | | | | | |
| 1-4 | Quintrione | 3 + 3 | Echinochloa | 60.2 | 15.8 | 90.5 | 66.5 | 24.0 |
| | | | phyllopogon | | | | | |
| 1-4 | Quintrione | 1.5 + 30 | Echinochloa | 41.5 | 66.7 | 92.6 | 80.5 | 12.1 |
| | | | phyllopogon | | | | | |
| 1-4 | Clopyralid | 6 + 1.2 | Monochoria | 28.6 | 56.3 | 83.5 | 68.8 | 14.7 |
| | | | vaginalis | | | | | |
| 1-4 | Clopyralid | 1 + 1 | Monochoria | 6.2 | 49.6 | 75.5 | 52.7 | 22.8 |
| | | | vaginalis | | | | | |
| 1-4 | Simetryn | 3 + 0.6 | Echinochloa | 60.2 | 20.8 | 85.6 | 68.5 | 17.1 |
| | | | phyllopogon | | | | | |
| 1-4 | Simetryn | 3 + 15 | Echinochloa | 60.2 | 45.3 | 93.1 | 78.2 | 14.9 |
| | | | phyllopogon | | | | | |
| 1-4 | Prometryn | 3 + 0.6 | Echinochloa | 60.2 | 21.5 | 90.6 | 68.8 | 21.8 |
| | | | phyllopogon | | | | | |
| 1-4 | Prometryn | 1 + 20 | Echinochloa | 36.6 | 68.2 | 96.6 | 79.8 | 16.8 |
| | | | phyllopogon | | | | | |
| 1-4 | Pyraclonil | 3 + 0.6 | Sagittaria | 60.3 | 9.3 | 90.6 | 64.0 | 26.6 |
| | | | trifolia L. | | | | | |
| 1-4 | Pyraclonil | 0.75 + 15 | Sagittaria | 16.3 | 72.5 | 92.6 | 77.0 | 15.6 |
| | | | trifolia L. | | | | | |
| 1-4 | Bromoxynil | 3 + 0.6 | Sagittaria | 60.3 | 6.4 | 86.9 | 62.8 | 24.1 |
| | octanoate | | trifolia L. | | | | | |
| 1-4 | Bromoxynil | 3 + 3 | Sagittaria | 60.3 | 12.5 | 92.3 | 65.3 | 27.0 |
| | octanoate | | trifolia L. | | | | | |
| 1-4 | Carfentrazone- | 3 + 0.2 | Echinochloa | 60.2 | 5.3 | 82.2 | 62.3 | 19.9 |
| | ethyl | | phyllopogon | | | | | |
| 1-4 | Carfentrazone- | 0.7 + 8 | Echinochloa | 25.1 | 73.6 | 93 | 80.2 | 12.8 |
| | ethyl | | phyllopogon | | | | | |
| 1-4 | Amicarbazone | 15 + 3 | Monochoria | 40.5 | 23.3 | 80.6 | 54.4 | 26.2 |
| | | | vaginalis | | | | | |
| 1-4 | Amicarbazone | 3.5 + 10 | Monochoria | 13.3 | 51.6 | 80.1 | 58.0 | 22.1 |
| | | | vaginalis | | | | | |
| 1-4 | Atrazine | 3 + 0.6 | Echinochloa | 60.2 | 2.3 | 83.6 | 61.1 | 22.5 |
| | | | phyllopogon | | | | | |
| 1-4 | Atrazine | 0.75 + 15 | Echinochloa | 30.3 | 30.6 | 70.6 | 51.6 | 19.0 |
| | | | phyllopogon | | | | | |
| 1-4 | Isoproturon | 3 + 0.6 | Echinochloa | 60.2 | 20.3 | 90.6 | 68.3 | 22.3 |
| | | | phyllopogon | | | | | |
| 1-4 | Isoproturon | 3 + 3 | Echinochloa | 60.2 | 40.2 | 94.3 | 76.2 | 18.1 |
| | | | phyllopogon | | | | | |
| 1-4 | Bromacil | 3 + 3 | Echinochloa | 60.2 | 12.8 | 87.6 | 65.3 | 22.3 |
| | | | phyllopogon | | | | | |
| 1-4 | Bromacil | 1.5 + 30 | Echinochloa | 41.5 | 59.7 | 93.7 | 76.4 | 17.3 |
| | | | phyllopogon | | | | | |
| 1-4 | Pyridate | 9 + 1.8 | Cyperus | 32.1 | 12.9 | 68.6 | 40.9 | 27.7 |
| | | | difformis | | | | | |
| 1-4 | Pyridate | 3 + 15 | Cyperus | 15.2 | 53.2 | 83.5 | 60.3 | 23.2 |
| | | | difformis | | | | | |
| 1-4 | Propanil | 9 + 1.8 | Cyperus | 32.1 | 9.2 | 63.6 | 38.3 | 25.3 |
| | | | difformis | | | | | |
| 1-4 | Propanil | 3 + 15 | Cyperus | 15.2 | 46.2 | 73.5 | 54.4 | 19.1 |
| | | | difformis | | | | | |
| 1-4 | Oxadiazon | 3 + 3 | Echinochloa | 60.2 | 14.3 | 90.5 | 65.9 | 24.6 |
| | | | phyllopogon | | | | | |
| 1-4 | Oxadiazon | 0.75 + 15 | Echinochloa | 30.3 | 41.3 | 86.6 | 59.1 | 27.5 |
| | | | phyllopogon | | | | | |
| 1-4 | Oxadiargyl | 3 + 0.6 | Echinochloa | 60.2 | 3.4 | 83.3 | 61.6 | 21.7 |
| | | | phyllopogon | | | | | |
| 1-4 | Oxadiargyl | 0.75 + 15 | Echinochloa | 30.3 | 35.3 | 78.6 | 54.9 | 23.7 |
| | | | phyllopogon | | | | | |
| 1-4 | Bentazone | 9 + 1.8 | Cyperus | 32.1 | 18.2 | 65.6 | 44.5 | 21.1 |
| | | | difformis | | | | | |
| 1-4 | Bentazone | 9 + 9 | Cyperus | 32.1 | 40.4 | 80.5 | 59.5 | 21.0 |
| | | | difformis | | | | | |
| 1-4 | Oxyfluorfen | 3 + 0.2 | Echinochloa | 60.2 | 6.7 | 86.7 | 62.9 | 23.8 |
| | | | phyllopogon | | | | | |
| 1-4 | Oxyfluorfen | 0.7 + 8 | Echinochloa | 25.1 | 76.5 | 95.4 | 82.4 | 13.0 |
| | | | phyllopogon | | | | | |
| 1-4 | Pentoxazone | 15 + 0.5 | Monochoria | 40.5 | 6.4 | 73.6 | 44.3 | 29.3 |
| | | | vaginalis | | | | | |
| 1-4 | Pentoxazone | 10 + 10 | Monochoria | 31.7 | 43.6 | 90.5 | 61.5 | 29.0 |
| | | | vaginalis | | | | | |
| 1-4 | Metribuzin | 3 + 0.6 | Echinochloa | 60.2 | 9.1 | 86.9 | 63.8 | 23.1 |
| | | | phyllopogon | | | | | |
| 1-4 | Metribuzin | 3 + 3 | Echinochloa | 60.2 | 32.3 | 90.6 | 73.1 | 17.5 |
| | | | phyllopogon | | | | | |
| 1-4 | Terbuthylazine | 3 + 0.6 | Echinochloa | 60.2 | 0.95 | 80.2 | 60.6 | 19.6 |
| | | | phyllopogon | | | | | |
| 1-4 | Terbuthylazine | 3 + 15 | Echinochloa | 60.2 | 18.6 | 91.4 | 67.6 | 23.8 |
| | | | phyllopogon | | | | | |
| 1-4 | Chlortoluron | 3 + 0.6 | Echinochloa | 60.2 | 18.3 | 85.6 | 67.5 | 18.1 |
| | | | phyllopogon | | | | | |
| 1-4 | Chlortoluron | 0.75 + 15 | Echinochloa | 30.3 | 57.6 | 91.5 | 70.4 | 21.1 |
| | | | phyllopogon | | | | | |
| 1-4 | Fluorochloridone | 3 + 3 | Echinochloa | 60.2 | 9.1 | 90.3 | 63.8 | 26.5 |
| | | | phyllopogon | | | | | |
| 1-4 | Fluorochloridone | 1.5 + 30 | Echinochloa | 41.5 | 52.3 | 87.7 | 72.1 | 15.6 |
| | | | phyllopogon | | | | | |
| 1-4 | Beflubutamid | 15 + 1.5 | Monochoria | 40.5 | 15.3 | 73.6 | 49.6 | 24.0 |
| | | | vaginalis | | | | | |
| 1-4 | Beflubutamid | 5 + 5 | Monochoria | 19.6 | 40.3 | 80.3 | 52.0 | 28.3 |
| | | | vaginalis | | | | | |
| 1-4 | Pyrazosulfuron- | 3 + 0.6 | Monochoria | 12.7 | 54.5 | 83.1 | 60.3 | 22.8 |
| | ethyl | | vaginalis | | | | | |
| 1-4 | Pyrazosulfuron- | 1 + 1 | Monochoria | 6.2 | 75.6 | 90.6 | 77.1 | 13.5 |
| | ethyl | | vaginalis | | | | | |
| 1-4 | Penoxsulam | 9 + 1.8 | Cyperus | 32.1 | 80.2 | 100 | 86.6 | 13.4 |
| | | | difformis | | | | | |
| 1-4 | Penoxsulam | 1 + 1 | Cyperus | 6.3 | 66.4 | 85.7 | 68.5 | 17.2 |
| | | | difformis | | | | | |
| 1-4 | Bispyribac- | 9 + 0.6 | Cyperus | 32.1 | 39.5 | 77.3 | 58.9 | 18.4 |
| | sodium | | difformis | | | | | |
| 1-4 | Bispyribac- | 1 + 1 | Cyperus | 6.3 | 70.4 | 88.6 | 72.3 | 16.3 |
| | sodium | | difformis | | | | | |
| 1-4 | Metazosulfuron | 6 + 1.2 | Monochoria | 28.6 | 30.3 | 81.5 | 68.8 | 12.7 |
| | | | vaginalis | | | | | |
| 1-4 | Metazosulfuron | 1 + 1 | Monochoria | 6.2 | 25.6 | 76.5 | 52.7 | 23.8 |
| | | | vaginalis | | | | | |
| 1-4 | Propyrisulfuron | 9 + 0.6 | Cyperus | 32.1 | 19.5 | 57.3 | 45.3 | 12.0 |
| | | | difformis | | | | | |
| 1-4 | Propyrisulfuron | 0.3 + 1.5 | Cyperus | 4.1 | 46.3 | 63.5 | 48.5 | 15.0 |
| | | | difformis | | | | | |
| 1-4 | Triafamone | 15 + 0.5 | Monochoria | 40.5 | 16.3 | 70.6 | 50.2 | 20.4 |
| | | | vaginalis | | | | | |
| 1-4 | Triafamone | 1 + 1 | Monochoria | 6.2 | 29.6 | 60.5 | 34.0 | 26.5 |
| | | | vaginalis | | | | | |
| 1-4 | Butralin | 3 + 3 | Cyperus | 15.2 | 16.4 | 58.5 | 29.1 | 29.4 |
| | | | difformis | | | | | |
| 1-4 | Butralin | 1.5 + 30 | Cyperus | 9.2 | 46.8 | 75.3 | 51.7 | 23.6 |
| | | | difformis | | | | | |
| 1-4 | Pendimethalin | 9 + 1.8 | Cyperus | 32.1 | 6.3 | 62.6 | 36.4 | 26.2 |
| | | | difformis | | | | | |
| 1-4 | Pendimethalin | 6 + 30 | Cyperus | 25.6 | 40.3 | 76.5 | 55.6 | 20.9 |
| | | | difformis | | | | | |
| 1-4 | Sanzuohuangcaotong | 15 + 3 | Monochoria | 40.5 | 21.5 | 79.6 | 53.3 | 26.3 |
| | ( [symbol] ) | | vaginalis | | | | | |
| 1-4 | Sanzuohuangcaotong | 6 + 6 | Monochoria | 28.6 | 40.1 | 83 | 57.2 | 25.8 |
| | ( [symbol] ) | | vaginalis | | | | | |
| 1-4 | Shuangzuocaotong | 3 + 0.6 | Echinochloa | 60.2 | 7.8 | 83.5 | 63.3 | 20.2 |
| | ( [symbol] ) | | phyllopogon | | | | | |
| 1-4 | Shuangzuocaotong | 1.6 + 8 | Echinochloa | 45.3 | 70.9 | 98.6 | 84.1 | 14.5 |
| | ( [symbol] ) | | phyllopogon | | | | | |
| 1-4 | Huanbifucaotong | 15 + 0.5 | Leptochloa | 45.6 | 9.2 | 68.7 | 50.6 | 18.1 |
| | ( [symbol] [symbol] ) | | chinensis | | | | | |
| 1-4 | Huanbifucaotong | 3 + 15 | Leptochloa | 18.6 | 76.2 | 100 | 80.6 | 19.4 |
| | ( [symbol] [symbol] ) | | chinensis | | | | | |
| 1-4 | Mesotrione | 9 + 0.6 | Cyperus | 32.1 | 29.5 | 67.3 | 52.1 | 15.2 |
| | | | difformis | | | | | |
| 1-4 | Mesotrione | 1 + 1 | Cyperus | 6.3 | 50.4 | 68.6 | 53.5 | 15.1 |
| | | | difformis | | | | | |
| 1-4 | Benzobicylon | 15 + 3 | Leptochloa | 45.6 | 26.8 | 76.3 | 60.2 | 16.1 |
| | | | chinensis | | | | | |
| 1-4 | Benzobicylon | 3 + 15 | Leptochloa | 18.6 | 68.2 | 98.6 | 74.1 | 24.5 |
| | | | chinensis | | | | | |
| 1-4 | Tefuryltrione | 9 + 1.8 | Cyperus | 32.1 | 63.2 | 89.6 | 75.0 | 14.6 |
| | | | difformis | | | | | |
| 1-4 | Tefuryltrione | 1 + 1 | Cyperus | 6.3 | 37.4 | 58.7 | 41.3 | 17.4 |
| | | | difformis | | | | | |
| 1-4 | Molinate | 3 + 3 | Echinochloa | 60.2 | 7.8 | 76.5 | 63.3 | 13.2 |
| | | | phyllopogon | | | | | |
| 1-4 | Molinate | 1.5 + 30 | Echinochloa | 41.5 | 40.6 | 78.5 | 65.3 | 13.2 |
| | | | phyllopogon | | | | | |
| 1-4 | Benthiocarb | 3 + 3 | Cyperus | 15.2 | 15.2 | 53.5 | 28.1 | 25.4 |
| | | | difformis | | | | | |
| 1-4 | Benthiocarb | 1.5 + 30 | Cyperus | 9.2 | 50.3 | 70.3 | 54.9 | 15.4 |
| | | | difformis | | | | | |
| 1-4 | Butachlor | 3 + 15 | Echinochloa | 60.2 | 19.3 | 85.5 | 67.9 | 17.6 |
| | | | phyllopogon | | | | | |
| 1-4 | Butachlor | 0.6 + 30 | Echinochloa | 20.5 | 39.7 | 65.2 | 52.1 | 13.1 |
| | | | phyllopogon | | | | | |
| 1-4 | Pretilachlor | 3 + 3 | Echinochloa | 60.2 | 15.2 | 85.3 | 66.2 | 19.1 |
| | | | phyllopogon | | | | | |
| 1-4 | Pretilachlor | 0.75 + 15 | Echinochloa | 30.3 | 43.2 | 80.5 | 60.4 | 20.1 |
| | | | phyllopogon | | | | | |
| 1-4 | Mefenacet | 3 + 3 | Echinochloa | 60.2 | 19.2 | 80.3 | 67.8 | 12.5 |
| | | | phyllopogon | | | | | |
| 1-4 | Mefenacet | 3 + 15 | Echinochloa | 60.2 | 46.7 | 96.1 | 78.8 | 17.3 |
| | | | phyllopogon | | | | | |
| 1-4 | Fentrazamide | 15 + 1.5 | Monochoria | 40.5 | 18.3 | 65.6 | 51.4 | 14.2 |
| | | | vaginalis | | | | | |
| 1-4 | Fentrazamide | 15 + 3 | Monochoria | 40.5 | 30.2 | 73.5 | 58.5 | 15.0 |
| | | | vaginalis | | | | | |
| 1-4 | Metolachlor | 3 + 3 | Echinochloa | 60.2 | 7.8 | 80.5 | 63.3 | 17.2 |
| | | | phyllopogon | | | | | |
| 1-4 | Metolachlor | 3 + 15 | Echinochloa | 60.2 | 15.3 | 87.5 | 66.3 | 21.2 |
| | | | phyllopogon | | | | | |
| 1-4 | Anilofos | 15 + 3 | Leptochloa | 45.6 | 30.8 | 100 | 62.4 | 37.6 |
| | | | chinensis | | | | | |
| 1-4 | Anilofos | 3 + 30 | Leptochloa | 18.6 | 62.6 | 100 | 69.6 | 30.4 |
| | | | chinensis | | | | | |
| 1-4 | Piperophos | 15 + 3 | Monochoria | 40.5 | 9.9 | 65.6 | 46.4 | 19.2 |
| | | | vaginalis | | | | | |
| 1-4 | Piperophos | 1 + 30 | Monochoria | 6.2 | 41.2 | 68.2 | 44.8 | 23.4 |
| | | | vaginalis | | | | | |
| 1-4 | Pyroxasulfone | 15 + 0.5 | Monochoria | 40.5 | 6.3 | 66.4 | 44.2 | 22.2 |
| | | | vaginalis | | | | | |
| 1-4 | Pyroxasulfone | 1.8 + 9 | Monochoria | 8.6 | 34.5 | 63.5 | 40.1 | 23.4 |
| | | | vaginalis | | | | | |
| 1-4 | Cyhalofop- | 3 + 3 | Echinochloa | 60.2 | 12.2 | 80.3 | 65.1 | 15.2 |
| | butyl | | phyllopogon | | | | | |
| 1-4 | Cyhalofop- | 0.75 + 15 | Echinochloa | 30.3 | 45.1 | 80.9 | 61.7 | 19.2 |
| | butyl | | phyllopogon | | | | | |
| 1-4 | Metamifop | 3 + 0.6 | Echinochloa | 60.2 | 6.8 | 83.5 | 62.9 | 20.6 |
| | | | phyllopogon | | | | | |
| 1-4 | Metamifop | 1.6 + 8 | Echinochloa | 45.3 | 60.9 | 96.6 | 78.6 | 18.0 |
| | | | phyllopogon | | | | | |
| 1-4 | Clomazone | 15 + 3 | Leptochloa | 45.6 | 16.8 | 80.6 | 54.7 | 25.9 |
| | | | chinensis | | | | | |
| 1-4 | Clomazone | 3 + 15 | Leptochloa | 18.6 | 53.6 | 90.4 | 62.2 | 28.2 |
| | | | chinensis | | | | | |
| 1-4 | Cinmethylin | 3 + 3 | Echinochloa | 60.2 | 13.2 | 82.3 | 65.5 | 16.8 |
| | | | phyllopogon | | | | | |
| 1-4 | Cinmethylin | 0.75 + 15 | Echinochloa | 30.3 | 54.5 | 85.7 | 68.3 | 17.4 |
| | | | phyllopogon | | | | | |
| 1-4 | Indanofan | 5 + 5 | Monochoria | 19.6 | 20.8 | 60.3 | 36.3 | 24.0 |
| | | | vaginalis | | | | | |
| 1-4 | Indanofan | 2.4 + 12 | Monochoria | 10.2 | 63.1 | 80 | 66.9 | 13.1 |
| | | | vaginalis | | | | | |
| 1-4 | Oxaziclomefone | 3 + 0.6 | Echinochloa | 60.2 | 12.5 | 83.1 | 65.2 | 17.9 |
| | | | phyllopogon | | | | | |
| 1-4 | Oxaziclomefone | 1 + 1 | Echinochloa | 36.6 | 18.7 | 70.6 | 48.5 | 22.1 |
| | | | phyllopogon | | | | | |

Note:
mu is a unit of land area in China, fifteen mus equal one hectare, i.e., 1 mu = 1/15 ha.

In addition, Table B1 lists specific combinations of other activity ingredients (i) and (ii), further illustrating the compositions of the present invention. The compounds in the column “activity Ingredient (i)/Compound No.” are listed in the index Table A1. The second column of Table B1 lists compounds of the particular activity ingredient (ii) (e.g., “Pyrazosulfuron-ethyl” in the first row). The remaining rows of Table B1 are constructed similarly.

TABLE B1
List of ingredients in the compositions

| Activity Ingredient (i)/ | |
|---|---|
| Compound. No. | Activity Ingredient (ii) |
| 1-3 | Pyrazosulfuron-ethyl |
| 1-3 | Penoxsulam |
| 1-3 | Bispyribac-sodium |
| 1-3 | Pyriminob ac-methyl |
| 1-3 | Metazosulfuron |
| 1-3 | Propyrisulfuron |
| 1-3 | Triafamone |
| 1-3 | Cyhalofop-butyl |
| 1-3 | Metamifop |
| 1-3 | Quinclorac |
| 1-3 | MCPA |
| 1-3 | 2,4-D |
| 1-3 | 2,4-D butyric acid |
| 1-3 | Fluroxypyr |
| 1-3 | Florpyrauxifen-benzyl |
| 1-3 | MCPA butyric acid |
| 1-3 | Dicamba |
| 1-3 | Quintrione |
| 1-3 | Clopyralid |
| 1-3 | Trichlopyr |
| 1-3 | Pendimethalin |
| 1-3 | Butralin |
| 1-3 | Benthiocarb |
| 1-3 | Molinate |
| 1-3 | Sanzuohuangcaotong [symbol] |
| 1-3 | Shuangzuocaotong [symbol] |
| 1-3 | Huanbifucaotong [symbol] |
| 1-3 | Mesotrione |
| 1-3 | Benzobicylon |
| 1-3 | Tefuryltrione |
| 1-3 | Diflufenican |
| 1-3 | Fluorochloridone |
| 1-3 | Beflubutamid |
| 1-3 | Carfentrazone-ethyl |
| 1-3 | Pyraclonil |
| 1-3 | Oxadiazon |
| 1-3 | Oxadiargyl |
| 1-3 | Oxyfluorfen |
| 1-3 | Pentoxazone |
| 1-3 | Butachlor |
| 1-3 | Pretilachlor |
| 1-3 | Mefenacet |
| 1-3 | Anilofos |
| 1-3 | Fentrazamide |
| 1-3 | Metolachlor |
| 1-3 | Piperophos |
| 1-3 | Pyroxasulfone |
| 1-3 | Simetryn |
| 1-3 | Prometryn |
| 1-3 | Amicarbazone |
| 1-3 | Isoproturon |
| 1-3 | Bromacil |
| 1-3 | Pyridate |
| 1-3 | Chlortoluron |
| 1-3 | Bentazone |
| 1-3 | Prop anil |
| 1-3 | Metribuzin |
| 1-3 | Atrazine |
| 1-3 | Bromoxynil |
| 1-3 | Bromoxynil octanoate |
| 1-3 | Terbuthylazine |
| 1-3 | Clomazone |
| 1-3 | Oxaziclomefone |
| 1-3 | Cinmethylin |
| 1-3 | Indanofan |

Table B2 is constructed in the same manner as the above Table B1, except that the first column “Activity Ingredient (i)/Comp. No.” are replaced by the corresponding “Activity Ingredient (i)/Comp. No.” column as shown below. The compounds in the “Activity Ingredient (i)/Comp. No.” column are listed in the Tables A1-A152. Accordingly, for example, in Table B2, the “Activity Ingredient (i)/Comp. No.” of the first column are all “1-5” (i.e., Compound 1-5 identified in the Table A1), and the first row below the column heading of Table B2 specifically discloses a mixture of Compound 1-5 and “Pyrazosulfuron-ethyl”. Tables B3 to B167 are similarly constructed.

| | “Activity | | “Activity | | “Activity | | “Activity |
|---|---|---|---|---|---|---|---|
| | Ingredient | | Ingredient | | Ingredient | | Ingredient |
| | (i)/Comp. | | (i)/Comp. | | (i)/Comp. | | (i)/Comp. |
| Table | No.” | Table | No.” | Table | No.” | Table | No.” |
| B2 | 1-5 | B3 | 1-6 | B4 | 1-7 | B5 | 1-8 |
| B6 | 1-9 | B7 | 1-10 | B8 | 1-11 | B9 | 1-12 |
| B10 | 1-13 | B11 | 1-14 | B12 | 1-15 | B13 | 1-16 |
| B14 | 1-17 | B15 | 1-18 | B16 | 1-19 | B17 | 1-20 |
| B18 | 1-21 | B19 | 1-22 | B20 | 1-23 | B21 | 1-24 |
| B22 | 1-25 | B23 | 1-26 | B24 | 1-27 | B25 | 1-28 |
| B26 | 1-29 | B27 | 1-30 | B28 | 1-31 | B29 | 1-32 |
| B30 | 1-33 | B31 | 1-34 | B32 | 1-35 | B33 | 1-36 |
| B34 | 1-37 | B35 | 1-38 | B36 | 1-39 | B37 | 1-40 |
| B38 | 1-41 | B39 | 1-42 | B40 | 1-43 | B41 | 1-44 |
| B42 | 1-45 | B43 | 1-46 | B44 | 1-47 | B45 | 1-48 |
| B46 | 1-49 | B47 | 1-50 | B48 | 1-51 | B49 | 1-52 |
| B50 | 1-53 | B51 | 1-54 | B52 | 1-55 | B53 | 1-56 |
| B54 | 1-57 | B55 | 1-58 | B56 | 1-59 | B57 | 1-60 |
| B58 | 1-61 | B59 | 1-62 | B60 | 1-63 | B61 | 1-64 |
| B62 | 1-65 | B63 | 1-66 | B64 | 1-67 | B65 | 1-68 |
| B66 | 1-69 | B67 | 1-70 | B68 | 1-71 | B69 | 1-72 |
| B70 | 1-73 | B71 | 1-74 | B72 | 1-75 | B73 | 1-76 |
| B74 | 1-77 | B75 | 1-78 | B76 | 1-79 | B77 | 1-80 |
| B78 | 1-81 | B79 | 1-82 | B80 | 1-83 | B81 | 1-84 |
| B82 | 1-85 | B83 | 1-86 | B84 | 1-87 | B85 | 1-88 |
| B86 | 1-89 | B87 | 1-90 | B88 | 1-91 | B89 | 1-92 |
| B90 | 1-93 | B91 | 1-94 | B92 | 1-95 | B93 | 1-96 |
| B94 | 1-97 | B95 | 1-98 | B96 | 1-99 | B97 | 1-100 |
| B98 | 1-101 | B99 | 1-102 | B100 | 1-103 | B101 | 1-104 |
| B102 | 1-105 | B103 | 1-106 | B104 | 1-107 | B105 | 1-108 |
| B106 | 1-109 | B107 | 1-110 | B108 | 1-111 | B109 | 1-112 |
| B110 | 1-113 | B111 | 1-114 | B112 | 1-115 | B113 | 1-116 |
| B114 | 1-117 | B115 | 1-118 | B116 | 1-119 | B117 | 1-120 |
| B118 | 1-121 | B119 | 1-122 | B120 | 1-123 | B121 | 1-124 |
| B122 | 1-125 | B123 | 1-126 | B124 | 1-127 | B125 | 1-128 |
| B126 | 1-129 | B127 | 1-130 | B128 | 1-131 | B129 | 1-132 |
| B130 | 1-133 | B131 | 1-134 | B132 | 1-135 | B133 | 1-136 |
| B134 | 1-137 | B135 | 1-138 | B136 | 1-139 | B137 | 1-140 |
| B138 | 1-141 | B139 | 1-142 | B140 | 1-143 | B141 | 1-144 |
| B142 | 1-145 | B143 | 1-146 | B144 | 1-147 | B145 | 1-148 |
| B146 | 2-3 | B147 | 2-4 | B148 | 4-4 | B149 | 6-4 |
| B150 | 8-4 | B151 | 10-4 | B152 | 14-4 | B153 | 17-4 |
| B154 | 17-5 | B155 | 18-4 | B156 | 20-2 | B157 | 20-4 |
| B158 | 21-4 | B159 | 21-5 | B160 | 22-4 | B161 | 24-4 |
| B162 | 96-4 | B163 | 96-5 | B164 | 101-4 | B165 | 101-5 |
| B166 | 103-4 | B167 | 103-5 | | | | |

At the same time, it is found after several tests that the other mixtures of activity ingredients (i) and (ii) of the present invention could produce similar herbicidal effects. In addition, the compounds and compositions of the present invention have good selectivity to many gramineae grasses such as zoysia japonica, bermuda grass, tall fescue, bluegrass, ryegrass and seashore paspalum etc, and are able to control many important grass weeds and broad-leaved weeds. The compounds also show excellent selectivity and commercial value in the tests on wheat, corn, rice, sugarcane, soybean, cotton, oil sunflower, potato, orchards and vegetables in different herbicide application methods.

1. An N-(1,3,4-oxadiazol-2-yl)arylcarboxamide compound represented by Formula I or a salt thereof, 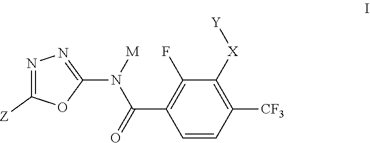
wherein, X represents O, S, SO, SO₂ or NR₁;
Y represents halogen, cyano, cyanoalkyl, carboxy, nitro, N(R₂)₂, -alkyl-N(R₂)₂, CON(R₂)₂, -alkyl-CON(R₂)₂, -alkyl-N(R₂)₃⁺I⁻, hydroxyalkyl, alkyl substituted by amino and carboxy, OR₃, SR₃, -alkyl-SOR₃, -alkyl-OR₃, -alkyl-SR₃, COR₃, COOR₃, -alkyl-COR₄, -alkyl-COOR₄, -alkyl-OCOR₃, Si(R₃)₃, -alkyl-O—Si(R₃)₃, -alkyl-O—N═C(R₃)₂, alkyl, haloalkyl, unsubstituted or substituted alkenyl, unsubstituted or substituted alkynyl, unsubstituted or substituted cycloalkyl, unsubstituted or substituted cycloalkylalkyl, unsubstituted or substituted heterocyclyl, unsubstituted or substituted aryl, unsubstituted or substituted heteroaryl, unsubstituted or substituted heterocyclylalkyl, unsubstituted or substituted arylalkyl, or unsubstituted or substituted heteroarylalkyl, wherein the substituted alkenyl or the substituted alkynyl is alkenyl or alkynyl substituted by at least one group selected from the group consisting of halogen, cyano, cycloalkyl, alkylcarbonyl, alkoxycarbonyl, alkoxy, alkylthio, alkylsulfinyl, alkylsulfonyl, and trialkylsilyl;
or, —X—Y represents an unsubstituted or substituted five- or six-membered heterocyclyl or heteroaryl, wherein the heterocyclyl or heteroaryl contains, besides C atom and N atom at 1-position, 0 to 3 of the following atoms or groups as a component of the ring: O, N, NR₁, SO₂, C═O;
Z represents hydrogen, halogen, cyano, OR₄, -alkyl-OR₄, —O-alkyl-N(R₅)₂, -alkyl-O-alkyl-N(R₅)₂, N(R₅)₂, -alkyl-(C═O)ₘ—N(R₅)₂, —NH-alkyl-N(R₅)₂, halogen-free or halogen-containing alkyl, halogen-free or halogen-containing alkenyl, halogen-free or halogen-containing alkynyl, halogen-free or halogen-containing alkoxycarbonyl, halogen-free or halogen-containing alkoxycarbonylalkyl, halogen-free or halogen-containing alkylcarbonyl, halogen-free or halogen-containing alkylcarbonylalkyl, halogen-free or halogen-containing alkylcarbonyloxyalkyl, halogen-free or halogen-containing alkylthio, halogen-free or halogen-containing alkylsulfinyl, halogen-free or halogen-containing alkylsulfonyl, unsubstituted or substituted cycloalkyl, unsubstituted or substituted heterocyclyl, unsubstituted or substituted aryl, unsubstituted or substituted heteroaryl, unsubstituted or substituted heterocyclylalkyl, unsubstituted or substituted arylalkyl, unsubstituted or substituted heteroarylalkyl, unsubstituted or substituted heterocyclylcarbonylalkyl, unsubstituted or substituted arylcarbonylalkyl, unsubstituted or substituted heteroarylcarbonylalkyl, unsubstituted or substituted heterocyclylcarbonyloxyalkyl, unsubstituted or substituted arylcarbonyloxyalkyl, or unsubstituted or substituted heteroarylcarbonyloxyalkyl;
M represents hydrogen, OR₆, SR₆, COR₆, COOR₆, OCOR₆, CON(R₇)₂, N(R₇)₂, NR₈COOR₆, NR₈CON(R₇)₂, -alkyl-R, halogen-free or halogen-containing alkyl, halogen-free or halogen-containing alkenyl, halogen-free or halogen-containing alkynyl, halogen-free or halogen-containing cycloalkyl, unsubstituted or substituted arylalkyl, or unsubstituted or substituted heteroarylalkyl;
R represents halogen-free or halogen-containing alkenyl, halogen-free or halogen-containing alkynyl, halogen-free or halogen-containing cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)—N(R₁₃)₂;
R₄, R₆, and R₁₁ each independently represent hydrogen, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing cycloalkenyl, halogen-free or halogen-containing alkoxyalkyl, unsubstituted or substituted heterocyclyl, unsubstituted or substituted heterocyclylalkyl, unsubstituted or substituted heterocyclyloxyalkyl, unsubstituted or substituted aryl, unsubstituted or substituted arylalkyl, unsubstituted or substituted aryloxyalkyl, unsubstituted or substituted heteroaryl, unsubstituted or substituted heteroarylalkyl, or unsubstituted or substituted heteroaryloxyalkyl;
R₃ and R₁₂ each independently represent halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing cycloalkenyl, halogen-free or halogen-containing alkoxyalkyl, unsubstituted or substituted heterocyclyl, unsubstituted or substituted heterocyclylalkyl, unsubstituted or substituted aryl, unsubstituted or substituted arylalkyl, unsubstituted or substituted heteroaryl, or unsubstituted or substituted heteroarylalkyl;
R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, alkyl, haloalkyl, alkenyl, haloalkenyl, alkynyl, haloalkynyl, cycloalkyl, cycloalkenyl, halocycloalkyl, alkoxyalkyl, cycloalkylalkyl, aryl, arylalkyl, aryloxy, aryloxyalkyl, arylalkyloxy, arylcarbonyl, aryl sulfonyl, heteroaryl, heteroarylalkyl, heteroaryloxy, heteroaryloxyalkyl, heteroarylalkyloxy, heteroarylcarbonyl, heteroarylsulfonyl, heterocyclyl, heterocyclylalkyl, heterocyclyloxy, heterocyclyloxyalkyl, heterocyclylalkyloxy, heterocyclylcarbonyl, heterocyclylsulfonyl, aryl-NR₂₁-alkyl, heteroaryl-NR₂₁-alkyl, heterocyclyl-NR₂₁-alkyl, wherein the last 35 groups as mentioned are each substituted by 0, 1, 2 or 3 groups selected from the group consisting of: cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and alkoxyalkoxycarbonyl;
R₂₁ independently represents hydrogen, alkyl, alkenyl, alkynyl, cycloalkyl, or cycloalkylalkyl;
R₂₂ independently represents alkyl, alkenyl, alkynyl, cycloalkyl, or cycloalkylalkyl;
m represents 0 or 1;
n and r each independently represent 0, 1 or 2; and
when X represents S or SO, Y is not methyl.

2. The N-(1,3,4-oxadiazol-2-yl)arylcarboxamide compound or a salt thereof according to claim 1, wherein
X represents O, S, SO, SO₂ or NR₁;
Y represents halogen, cyano, cyano-(C₁-C₈)alkyl, carboxy, nitro, N(R₂)₂, —(C₁-C₈)alkyl-N(R₂)₂, CON(R₂)₂, —(C₁-C₈)alkyl-CON(R₂)₂, —(C₁-C₈)alkyl-N(R₂)₃⁺I⁻, hydroxy(C₁-C₈)alkyl, (C₁-C₈)alkyl substituted by amino and carboxy, OR₃, SR₃, —(C₁-C₈)alkyl-SOR₃, —(C₁-C₈)alkyl-OR₃, —(C₁-C₈)alkyl-SR₃, COR₃, COOR₃, —(C₁-C₈)alkyl-COR₄, —(C₁-C₈)alkyl-COOR₄, —(C₁-C₈)alkyl-OCOR₃, Si(R₃)₃, —(C₁-C₈)alkyl-O—Si(R₃)₃, —(C₁-C₈)alkyl-O—N═C(R₃)₂, C₁-C₈alkyl, halo C₁-C₈ alkyl; unsubstituted or substituted C₂-C₈ alkenyl, unsubstituted or substituted C₂-C₈ alkynyl, unsubstituted or substituted C₃-C₈ cycloalkyl, unsubstituted or substituted C₃-C₈ cycloalkyl C₁-C₈ alkyl, unsubstituted or substituted heterocyclyl, unsubstituted or substituted aryl, unsubstituted or substituted heteroaryl, unsubstituted or substituted heterocyclyl C₁-C₈ alkyl, unsubstituted or substituted aryl C₁-C₈ alkyl, unsubstituted or substituted heteroaryl C₁-C₈ alkyl, wherein the substituted C₂-C₈ alkenyl or the substituted C₂-C₈ alkynyl is C₂-C₈ alkenyl or C₂-C₈ alkynyl substituted by at least one group selected from the group consisting of halogen, cyano, C₃-C₈ cycloalkyl, C₁-C₈ alkylcarbonyl, C₁-C₈ alkoxycarbonyl, C₁-C₈ alkoxy, C₁-C₈ alkylthio, C₁-C₈ alkylsulfinyl, C₁-C₈ alkylsulfonyl and tri(C₁-C₈)alkylsilyl, the substituted C₃-C₈ cycloalkyl or the substituted (C₃-C₈)cycloalkyl(C₁-C₈)alkyl is C₃-C₈ cycloalkyl or (C₃-C₈)cycloalkyl(C₁-C₈)alkyl substituted by at least one group selected from the group consisting of C₁-C₈ alkyl, halogen and phenyl;
or, —X—Y represents 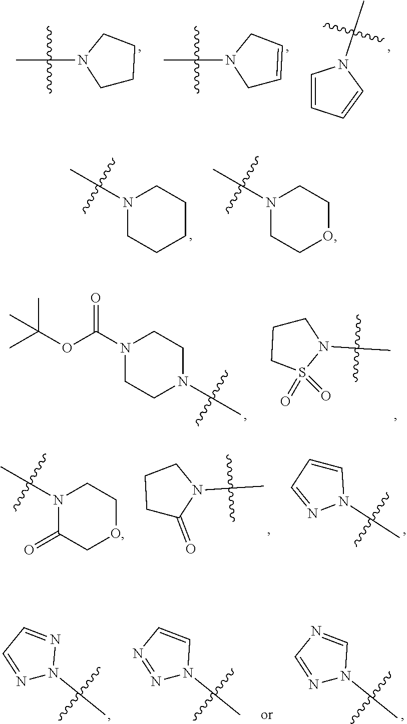
which is unsubstituted or substituted by at least one group selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing alkoxy, halogen-free or halogen-containing alkylthio, halogen-free or halogen-containing alkoxyalkyl, halogen-free or halogen-containing alkylthioalkyl, halogen-free or halogen-containing alkylcarbonyl, halogen-free or halogen-containing alkoxycarbonyl, halogen-free or halogen-containing alkylsulfinyl, halogen-free or halogen-containing alkylsulfonyl, halogen-free or halogen-containing alkylamino, halogen-free or halogen-containing dialkylamino, halogen-free or halogen-containing alkylacyloxy, wherein the substituted phenyl, the substituted benzyl or the substituted phenoxy is phenyl, benzyl or phenoxy substituted by at least one group selected from the group consisting of halogen, alkyl and alkoxy;
Z represents hydrogen, halogen, cyano, OR₄, —(C₁-C₈)alkyl-OR₄, —O—(C₁-C₈)alkyl-N(R₅)₂, —(C₁-C₈)alkyl-O—(C₁-C₈)alkyl-N(R₅)₂, N(R₅)₂, —(C₁-C₈)alkyl-(C═O)ₘ—N(R₅)₂, —NH—(C₁-C₈)alkyl-N(R₅)₂, halogen-free or halogen-containing C₁-C₈ alkyl, halogen-free or halogen-containing C₂-C₈ alkenyl, halogen-free or halogen-containing C₂-C₈ alkynyl, halogen-free or halogen-containing C₁-C₈alkoxy carbonyl, halogen-free or halogen-containing C₁-C₈alkoxycarbonyl C₁-C₈alkyl, halogen-free or halogen-containing C₁-C₈alkyl carbonyl, halogen-free or halogen-containing C₁-C₈alkyl carbonyl C₁-C₈alkyl, halogen-free or halogen-containing C₁-C₈alkyl carbonyloxy C₁-C₈alkyl, halogen-free or halogen-containing C₁-C₈ alkylthio, halogen-free or halogen-containing C₁-C₈alkylsulfinyl, halogen-free or halogen-containing C₁-C₈alkylsulfonyl, unsubstituted or substituted C₃-C₈ cycloalkyl, unsubstituted or substituted heterocyclyl, unsubstituted or substituted aryl, unsubstituted or substituted heteroaryl, unsubstituted or substituted heterocyclyl C₁-C₈alkyl, unsubstituted or substituted aryl C₁-C₈alkyl, unsubstituted or substituted heteroaryl C₁-C₈alkyl, unsubstituted or substituted heterocyclylcarbonyl C₁-C₈alkyl, unsubstituted or substituted arylcarbonyl C₁-C₈alkyl, unsubstituted or substituted heteroarylcarbonyl C₁-C₈alkyl, unsubstituted or substituted heterocyclylcarbonyloxy C₁-C₈alkyl, unsubstituted or substituted arylcarbonyloxy C₁-C₈alkyl, or unsubstituted or substituted heteroarylcarbonyloxy C₁-C₈alkyl, wherein the substituted C₃-C₈ cycloalkyl is C₃-C₈ cycloalkyl substituted by at least one group selected from the group consisting of C₁-C₈ alkyl, halogen and phenyl;
M represents hydrogen, OR₆, SR₆, COR₆, COOR₆, OCOR₆, CON(R₇)₂, N(R₇)₂, NR₈COOR₆, NR₈CON(R₇)₂, —(C₁-C₈)alkyl-R, halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, unsubstituted or substituted aryl C₁-C₈alkyl, or unsubstituted or substituted heteroaryl C₁-C₈ alkyl;
R represents halogen-free or halogen-containing C₂-C₈alkenyl, halogen-free or halogen-containing C₂-C₈alkynyl, halogen-free or halogen-containing C₃-C₈cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)—N(R₁₃)₂;
R₄, R₆ and Ru each independently represent hydrogen, halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, halogen-free or halogen-containing C₃-C₈cycloalkyl C₁-C₈alkyl, halogen-free or halogen-containing C₃-C₈cycloalkenyl, halogen-free or halogen-containing C₁-C₈alkoxy C₁-C₈alkyl, unsubstituted or substituted heterocyclyl, unsubstituted or substituted heterocyclyl C₁-C₈alkyl, unsubstituted or substituted heterocyclyloxy C₁-C₈alkyl, unsubstituted or substituted aryl, unsubstituted or substituted aryl C₁-C₈alkyl, unsubstituted or substituted aryloxy C₁-C₈alkyl, unsubstituted or substituted heteroaryl, unsubstituted or substituted heteroaryl C₁-C₈alkyl, unsubstituted or substituted heteroaryloxy C₁-C₈alkyl;
R₃ and R₁₂ each independently represent halogen-free or halogen-containing C₁-C₈alkyl, halogen-free or halogen-containing C₂-C₈alkenyl, halogen-free or halogen-containing C₂-C₈alkynyl, halogen-free or halogen-containing C₃-C₈cycloalkyl, halogen-free or halogen-containing C₃-C₈cycloalkyl C₁-C₈alkyl, halogen-free or halogen-containing C₃-C₈cycloalkenyl, halogen-free or halogen-containing C₁-C₈alkoxy C₁-C₈alkyl, unsubstituted or substituted heterocyclyl, unsubstituted or substituted heterocyclyl C₁-C₈alkyl, unsubstituted or substituted aryl, unsubstituted or substituted aryl C₁-C₈alkyl, unsubstituted or substituted heteroaryl, unsubstituted or substituted heteroaryl C₁-C₈alkyl;
R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, C₁-C₈alkyl, haloC₁-C₈alkyl, C₂-C₈alkenyl, haloC₂-C₈alkenyl, C₂-C₈alkynyl, haloC₂-C₈alkynyl, C₃-C₈cycloalkyl, C₃-C₈cycloalkenyl, haloC₃-C₈cycloalkyl, C₁-C₈alkoxy C₁-C₈alkyl, C₃-C₈cycloalkyl C₁-C₈alkyl, aryl, aryl C₁-C₈alkyl, aryloxy, aryloxy C₁-C₈alkyl, aryl C₁-C₈alkyloxy, arylcarbonyl, aryl sulfonyl, heteroaryl, heteroaryl C₁-C₈alkyl, heteroaryloxy, heteroaryloxy C₁-C₈alkyl, heteroaryl C₁-C₈alkyloxy, heteroarylcarbonyl, heteroarylsulfonyl, heterocyclyl, heterocyclyl C₁-C₈alkyl, heterocyclyloxy, heterocyclyloxy C₁-C₈alkyl, heterocyclyl C₁-C₈alkyloxy, heterocyclylcarbonyl, heterocyclylsulfonyl, aryl-NR₂₁—(C₁-C₈)alkyl, heteroaryl-NR₂₁—(C₁-C₈)alkyl, or heterocyclyl-NR₂₁—(C₁-C₈)alkyl, wherein the last 35 groups as mentioned each are independently substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₈alkoxy C₁-C₈alkoxycarbonyl;
R₂₁ independently represents hydrogen, C₁-C₈alkyl, C₂-C₈alkenyl, C₂-C₈alkynyl, C₃-C₈cycloalkyl, or C₃-C₈cycloalkyl C₁-C₈alkyl;
R₂₂ independently represents C₁-C₈alkyl, C₂-C₈alkenyl, C₂-C₈alkynyl, C₃-C₈cycloalkyl, or C₃-C₈cycloalkyl C₁-C₈alkyl;
m represents 0 or 1;
n and r each independently represent 0, 1 or 2;
wherein, the “heterocyclyl” refers to 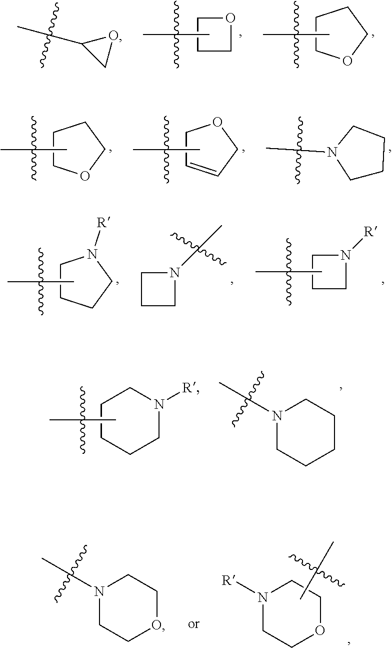
which has 0, 1 or 2 oxo groups; the “aryl” refers to phenyl or naphthyl, the “heteroaryl” refers to 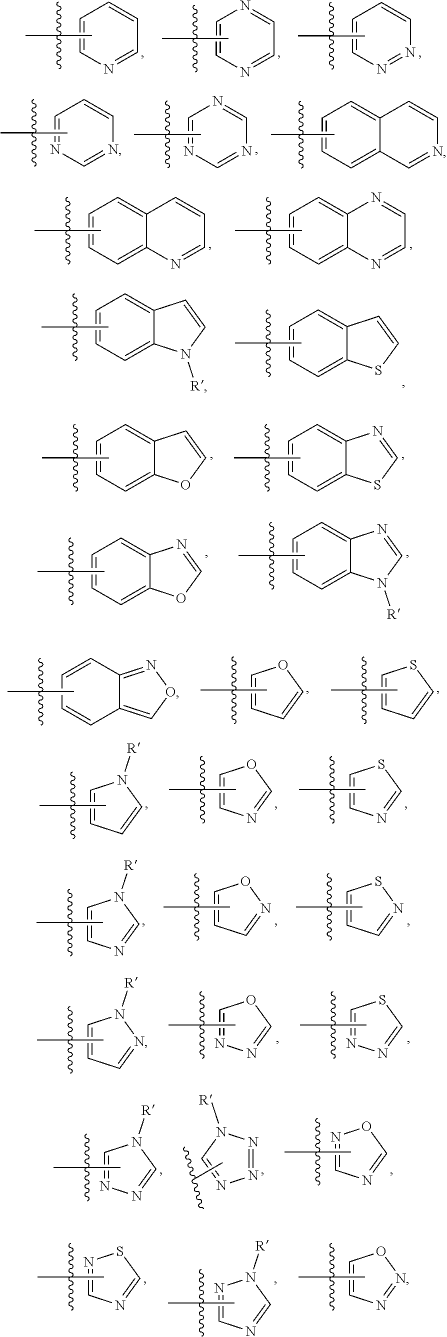 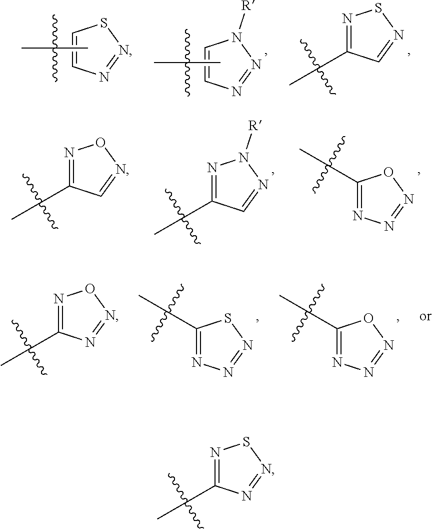
wherein the heterocyclyl, the aryl or the heteroaryl is unsubstituted or substituted by at least one group selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-free or halogen-containing alkyl, halogen-free or halogen-containing alkenyl, halogen-free or halogen-containing alkynyl, halogen-free or halogen-containing cycloalkyl, halogen-free or halogen-containing cycloalkylalkyl, halogen-free or halogen-containing OR″, halogen-free or halogen-containing SR″, halogen-free or halogen-containing -alkyl-OR″, halogen-free or halogen-containing -alkyl-SR″, halogen-free or halogen-containing COR″, halogen-free or halogen-containing COOR″, halogen-free or halogen-containing COSR″, halogen-free or halogen-containing SOR″, halogen-free or halogen-containing SO₂R″, halogen-free or halogen-containing OCOR″, halogen-free or halogen-containing SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted with at least one group selected from the group consisting of halogen, alkyl and alkoxy, the substituted amino or the substituted aminocarbonyl is amino or aminocarbonyl substituted by one or two groups selected from the group consisting of hydrogen, alkyl, alkenyl, alkynyl, cycloalkyl, cycloalkylalkyl, COR″, SO₂R″, and OR″;
R′ independently represents hydrogen, nitro, hydroxy, amino, halogen-containing or halogen-free alkyl, halogen-containing or halogen-free alkenyl, halogen-containing or halogen-free alkynyl, halogen-containing or halogen-free cycloalkyl, halogen-containing or halogen-free cycloalkenyl, halogen-containing or halogen-free cycloalkylalkyl, halogen-containing or halogen-free alkoxy, halogen-containing or halogen-free alkenyloxy, halogen-containing or halogen-free alkynyloxy, halogen-containing or halogen-free cycloalkyloxy, halogen-containing or halogen-free alkoxyalkyl, halogen-containing or halogen-free alkoxycarbonyl, halogen-containing or halogen-free alkylthiocarbonyl, halogen-containing or halogen-free alkylsulfonyl, halogen-containing or halogen-free alkylsulfonylalkyl, halogen-containing or halogen-free alkylcarbonyl, halogen-containing or halogen-free alkylcarbonylalkyl, halogen-containing or halogen-free alkylacyloxy, halogen-containing or halogen-free alkylamino, halogen-containing or halogen-free alkylaminocarbonyl, halogen-containing or halogen-free alkoxyaminocarbonyl, halogen-containing or halogen-free alkoxycarbonylalkyl, halogen-containing or halogen-free alkylaminocarbonylalkyl, halogen-containing or halogen-free trialkylsilyl, or halogen-containing or halogen-free dialkylphosphonyl;
R″ independently represents hydrogen, halogen-containing or halogen-free alkyl, halogen-containing or halogen-free alkenyl, halogen-containing or halogen-free alkynyl, halogen-containing or halogen-free cycloalkyl, or halogen-containing or halogen-free cycloalkylalkyl.

3. The N-(1,3,4-oxadiazol-2-yl)arylcarboxamide compound or a salt thereof according to claim 1, wherein
X represents O, S, SO, SO₂ or NR₁;
Y represents halogen, cyano, cyano C₁-C₆alkyl, carboxy, nitro, N(R₂)₂, —(C₁-C₆)alkyl-N(R₂)₂, CON(R₂)₂, —(C₁-C₆)alkyl-CON(R₂)₂, —(C₁-C₆)alkyl-N(R₂)₃⁺I⁻, hydroxy C₁-C₆alkyl, C₁-C₆alkyl substituted by amino and carboxy, OR₃, SR₃, —(C₁-C₆)alkyl-SOR₃, —(C₁-C₆)alkyl-OR₃, —(C₁-C₆)alkyl-SR₃, COR₃, COOR₃, —(C₁-C₆)alkyl-COR₄, —(C₁-C₆)alkyl-COOR₄, —(C₁-C₆)alkyl-OCOR₃, Si(R₃)₃, —(C₁-C₆)alkyl-O—Si(R₃)₃, —(C₁-C₆)alkyl-O—N═C(R₃)₂, C₁-C₆alkyl, haloC₁-C₆alkyl, unsubstituted or substituted C₂-C₆ alkenyl, unsubstituted or substituted C₂-C₆ alkynyl, unsubstituted or substituted C₃-C₆ cycloalkyl, unsubstituted or substituted C₃-C₆cycloalkyl unsubstituted or substituted heterocyclyl, unsubstituted or substituted aryl, unsubstituted or substituted heteroaryl, unsubstituted or substituted heterocyclyl C₁-C₆alkyl, unsubstituted or substituted aryl C₁-C₆alkyl, unsubstituted or substituted heteroaryl C₁-C₆alkyl, wherein the substituted C₂-C₆ alkenyl or the substituted C₂-C₆ alkynyl is C₂-C₆ alkenyl or C₂-C₆ alkynyl substituted by at least one group selected from the group consisting of halogen, cyano, C₃-C₆ cycloalkyl, C₁-C₆ alkyl carbonyl, C₁-C₆ alkoxy carbonyl, C₁-C₆ alkoxy, C₁-C₆ alkylthio, C₁-C₆ alkyl sulfinyl, C₁-C₆ alkyl sulfonyl and tri(C₁-C₆)alkylsilyl, the substituted C₃-C₆ cycloalkyl or the substituted C₃-C₆cycloalkyl C₁-C₆alkyl is C₃-C₆ cycloalkyl or C₃-C₆cycloalkyl C₁-C₆alkyl substituted by at least one group selected from the group consisting of C₁-C₆ alkyl, halogen and phenyl;
or, —X—Y represents 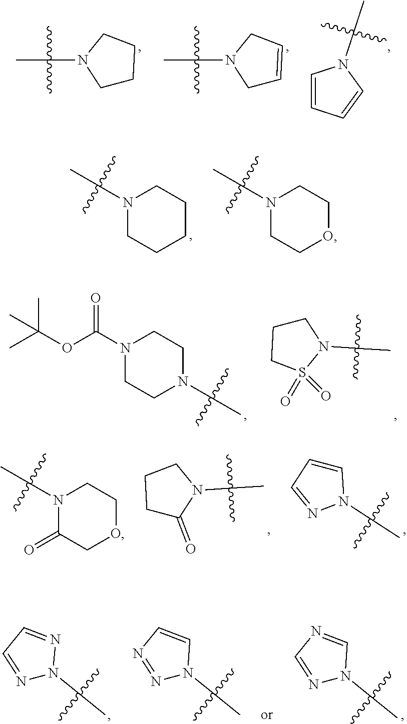
which is unsubstituted or substituted by 1, 2 or 3 groups selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-containing or halogen-free C₁-C₈ alkyl, halogen-containing or halogen-free C₂-C₈ alkenyl, halogen-containing or halogen-free C₂-C₈ alkynyl, halogen-containing or halogen-free C₃-C₈ cycloalkyl, halogen-containing or halogen-free C₃-C₈cycloalkyl C₁-C₈alkyl, halogen-containing or halogen-free C₁-C₈ alkoxy, halogen-containing or halogen-free C₁-C₈ alkylthio, halogen-containing or halogen-free C₁-C₈alkoxy C₁-C₈alkyl, halogen-containing or halogen-free C₁-C₈alkylthio C₁-C₈alkyl, halogen-containing or halogen-free C₁-C₈alkyl carbonyl, halogen-containing or halogen-free C₁-C₈alkoxy carbonyl, halogen-containing or halogen-free C₁-C₈alkyl sulfinyl, halogen-containing or halogen-free C₁-C₈alkyl sulfonyl, halogen-containing or halogen-free C₁-C₈alkyl amino, halogen-containing or halogen-free di(C₁-C₈)alkyl amino, and halogen-containing or halogen-free C₁-C₈alkyl acyloxy, wherein the substituted phenyl, the substituted benzyl or the substituted phenoxy is phenyl, benzyl or phenoxy substituted by 1, 2 or 3 groups selected from the group consisting of halogen, C₁-C₈ alkyl and C₁-C₈ alkoxy;
Z represents hydrogen, halogen, cyano, OR₄, —(C₁-C₆)alkyl-OR₄, —O—(C₁-C₆)alkyl-N(R₅)₂, —(C₁-C₆)alkyl-O—(C₁-C₆)alkyl-N(R₅)₂, N(R₅)₂, —(C₁-C₆)alkyl-(C═O)ₘ—N(R₅)₂, —NH—(C₁-C₆)alkyl-N(R₅)₂, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₁-C₆alkoxy carbonyl, halogen-free or halogen-containing C₁-C₆alkoxy carbonyl C₁-C₆alkyl, halogen-free or halogen-containing C₁-C₆alkyl carbonyl, halogen-free or halogen-containing C₁-C₆alkyl carbonyl C₁-C₆alkyl, halogen-free or halogen-containing C₁-C₆alkyl carbonyloxy C₁-C₆alkyl, halogen-free or halogen-containing C₁-C₆ alkylthio, halogen-free or halogen-containing C₁-C₆alkyl sulfinyl, halogen-free or halogen-containing C₁-C₆alkyl sulfonyl, unsubstituted or substituted C₃-C₆ cycloalkyl, unsubstituted or substituted heterocyclyl, unsubstituted or substituted aryl, unsubstituted or substituted heteroaryl, unsubstituted or substituted heterocyclyl C₁-C₆alkyl, unsubstituted or substituted aryl C₁-C₆alkyl, unsubstituted or substituted heteroaryl C₁-C₆alkyl, unsubstituted or substituted heterocyclyl carbonyl C₁-C₆alkyl, unsubstituted or substituted aryl carbonyl C₁-C₆alkyl, unsubstituted or substituted heteroaryl carbonyl C₁-C₆alkyl, unsubstituted or substituted heterocyclyl carbonyloxy C₁-C₆alkyl, unsubstituted or substituted aryl carbonyloxy C₁-C₆alkyl, or unsubstituted or substituted heteroaryl carbonyloxy C₁-C₆alkyl, wherein the substituted C₃-C₆ cycloalkyl is C₃-C₆ cycloalkyl substituted by at least one group selected from the group consisting of C₁-C₆ alkyl, halogen and phenyl;
M represents hydrogen, OR₆, SR₆, COR₆, COOR₆, OCOR₆, CON(R₇)₂, N(R₇)₂, NR₈COOR₆, NR₈CON(R₇)₂, —(C₁-C₆)alkyl-R, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, unsubstituted or substituted aryl C₁-C₆alkyl, or unsubstituted or substituted heteroaryl C₁-C₆alkyl;
R represents halogen-free or halogen-containing C₂-C₆ alkenyl, halogen-free or halogen-containing C₂-C₆ alkynyl, halogen-free or halogen-containing C₃-C₆ cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, —O—(C═O)—N(R₁₃)₂;
R₄, R₆ and Ru each independently represent hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆cycloalkyl C₁-C₆alkyl, halogen-free or halogen-containing C₃-C₆ cycloalkenyl, C₁-C₆alkoxy C₁-C₆alkyl, unsubstituted or substituted heterocyclyl, unsubstituted or substituted heterocyclyl C₁-C₆alkyl, unsubstituted or substituted heterocyclyloxy C₁-C₆alkyl, unsubstituted or substituted aryl, unsubstituted or substituted aryl C₁-C₆alkyl, unsubstituted or substituted aryloxy C₁-C₆alkyl, unsubstituted or substituted heteroaryl, unsubstituted or substituted heteroaryl C₁-C₆alkyl, or unsubstituted or substituted heteroaryloxy C₁-C₆alkyl;
R₃ and R₁₂ each independently represent halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆cycloalkyl C₁-C₆alkyl, halogen-free or halogen-containing C₃-C₆ cycloalkenyl, halogen-free or halogen-containing C₁-C₆alkoxy C₁-C₆alkyl, unsubstituted or substituted heterocyclyl, unsubstituted or substituted heterocyclyl C₁-C₆alkyl, unsubstituted or substituted aryl, unsubstituted or substituted aryl C₁-C₆alkyl, unsubstituted or substituted heteroaryl, unsubstituted or substituted heteroaryl C₁-C₆alkyl;
R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₂₁, S(O)ᵣR₂₂, C₁-C₆ alkyl, halo C₁-C₆alkyl, C₂-C₆ alkenyl, halo C₂-C₆alkenyl, C₂-C₆ alkynyl, halo C₂-C₆alkynyl, C₃-C₆ cycloalkyl, C₃-C₆ cycloalkenyl, halo C₃-C₆cycloalkyl, C₁-C₆alkoxy C₁-C₆alkyl, C₃-C₆cycloalkyl C₁-C₆alkyl, aryl, aryl C₁-C₆alkyl, aryloxy, aryloxy C₁-C₆alkyl, aryl C₁-C₆alkyloxy, arylcarbonyl, aryl sulfonyl, heteroaryl, heteroaryl C₁-C₆alkyl, heteroaryloxy, heteroaryloxy C₁-C₆alkyl, heteroaryl C₁-C₆alkyloxy, heteroarylcarbonyl, heteroarylsulfonyl, heterocyclyl, heterocyclyl C₁-C₆alkyl, heterocyclyloxy, heterocyclyloxy C₁-C₆alkyl, heterocyclyl C₁-C₆alkyloxy, heterocyclylcarbonyl, heterocyclylsulfonyl, aryl-NR₂₁—(C₁-C₆)alkyl, heteroaryl-NR₂₁—(C₁-C₆)alkyl, heterocyclyl-NR₂₁—(C₁-C₆)alkyl, wherein the last 35 groups as mentioned are each substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₆alkoxy C₁-C₆alkoxy carbonyl;
R₂₁ independently represents hydrogen, C₁-C₆ alkyl, C₂-C₆ alkenyl, C₂-C₆ alkynyl, C₃-C₆ cycloalkyl, or C₃-C₆cycloalkyl C₁-C₆alkyl;
R₂₂ independently represents C₁-C₆ alkyl, C₂-C₆ alkenyl, C₂-C₆ alkynyl, C₃-C₆ cycloalkyl, or C₃-C₆cycloalkyl C₁-C₆alkyl;
m represents 0 or 1;
n and r each independently represent 0, 1 or 2;
wherein, the “heterocyclyl” refers to 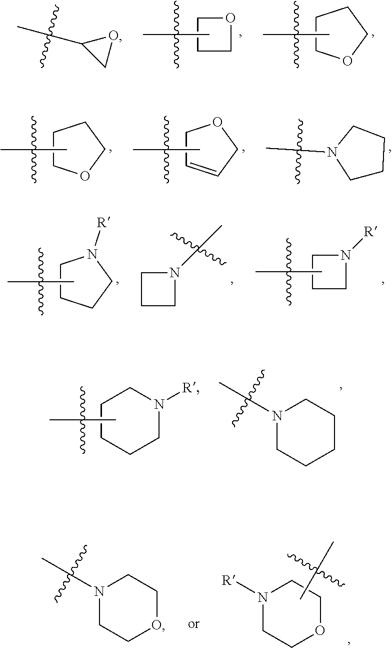
which has 0, 1 or 2 oxo groups; the “aryl” refers to phenyl or naphthyl, and “heteroaryl” refers to 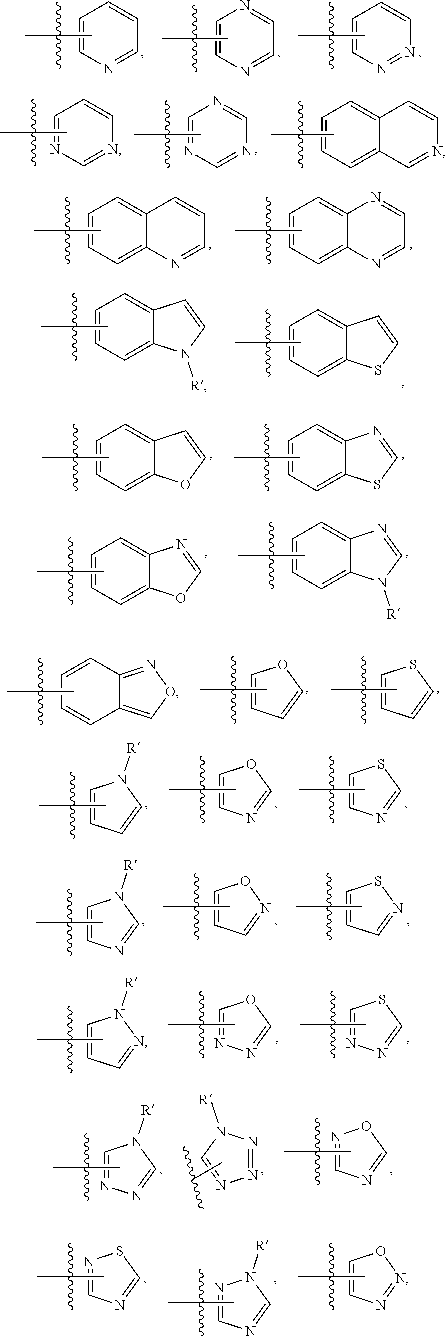 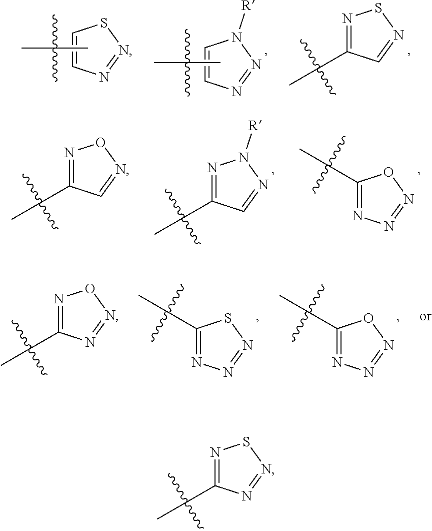
which is unsubstituted or substituted by 0, 1, 2 or 3 groups selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-containing or halogen-free C₁-C₈ alkyl, halogen-containing or halogen-free C₂-C₈ alkenyl, halogen-containing or halogen-free C₂-C₈ alkynyl, halogen-containing or halogen-free C₃-C₈ cycloalkyl, halogen-containing or halogen-free C₃-C₈ cycloalkyl C₁-C₈ alkyl, halogen-containing or halogen-free OR″, halogen-containing or halogen-free SR″, halogen-containing or halogen-free —(C₁-C₈)alkyl-OR″, halogen-containing or halogen-free —(C₁-C₈)alkyl-SR″, halogen-containing or halogen-free COR″, halogen-containing or halogen-free COOR″, halogen-containing or halogen-free COSR″, halogen-containing or halogen-free SOR″, halogen-containing or halogen-free SO₂R″, halogen-containing or halogen-free OCOR″, halogen-containing or halogen-free SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted by 1, 2 or 3 groups selected from halogen, C₁-C₈ alkyl and C₁-C₈ alkoxy, the substituted amino or the substituted aminocarbonyl is amino or aminocarbonyl substituted by one or two groups selected from hydrogen, C₁-C₈ alkyl, C₂-C₈ alkenyl, C₂-C₈ alkynyl, C₃-C₈ cycloalkyl, C₃-C₈cycloalkyl C₁-C₈alkyl, COR″, SO₂R″ and OR″;
R′ independently represents hydrogen, nitro, hydroxy, amino, fluorine-, chlorine-, bromine-free or fluorine-, chlorine-, bromine-containing C₁-C₈ alkyl, fluorine-, chlorine-, bromine-free or fluorine-, chlorine-, bromine-containing C₂-C₈ alkenyl, fluorine-, chlorine-, bromine-free or fluorine-, chlorine-, bromine-containing C₂-C₈ alkynyl, fluorine-, chlorine-, bromine-free or fluorine-, chlorine-, bromine-containing C₃-C₈ cycloalkyl, fluorine-, chlorine-, bromine-free or fluorine-, chlorine-, bromine-containing C₃-C₈ cycloalkenyl, fluorine-, chlorine-, bromine-free or fluorine-, chlorine-, bromine-containing C₃-C₈cycloalkyl C₁-C₈alkyl, fluorine-, chlorine-, bromine-free or fluorine-, chlorine-, bromine-containing C₁-C₈alkoxy, fluorine-, chlorine-, bromine-free or fluorine-, chlorine-, bromine-containing C₂-C₈ alkenyloxy, fluorine-, chlorine-, bromine-free or fluorine-, chlorine-, bromine-containing C₂-C₈ alkynyloxy, fluorine-, chlorine-, bromine-free or fluorine-, chlorine-, bromine-containing C₃-C₈ cycloalkyloxy, fluorine-, chlorine-, bromine-free or fluorine-, chlorine-, bromine-containing C₁-C₈alkoxy C₁-C₈alkyl, fluorine-, chlorine-, bromine-free or fluorine-, chlorine-, bromine-containing C₁-C₈alkoxy carbonyl, fluorine-, chlorine-, bromine-free or fluorine-, chlorine-, bromine-containing C₁-C₈alkylthio carbonyl, fluorine-, chlorine-, bromine-free or fluorine-, chlorine-, bromine-containing C₁-C₈alkylsulfonyl, fluorine-, chlorine-, bromine-free or fluorine-, chlorine-, bromine-containing C₁-C₈alkylsulfonyl C₁-C₈alkyl, fluorine-, chlorine-, bromine-free or fluorine-, chlorine-, bromine-containing C₁-C₈alkyl carbonyl, fluorine-, chlorine-, bromine-free or fluorine-, chlorine-, bromine-containing C₁-C₈alkyl carbonyl C₁-C₈alkyl, fluorine-, chlorine-, bromine-free or fluorine-, chlorine-, bromine-containing C₁-C₈alkyl acyloxy, fluorine-, chlorine-, bromine-free or fluorine-, chlorine-, bromine-containing C₁-C₈alkylamino, fluorine-, chlorine-, bromine-free or fluorine-, chlorine-, bromine-containing C₁-C₈alkylamino carbonyl, fluorine-, chlorine-, bromine-free or fluorine-, chlorine-, bromine-containing C₁-C₈alkoxyamino carbonyl, fluorine-, chlorine-, bromine-free or fluorine-, chlorine-, bromine-containing C₁-C₈alkoxy carbonyl C₁-C₈alkyl, fluorine-, chlorine-, bromine-free or fluorine-, chlorine-, bromine-containing C₁-C₈alkylamino carbonyl C₁-C₈alkyl, fluorine-, chlorine-, bromine-free or fluorine-, chlorine-, bromine-containing tri(C₁-C₈)alkylsilyl, or fluorine-, chlorine-, bromine-free or fluorine-, chlorine-, bromine-containing di(C₁-C₈)alkylphosphonyl;
R″ independently represents hydrogen, halogen-free or halogen-containing C₁-C₈ alkyl, halogen-free or halogen-containing C₂-C₈ alkenyl, halogen-free or halogen-containing C₂-C₈ alkynyl, halogen-free or halogen-containing C₃-C₈ cycloalkyl or halogen-free or halogen-containing C₃-C₈cycloalkyl C₁-C₈alkyl.

4. The N-(1,3,4-oxadiazol-2-yl)arylcarboxamide compound or a salt thereof according to claim 1, wherein
X represents O, S, SO, SO₂ or NR₁;
Y represents halogen, cyano, cyano C₁-C₂ alkyl, carboxy, nitro, N(R₂)₂, —(C₁-C₂)alkyl-N(R₂)₂, CON(R₂)₂, —(C₁-C₂)alkyl-CON(R₂)₂, —(C₁-C₂)alkyl-N(R₂)₃⁺I⁻, hydroxy C₁-C₂ alkyl, C₁-C₂ alkyl substituted by amino and carboxy, OR₃, SR₃, —(C₁-C₂)alkyl-SOR₃, —(C₁-C₂)alkyl-OR₃, —(C₁-C₂)alkyl-SR₃, COR₃, COOR₃, —(C₁-C₂)alkyl-COR₄, —(C₁-C₂)alkyl-COOR₄, —(C₁-C₂)alkyl-OCOR₃, Si(R₃)₃, —(C₁-C₂)alkyl-O—Si(R₃)₃, —(C₁-C₂)alkyl-O—N═C(R₃)₂, C₁-C₆ alkyl, halo C₁-C₆ alkyl; unsubstituted or substituted C₂-C₆ alkenyl, unsubstituted or substituted C₂-C₆ alkynyl, unsubstituted or substituted C₃-C₆ cycloalkyl, unsubstituted or substituted C₃-C₆ cycloalkyl C₁-C₂ alkyl, unsubstituted or substituted heterocyclyl, unsubstituted or substituted aryl, unsubstituted or substituted heteroaryl, unsubstituted or substituted heterocyclyl C₁-C₂ alkyl, unsubstituted or substituted aryl C₁-C₂ alkyl, or unsubstituted or substituted heteroaryl C₁-C₂ alkyl, wherein the substituted C₂-C₆ alkenyl or substituted C₂-C₆ alkynyl is C₂-C₆ alkenyl or C₂-C₆ alkynyl substituted by at least one group selected from halogen, cyano, C₃-C₆ cycloalkyl, C₁-C₆ alkyl carbonyl, C₁-C₆ alkoxy carbonyl, C₁-C₆ alkoxy, C₁-C₆ alkylthio, C₁-C₆ alkylsulfinyl, C₁-C₆ alkylsulfonyl, and tri(C₁-C₆)alkylsilyl, the substituted C₃-C₆ cycloalkyl or the substituted C₃-C₆ cycloalkyl C₁-C₂ alkyl is C₃-C₆ cycloalkyl or C₃-C₆ cycloalkyl C₁-C₂ alkyl substituted by at least one group selected from C₁-C₆ alkyl, halogen and phenyl;
or, —X—Y represents 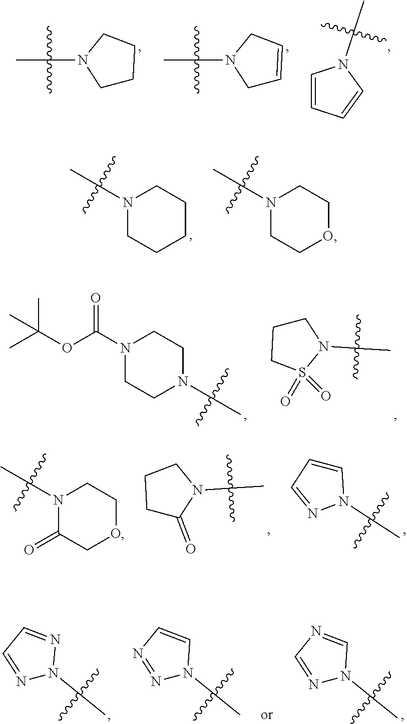
which is unsubstituted or substituted by 1, 2 or 3 groups selected from the group consisting of halogen, nitro, cyano, hydroxy, carboxy, amino, thiol, formyl, unsubstituted or substituted phenyl, unsubstituted or substituted benzyl, unsubstituted or substituted phenoxy, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₁-C₆ alkoxy, halogen-free or halogen-containing C₁-C₆ alkylthio, halogen-free or halogen-containing C₁-C₆ alkoxy C₁-C₂ alkyl, halogen-free or halogen-containing C₁-C₆ alkylthio C₁-C₂ alkyl, halogen-free or halogen-containing C₁-C₆ alkyl carbonyl, halogen-free or halogen-containing C₁-C₆ alkoxy carbonyl, halogen-free or halogen-containing C₁-C₆ alkyl sulfinyl, halogen-free or halogen-containing C₁-C₆ alkyl sulfonyl, halogen-free or halogen-containing C₁-C₆ alkyl amino, halogen-free or halogen-containing di(C₁-C₆)alkyl amino, or halogen-free or halogen-containing C₁-C₆ alkyl acyloxy, wherein the substituted phenyl, the substituted benzyl or the substituted phenoxy is phenyl, benzyl or phenoxy substituted by 1, 2 or 3 groups selected from halogen, C₁-C₆ alkyl, and C₁-C₆ alkoxy;
Z represents hydrogen, halogen, cyano, OR₄, —(C₁-C₂)alkyl-OR₄, —O—(C₁-C₂)alkyl-N(R₅)₂, —(C₁-C₂)alkyl-O—(C₁-C₂)alkyl-N(R₅)₂, N(R₅)₂, —(C₁-C₂)alkyl-(C═O)ₘ—N(R₅)₂, —NH—(C₁-C₂)alkyl-N(R₅)₂, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₁-C₆ alkoxy carbonyl, halogen-free or halogen-containing C₁-C₆ alkoxy carbonyl C₁-C₂ alkyl, halogen-free or halogen-containing C₁-C₆ alkyl carbonyl, halogen-free or halogen-containing C₁-C₆ alkyl carbonyl C₁-C₂ alkyl, halogen-free or halogen-containing C₁-C₆ alkyl carbonyloxy C₁-C₂ alkyl, halogen-free or halogen-containing C₁-C₆ alkylthio, halogen-free or halogen-containing C₁-C₆ alkyl sulfinyl, halogen-free or halogen-containing C₁-C₆ alkyl sulfonyl, unsubstituted or substituted C₃-C₆ cycloalkyl, unsubstituted or substituted heterocyclyl, unsubstituted or substituted aryl, unsubstituted or substituted heteroaryl, unsubstituted or substituted heterocyclyl C₁-C₂ alkyl, unsubstituted or substituted aryl C₁-C₂ alkyl, unsubstituted or substituted heteroaryl C₁-C₂ alkyl, unsubstituted or substituted heterocyclylcarbonyl C₁-C₂ alkyl, unsubstituted or substituted aryl carbonyl C₁-C₂ alkyl, unsubstituted or substituted heteroaryl carbonyl C₁-C₂ alkyl, unsubstituted or substituted heterocyclyl carbonyloxy C₁-C₂ alkyl, unsubstituted or substituted aryl carbonyloxy C₁-C₂ alkyl, or unsubstituted or substituted heteroaryl carbonyloxy C₁-C₂ alkyl, wherein the substituted C₃-C₆ cycloalkyl is C₃-C₆ cycloalkyl substituted by at least one group selected from C₁-C₆ alkyl, halogen and phenyl;
M represents hydrogen, OR₆, SR₆, COR₆, COOR₆, OCOR₆, CON(R₇)₂, N(R₇)₂, NR₈COOR₆, NR₈CON(R₇)₂, —(C₁-C₂)alkyl-R, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, unsubstituted or substituted aryl C₁-C₂ alkyl, or unsubstituted or substituted heteroaryl C₁-C₂ alkyl;
R represents halogen-free or halogen-containing C₂-C₆ alkenyl, halogen-free or halogen-containing C₂-C₆ alkynyl, halogen-free or halogen-containing C₃-C₆ cycloalkyl, CN, OR₁₁, OCOR₁₁, COOR₁₁, COR₁₁, —O—(C═O)—O—R₁₁, OSO₂R₁₂, SO₂OR₁₁, —S(O)ₙR₁₂, N(R₁₃)₂, CON(R₁₃)₂, SO₂N(R₁₃)₂, NR₁₄COR₁₁, NR₁₄SO₂R₁₂, or —O—(C═O)—N(R₁₃)₂;
R₄, R₆ and R₁₄ each independently represent hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₃-C₆ cycloalkenyl, halogen-free or halogen-containing C₁-C₆ alkoxy C₁-C₂ alkyl, unsubstituted or substituted heterocyclyl, unsubstituted or substituted heterocyclyl C₁-C₂ alkyl, unsubstituted or substituted heterocyclyloxy C₁-C₂ alkyl, unsubstituted or substituted aryl, unsubstituted or substituted aryl C₁-C₂ alkyl, unsubstituted or substituted aryloxy C₁-C₂ alkyl, unsubstituted or substituted heteroaryl, unsubstituted or substituted heteroaryl C₁-C₂ alkyl, or unsubstituted or substituted heteroaryloxy C₁-C₂ alkyl;
R₃ and R₁₂ each independently represent halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl, halogen-free or halogen-containing C₃-C₆ cycloalkyl C₁-C₂ alkyl, halogen-free or halogen-containing C₃-C₆ cycloalkenyl, halogen-free or halogen-containing C₁-C₆ alkoxy C₁-C₂ alkyl, unsubstituted or substituted heterocyclyl, unsubstituted or substituted heterocyclyl C₁-C₂ alkyl, unsubstituted or substituted aryl, unsubstituted or substituted aryl C₁-C₂ alkyl, unsubstituted or substituted heteroaryl, or unsubstituted or substituted heteroaryl C₁-C₂ alkyl;
R₁, R₂, R₅, R₇, R₈, R₁₃, and R₁₄ each independently represent hydrogen, nitro, alkoxyaminocarbonyl, trialkylsilyl, dialkylphosphonyl, N(R₂₁)₂, CON(R₂₁)₂, OR₂₁, COR₂₁, CO₂R₂₁, COSR₂₁, OCOR₁₁, S(O)ᵣR₂₂, C₁-C₆ alkyl, halo C₁-C₆ alkyl, C₂-C₆ alkenyl, halo C₂-C₆ alkenyl, C₂-C₆ alkynyl, halo C₂-C₆ alkynyl, C₃-C₆ cycloalkyl, C₃-C₆ cycloalkenyl, halo C₃-C₆ cycloalkyl, C₁-C₆ alkoxy C₁-C₂ alkyl, C₃-C₆ cycloalkyl C₁-C₂ alkyl, aryl, aryl C₁-C₂ alkyl, aryloxy, aryloxy C₁-C₂ alkyl, aryl C₁-C₂ alkyloxy, arylcarbonyl, arylsulfonyl, heteroaryl, heteroaryl C₁-C₂ alkyl, heteroaryloxy, heteroaryloxy C₁-C₂ alkyl, heteroaryl C₁-C₂ alkyloxy, heteroaryl carbonyl, heteroaryl sulfonyl, heterocyclyl, heterocyclyl C₁-C₂ alkyl, heterocyclyloxy, heterocyclyloxy C₁-C₂ alkyl, heterocyclyl C₁-C₂ alkyloxy, heterocyclyl carbonyl, heterocyclyl sulfonyl, aryl-NR₂₁—(C₁-C₂)alkyl, heteroaryl-NR₂₁—(C₁-C₂)alkyl, heterocyclyl-NR₂₁—(C₁-C₂)alkyl, wherein the last 35 groups as mentioned are each substituted by 0, 1, 2 or 3 groups selected from the group consisting of cyano, halogen, nitro, cyanothio, OR₂₁, S(O)ᵣR₂₂, N(R₂₁)₂, NR₂₁OR₂₁, COR₂₁, OCOR₂₁, SCOR₂₂, NR₂₁COR₂₁, NR₂₁SO₂R₂₂, CO₂R₂₁, COSR₂₁, CON(R₂₁)₂ and C₁-C₆ alkoxy C₁-C₂ alkoxy carbonyl;
R₂₁ independently represents hydrogen, C₁-C₆ alkyl, C₂-C₆ alkenyl, C₂-C₆ alkynyl, C₃-C₆ cycloalkyl, or C₃-C₆ cycloalkyl C₁-C₂ alkyl;
R₂₂ independently represents C₁-C₆ alkyl, C₂-C₆ alkenyl, C₂-C₆ alkynyl, C₃-C₆ cycloalkyl, or C₃-C₆cycloalkyl C₁-C₂ alkyl;
m represents 0 or 1;
n and r each independently represent 0, 1 or 2;
wherein, the “heterocyclyl” refers to 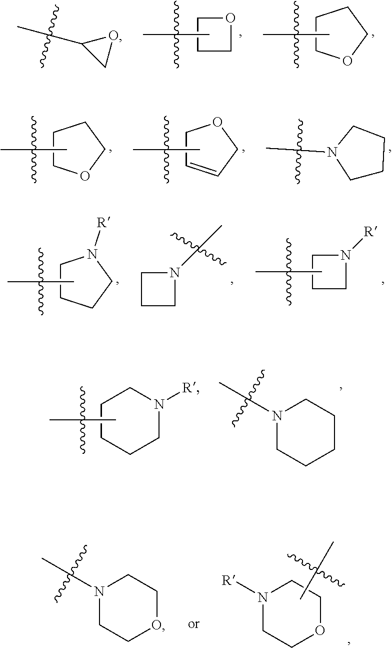
which has 0, 1 or 2 oxo groups, the “aryl” refers to phenyl or naphthyl, and the “heteroaryl” refers to 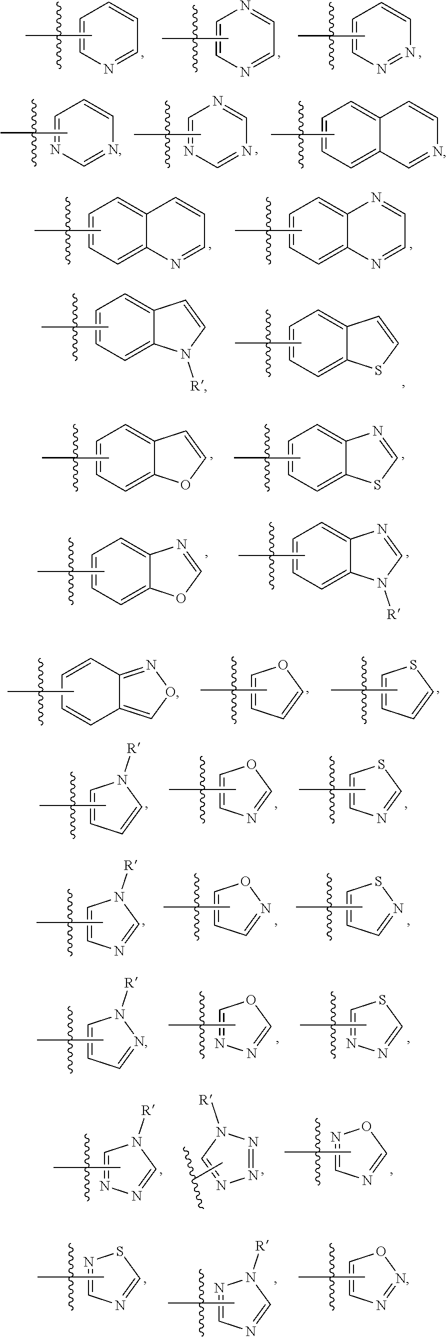 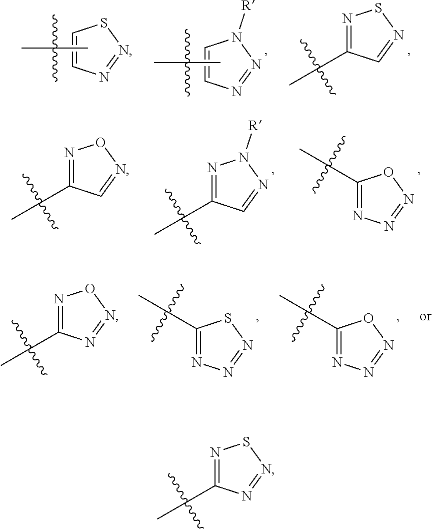
which is unsubstituted or substituted by 0, 1, 2 or 3 groups selected from the group consisting of halogen, nitro, cyano, cyanothio, hydroxy, carboxy, thiol, formyl, unsubstituted or substituted phenyl, unsubstituted or substituted benzyl, unsubstituted or substituted phenoxy, halogen-containing or halogen-free C₁-C₆ alkyl, halogen-containing or halogen-free C₂-C₆ alkenyl, halogen-containing or halogen-free C₂-C₆ alkynyl, halogen-containing or halogen-free C₃-C₆ cycloalkyl, halogen-containing or halogen-free C₃-C₆ cycloalkyl C₁-C₂ alkyl, halogen-containing or halogen-free OR″, halogen-containing or halogen-free SR″, halogen-containing or halogen-free —(C₁-C₂)alkyl-OR″, halogen-containing or halogen-free —(C₁-C₂ alkyl-SR″, halogen-containing or halogen-free COR″, halogen-containing or halogen-free COOR″, halogen-containing or halogen-free COSR″, halogen-containing or halogen-free SOR″, halogen-containing or halogen-free SO₂R″, halogen-containing or halogen-free OCOR″, halogen-containing or halogen-free SCOR″, substituted amino, substituted aminocarbonyl, —OCH₂CH₂—, —OCH₂O—, and —OCH₂CH₂O—, wherein the substituted phenyl, the substituted benzyl or the substituted phenoxy is phenyl, benzyl or phenoxy substituted by 1, 2 or 3 groups selected from halogen, C₁-C₆ alkyl and C₁-C₆ alkoxy, the substituted amino or the substituted aminocarbonyl is amino or aminocarbonyl substituted by one or two groups selected from hydrogen, C₁-C₆ alkyl, C₂-C₆ alkenyl, C₂-C₆ alkynyl, C₃-C₆ cycloalkyl, C₃-C₆ cycloalkyl C₁-C₂ alkyl, COR″, SO₂R″ and OR″;
R′ independently represents hydrogen, nitro, hydroxy, amino, fluorine-, chlorine-, bromine-free or fluorine-, chlorine-, bromine-containing C₁-C₆ alkyl, fluorine-, chlorine-, bromine-free or fluorine-, chlorine-, bromine-containing C₂-C₆ alkenyl, fluorine-, chlorine-, bromine-free or fluorine-, chlorine-, bromine-containing C₂-C₆ alkynyl, fluorine-, chlorine-, bromine-free or fluorine-, chlorine-, bromine-containing C₃-C₆ cycloalkyl, fluorine-, chlorine-, bromine-free or fluorine-, chlorine-, bromine-containing C₃-C₆ cycloalkenyl, fluorine-, chlorine-, bromine-free or fluorine-, chlorine-, bromine-containing C₃-C₈ cycloalkyl C₁-C₂ alkyl, fluorine-, chlorine-, bromine-free or fluorine-, chlorine-, bromine-containing C₁-C₆ alkoxy, fluorine-, chlorine-, bromine-free or fluorine-, chlorine-, bromine-containing C₂-C₆ alkenyloxy, fluorine-, chlorine-, bromine-free or fluorine-, chlorine-, bromine-containing C₂-C₆ alkynyloxy, fluorine-, chlorine-, bromine-free or fluorine-, chlorine-, bromine-containing C₃-C₆ cycloalkyloxy, fluorine-, chlorine-, bromine-free or fluorine-, chlorine-, bromine-containing C₁-C₆ alkoxy C₁-C₂ alkyl, fluorine-, chlorine-, bromine-free or fluorine-, chlorine-, bromine-containing C₁-C₆ alkoxy carbonyl, fluorine-, chlorine-, bromine-free or fluorine-, chlorine-, bromine-containing C₁-C₆ alkylthio carbonyl, fluorine-, chlorine-, bromine-free or fluorine-, chlorine-, bromine-containing C₁-C₆alkylsulfonyl, fluorine-, chlorine-, bromine-free or fluorine-, chlorine-, bromine-containing C₁-C₆ alkylsulfonyl C₁-C₂ alkyl, fluorine-, chlorine-, bromine-free or fluorine-, chlorine-, bromine-containing C₁-C₆ alkyl carbonyl, fluorine-, chlorine-, bromine-free or fluorine-, chlorine-, bromine-containing C₁-C₆ alkyl carbonyl C₁-C₂ alkyl, fluorine-, chlorine-, bromine-free or fluorine-, chlorine-, bromine-containing C₁-C₆ alkyl acyloxy, fluorine-, chlorine-, bromine-free or fluorine-, chlorine-, bromine-containing C₁-C₆ alkylamino, fluorine-, chlorine-, bromine-free or fluorine-, chlorine-, bromine-containing C₁-C₆ alkylamino carbonyl, fluorine-, chlorine-, bromine-free or fluorine-, chlorine-, bromine-containing C₁-C₆ alkoxyamino carbonyl, fluorine-, chlorine-, bromine-free or fluorine-, chlorine-, bromine-containing C₁-C₆ alkoxy carbonyl C₁-C₂ alkyl, fluorine-, chlorine-, bromine-free or fluorine-, chlorine-, bromine-containing C₁-C₆ alkylamino carbonyl C₁-C₂ alkyl, fluorine-, chlorine-, bromine-free or fluorine-, chlorine-, bromine-containing tri(C₁-C₆)alkylsilyl, or fluorine-, chlorine-, bromine-free or fluorine-, chlorine-, bromine-containing di(C₁-C₆)alkylphosphonyl;
R″ independently represents hydrogen, halogen-free or halogen-containing C₁-C₆ alkyl, halogen-free or halogen-containing C₂-C₆ alkenyl, halogen-free or halogen-containing C₂-C₆ alkynyl, halogen-free or halogen-containing C₃-C₆ cycloalkyl or halogen-free or halogen-containing C₃-C₆ cycloalkyl C₁-C₂ alkyl.

5. The N-(1,3,4-oxadiazol-2-yl)arylcarboxamide compound or a salt thereof according to claim 1, wherein
X represents S, SO or SO₂;
Y represents C₂-C₈ alkyl, halo C₁-C₈ alkyl, C₂-C₈ alkenyl, C₂-C₈ alkynyl, C₁-C₈ alkoxy C₁-C₈ alkyl, hydroxy C₁-C₈ alkyl, cyano, cyano C₁-C₈ alkyl, amino C₁-C₈ alkyl, C₁-C₈ alkyl amino C₁-C₈ alkyl, C₁-C₈ alkyl carbonyl, C₃-C₈ cycloalkyl, C₃-C₈ cycloalkyl C₁-C₈ alkyl, di(C₁-C₈)alkylamino carbonyl C₁-C₈ alkyl, C₁-C₈ alkyl carbonyl C₁-C₈ alkyl, C₁-C₈ alkoxy carbonyl C₁-C₈ alkyl, C₁-C₈ alkyl carbonyloxy C₁-C₈ alkyl, C₁-C₈ alkyl sulfinyl C₁-C₈ alkyl, tri(C₁-C₈)alkylsiloxy C₁-C₈ alkyl, heterocyclyl, heterocyclyl C₁-C₈ alkyl, phenyl, heteroaryl, heteroaryl C₁-C₈ alkyl, —(C₁-C₈)alkyl-O—N═C(R′″)₂;
wherein the heterocyclyl is 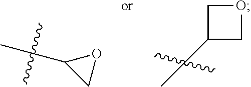
the heteroaryl is 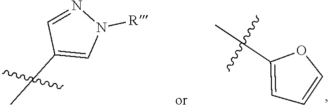
which is unsubstituted or substituted by C₁-C₈ alkyl; and R′″ independently represents C₁-C₈ alkyl;
Z represents hydrogen, or C₁-C₈ alkyl;
M represents hydrogen, or C₁-C₈ alkoxy carbonyloxy C₁-C₈ alkyl;
preferably, X represents S, SO or SO₂;
Y represents C₂-C₆ alkyl, halo C₁-C₆ alkyl, C₂-C₆ alkenyl, C₂-C₆ alkynyl, C₁-C₆ alkoxy C₁-C₆ alkyl, hydroxy C₁-C₆ alkyl, cyano, cyano C₁-C₆ alkyl, amino C₁-C₆ alkyl, C₁-C₆ alkylamino C₁-C₆ alkyl, C₁-C₆ alkyl carbonyl, C₃-C₆ cycloalkyl, C₃-C₆ cycloalkyl C₁-C₆ alkyl, di(C₁-C₆)alkylamino carbonyl C₁-C₆ alkyl, C₁-C₆ alkyl carbonyl C₁-C₆ alkyl, C₁-C₆ alkoxy carbonyl C₁-C₆ alkyl, C₁-C₆ alkyl carbonyloxy C₁-C₆ alkyl, C₁-C₆ alkyl sulfinyl C₁-C₆ alkyl, tri(C₁-C₆)alkylsiloxy C₁-C₆ alkyl, heterocyclyl, heterocyclyl C₁-C₆ alkyl, phenyl, heteroaryl, heteroaryl C₁-C₆ alkyl, —(C₁-C₆)alkyl-O—N═C(R′″)₂;
wherein, the heterocyclyl is 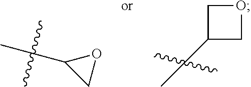
the heteroaryl is 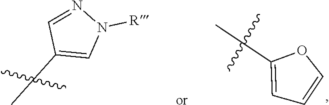
which is unsubstituted or substituted by C₁-C₆ alkyl; and R′″ independently represents C₁-C₆ alkyl;
Z represents hydrogen, or C₁-C₆ alkyl;
M represents hydrogen, or C₁-C₆ alkoxy carbonyloxy C₁-C₆ alkyl.

6. The N-(1,3,4-oxadiazol-2-yl)arylcarboxamide compound or a salt thereof according to claim 1, wherein the compound is 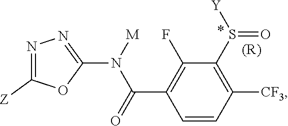
wherein Y, Z and M are as defined in any one of claims 1 to 5; preferably, the compound is any one compound selected from the compounds in Tables A1 to A151 and Tables 1 to 3.

7. A method for preparing the N-(1,3,4-oxadiazol-2-yl)arylcarboxamide compound or a salt thereof according to claim 1, wherein
a) when X represents Q in the Formula I, the method comprises the following steps:
reacting the compound represented by Formula V with a compound represented by VI to obtain a compound represented by Formula I-1, wherein the reaction equation thereof is as follows: 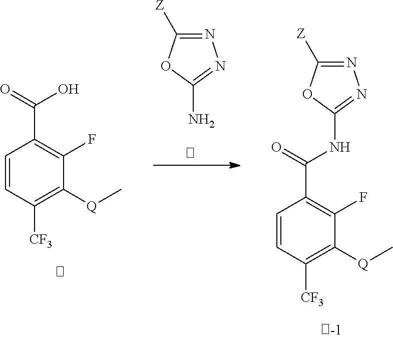
wherein, Q represents O, S, NR₁;
alternatively, b) when X represents S in Formula I, the method comprises the following steps:
reacting a compound represented by Formula VII with Hal-Y to prepare a compound represented by Formula I-3, wherein the reaction equation thereof is as follows: 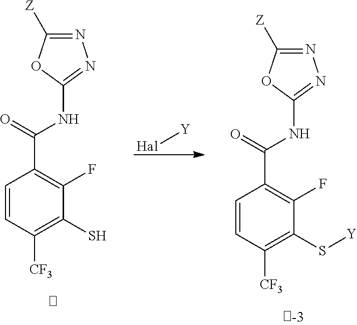
wherein Hal represents halogen, preferably fluorine, chlorine or bromine;
alternatively, c) when X represents SO, or SO₂ in Formula I, the method comprises the following steps:
reacting a compound represented by Formula I-3 with a peroxide to prepare a compound represented by Formula I-4 and/or 1-5, wherein the reaction equation thereof is as follows: 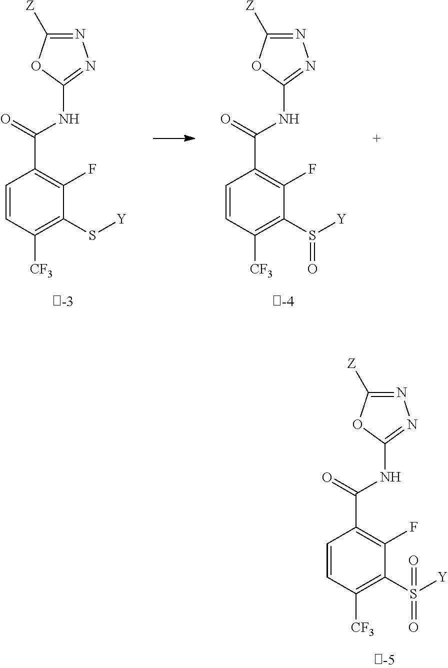 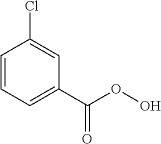 [SMILES: O=C(OO)c1cccc(Cl)c1]
alternatively, d) when M is not hydrogen in Formula I, the method comprises the following steps:
reacting compounds represented by Formulas I-1, I-4 or I-5 with Hal-M to prepare compounds represented by Formulas I-6, I-7, or I-8, respectively, wherein the reaction equations thereof are as follows:

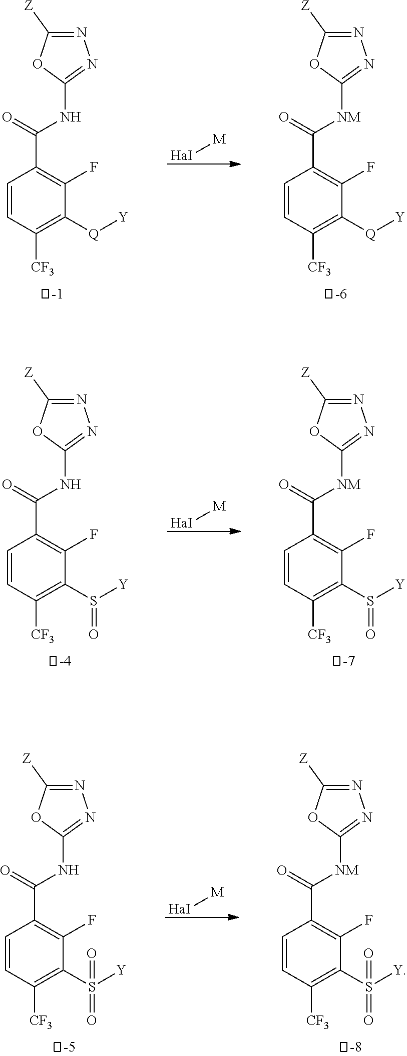

8. A herbicidal composition, in comprising the N-(1,3,4-oxadiazol-2-yl)arylcarboxamide compound represented by Formula I or a salt thereof according to claim 1.

9. A method for controlling a weed comprising: applying at least one of the N-(1,3,4-oxadiazol-2-yl)arylcarboxamide compound or a salt thereof according to claim 1 or a herbicidal composition comprising the N-(1,3,4-oxadiazol-2-yl)arylcarboxamide compound represented by Formula I or a salt thereof according to claim 1 in an herbicidally effective amount on a plant or in a weed area.

10. (canceled)

11. The method according to claim 7, wherein:
in a), the reaction is carried out in the presence of a halogenating agent, a catalyst and a solvent, the halogenating agent is SOCl₂, the catalyst is 4-dimethylaminopyridine, and the solvent is pyridine, the reaction is conducted at a temperature of 0 to 50° C.;
in b), the reaction is carried out in the presence of a base and a solvent, the base is one base or two bases in combination selected from the group consisting of potassium carbonate and sodium carbonate, the solvent is one solvent or more solvents in combination selected from the group consisting of DCM, DCE, ACN, THF, and DMF, the reaction is conducted at a temperature of 0 to 25° C.;
in c), the peroxide is 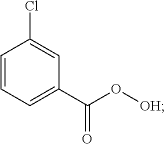 [SMILES: O=C(OO)c1cccc(Cl)c1]
the reaction is carried out in the presence of a solvent;
the solvent is one solvent or two solvents in combination selected from the group consisting of DCM and DCE, and the reaction is conducted at a temperature of 0 to 50° C.;
in d), the reaction is carried out in the presence of a base and a solvent, the base is one base or two bases in combination selected from the group consisting of potassium carbonate and sodium carbonate, the solvent is one solvent or more solvents in combination selected from the group consisting of DCM, DCE, ACN, THF, and DMF, the reaction is conducted at a temperature of 0 to 100° C.; or the reaction is carried out in the presence of a catalyst and a solvent, the catalyst is 4-dimethylaminopyridine, the solvent is pyridine, and the reaction is conducted at a temperature of 0 to 80° C.

12. The herbicidal composition according to claim 8, wherein the herbicidal composition further comprises one or more additional herbicides and/or safeners.

13. The herbicidal composition according to claim 12, wherein the herbicidal composition further comprises an agrochemically acceptable formulation auxiliary.

14. The herbicidal composition according to claim 12, wherein the additional herbicide is selected from the following compounds or salt or ester derivatives thereof:
a) ALS inhibitors: Pyrazosulfuron-ethyl, Penoxsulam, Bispyribac-sodium, Pyriminobac-methyl, Metazosulfuron, Propyrisulfuron, Triafamone;
b) ACCase inhibitors: Cyhalofop-butyl, Metamifop;
c) hormones inhibitors: Quinclorac, MCPA, 2,4-D, 2,4-D butyric acid, Fluroxypyr, Florpyrauxifen-benzyl, MCPA butyric acid, Dicamba, Quintrione, Clopyralid, Trichlopyr;
d) cell division inhibitors: Pendimethalin, Butralin;
e) lipid synthesis (non-ACC) inhibitors: Benthiocarb, Molinate;
f) HPPD inhibitors: Sanzuohuangcaotong ( [symbol] ), Shuangzuocaotong ( [symbol] ) Huanbifucaotong ( [symbol] ), Mesotrione, Benzobicylon, Tefuryltrione;
g) PDS inhibitors: Diflufenican, Fluorochloridone, Beflubutamid;
h) PPO inhibitors: Carfentrazone-ethyl, Pyraclonil, Oxadiazon, Oxadiargyl, Oxyfluorfen, Pentoxazone;
i) Long-chain fatty acid synthesis inhibitors: Butachlor, Pretilachlor, Mefenacet, Anilofos, Fentrazamide, Metolachlor, Piperophos, Pyroxasulfone;
j) PSII inhibitors: Simetryn, Prometryn, Amicarbazone, Isoproturon, Bromacil, Pyridate, Chlortoluron, Bentazone, Propanil, Metribuzin, Atrazine, Bromoxynil, Bromoxynil octanoate, Terbuthylazine;
k) DOXP inhibitors: Clomazone;
l) Others: Oxaziclomefone, Cinmethylin, Indanofan.

15. The herbicidal composition according to claim 8, wherein the herbicidal composition further comprises an agrochemically acceptable formulation auxiliary.

16. The method according to claim 9, wherein the plant is rice, and the weed is a gramineous weed, a broad-leaved weed, or a cyperaceae weed.

17. The method according to claim 16, wherein the rice is indica rice or japonica rice.

18. The method according to claim 16, wherein the weed is Echinochloa phyllopogon, Leptochloa chinensis, Echinochloa crusgali, Digitaria sanguinalis, Setaria viridis, Monochoria vaginalis, Sagittaria trifolia, Abutilon theophrasti, Amaranthus retroflexus, Stellaria media, Cyperus difformis, or Scirpus juncoides.
